SEQUENCE LISTING (343 sequences)

SEQ ID NO: 1 - RNA, 22 nt, Homo sapiens
uagcaccaucugaaaucgguua

SEQ ID NO: 2 - RNA, 23 nt, Homo sapiens
uagcaccauuugaaaucaguguu

SEQ ID NO: 3 - RNA, 22 nt, Homo sapiens
uagcaccauuugaaaucgguua

SEQ ID NO: 4 - RNA, 23 nt, Homo sapiens
ugcaccaugguugucugagcaug

SEQ ID NO: 5 - RNA, 21 nt, Homo sapiens
uacuuggaaaggcaucaguug

SEQ ID NO: 6 - RNA, 22 nt, Homo sapiens
aacaauauccuggugcugagug

SEQ ID NO: 7 - RNA, 22 nt, Homo sapiens
agaucgaccguguuauauucgc

SEQ ID NO: 8 - RNA, 20 nt, Homo sapiens
cggcucugggucugugggga

SEQ ID NO: 9 - RNA, 21 nt, Homo sapiens
aauauuauacagucaaccucu

SEQ ID NO: 10 - RNA, 22 nt, Homo sapiens
cagugcaauguuaaaagggcau

SEQ ID NO: 11 - RNA, 24 nt, Homo sapiens
uggcccugacugaagaccagcagu

SEQ ID NO: 12 - RNA, 22 nt, Homo sapiens
agaucgaccguguuauauucgc

SEQ ID NO: 13 - RNA, 20 nt, Homo sapiens
uaaagagcccuguggagaca

SEQ ID NO: 14 - RNA, 24 nt, Homo sapiens
uggggagcugaggcucugggggug

SEQ ID NO: 15 - RNA, 17 nt, Homo sapiens
gugggggagaggcuguc

SEQ ID NO: 16 - RNA, 22 nt, Homo sapiens
ugagguaguagguuguaugguu

SEQ ID NO: 17 - RNA, 22 nt, Homo sapiens
uagcaccaucugaaaucgguua

SEQ ID NO: 18 - RNA, 23 nt, Homo sapiens
uagcaccauuugaaaucaguguu

SEQ ID NO: 19 - RNA, 22 nt, Homo sapiens
acaguagucugcacauugguua

SEQ ID NO: 20 - RNA, 22 nt, Homo sapiens
cugugcgugugacagcggcuga

SEQ ID NO: 21 - RNA, 21 nt, Homo sapiens
agcuacaucuggcuacugggu

SEQ ID NO: 22 - RNA, 21 nt, Homo sapiens
agggcccccccucaauccugu

SEQ ID NO: 23 - RNA, 23 nt, Homo sapiens
ccuaguagguguccaguaagugu

SEQ ID NO: 24 - RNA, 21 nt, Homo sapiens
gccccugggccuauccuagaa

SEQ ID NO: 25 - RNA, 21 nt, Homo sapiens
aggggugcuaucugugauuga

SEQ ID NO: 26 - RNA, 22 nt, Homo sapiens
gccugcugggguggaaccuggu

SEQ ID NO: 27 - RNA, 21 nt, Homo sapiens
aacauagaggaaauuccacgu

SEQ ID NO: 28 - RNA, 23 nt, Homo sapiens
ugaggggcagagagcgagacuuu

SEQ ID NO: 29 - RNA, 22 nt, Homo sapiens
uuuugcaauauguuccugaaua

SEQ ID NO: 30 - RNA, 23 nt, Homo sapiens
uagugcaauauugcuuauagggu

SEQ ID NO: 31 - RNA, 21 nt, Homo sapiens
uuuugcaccuuuuggagugaa

SEQ ID NO: 32 - RNA, 22 nt, Homo sapiens
caaagugccucccuuuagagug

SEQ ID NO: 33 - RNA, 22 nt, Homo sapiens
caaaaaucucaauuacuuuugc

SEQ ID NO: 34 - RNA, 22 nt, Homo sapiens
caaaaaccacaguuucuuuugc

SEQ ID NO: 35 - RNA, 22 nt, Homo sapiens
aaaaguauuugcggguuuuguc

SEQ ID NO: 36 - RNA, 23 nt, Homo sapiens
guuugcacgggugggccuugucu

SEQ ID NO: 37 - RNA, 22 nt, Homo sapiens
uaugcauuguauuuuuaggucc

SEQ ID NO: 38 - RNA, 21 nt, Homo sapiens
ggcuagcaacagcgcuuaccu

SEQ ID NO: 39 - RNA, 22 nt, Homo sapiens
aaccagcaccccaacuuuggac

SEQ ID NO: 40 - RNA, 24 nt, Homo sapiens
acugggggcuuucgggcucugcgu

SEQ ID NO: 41 - RNA, 21 nt, Homo sapiens
aauauuauacagucaaccucu

SEQ ID NO: 42 - RNA, 21 nt, Homo sapiens
cuuccgccccgccgggcgucg

SEQ ID NO: 43 - RNA, 20 nt, Homo sapiens
cggcucugggucugugggga

SEQ ID NO: 44 - RNA, 23 nt, Homo sapiens
ugcaccaugguugucugagcaug

SEQ ID NO: 45 - RNA, 20 nt, Homo sapiens
ggggagcuguggaagcagua

SEQ ID NO: 46 - RNA, 22 nt, Homo sapiens
aaauuauuguacaucggaugag

SEQ ID NO: 47 - RNA, 21 nt, Homo sapiens
aagugaucuaaaggccuacau

SEQ ID NO: 48 - RNA, 19 nt, Homo sapiens
uggauuuuuggaucaggga

SEQ ID NO: 49 - RNA, 24 nt, Homo sapiens
uggcccugacugaagaccagcagu

SEQ ID NO: 50 - RNA, 22 nt, Homo sapiens
caaaaaccacaguuucuuuugc

SEQ ID NO: 51 - RNA, 24 nt, Homo sapiens
acugggggcuuucgggcucugcgu

SEQ ID NO: 52 - RNA, 23 nt, Homo sapiens
guuugcacgggugggccuugucu

SEQ ID NO: 53 - RNA, 20 nt, Homo sapiens
cggcucugggucugugggga

SEQ ID NO: 54 - RNA, 21 nt, Homo sapiens
aggggugcuaucugugauuga

SEQ ID NO: 55 - RNA, 21 nt, Homo sapiens
gccccugggccuauccuagaa

SEQ ID NO: 56 - RNA, 21 nt, Homo sapiens
guucaaauccagaucuauaac

SEQ ID NO: 57 - RNA, 22 nt, Homo sapiens
caaaaaucucaauuacuuuugc

SEQ ID NO: 58 - RNA, 20 nt, Homo sapiens
cggcucugggucugugggga

SEQ ID NO: 59 - RNA, 22 nt, Homo sapiens
cugugcgugugacagcggcuga

SEQ ID NO: 60 - RNA, 22 nt, Homo sapiens
caaaaaccacaguuucuuuugc

SEQ ID NO: 61 - RNA, 22 nt, Homo sapiens
aaaaguauuugcggguuuuguc

SEQ ID NO: 62 - RNA, 21 nt, Homo sapiens
aacauagaggaaauuccacgu

SEQ ID NO: 63 - RNA, 22 nt, Homo sapiens
caaagugccucccuuuagagug

SEQ ID NO: 64 - RNA, 24 nt, Homo sapiens
acugggggcuuucgggcucugcgu

SEQ ID NO: 65 - RNA, 22 nt, Homo sapiens
aaccagcaccccaacuuuggac

SEQ ID NO: 66 - RNA, 21 nt, Homo sapiens
cuuccgccccgccgggcgucg

SEQ ID NO: 67 - RNA, 21 nt, Homo sapiens
augauccaggaaccugccucu

SEQ ID NO: 68 - RNA, 24 nt, Homo sapiens
uggcccugacugaagaccagcagu

SEQ ID NO: 69 - RNA, 23 nt, Homo sapiens
ccuaguagguguccaguaagugu

SEQ ID NO: 70 - RNA, 23 nt, Homo sapiens
guuugcacgggugggccuugucu

SEQ ID NO: 71 - RNA, 21 nt, Homo sapiens
aagugaucuaaaggccuacau

SEQ ID NO: 72 - RNA, 23 nt, Homo sapiens
aguauguucuuccaggacagaac

SEQ ID NO: 73 - RNA, 21 nt, Homo sapiens
aauauuauacagucaaccucu

SEQ ID NO: 74 - RNA, 21 nt, Homo sapiens
aggggugcuaucugugauuga

SEQ ID NO: 75 - RNA, 20 nt, Homo sapiens
ggggagcuguggaagcagua

SEQ ID NO: 76 - RNA, 21 nt, Homo sapiens
gccccugggccuauccuagaa

SEQ ID NO: 77 - RNA, 22 nt, Homo sapiens
uaugcauuguauuuuuaggucc

SEQ ID NO: 78 - RNA, 22 nt, Homo sapiens
caaaaaucucaauuacuuuugc

SEQ ID NO: 79 - RNA, 23 nt, Homo sapiens
cucuugagggaagcacuuucugu

SEQ ID NO: 80 - RNA, 22 nt, Homo sapiens
cugugcgugugacagcggcuga

SEQ ID NO: 81 - RNA, 21 nt, Homo sapiens
ggcuagcaacagcgcuuaccu

SEQ ID NO: 82 - RNA, 22 nt, Homo sapiens
uggcagugucuuagcugguugu

SEQ ID NO: 83 - RNA, 21 nt, Homo sapiens
aacauagaggaaauuccacgu

SEQ ID NO: 84 - RNA, 20 nt, Homo sapiens
uaaagagcccuguggagaca

SEQ ID NO: 85 - RNA, 22 nt, Homo sapiens
caaagugccucccuuuagagug

SEQ ID NO: 86 - RNA, 24 nt, Homo sapiens
acugggggcuuucgggcucugcgu

SEQ ID NO: 87 - RNA, 22 nt, Homo sapiens
auaauacaugguuaaccucuuu

SEQ ID NO: 88 - RNA, 20 nt, Homo sapiens
cggcucugggucugugggga

SEQ ID NO: 89 - RNA, 24 nt, Homo sapiens
uggcccugacugaagaccagcagu

SEQ ID NO: 90 - RNA, 23 nt, Homo sapiens
guuugcacgggugggccuugucu

SEQ ID NO: 91 - RNA, 23 nt, Homo sapiens
uuuggcacuagcacauuuuugcu

SEQ ID NO: 92 - RNA, 23 nt, Homo sapiens
caagucuuauuugagcaccuguu

SEQ ID NO: 93 - RNA, 21 nt, Homo sapiens
aagugaucuaaaggccuacau

SEQ ID NO: 94 - RNA, 20 nt, Homo sapiens
cggcucugggucugugggga

SEQ ID NO: 95 - RNA, 21 nt, Homo sapiens
agggcccccccucaauccugu

SEQ ID NO: 96 - RNA, 22 nt, Homo sapiens
aacaauauccuggugcugagug

SEQ ID NO: 97 - RNA, 22 nt, Homo sapiens
gccugcugggguggaaccuggu

SEQ ID NO: 98 - RNA, 20 nt, Homo sapiens
cggcucugggucugugggga

SEQ ID NO: 99 - RNA, 21 nt, Homo sapiens
aggggugcuaucugugauuga

SEQ ID NO: 100 - RNA, 20 nt, Homo sapiens
ggggagcuguggaagcagua

SEQ ID NO: 101 - RNA, 21 nt, Homo sapiens
uacaguacugugauaacugaa

SEQ ID NO: 102 - RNA, 21 nt, Homo sapiens
uaacagucuccagucacggcc

SEQ ID NO: 103 - RNA, 21 nt, Homo sapiens
agggcccccccucaauccugu

SEQ ID NO: 104 - RNA, 21 nt, Homo sapiens
gccccugggccuauccuagaa

SEQ ID NO: 105 - RNA, 21 nt, Homo sapiens
agggcccccccucaauccugu

SEQ ID NO: 106 - RNA, 21 nt, Homo sapiens
guucaaauccagaucuauaac

SEQ ID NO: 107 - RNA, 22 nt, Homo sapiens
uggugggccgcagaacaugugc

SEQ ID NO: 108 - RNA, 22 nt, Homo sapiens
caaaaaucucaauuacuuuugc

SEQ ID NO: 109 - RNA, 20 nt, Homo sapiens
cggcucugggucugugggga

SEQ ID NO: 110 - RNA, 22 nt, Homo sapiens
caaaaaccacaguuucuuuugc

SEQ ID NO: 111 - RNA, 22 nt, Homo sapiens
aaaaguauuugcggguuuuguc

SEQ ID NO: 112 - RNA, 22 nt, Homo sapiens
cugaagcucagagggcucugau

SEQ ID NO: 113 - RNA, 20 nt, Homo sapiens
uaaagagcccuguggagaca

SEQ ID NO: 114 - RNA, 21 nt, Homo sapiens
ggcuagcaacagcgcuuaccu

SEQ ID NO: 115 - RNA, 20 nt, Homo sapiens
uaaagagcccuguggagaca

SEQ ID NO: 116 - RNA, 22 nt, Homo sapiens
cauugcacuugucucggucuga

SEQ ID NO: 117 - RNA, 21 nt, Homo sapiens
aacauagaggaaauuccacgu

SEQ ID NO: 118 - RNA, 22 nt, Homo sapiens
caaagugccucccuuuagagug

SEQ ID NO: 119 - RNA, 22 nt, Homo sapiens
caaaaaccacaguuucuuuugc

SEQ ID NO: 120 - RNA, 22 nt, Homo sapiens
uggagagaaaggcaguuccuga

SEQ ID NO: 121 - RNA, 24 nt, Homo sapiens
acugggggcuuucgggcucugcgu

SEQ ID NO: 122 - RNA, 17 nt, Homo sapiens
gugggggagaggcuguc

SEQ ID NO: 123 - RNA, 23 nt, Homo sapiens
ugaggggcagagagcgagacuuu

SEQ ID NO: 124 - RNA, 22 nt, Homo sapiens
agaucgaccguguuauauucgc

SEQ ID NO: 125 - RNA, 22 nt, Homo sapiens
ugagguaguagguuguaugguu

SEQ ID NO: 126 - RNA, 22 nt, Homo sapiens
uuauaaagcaaugagacugauu

SEQ ID NO: 127 - RNA, 21 nt, Homo sapiens
aggggugcuaucugugauuga

SEQ ID NO: 128 - RNA, 22 nt, Homo sapiens
uaugcauuguauuuuuaggucc

SEQ ID NO: 129 - RNA, 22 nt, Homo sapiens
aaccagcaccccaacuuuggac

SEQ ID NO: 130 - RNA, 22 nt, Homo sapiens
uugcauagucacaaaagugauc

SEQ ID NO: 131 - RNA, 24 nt, Homo sapiens
uggggagcugaggcucugggggug

SEQ ID NO: 132 - RNA, 23 nt, Homo sapiens
ugaggggcagagagcgagacuuu

SEQ ID NO: 133 - RNA, 21 nt, Homo sapiens
augauccaggaaccugccucu

SEQ ID NO: 134 - RNA, 22 nt, Homo sapiens
acaggugagguucuugggagcc

SEQ ID NO: 135 - RNA, 24 nt, Homo sapiens
uggcccugacugaagaccagcagu

SEQ ID NO: 136 - RNA, 23 nt, Homo sapiens
caguaacaaagauucauccuugu

SEQ ID NO: 137 - RNA, 23 nt, Homo sapiens
ucuuugguuaucuagcuguauga

SEQ ID NO: 138 - RNA, 23 nt, Homo sapiens
guuugcacgggugggccuugucu

SEQ ID NO: 139 - RNA, 23 nt, Homo sapiens
caagucuuauuugagcaccuguu

SEQ ID NO: 140 - RNA, 22 nt, Homo sapiens
uuuugcaauauguuccugaaua

SEQ ID NO: 141 - RNA, 22 nt, Homo sapiens
uccgagccugggucucccucuu

SEQ ID NO: 142 - RNA, 21 nt, Homo sapiens
aagugaucuaaaggccuacau

SEQ ID NO: 143 - RNA, 22 nt, Homo sapiens
caaaaaucucaauuacuuuugc

SEQ ID NO: 144 - RNA, 20 nt, Homo sapiens
cggcucugggucugugggga

SEQ ID NO: 145 - RNA, 17 nt, Homo sapiens
gugggggagaggcuguc

SEQ ID NO: 146 - RNA, 25 nt, Homo sapiens
ugggaacggguuccggcagacgcug

SEQ ID NO: 147 - RNA, 21 nt, Homo sapiens
agcuacaucuggcuacugggu

SEQ ID NO: 148 - RNA, 21 nt, Homo sapiens
agggcccccccucaauccugu

SEQ ID NO: 149 - RNA, 22 nt, Homo sapiens
aacaauauccuggugcugagug

SEQ ID NO: 150 - RNA, 22 nt, Homo sapiens
gccugcugggguggaaccuggu

SEQ ID NO: 151 - RNA, 22 nt, Homo sapiens
aaaaguacuugcggauuuugcu

SEQ ID NO: 152 - RNA, 23 nt, Homo sapiens
aguauguucuuccaggacagaac

SEQ ID NO: 153 - RNA, 21 nt, Homo sapiens
guucaaauccagaucuauaac

SEQ ID NO: 154 - RNA, 21 nt, Homo sapiens
aauauuauacagucaaccucu

SEQ ID NO: 155 - RNA, 21 nt, Homo sapiens
aggggugcuaucugugauuga

SEQ ID NO: 156 - RNA, 20 nt, Homo sapiens
ggggagcuguggaagcagua

SEQ ID NO: 157 - RNA, 21 nt, Homo sapiens
uacaguacugugauaacugaa

SEQ ID NO: 158 - RNA, 21 nt, Homo sapiens
uaacagucuccagucacggcc

SEQ ID NO: 159 - RNA, 21 nt, Homo sapiens
agggcccccccucaauccugu

SEQ ID NO: 160 - RNA, 21 nt, Homo sapiens
gccccugggccuauccuagaa

SEQ ID NO: 161 - RNA, 21 nt, Homo sapiens
agggcccccccucaauccugu

SEQ ID NO: 162 - RNA, 22 nt, Homo sapiens
caaaaaucucaauuacuuuugc

SEQ ID NO: 163 - RNA, 23 nt, Homo sapiens
cucuugagggaagcacuuucugu

SEQ ID NO: 164 - RNA, 22 nt, Homo sapiens
aaggagcucacagucuauugag

SEQ ID NO: 165 - RNA, 20 nt, Homo sapiens
cggcucugggucugugggga

SEQ ID NO: 166 - RNA, 21 nt, Homo sapiens
uggcagggaggcugggagggg

SEQ ID NO: 167 - RNA, 22 nt, Homo sapiens
cugugcgugugacagcggcuga

SEQ ID NO: 168 - RNA, 22 nt, Homo sapiens
caaaaaccacaguuucuuuugc

SEQ ID NO: 169 - RNA, 22 nt, Homo sapiens
aaaaguauuugcggguuuuguc

SEQ ID NO: 170 - RNA, 23 nt, Homo sapiens
ugcaccaugguugucugagcaug

SEQ ID NO: 171 - RNA, 22 nt, Homo sapiens
cugaagcucagagggcucugau

SEQ ID NO: 172 - RNA, 22 nt, Homo sapiens
uagcaccaucugaaaucgguua

SEQ ID NO: 173 - RNA, 21 nt, Homo sapiens
ggcuagcaacagcgcuuaccu

SEQ ID NO: 174 - RNA, 23 nt, Homo sapiens
caguaacaaagauucauccuugu

SEQ ID NO: 175 - RNA, 19 nt, Homo sapiens
uggauuuuuggaucaggga

SEQ ID NO: 176 - RNA, 22 nt, Homo sapiens
cauugcacuugucucggucuga

SEQ ID NO: 177 - RNA, 22 nt, Homo sapiens
uggcagugucuuagcugguugu

SEQ ID NO: 178 - RNA, 21 nt, Homo sapiens
aacauagaggaaauuccacgu

SEQ ID NO: 179 - RNA, 23 nt, Homo sapiens
aaggagcuuacaaucuagcuggg

SEQ ID NO: 180 - RNA, 21 nt, Homo sapiens
aggggugcuaucugugauuga

SEQ ID NO: 181 - RNA, 21 nt, Homo sapiens
gccccugggccuauccuagaa

SEQ ID NO: 182 - RNA, 22 nt, Homo sapiens
uaugcauuguauuuuuaggucc

SEQ ID NO: 183 - RNA, 21 nt, Homo sapiens
uggaggagaaggaaggugaug

SEQ ID NO: 184 - RNA, 18 nt, Homo sapiens
aaagcuggguugagaagg

SEQ ID NO: 185 - RNA, 20 nt, Homo sapiens
cggcucugggucugugggga

SEQ ID NO: 186 - RNA, 22 nt, Homo sapiens
aaaaguauuugcggguuuuguc

SEQ ID NO: 187 - RNA, 22 nt, Homo sapiens
caaagugccucccuuuagagug

SEQ ID NO: 188 - RNA, 22 nt, Homo sapiens
uggagagaaaggcaguuccuga

SEQ ID NO: 189 - RNA, 24 nt, Homo sapiens
acugggggcuuucgggcucugcgu

SEQ ID NO: 190 - RNA, 17 nt, Homo sapiens
gugggggagaggcuguc

SEQ ID NO: 191 - RNA, 23 nt, Homo sapiens
ugaggggcagagagcgagacuuu

SEQ ID NO: 192 - RNA, 22 nt, Homo sapiens
agaucgaccguguuauauucgc

SEQ ID NO: 193 - RNA, 22 nt, Homo sapiens
ugagguaguagguuguaugguu

SEQ ID NO: 194 - RNA, 22 nt, Homo sapiens
uuauaaagcaaugagacugauu

SEQ ID NO: 195 - RNA, 21 nt, Homo sapiens
aggggugcuaucugugauuga

SEQ ID NO: 196 - RNA, 22 nt, Homo sapiens
uaugcauuguauuuuuaggucc

SEQ ID NO: 197 - RNA, 22 nt, Homo sapiens
aaccagcaccccaacuuuggac

SEQ ID NO: 198 - RNA, 22 nt, Homo sapiens
uugcauagucacaaaagugauc

SEQ ID NO: 199 - RNA, 24 nt, Homo sapiens
uggggagcugaggcucugggggug

SEQ ID NO: 200 - RNA, 23 nt, Homo sapiens
ugaggggcagagagcgagacuuu

SEQ ID NO: 201 - RNA, 21 nt, Homo sapiens
augauccaggaaccugccucu

SEQ ID NO: 202 - RNA, 22 nt, Homo sapiens
acaggugagguucuugggagcc

SEQ ID NO: 203 - RNA, 24 nt, Homo sapiens
uggcccugacugaagaccagcagu

SEQ ID NO: 204 - RNA, 23 nt, Homo sapiens
caguaacaaagauucauccuugu

SEQ ID NO: 205 - RNA, 23 nt, Homo sapiens
guuugcacgggugggccuugucu

SEQ ID NO: 206 - RNA, 22 nt, Homo sapiens
uuuugcaauauguuccugaaua

SEQ ID NO: 207 - RNA, 22 nt, Homo sapiens
uccgagccugggucucccucuu

SEQ ID NO: 208 - RNA, 21 nt, Homo sapiens
aagugaucuaaaggccuacau

SEQ ID NO: 209 - RNA, 22 nt, Homo sapiens
caaaaaucucaauuacuuuugc

SEQ ID NO: 210 - RNA, 20 nt, Homo sapiens
cggcucugggucugugggga

SEQ ID NO: 211 - RNA, 17 nt, Homo sapiens
gugggggagaggcuguc

SEQ ID NO: 212 - RNA, 25 nt, Homo sapiens
ugggaacggguuccggcagacgcug

SEQ ID NO: 213 - RNA, 21 nt, Homo sapiens
agcuacaucuggcuacugggu

SEQ ID NO: 214 - RNA, 21 nt, Homo sapiens
agggcccccccucaauccugu

SEQ ID NO: 215 - RNA, 22 nt, Homo sapiens
gccugcugggguggaaccuggu

SEQ ID NO: 216 - RNA, 22 nt, Homo sapiens
aaaaguacuugcggauuuugcu

SEQ ID NO: 217 - RNA, 23 nt, Homo sapiens
aguauguucuuccaggacagaac

SEQ ID NO: 218 - RNA, 21 nt, Homo sapiens
guucaaauccagaucuauaac

SEQ ID NO: 219 - RNA, 21 nt, Homo sapiens
aauauuauacagucaaccucu

SEQ ID NO: 220 - RNA, 21 nt, Homo sapiens
aggggugcuaucugugauuga

SEQ ID NO: 221 - RNA, 20 nt, Homo sapiens
ggggagcuguggaagcagua

SEQ ID NO: 222 - RNA, 21 nt, Homo sapiens
agggcccccccucaauccugu

SEQ ID NO: 223 - RNA, 21 nt, Homo sapiens
gccccugggccuauccuagaa

SEQ ID NO: 224 - RNA, 21 nt, Homo sapiens
agggcccccccucaauccugu

SEQ ID NO: 225 - RNA, 22 nt, Homo sapiens
caaaaaucucaauuacuuuugc

SEQ ID NO: 226 - RNA, 23 nt, Homo sapiens
cucuugagggaagcacuuucugu

SEQ ID NO: 227 - RNA, 22 nt, Homo sapiens
aaggagcucacagucuauugag

SEQ ID NO: 228 - RNA, 20 nt, Homo sapiens
cggcucugggucugugggga

SEQ ID NO: 229 - RNA, 21 nt, Homo sapiens
uggcagggaggcugggagggg

SEQ ID NO: 230 - RNA, 22 nt, Homo sapiens
cugugcgugugacagcggcuga

SEQ ID NO: 231 - RNA, 22 nt, Homo sapiens
caaaaaccacaguuucuuuugc

SEQ ID NO: 232 - RNA, 22 nt, Homo sapiens
aaaaguauuugcggguuuuguc

SEQ ID NO: 233 - RNA, 22 nt, Homo sapiens
uagcaccaucugaaaucgguua

SEQ ID NO: 234 - RNA, 21 nt, Homo sapiens
ggcuagcaacagcgcuuaccu

SEQ ID NO: 235 - RNA, 19 nt, Homo sapiens
uggauuuuuggaucaggga

SEQ ID NO: 236 - RNA, 22 nt, Homo sapiens
uggcagugucuuagcugguugu

SEQ ID NO: 237 - RNA, 21 nt, Homo sapiens
aacauagaggaaauuccacgu

SEQ ID NO: 238 - RNA, 23 nt, Homo sapiens
aaggagcuuacaaucuagcuggg

SEQ ID NO: 239 - RNA, 22 nt, Homo sapiens
caaagugccucccuuuagagug

SEQ ID NO: 240 - RNA, 24 nt, Homo sapiens
acugggggcuuucgggcucugcgu

SEQ ID NO: 241 - RNA, 22 nt, Homo sapiens
ugagguaguagguuguaugguu

SEQ ID NO: 242 - RNA, 23 nt, Homo sapiens
uagugcaauauugcuuauagggu

SEQ ID NO: 243 - RNA, 22 nt, Homo sapiens
acaggugagguucuugggagcc

SEQ ID NO: 244 - RNA, 22 nt, Homo sapiens
uuuugcaauauguuccugaaua

SEQ ID NO: 245 - RNA, 21 nt, Homo sapiens
agcuacaucuggcuacugggu

SEQ ID NO: 246 - RNA, 21 nt, Homo sapiens
agggcccccccucaauccugu

SEQ ID NO: 247 - RNA, 22 nt, Homo sapiens
uagcaccaucugaaaucgguua

SEQ ID NO: 248 - RNA, 23 nt, Homo sapiens
uagcaccauuugaaaucaguguu

SEQ ID NO: 249 - RNA, 22 nt, Homo sapiens
gccugcugggguggaaccuggu

SEQ ID NO: 250 - RNA, 21 nt, Homo sapiens
aauauuauacagucaaccucu

SEQ ID NO: 251 - RNA, 21 nt, Homo sapiens
aggggugcuaucugugauuga

SEQ ID NO: 252 - RNA, 20 nt, Homo sapiens
ggggagcuguggaagcagua

SEQ ID NO: 253 - RNA, 21 nt, Homo sapiens
gccccugggccuauccuagaa

SEQ ID NO: 254 - RNA, 22 nt, Homo sapiens
uaacacugucugguaacgaugu

SEQ ID NO: 255 - RNA, 22 nt, Homo sapiens
caaaaaucucaauuacuuuugc

SEQ ID NO: 256 - RNA, 23 nt, Homo sapiens
ugcaccaugguugucugagcaug

SEQ ID NO: 257 - RNA, 22 nt, Homo sapiens
uagcaccaucugaaaucgguua

SEQ ID NO: 258 - RNA, 19 nt, Homo sapiens
uggauuuuuggaucaggga

SEQ ID NO: 259 - RNA, 23 nt, Homo sapiens
uucauuugguauaaaccgcgauu

SEQ ID NO: 260 - RNA, 21 nt, Homo sapiens
aacauagaggaaauuccacgu

SEQ ID NO: 261 - RNA, 22 nt, Homo sapiens
caaagugccucccuuuagagug

SEQ ID NO: 262 - RNA, 24 nt, Homo sapiens
acugggggcuuucgggcucugcgu

SEQ ID NO: 263 - RNA, 22 nt, Homo sapiens
ugagguaguagguuguaugguu

SEQ ID NO: 264 - RNA, 22 nt, Homo sapiens
aaccagcaccccaacuuuggac

SEQ ID NO: 265 - RNA, 21 nt, Homo sapiens
cuuccgccccgccgggcgucg

SEQ ID NO: 266 - RNA, 23 nt, Homo sapiens
ugaggggcagagagcgagacuuu

SEQ ID NO: 267 - RNA, 23 nt, Homo sapiens
uagugcaauauugcuuauagggu

SEQ ID NO: 268 - RNA, 24 nt, Homo sapiens
uggcccugacugaagaccagcagu

SEQ ID NO: 269 - RNA, 23 nt, Homo sapiens
ccuaguagguguccaguaagugu

SEQ ID NO: 270 - RNA, 23 nt, Homo sapiens
guuugcacgggugggccuugucu

SEQ ID NO: 271 - RNA, 22 nt, Homo sapiens
uuuugcaauauguuccugaaua

SEQ ID NO: 272 - RNA, 21 nt, Homo sapiens
aagugaucuaaaggccuacau

SEQ ID NO: 273 - RNA, 20 nt, Homo sapiens
cggcucugggucugugggga

SEQ ID NO: 274 - RNA, 21 nt, Homo sapiens
agcuacaucuggcuacugggu

SEQ ID NO: 275 - RNA, 21 nt, Homo sapiens
agggcccccccucaauccugu

SEQ ID NO: 276 - RNA, 22 nt, Homo sapiens
uagcaccaucugaaaucgguua

SEQ ID NO: 277 - RNA, 23 nt, Homo sapiens
uagcaccauuugaaaucaguguu

SEQ ID NO: 278 - RNA, 22 nt, Homo sapiens
gccugcugggguggaaccuggu

SEQ ID NO: 279 - RNA, 21 nt, Homo sapiens
aauauuauacagucaaccucu

SEQ ID NO: 280 - RNA, 21 nt, Homo sapiens
aggggugcuaucugugauuga

SEQ ID NO: 281 - RNA, 20 nt, Homo sapiens
ggggagcuguggaagcagua

SEQ ID NO: 282 - RNA, 21 nt, Homo sapiens
agggcccccccucaauccugu

SEQ ID NO: 283 - RNA, 21 nt, Homo sapiens
gccccugggccuauccuagaa

SEQ ID NO: 284 - RNA, 21 nt, Homo sapiens
agggcccccccucaauccugu

SEQ ID NO: 285 - RNA, 22 nt, Homo sapiens
uaugcauuguauuuuuaggucc

SEQ ID NO: 286 - RNA, 23 nt, Homo sapiens
ugcaccaugguugucugagcaug

SEQ ID NO: 287 - RNA, 22 nt, Homo sapiens
caaaaaucucaauuacuuuugc

SEQ ID NO: 288 - RNA, 22 nt, Homo sapiens
cugugcgugugacagcggcuga

SEQ ID NO: 289 - RNA, 22 nt, Homo sapiens
caaaaaccacaguuucuuuugc

SEQ ID NO: 290 - RNA, 22 nt, Homo sapiens
aaaaguauuugcggguuuuguc

SEQ ID NO: 291 - RNA, 23 nt, Homo sapiens
ugcaccaugguugucugagcaug

SEQ ID NO: 292 - RNA, 22 nt, Homo sapiens
uagcaccaucugaaaucgguua

SEQ ID NO: 293 - RNA, 22 nt, Homo sapiens
uagcaccaucugaaaucgguua

SEQ ID NO: 294 - RNA, 21 nt, Homo sapiens
ggcuagcaacagcgcuuaccu

SEQ ID NO: 295 - RNA, 19 nt, Homo sapiens
uggauuuuuggaucaggga

SEQ ID NO: 296 - RNA, 21 nt, Homo sapiens
aacauagaggaaauuccacgu

SEQ ID NO: 297 - RNA, 22 nt, Homo sapiens
caaagugccucccuuuagagug

SEQ ID NO: 298 - RNA, 24 nt, Homo sapiens
acugggggcuuucgggcucugcgu

SEQ ID NO: 299 - RNA, 22 nt, Homo sapiens
aaccagcaccccaacuuuggac

SEQ ID NO: 300 - RNA, 21 nt, Homo sapiens
cuuccgccccgccgggcgucg

SEQ ID NO: 301 - RNA, 21 nt, Homo sapiens
augauccaggaaccugccucu

SEQ ID NO: 302 - RNA, 24 nt, Homo sapiens
uggcccugacugaagaccagcagu

SEQ ID NO: 303 - RNA, 23 nt, Homo sapiens
ccuaguagguguccaguaagugu

SEQ ID NO: 304 - RNA, 23 nt, Homo sapiens
guuugcacgggugggccuugucu

SEQ ID NO: 305 - RNA, 21 nt, Homo sapiens
aagugaucuaaaggccuacau

SEQ ID NO: 306 - RNA, 23 nt, Homo sapiens
aguauguucuuccaggacagaac

SEQ ID NO: 307 - RNA, 21 nt, Homo sapiens
aauauuauacagucaaccucu

SEQ ID NO: 308 - RNA, 21 nt, Homo sapiens
aggggugcuaucugugauuga

SEQ ID NO: 309 - RNA, 20 nt, Homo sapiens
ggggagcuguggaagcagua

SEQ ID NO: 310 - RNA, 21 nt, Homo sapiens
gccccugggccuauccuagaa

SEQ ID NO: 311 - RNA, 22 nt, Homo sapiens
uaugcauuguauuuuuaggucc

SEQ ID NO: 312 - RNA, 22 nt, Homo sapiens
caaaaaucucaauuacuuuugc

SEQ ID NO: 313 - RNA, 23 nt, Homo sapiens
cucuugagggaagcacuuucugu

SEQ ID NO: 314 - RNA, 22 nt, Homo sapiens
cugugcgugugacagcggcuga

SEQ ID NO: 315 - RNA, 21 nt, Homo sapiens
ggcuagcaacagcgcuuaccu

SEQ ID NO: 316 - RNA, 22 nt, Homo sapiens
uggcagugucuuagcugguugu

SEQ ID NO: 317 - RNA, 21 nt, Homo sapiens
aacauagaggaaauuccacgu

SEQ ID NO: 318 - DNA, 23 bp, Artificial Sequence
  Synthetic primer
tccatatgtgttcctcttgttct

SEQ ID NO: 319 - DNA, 23 bp, Artificial Sequence
  Synthetic primer
ttatgggtgtctgtaaggaaaaa

SEQ ID NO: 320 - DNA, 22 bp, Artificial Sequence
  Synthetic primer
cctgactcagctaatgtcacaa

SEQ ID NO: 321 - DNA, 23 bp, Artificial Sequence
  Synthetic primer
gcagcttgtgcagtaagtttctt

SEQ ID NO: 322 - DNA, 20 bp, Artificial Sequence
  Synthetic primer
ggccattttggtgcttattc

SEQ ID NO: 323 - DNA, 26 bp, Artificial Sequence
  Synthetic primer
cagaaccaagttttattttgtagtcg

SEQ ID NO: 324 - DNA, 20 bp, Artificial Sequence
  Synthetic primer
caatgagcaccaccatcaat

SEQ ID NO: 325 - DNA, 23 bp, Artificial Sequence
  Synthetic primer
ttggaagtcaaacaaaactcaca

SEQ ID NO: 326 - DNA, 25 bp, Artificial Sequence
  Synthetic primer
tctaaatcttgtgctagaaaaagca

SEQ ID NO: 327 - DNA, 22 bp, Artificial Sequence
  Synthetic primer
ctttgaacaatgaaaagccttg

SEQ ID NO: 328 - DNA, 21 bp, Artificial Sequence
  Synthetic primer
tcaccatccagagaccaaata

SEQ ID NO: 329 - DNA, 23 bp, Artificial Sequence
  Synthetic primer
caaagtgattttggctgagtaaa

SEQ ID NO: 330 - DNA, 23 bp, Artificial Sequence
  Synthetic primer
ataaatcagtgcctggtgttctg

SEQ ID NO: 331 - DNA, 21 bp, Artificial Sequence
  Synthetic primer
atgagaatgcaaagaggaaca

SEQ ID NO: 332 - DNA, 20 bp, Artificial Sequence
  Synthetic primer
cctctccactgccttcgata

SEQ ID NO: 333 - DNA, 20 bp, Artificial Sequence
  Synthetic primer
cctcgaacagatgccacaat

SEQ ID NO: 334 - DNA, 20 bp, Artificial Sequence
  Synthetic primer
tttctgctcctgacgtgttg

SEQ ID NO: 335 - DNA, 21 bp, Artificial Sequence
  Synthetic primer
tgagtgagaagcaccaggttt

SEQ ID NO: 336 - DNA, 19 bp, Artificial Sequence
  Synthetic primer
ctcagggtgcacacaggat

SEQ ID NO: 337 - DNA, 20 bp, Artificial Sequence
  Synthetic primer
cacgctccaacaaaatgtca

SEQ ID NO: 338 - DNA, 20 bp, Artificial Sequence
  Synthetic primer
ctctttaaccctccccttcg

SEQ ID NO: 339 - DNA, 20 bp, Artificial Sequence
  Synthetic primer
gaggggaaatgacatctgga

SEQ ID NO: 340 - DNA, 21 bp, Artificial Sequence
  Synthetic primer
gccacacttcaggctcttctc

SEQ ID NO: 341 - DNA, 49 bp, Artificial Sequence
  Synthetic primer
gagaagagcctgaagtgtggccgacaacgggcagcccaaagcagggctg

SEQ ID NO: 342 - DNA, 51 bp, Artificial Sequence
  Synthetic primer
catagatttaagtgaatacattaacatgcggtaaaatgaaaattctaaccc

SEQ ID NO: 343 - DNA, 48 bp, Artificial Sequence
  Synthetic primer
tgtattcacttaaatctatgtaccgcatttgtctccaggcagaacaac

(19) United States
(12) Patent Application Publication
(10) Pub. No.: US 2017/0218454 A1
(43) Pub. Date: Aug. 3, 2017

(54) Products and Methods Relating to Micro RNAS and Cancer

(71) Applicant: INSTITUTE FOR SYSTEMS BIOLOGY, Seattle, WA (US)

(72) Inventors: Christopher L. Plaisier, Seattle, WA (US); Nitin S. Baliga, Seattle, WA (US)

(21) Appl. No.: 14/901,707

(22) PCT Filed: Jun. 26, 2014
(86) PCT No.: PCT/US14/44385

§ 371 Date: Oct. 5, 2016

Related U.S. Application Data
(60) Provisional application No. 61/840,255, filed on Jun. 27, 2013.
(60) Provisional application No. 61/888,346, filed on Oct. 8, 2013.

(51) Int. Cl. C12Q 1/68; G06F 19/18; G06F 19/00; G06F 19/20

(52) U.S. Cl. CPC C12Q 1/6886; G06F 19/20; G06F 19/18; G06F 19/345; G06F 19/3431; C12Q 2600/106; C12Q 2600/178; C12Q 2600/158

(57) ABSTRACT

The invention encompasses products and methods relating to microRNAs involved in various cancers.

### STATEMENT OF GOVERNMENT INTEREST

This invention was made with U.S. Government support under NIH (P50GM076547 and 1R01GM077398-01A2), DoE (DE-FG02-04ER64685)and NSF (DBI-0640950). The U.S. Government has certain rights in the invention.

### FIELD OF THE INVENTION

The invention encompasses products and methods relating to microRNAs involved in various cancers.

### BACKGROUND

MicroRNAs (miRNAs) mediate degradation (Baek et al. 2008) or translational repression (Selbach et al. 2008) of gene transcripts associated with an array of biological processes including many of the hallmarks of cancer (Dalmay and Edwards 2006; D Hanahan and R A Weinberg 2000; Douglas Hanahan and Robert A Weinberg 2011; Ruan et al. 2009). Not surprisingly, dysregulated miRNAs can be readily detected in tumor biopsies (Jiang et al. 2009) and are known to be diagnostic and prognostic indicators (Zen and Chen-Yu Zhang 2010). In some cases miRNAs have also been shown to be potential therapeutic targets (Garofalo and Croce 2011; Nana-Sinkam and Croce 2011). Conservative estimates suggest that each human miRNA regulates several hundred transcripts (Baek et al. 2008; Selbach et al. 2008) and thus miRNA mediated regulation results in statistically significant gene co-expression signatures that are readily discovered through transcriptome profiling (Brueckner et al. 2007; Ceppi et al. 2009; Tsung-Cheng Chang et al. 2007; Fasanaro et al. 2009; Frankel et al. 2008; Georges et al. 2008; Grimson et al. 2007; Lin He et al. 2007; Hendrickson et al. 2008; Charles D Johnson et al. 2007; Karginov et al. 2007; Lee P Lim et al. 2005; Linsley et al. 2007; Malzkorn et al. 2010; Ozen et al. 2008; Sengupta et al. 2008; Tan et al. 2009; Tsai et al. 2009; Valastyan et al. 2009; Wang-Xia Wang et al. 2010; Xiaowei Wang and Xiaohui Wang 2006; Frank Weber et al. 2006).

There are two commonly used strategies to identify the miRNA regulator(s) responsible for the observed co-expression of a set of genes: 1) enrichment of predicted 3′ UTR binding sites for a known miRNA (Betel et al. 2010, 2008; Friedman et al. 2009; Kertesz et al. 2007); or 2) de novo identification of a 3′ UTR motif that is complementary to a seed sequence of a miRNA in miRBase (Fan et al. 2009; Goodarzi et al. 2009; Kozomara and Griffiths-Jones 2011; Linhart et al. 2008). Algorithms utilizing the first strategy incorporate some combination of seed complementarity, cross-species conservation, and thermodynamic properties of the binding site. These algorithms include PITA (Kertesz et al. 2007), TargetScan (Friedman et al. 2009), and both miRanda (Betel et al. 2008) and miRSVR (Betel et al. 2010) from microlMA.org. While the combined modeling of two or more miRNA-binding properties within these algorithms boosts signal, the multiple hypotheses testing required to identify bona fide miRNA-binding sites unfortunately also simultaneously leads to high false negative rates (−32-52%) (Sethupathy et al. 2006).

Despite some progress in assessing the risk of cancer, a need exists for accurate methods of assessing such risks or developing conditions. Treatment of pre-cancer with drugs could postpone or prevent cancer; yet few pre-cancer patients are treated. A major reason is that no simple and unambiguous laboratory test exists to determine the actual risk of an individual to develop cancer. Thus, there remains a need in the art for methods of identifying, diagnosing, and treating these individuals.

### BRIEF SUMMARY

The present application provides prognostic methods for determining risk for developing cancer or predicting progression of cancer, and for predicting response to a drug or treatment regimen; diagnostic methods for identifying type(s) of cancer and for identifying a response to a drug or monitor a treatment regimen; therapeutic methods for directing appropriate treatments for patients at risk of progression, for directing appropriate treatments for patients with an identified type of cancer, for administering a drug that increases a miRNA useful for the treatment of cancer and for administering a drug to inhibit a miRNA identified as being involved in causing or exacerbating cancer; computer systems based on algorithms useful in the prognostic, diagnostic and/or therapeutic methods; miRNA products (including, but not limited to, products useful as biomarkers) and panels (i.e., sets of miRNA products); and products (e.g., arrays or kits of reagents) to detect miRNAs or panels of miRNAs and methods of using the detection products.

### BRIEF DESCRIPTION OF THE DRAWINGS

FIG. 1. Overview of Weeder-miRvestigator tandem developed to identify miRNAs driving co-expression of transcripts. Quantitative assays of the transcriptome are used to identify gene co-expression signatures comprised of genes with significantly similar gene expression profiles. The 3′ UTR sequences for the co-expressed genes are then extracted from the genome and used as input into the Weeder algorithm. The Weeder algorithm searches the 3′ UTR sequences for an over-represented motif which is turned into a miRvestigator hidden Markov model (HMM). All of the miRNA seed sequences from the miRNA repository miRBase are compared to the HMM model of the over-represented sequence motif using the Viterbi algorithm. The miRNA seed sequence with the most significant complementarity p-value is the most likely miRNA driving the co-expression signature and a hypothesis that can be tested experimentally.

FIG. 2. The sensitivity and specificity of the miRvestigator algorithm and framework is estimated using simulated datasets. A. The ROC AUC was computed by simulating miR-1 motifs across a range of motif entropies. Shown are the ROC AUC for the consensus matched to 8 bp miRNA seed sequences from miRBase using regular expression and the miRvestigator IIMM derived scoring metrics Viterbi P-value. B. We then tested the sensitivity and specificity of coupling de novo motif detection algorithm Weeder to the miRvestigator (FIG. 1) by applying them to 30 simulated sequences with varying levels of inserted miR-1 seed sequence (0 to 100%). C. Histogram of Weeder identified miRNA binding sites for whole transcripts where transcripts are centered on the stop codon (0 bp). Instances of miRNA binding sites were either stratified based upon their complementarity to the motif identified by Weeder (8 bp, 7 bp or 6 bp) or the combination of all complementarities. As described by the gene structure below the histogram upstream of the stop codon are the 5′ UTR and coding regulatory regions, and downstream is the 3′ UTR. In the gene structure below the histogram the coding sequences is a wider grey box, the start codon is a green arrow, and the stop codon is a red stop sign. D. Significance of the enrichment of miRNA binding sites per 1 Kbp was computed as a meta statistic are shown for each gene region and each stratified site complementarity.

FIG. 3. A. Determining the optimal method(s) (most sensitive and specific) to infer miRNA mediated regulation from co-expressed genes. The methods tested were: 1) Weeder coupled to miRvestigator (Weeder-miRvestigator) (black line), 2) enrichment of PITA predicted milMA target genes (blue line), 3) enrichment of TargetScan predicted target genes (green line), 4) enrichment of miRSVR predicted target genes (orange line), and 5) enrichment of miRanda predicted target genes (red line). B. Overlap of co-expression signatures between putative miRNA regulators predicted by the three methods (Weeder-miRvestigator, PITA and TargetScan) in FIRM. Pairwise overlap of co-expression signatures between methods is statistically significant (Weeder-miRvestigator vs. PITA=0.045; Weeder-miRvestigator vs. TargetScan=0.019; PITA vs. TargetScan=7.4×10⁻²²). All three methods identified miR [symbol] 29a/b/c as the regulator for the lung adenocarcinoma co-expression signature AD Lung Beer 31.

FIG. 4. Metastatic and cross cancer-miRNA regulatory networks. Hierarchy of filters applied to cancer-miRNA regulatory network to produce both the metastatic and cross cancer miRNA regulatory networks is depicted above the networks, and a legend for the networks can be found in the upper right corner. Nodes are cancers (purple octagons), co-expression signatures (orange circles), inferred miRNAs (red diamonds), or hallmarks of cancer (green parallelogram). Orange edges describe the cancer where a co-expression signature was observed, blue edges link a putative miRNA regulator to a co-expression signature (putative miRNA regulation from cancer miRNA regulatory network), and red edges link putative miRNAs to the hallmarks of cancer based upon functional enrichment of the co-expression signatures they regulate (GO term semantic similarity). Thicker dashed edges indicate experimental validation for the inferred relationship. A. Metastatic cancer-miRNA regulatory network was filtered for the sake of space to show only cancers with at least one predicted regulatory interactions that has been validated. B. Cross cancer-miRNA regulatory network was generated by identifying miRNAs with more than one co-expression signature that are functionally enriched for the same GO terms that are sufficiently similar to GO terms characterizing the hallmarks of cancer.

FIG. 5. Luciferase reporter assay validation of miRNA binding site predictions from FIRM. A. Deletion of miR-29 binding sites ablates response to miR-29a mimic. The wild type 3′ UTRs are MMP2 and SPARC. The miR-29 binding site deleted 3′ UTRs are MMP2 Δ and SPARC Δ. The deletions have a slight increase in normalized luminescence over their corresponding vector control which is similar to what is observed for the negative control HIST1H2AC which doesn't have a miR-29 binding site. B. Dose response curves for COL3A1 and SPARC titrating the amounts of miR-29a mimic (50 nM, 5 nM, 500 pM, 50 pM and 5 pM).

FIG. 6. Summary of FIRM predictions for the miR-29a/b/c and miR-767-5p cancer-miRNA regulatory subnetwork. This subnetwork is included in both the metastatic- and cross-cancer miRNA regulatory networks. The network is laid out hierarchically with from the top down cancers, miRNAs, co-expression signatures, genes that were experimentally validated through luciferase assays, significantly enriched GO biological process terms for the co-expression signature, and finally the GO terms associated hallmarks of cancers. On the left side we show the FIRM integration strategy which is a flow of information through this hierarchy where the red arrows indicate a FIRM prediction. The meanings of the FIRM predictions are described on the right side where inference of a miRNA regulating a cancer co-expression signature predicts that the miRNA is dysregulated in that cancer. This same inference predicts that the miRNA regulates the genes in the signature which can be tested experimentally. Functional enrichment of GO term annotations among the co-regulated genes predicts the effect of regulating this set of genes and association of the enriched GO terms with hallmarks of cancer predicts the oncogenic processes that might be affected.

FIG. 7 is a flowchart showing how cancer gene expression signatures are used to identify cancer miRNA regulatory networks according to various methods described herein.

FIG. 8 is a flow diagram representing an exemplary FIRM method 800.

FIG. 9 is a flow diagram representing an exemplary method 900 for performing de novo identification of one or more 3′ UTR motifs that are complementary to seed sequences of miRNA stored on a memory device (i.e., an exemplary method corresponding to the block 802).

FIG. 10 is a flow diagram representing an exemplary method 1000 for identifying enriched predicted miRNA binding sites (i.e., an exemplary method corresponding to the block 804).

FIG. 11 is a flowchart showing how the identification of cancer miRNA regulatory networks leads therapeutic options according to methods described herein.

FIG. 12 is a panel of miRNAs involved in oncogenic processes across diverse cancers.

FIG. 13 is a panel of miRNAs involved in cancer metastasis and tissue invasion.

FIG. 14 shows miRNAs variously involved in sustained angiogenesis, tumor-promoting inflammation, self-sufficiency in growth signals, reprogramming energy metabolism, evading apoptosis, genome instability and mutation, limitless replicative potential, evading immune detection, and insensitivity to anti-growth signals in a number of cancers.

FIG. 15 is an alignment of miR-767-5p, miR-29a, miR-29c and miR-29b.

### DESCRIPTION

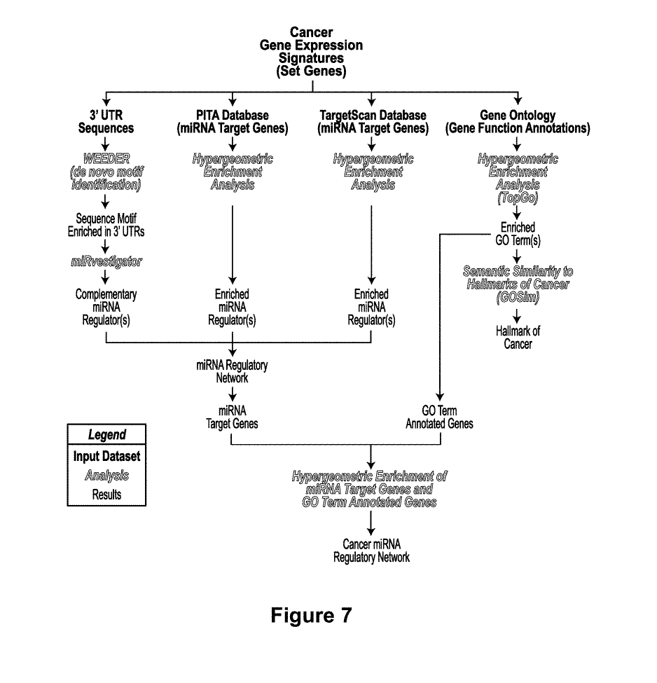

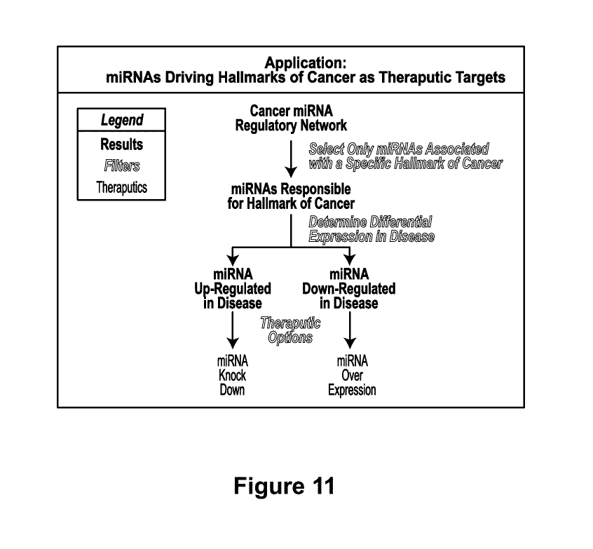

In a first aspect, a Framework for Inference of Regulation by miRNAs (FIRM) is provided. FIRM integrates three best performing algorithms to infer miRNA that mediate regulation from co-expression signatures. In an exemplary embodiment, FIRM limits the Weeder-miRvestigator method to only those inferences of miRNA mediated regulation with a perfect 7- or 8-mer miRvestigator complementarity p-value (p-value=6.1×10⁻⁵ or 1.5×10⁻⁵, respectively) to a miRNA seed in miRBase. Inferences of miRNA mediated regulation from the PITA and TargetScan enrichment of predicted miRNA target genes methods are filtered to include only those with Benjamini-Hochberg FDR=0.00. FIRM produces a listing (i.e., a panel) of all co-expression signatures predicted to be regulated by an miRNA. See also, the embodiments represented in FIGS. 7 and 11.

FIRM is, at the most basic level, an assemblage of methods combined to produce a data set of co-expression signatures predicted to be regulated by one or more miRNAs. The methods are performed by one or more computer processors executing one or more sets of instructions. The instructions may be hard-encoded into the processor, as in an application-specific integrated circuit (ASIC), may be semi-permanently encoded into the processor, as is the case in, for example, a field-programmable gate array (FPGA), or may be stored on a memory device and executed by a general purpose processor that, after retrieving the instructions from the memory device, becomes a special purpose processor programmed to perform the methods. Generally, the methods may be stored (or encoded, in hardware implementations such as ASICs and FPGAs) as one or more modules or routines. While described below with respect to three methods (and, accordingly, three modules or routines), the methods of which FIRM is comprised may form more than three routines or fewer than three routines. Additionally, individual steps of the methods need not necessarily be performed in the order described. That is, unless a data dependency exists between two steps, it is possible—as will be understood—for steps to be performed in orders other than those described. Further, any particular step may, as will also be understood, represent one or more sub-steps, operations, functions, etc. As but one illustrative example, any particular method step may include retrieving input data from memory, performing one or more processing steps on the data, and storing one or more outputs to the memory.

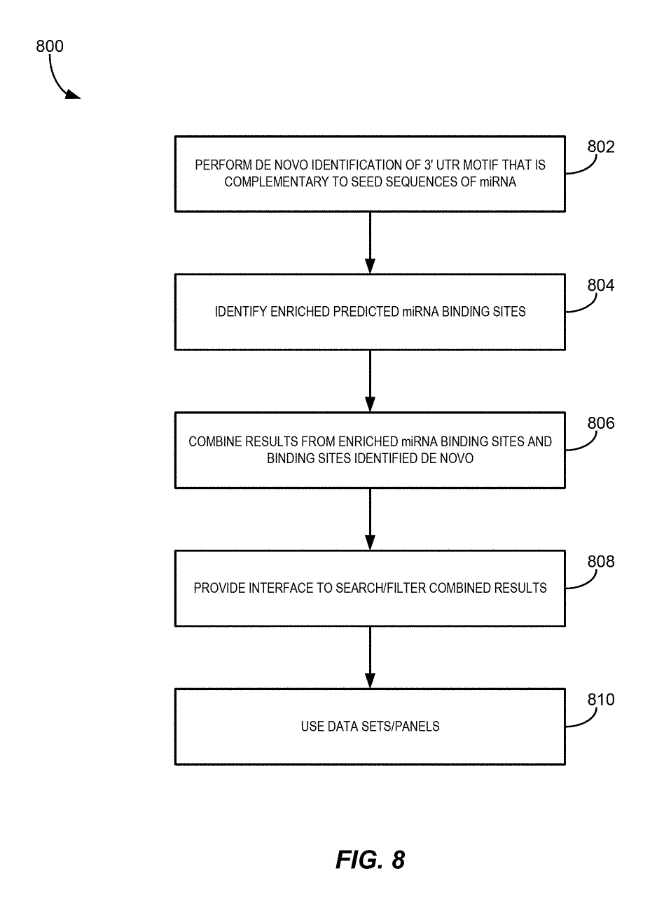

FIG. 8 depicts a flow diagram representing an exemplary FIRM method 800. Generally, the method 800 integrates algorithms to accurately identify the miRNA most likely implicated in the co-regulation of a set of genes represented in a set of genetic expression signatures. Using a first algorithm, the processor performs de novo identification of one or more 3′ UTR motifs that are complementary to seed sequences of miRNA stored on a memory device (block 802). Using a second algorithm, the processor also identifies enriched predicted miRNA binding sites determined from data produced by one or more (two, in an embodiment) of a variety of sub-algorithms such as PITA, TargetScan, miRanda, and miRSVR, etc. (block 804). The results of the blocks 802 and 804 are combined (block 806) as the union of the miRNA to gene co-expression signature predictions.

An interface is optionally provided to allow one or more users to access the combined results (block 808). In one embodiment, the interface takes the form of a Web page available via a network connection (e.g., the Internet), allowing one or more users to access, search, and filter the combined data from any web-enabled device (e.g., workstations, laptop computers, smart phones, tablet devices, etc.). In another embodiment, the interface takes the form of an additional routine operating on a processor (the same processor or a different processor) communicatively connected to a memory on which the combined results are stored. For example, the interface routine may execute on a computing device and, via a network, may access/retrieve the combined results from a database or memory device located remotely. Alternatively, the interface routine may execute on the processor executing the routines related to blocks 802-806.

In any event, the combined data may later be used for any purpose as generally described throughout the remainder of this application (block 810).

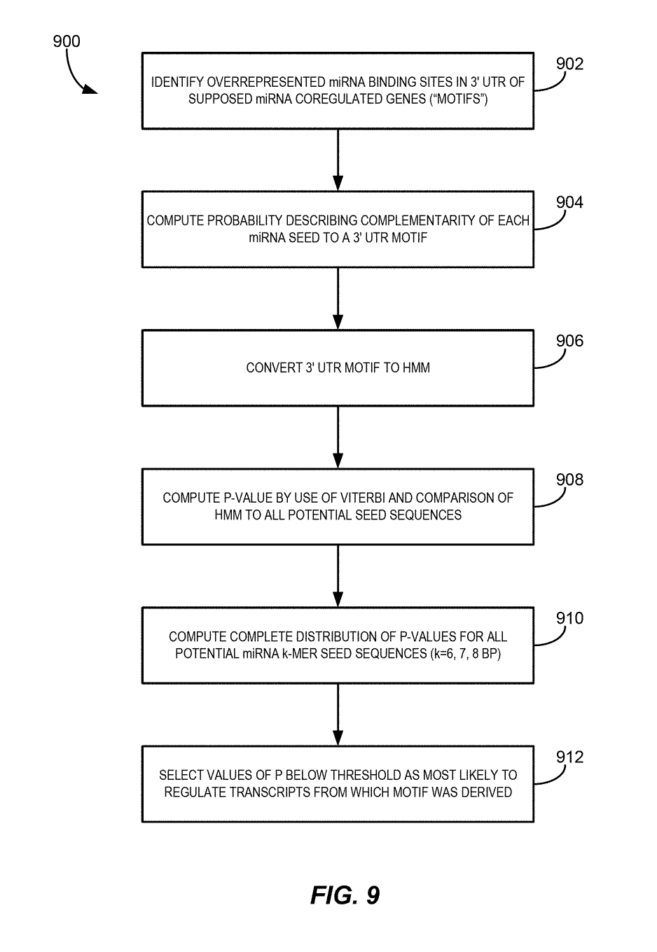

FIG. 9 depicts a flow diagram representing an exemplary method 900 for performing de novo identification of one or more 3′ UTR motifs that are complementary to seed sequences of miRNA stored on a memory device (i.e., an exemplary method corresponding to the block 802). The exemplary method 900 corresponds generally to the miRvestigator algorithm. Overrepresented miRNA binding sites in 3′ UTR of supposed miRNA co-regulated genes (“motifs”) are identified (block 902). For each miRNA seed, the probability describing the complementarity of the miRNA seed to a 3′ UTR motif is computed (block 904). The resulting 3′ UTR motifs are converted to a hidden Markov model (HMM) (block 906). The processor uses the Viterbi algorithm to provide a complementarity p-value by comparing the HMM to all potential seed sequences in a set (e.g., miRBase) (block 908). The complete distribution of complementarity probabilies for all potential miRNA k-mer seed sequences (k=6, 7, or 8 bp) is exhaustively computed (block 910). miRNAs having the smallest complementarity p-values (e.g., below a pre-determined threshold) are selected as most likely to regulate the set of transcripts from which the 3′ UTR motif was derived (block 912). In an embodiment, the threshold is based upon the smallest possible p-value given the size of the search space. For example, for an 8 bp motif, the smallest p-value is 1/4⁸, or 1.5×10⁻⁵, for a 6 bp motif the smallest p-value would be 1/4⁶, or 2.4×10⁻⁴, etc. The threshold is a quality metric that demonstrates the certainty that a particular miRNA is the driving factor for a particular hallmark of cancer. Other thresholds could be used depending on the type of study being conducted.

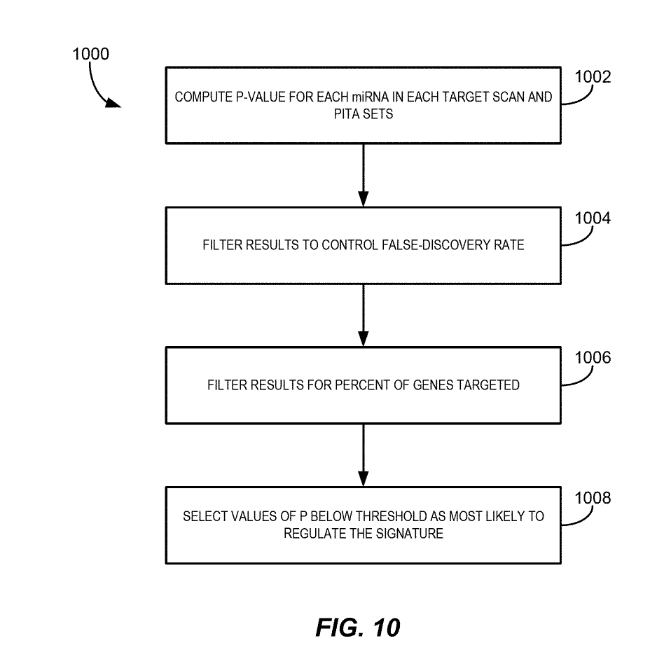

FIG. 10 depicts a flow diagram representing an exemplary method 1000 for identifying enriched predicted miRNA binding sites (i.e., an exemplary method corresponding to the block 804). Data produced by operation of one or more miRNA target gene prediction algorithms (e.g., PITA, TargetScan, miRanda, miRSVR) are analyzed by calculating the hypergeometric p-value for each miRNA in each set of data (block 1002). The sets of data may be stored locally on a memory device and/or may be stored remotely and accessed via a network connection. In any event, in FIG. 10, for example, hypergeometric p-values are calculated for each miRNA in the TargetScan and PITA data sets. The results are optionally filtered to control the false discovery rate (e.g., to be equal to or less than a predetermined value, e.g., 0.001) (block 1004). In an embodiment, the Benjamini-Hochberg False Discovery Rate Procedure (BHFDR) is implemented. Other methods may be used, alternatively or additionally, to control the false discovery rate. The results are optionally filtered to exclude results for which less than a pre-determined portion (e.g., 10 percent) of the genes are targeted by the specific miRNA (block 1006). Further, in some embodiments, the results are filtered based upon the presence of a particular miRNA in the tissue of interest. miRNAs having the smallest hypergeometic p-values (e.g., below a pre-determined threshold) are selected as most likely to regulate the signature (block 1008). Alternatively, in other embodiments the top set of results are selected. In still other embodiments, results with BHFDR corrected p-values below a threshold (e.g., below 0.05) could be selected. The individual miRNAs are sometimes referred to herein as “biomarkers” and sets of miRNAs identified are sometimes referred to as “panels” herein.

By “statistically significant”, it is meant that the inference is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

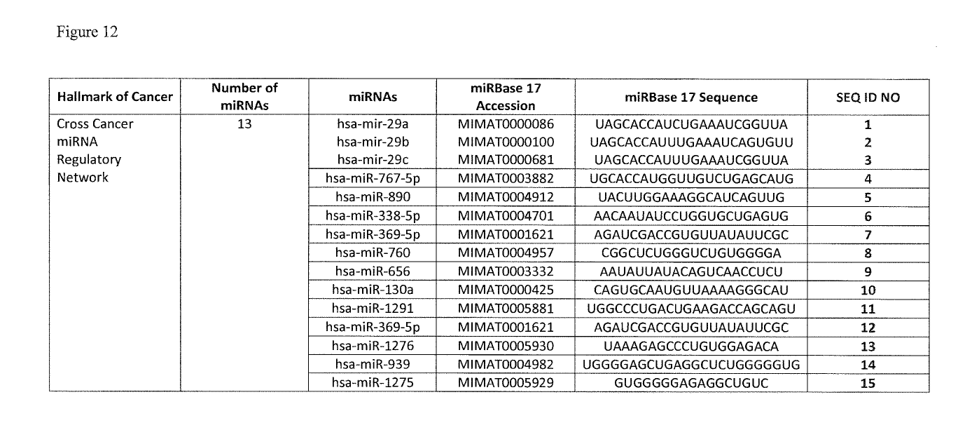

In another aspect, miRNAs are described herein as associated with particular cancers or cancer characteristics. The miRNAs can be measured in an individual and used to evaluate the risk that an individual will develop cancer in the future, for example, the risk that an individual will develop cancer in the next 1, 2, 2.5, 5, 7.5, or 10 years. As used herein, “measuring” includes at least “detecting” a biomarker, but can also include determining the level/quantity of a biomarker. Exemplary miRNAs are shown in the figures. The miRNAs can be employed for methods, kits, computer readable media, systems, and other aspects of the invention which employ individual miRNAs or sets of miRNAs. A panel of miRNAs may comprise one or more miRNAs. MicroRNAs are set out in FIGS. 12 (showing the miRNAs miR-29a/b/c, miR-130a, miR-296-5p, miR-338-5p, miR-369-5p, miR-656, miR-760, miR-767-5p, miR-890, miR-1275, miR-1276 and miR-1291 forming a cross-cancer miRNA regulatory network), 13 (showing the miRNAs forming a metastatic cancer miRNA regulatory network), and 14 (showing the miRNAs forming a sustained angiogenesis miRNA regulatory network, a tumor-promoting inflammation miRNA regulatory network, miRNAs involved in self-sufficiency in growth signals, miRNAs involved in reprogramming energy metabolism, miRNAs involved in evading apoptosis, miRNAs involved in genome instability and mutation, miRNAs involved in limitless replicative potential, miRNAs involved in evading immune detection and miRNAs involved in insensitivity to anti-growth signals).

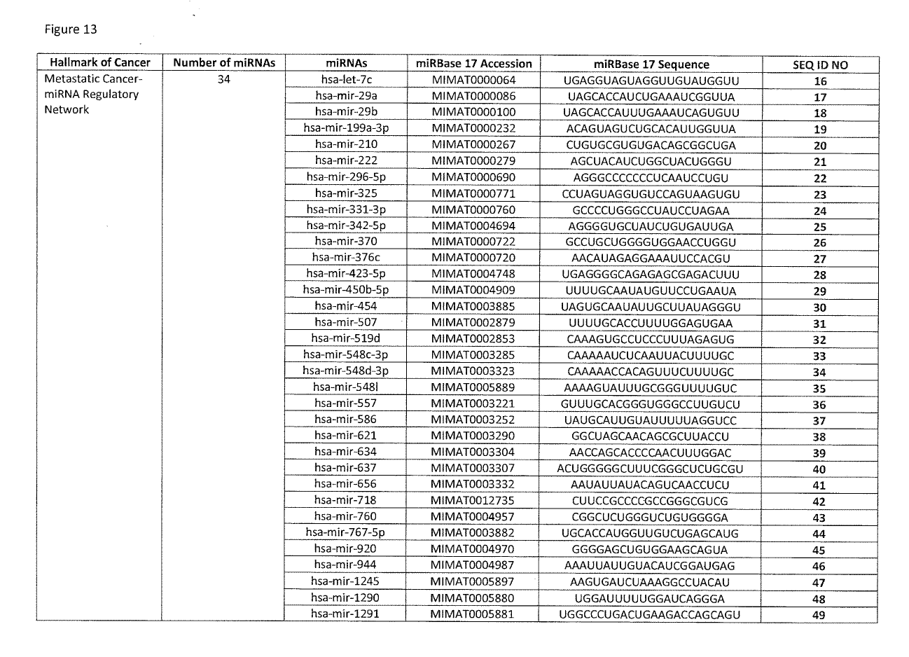

In still another aspect, methods of calculating a risk score for developing cancer are provided, comprising (a) obtaining inputs about an individual comprising the level of biomarkers in at least one biological sample from said individual; and (b) calculating a cancer risk score from said inputs; wherein said biomarkers comprise one or more biomarkers selected from FIGS. 12, 13 and 14.

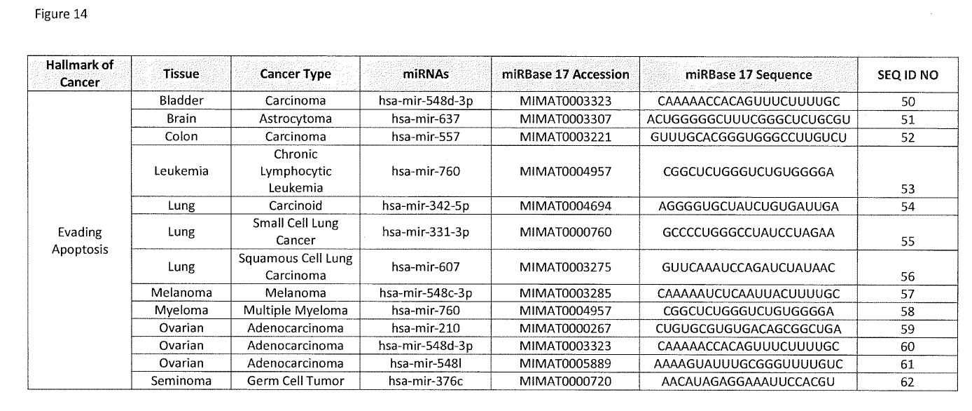

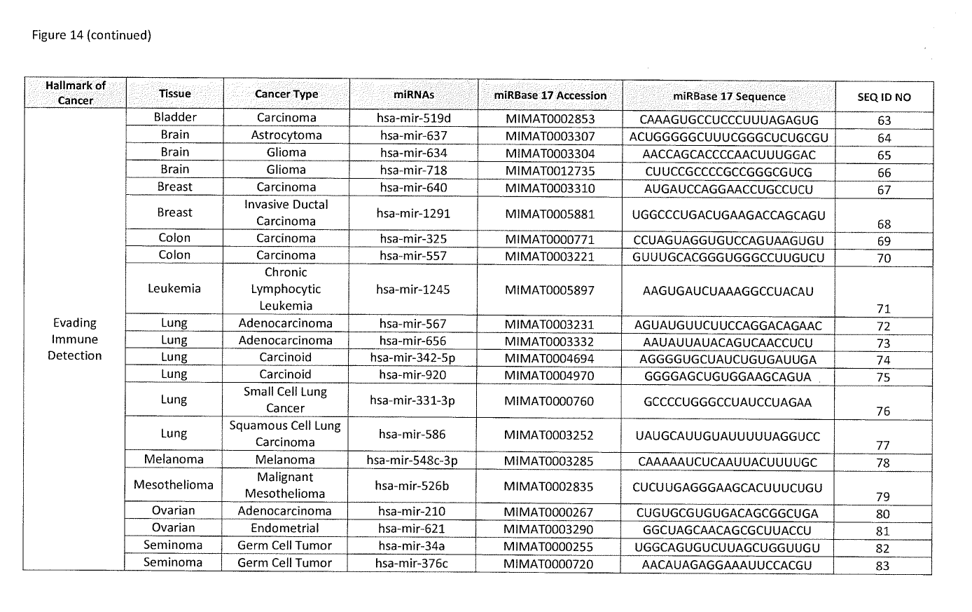

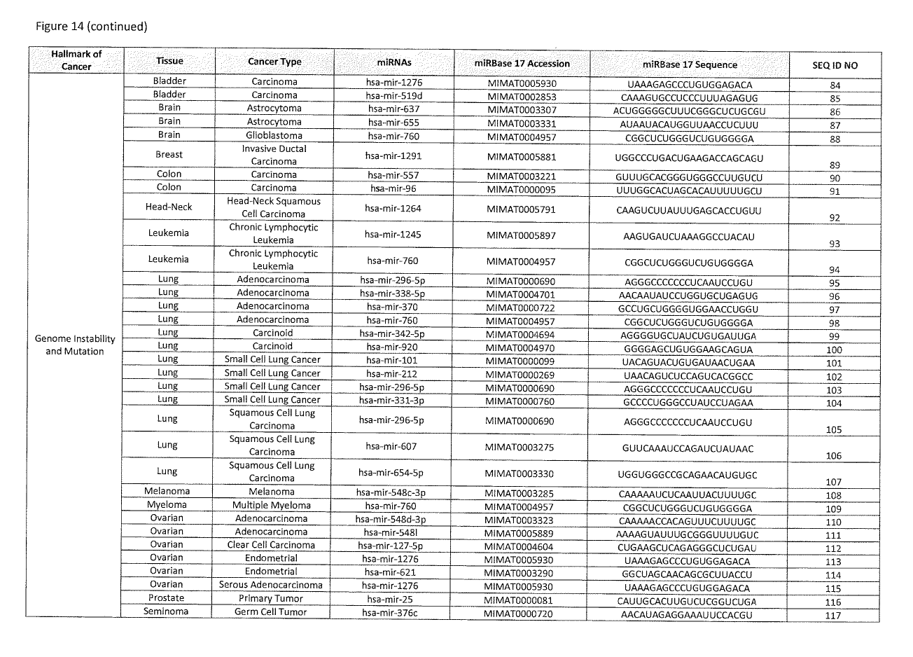

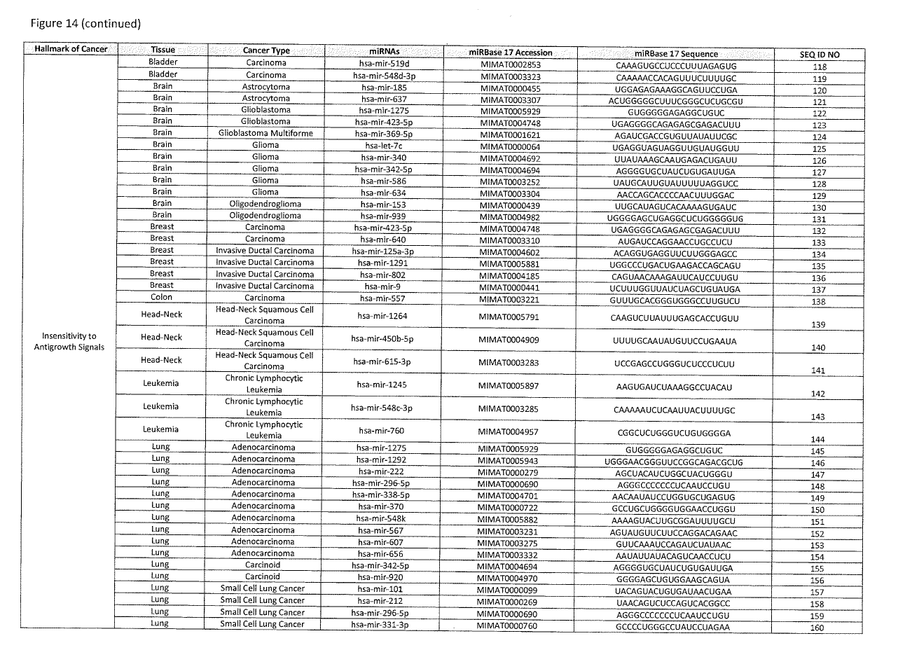

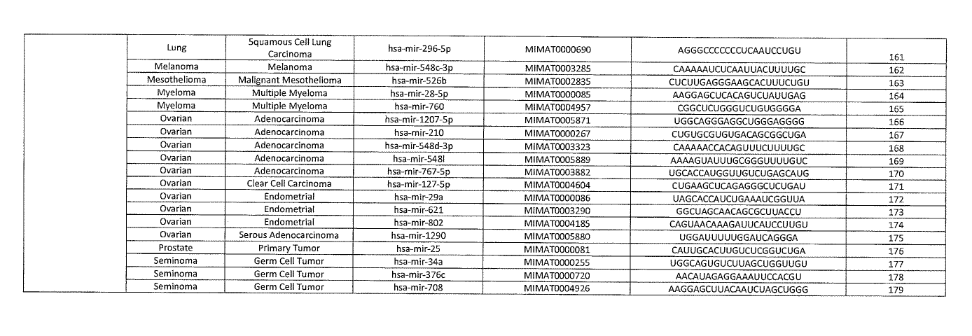

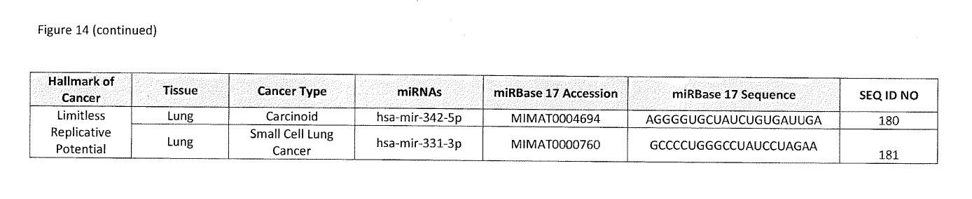

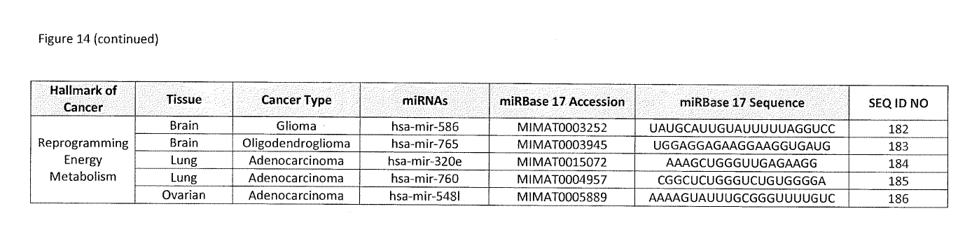

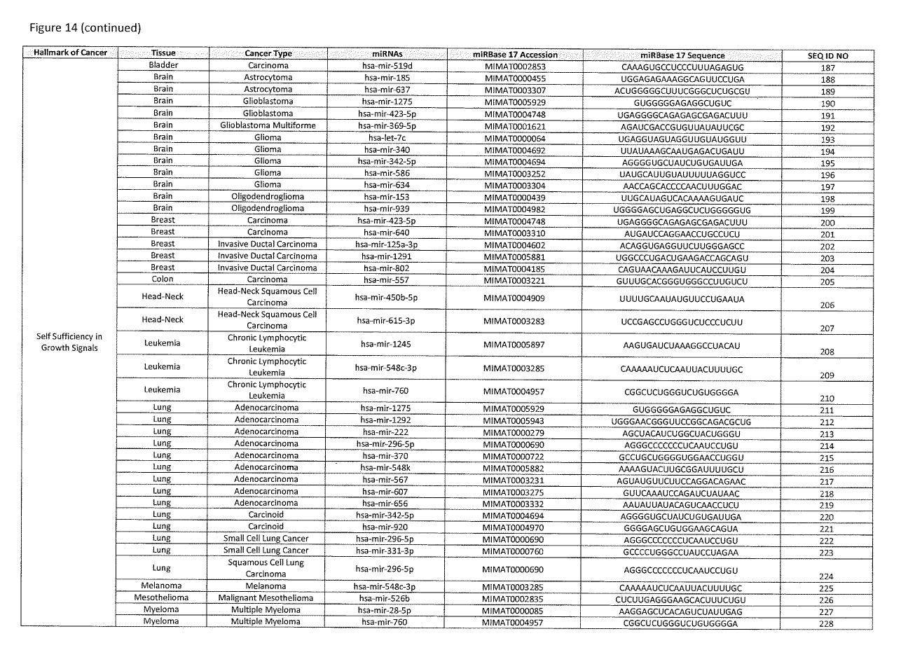

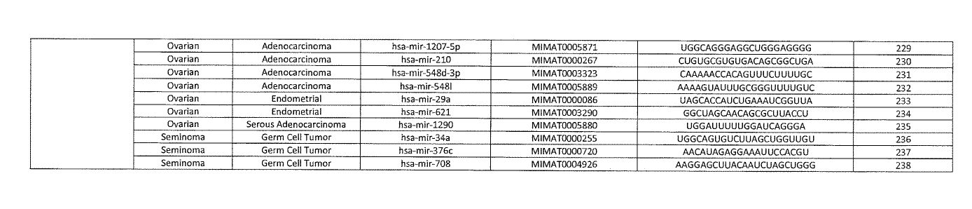

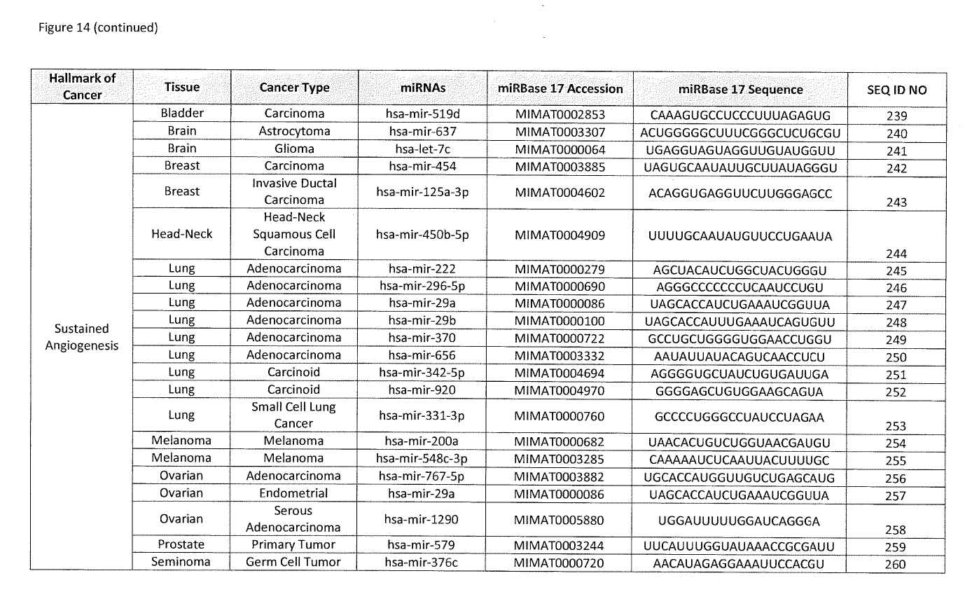

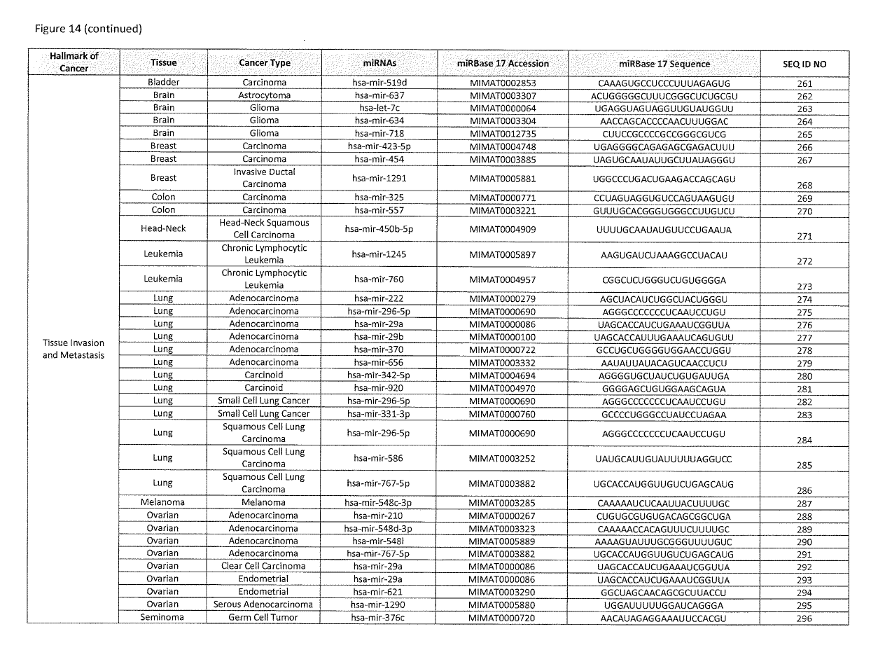

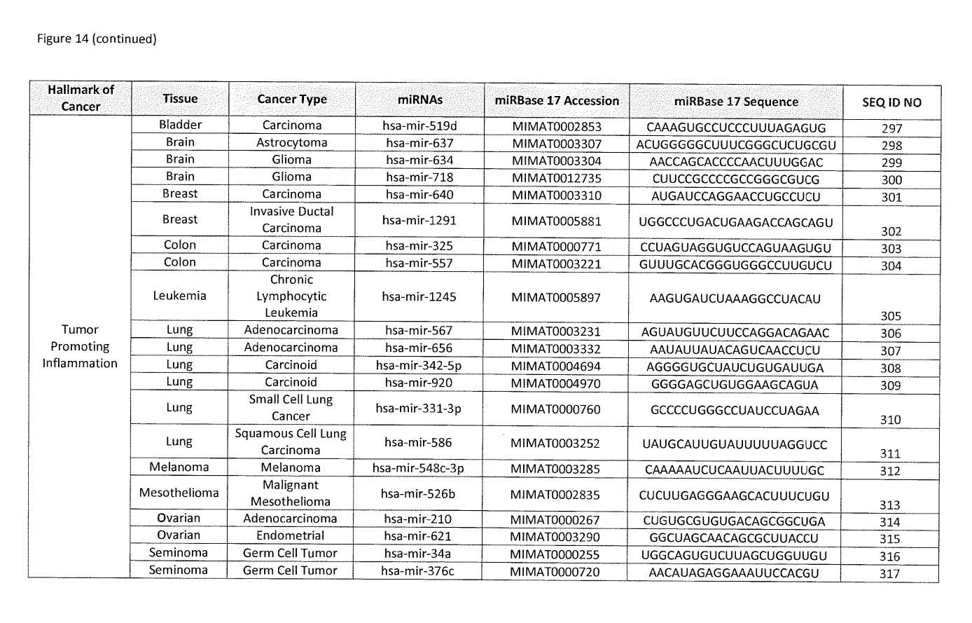

Cancers include, but are not limited to, cancers such as those set out in FIG. 14. These cancers include, but are not limited to, cancers of the bladder, brain, colon, blood, lung, skin, ovary, testes, breast, head, neck and prostate.

In yet another aspect of evaluating risk for developing cancer, the method comprises: (a) obtaining biomarker measurement data, wherein the biomarker measurement data is representative of measurements of biomarkers in at least one biological sample from an individual; and (b) evaluating risk for developing cancer based on an output from a model, wherein the model is executed based on an input of the biomarker measurement data; wherein the biomarkers comprise one or more biomarkers selected from FIGS. 12, 13 and 14.

In an additional aspect, the invention is method of evaluating risk for developing cancer comprising: obtaining biomarker measurements from at least one biological sample from an individual who is a subject that has not been previously diagnosed as having cancer, comparing the biomarker measurement to normal control levels; and evaluating the risk for the individual developing a cancer from the comparison; wherein the biomarkers are defined as set forth in the preceding paragraph.

Similarly, methods are provided of evaluating risk for developing cancer, the method comprising: obtaining biomarker measurement data, wherein the biomarker measurement data is representative of measurements of biomarkers in at least one biological sample from an individual; and evaluating risk for developing cancer based on an output from a model, wherein the model is executed based on an input of the biomarker measurement data; wherein said biomarkers are defined as above.

In some embodiments, the step of evaluating risk comprises computing an index value using the model based on the biomarker measurement data, wherein the index value is correlated with risk of developing cancer in the subject. In some embodiments, evaluating risk comprises normalizing the biomarker measurement data to reference values.

In another aspect, a method of calculating a risk score for cancer progression is provided, comprising (a) obtaining inputs about an individual suffering from cancer comprising the level of biomarkers in at least one biological sample from said individual; and (b) calculating a cancer risk score from said inputs; wherein said biomarkers comprise one or more biomarkers selected from FIGS. 12, 13 and 14.

In some embodiments of the methods disclosed herein, the obtaining biomarker measurement data step comprises measuring the level of at least one of the biomarkers in at least one biological sample from said individual. Optionally, the method includes a step (prior to the step of obtaining biomarker measurement data) of obtaining at least one biological sample from the individual.

In some embodiments, at least one biomarker input is obtained from one or more biological samples collected from the individual, such as from a blood sample, saliva sample, urine sample, cerebrospinal fluid sample, sample of another bodily fluid, or other biological sample including, but not limited to, those described herein.

In some embodiments, at least one biomarker input is obtained from a preexisting record, such as a record stored in a database, data structure, other electronic medical record, or paper, microfiche, or other non-electronic record.

In some embodiments, the biomarkers comprise at least one, at least two, at least three, at least four, at least five, at least six, at least seven, at least eight, at least nine, at least ten, at least eleven, at least twelve, at least thirteen, or more (up to all or all) biomarkers selected from FIG. 12, 13 and/or 14.

In another aspect, the invention embraces a method comprising advising an individual of said individual's risk of developing cancer or risk of cancer progression, wherein said risk is based on factors comprising a cancer risk score, and wherein said cancer risk score is calculated as described above. The advising can be performed by a health care practitioner, including, but not limited to, a physician, nurse, nurse practitioner, pharmacist, pharmacist's assistant, physician's assistant, laboratory technician, dietician, or nutritionist, or by a person working under the direction of a health care practitioner. The advising can be performed by a health maintenance organization, a hospital, a clinic, an insurance company, a health care company, or a national, federal, state, provincial, municipal, or local health care agency or health care system. The health care practitioner or person working under the direction of a health care practitioner obtains the medical history of the individual from the individual or from the medical records of the individual. The advising can be done automatically, for example, by a computer, microprocessor, or dedicated device for delivering such advice. The advising can be done by a health care practitioner or a person working under the direction of a health care practitioner via a computer, such as by electronic mail or text message.

In some embodiments of the invention, the cancer risk score is calculated automatically. The cancer risk score can be calculated by a computer, a calculator, a programmable calculator, or any other device capable of computing, and can be communicated to the individual by a health care practitioner, including, but not limited to, a physician, nurse, nurse practitioner, pharmacist, pharmacist's assistant, physician's assistant, laboratory technician, dietician, or nutritionist, or by a person working under the direction of a health care practitioner, or by an organization such as a health maintenance organization, a hospital, a clinic, an insurance company, a health care company, or a national, federal, state, provincial, municipal, or local health care agency or health care system, or automatically, for example, by a computer, microprocessor, or dedicated device for delivering such advice.

In another embodiment, methods providing two or more cancer risk scores to a person, organization, or database are disclosed, where the two or more cancer risk scores are derived from biomarker information representing the biomarker status of the individual at two or more points in time. In any of the foregoing embodiments, the entity performing the method can receive consideration for performing any one or more steps of the methods described.

In another aspect, a method is provided of ranking or grouping a population of individuals, comprising obtaining a cancer risk score for individuals comprised within said population, wherein said cancer risk score is calculated as described above; and ranking individuals within the population relative to the remaining individuals in the population or dividing the population into at least two groups, based on factors comprising said obtained cancer risk scores. The ranking or grouping of the population of individuals can be utilized for one or more of the following purposes: to determine an individual's eligibility for health insurance; an individual's premium for health insurance; to determine an individual's premium for membership in a health care plan, health maintenance organization, or preferred provider organization; to assign health care practitioners to an individual in a health care plan, health maintenance organization, or preferred provider organization; to recommend therapeutic intervention or lifestyle intervention to an individual or group of individuals; to manage the health care of an individual or group of individuals; to monitor the health of an individual or group of individuals; or to monitor the health care treatment, therapeutic intervention, or lifestyle intervention for an individual or group of individuals.

In another aspect, a panel of biomarkers is provided comprising biomarkers selected from FIG. 12, 13 and/or 14. Exemplary panel embodiments contemplated are a panel comprising one, two or more (up to all or all) miRNAs in FIG. 12; a panel comprising one, two or more (up to all or all) miRNAs in claim 13; a panel comprising one, two or more (up to all or all) miRNAs in FIG. 14 associated with sustained angiogenesis; a panel comprising one, two or more (up to all or all) miRNAs in FIG. 14 associated with tumor-promoting inflammation; a panel comprising one, two or more (up to all or all) miRNAs in FIG. 14 associated with self-sufficiency in growth signals; a panel comprising one, two or more (up to all or all) miRNAs in FIG. 14 associated with reprogramming energy metabolism; a panel comprising one, two or more (up to all or all) miRNAs in FIG. 14 associated with evading apoptosis; a panel comprising one, two or more (up to all or all) miRNAs in FIG. 14 associated with genome instability and mutation; a panel comprising one, two or more (up to all or all) miRNAs in FIG. 14 associated with limitless replicative potential; a panel comprising one, two or more (up to all or all) miRNAs in FIG. 14 associated with evading immune detection; a panel comprising one, two or more (up to all or all) miRNAs in FIG. 14 associated with insensitivity to anti-growth signals; and panels including one, two or more (up to all or all) miRNAs in FIG. 14 respectively associated with a particular tissue or type of cancer [e.g., a panel comprising one, two or more (up to all or all) miRNAs in FIG. 14 associated with a colon cancer; or panel comprising one, two or more (up to all or all) miRNAs in FIG. 14 associated with a carcinoma]. Panels representing every possible combination of miRNAs in FIGS. 12, 13 and 14 are specifically contemplated.

In another aspect, one or more data structures or databases are provided comprising values for one or more biomarkers in FIGS. 12, 13 and 14. A machine-readable storage medium can comprise a data storage material encoded with machine readable data or data arrays which, when using a machine programmed with instructions for using said data, is capable of use for a variety of purposes, such as, without limitation, subject information relating to cancer risk factors over time or in response to cancer-modulating drug therapies, drug discovery, and the like. Measurements of effective amounts of the biomarkers of the invention and/or the resulting evaluation of risk from those biomarkers can implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

In another aspect, diagnostic test systems are provided comprising (1) means for obtaining test results comprising levels of multiple biomarkers in at least one biological sample; (2) means for collecting and tracking test results for one or more individual biological sample; (3) means for calculating an index value from inputs, wherein said inputs comprise measured levels of biomarkers, and further wherein said measured levels of biomarkers comprise the levels of one or more biomarkers selected from FIGS. 12, 13 and 14; and (4) means for reporting said index value. In some embodiments, said index value is a cancer risk score; the cancer risk score can be calculated according to any of the methods described herein. The means for collecting and tracking test results for one or more individuals can comprise a data structure or database. The means for calculating a cancer risk score can comprise a computer, microprocessor, programmable calculator, dedicated device, or any other device capable of calculating the cancer risk score. The means for reporting the cancer risk score can comprise a visible display, an audio output, a link to a data structure or database, or a printer.

A diagnostic system is any system capable of carrying out the methods of the invention, including computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

In some embodiments, a diagnostic test system comprises: means for obtaining test results data representing levels of multiple biomarkers in at least one biological sample; means for collecting and tracking test results data for one or more individual biological samples; means for computing an index value from biomarker measurement data, wherein said biomarker measurement data is representative of measured levels of biomarkers, and further wherein said measured levels of biomarkers comprise the levels of a set or panel of biomarkers as defined elsewhere herein; and means for reporting said index value. In some variations of the diagnostic test system, the index value is a cancer risk score. In some preferred variations, the cancer risk score is computed according to the methods described herein for computing such scores. In some variations, the means for collecting and tracking test results data representing for one or more individuals comprises a data structure or database. In some variations, the means for computing a cancer risk score comprises a computer or microprocessor. In some variations, the means for reporting the cancer risk score comprises a visible display, an audio output, a link to a data structure or database, or a printer.

In some embodiments, a medical diagnostic test system for evaluating risk for developing a cancer or risk for cancer progression, the system comprises: a data collection tool adapted to collect biomarker measurement data representative of measurements of biomarkers in at least one biological sample from an individual; and an analysis tool comprising a statistical analysis engine adapted to generate a representation of a correlation between a risk for developing a cancer and measurements of the biomarkers, wherein the representation of the correlation is adapted to be executed to generate a result; and an index computation tool adapted to analyze the result to determine the individual's risk for developing a cancer or for cancer progression, and represent the result as an index value; wherein said biomarkers are defined as a set or panel as described elsewhere herein. In some variations, the analysis tool comprises a first analysis tool comprising a first statistical analysis engine, the system further comprising a second analysis tool comprising a second statistical analysis engine adapted to select the representation of the correlation between the risk for developing a cancer or risk for cancer progression and measurements of the biomarkers from among a plurality of representations capable of representing the correlation. In some variations, the system further comprising a reporting tool adapted to generate a report comprising the index value.

In some embodiments, a system for diagnosing susceptibility to cancer in a human subject comprises (a) at least one processor; (b) at least one computer-readable medium; (c) a susceptibility database operatively coupled to a computer-readable medium of the system and containing information associating measurements of one or more biomarkers selected from FIGS. 12, 13 and 14 and cancer in a population of humans; (d) a measurement tool that receives an input about the human subject and generates information from the input about one or more biomarkers selected from FIGS. 12, 13 and 14 from the human subject; and (e) an analysis tool (routine) that (i) is operatively coupled to the susceptibility database and the measurement tool, (ii) is stored on a computer-readable medium of the system, (iii) is adapted to be executed on a processor of the system, to compare the information about the human subject with the information about the population in the susceptibility database and generate a conclusion with respect to susceptibility to cancer in the human subject.

In some embodiments, a system for diagnosing cancer in a human subject comprises (a) at least one processor; (b) at least one computer-readable medium; (c) a susceptibility database operatively coupled to a computer-readable medium of the system and containing information associating measurements of biomarkers selected from FIGS. 12, 13 and 14 and cancer in a population of humans; (d) a measurement tool that receives an input about the human subject and generates information from the input about one or more biomarkers selected from FIGS. 12, 13 and 14 from the human subject; and (e) an analysis tool (routine) that (i) is operatively coupled to the susceptibility database and the measurement tool, (ii) is stored on a computer-readable medium of the system, (iii) is adapted to be executed on a processor of the system, to compare the information about the human subject with the information about the population in the susceptibility database and generate a conclusion with respect to the presence of cancer in the human subject. In some embodiments, the biomarkers are measured by amplification or by hybridization to a microarray.

In the systems in the preceding two paragraphs, the input about the human subject can be a biological sample from the human subject, and the measurement tool comprises a tool to measure one or more biomarkers selected from FIGS. 12, 13 and 14 in the biological sample, thereby generating biomarker measurements from a human subject. In some embodiments, the systems further comprise a communication tool operatively coupled to the analysis tool, stored on a computer-readable medium of the system and adapted to be executed on a processor of the system to generate a communication for the human subject, or a medical practitioner for the subject, containing the conclusion with respect to cancer for the subject.

In some embodiments of systems comprising a communication tool operatively connected to the analysis tool or routine, the systems comprise a routine stored on a computer-readable medium of the system and adapted to be executed on a processor of the system, to: generate a communication containing the conclusion; and transmit the communication to the subject or the medical practitioner, or enable the subject or medical practitioner to access the communication.

In some embodiments, any of the systems comprise a medical protocol database operatively connected to a computer-readable medium of the system and containing information correlating the conclusion and medical protocols for human subjects at risk for or suffering from cancer; and a medical protocol tool (or routine), operatively connected to the medical protocol database and the analysis tool or routine, stored on a computer-readable medium of the system, and adapted to be executed on a processor of the system, to compare the conclusion from the analysis routine with respect to cancer for the subject and the medical protocol database, and generate a protocol report with respect to the probability that one or more medical protocols in the database will reduce susceptibility to cancer, delay onset of cancer, increase the likelihood of detecting cancer at an early stage to facilitate early treatment or treat the cancer. Where the communication tool is operatively connected to the medical protocol tool or routine, the system may generate a communication that further includes the protocol report.

Yet another aspect is a computer readable medium having computer executable instructions for evaluating risk for developing a cancer, the computer readable medium comprising: a routine, stored on the computer readable medium and adapted to be executed by a processor, to store biomarker measurement data representing a set or panel of biomarkers; and a routine stored on the computer readable medium and adapted to be executed by a processor to analyze the biomarker measurement data to evaluate a risk for developing a cancer or for risk of cancer progression. The panels of biomarkers are defined as described in any of the preceding paragraphs.

Still another aspect is a method developing a model for evaluation of risk for developing a cancer or for cancer progression, the method comprising: obtaining biomarker measurement data, wherein the biomarker measurement data is representative of measurements of biomarkers from a population and includes endpoints of the population; inputting the biomarker measurement data of at least a subset of the population into a model; training the model for endpoints using the inputted biomarker measurement data to derive a representation of a correlation between a risk of developing a cancer or for cancer progression and measurements of biomarkers in at least one biological sample from an individual; wherein said biomarkers for which measurement data is obtained comprise a set or panel of markers of the invention as defined elsewhere herein.

Another aspect is a kit comprising reagents for measuring a panel of biomarkers, wherein the panel of biomarkers are defined as described in any of the preceding paragraphs, or in a figures, or in other descriptions of preferred panels of markers found herein. In some embodiments, such reagents are packaged together. In some embodiments, the reagents are primers used to amplify miRNA(s) in a panel. In some embodiments, the reagents are DNA arrays that hybridize to miRNA(s) in a panel. In some embodiments, the kit further includes an analysis program for evaluating risk of an individual developing a cancer from measurements of the group of biomarkers from at least one biological sample from the individual.

In measuring miRNA, an amplification reaction using appropriate primers as reagents may be done quantitatively, and the amount of amplified RNA can then be determined with an appropriate probe with a detectable label. The probe may be an oligonucleotide including oligos with nonnative linkages such as phosphothiolate or phosphoramidate, or a peptide nucleic acid (PNA). Nonnative bases may also be included. Thus, a kit may comprise a reagent for an assay which reagent is specific for the miRNA(s), as well as additional reagents needed in order to quantitate the results. Specific miRNA levels can also be measured using general molecular biology techniques commonly known in the art such as Northern blot, quantitative reverse transcription polymerase chain reaction (qRT-PCR), next-generation sequencing or microarray. qRT-PCR is a more sensitive and efficient procedure detect specific messenger RNA or microRNA. The RNA sample is first reverse transcribed, the target sequences can then be amplified using thermostable DNA polymerase. The concentration of a particular RNA sequence in a sample can be determined by examining the amount of amplified products. Microarray technology allows simultaneous measurement of the concentrations of multiple RNA species. Oligonucleotides complementary to specific miRNA sequences are immobilized on solid support. The RNA in the sample is labeled with ColorMatrix™ or florescent dye. After subsequent hybridization of the labeled material to the solid support, the intensities of fluorescent for ColorMatrix™ dye remaining on the solid support determines the concentrations of specific RNA sequences in the samples. The concentration of specific miRNA species can also be determined by NanoString™ nCounter™ system which provides direct digital readout of the number of RNA molecules in the sample without the use of amplification. NanoString™ technology involves mixing the RNA sample with pairs of capture and reporter probes, tailored to each RNA sequence of interest. After hybridization and washing away excess probes, probe-bound target nucleic acids are stretched on a surface and scanned to detect fluorescent-barcodes of the reporter probes. This allows for up to 1000-plex measurement with high sensitivity and without amplification bias. Technologies such as electrochemical biosensor arrays, surface plasma resonance and other targeted capture assays can also be utilized to quantify molecular markers simultaneously by measuring changes in electro-current, light absorption, fluorescence, or enzymatic substrates reactions.

Another aspect includes methods for the prophylactic treatment of a subject at risk for a cancer according to procedures described herein. In some embodiments, the invention includes a method of prophylaxis for cancer comprising: obtaining risk score data representing a cancer risk score for an individual, wherein the cancer risk score is computed according to a method or improvement of the invention; and generating prescription treatment data representing a prescription for a treatment regimen to delay or prevent the onset of cancer to an individual identified by the cancer risk score as being at elevated risk for cancer. In some embodiments, a method of prophylaxis for cancer comprises: evaluating risk, for at least one subject, of developing a cancer according to the method or improvement of the invention; and treating a subject identified as being at elevated risk for a cancer with a treatment regimen to delay or prevent the onset of cancer.

Another aspect includes methods for the therapeutic treatment of a subject indentified as having a cancer according to procedures described herein.

In some embodiments, methods for the prophylactic or therapeutic treatment of a subject comprise administering a drug that increases the amount of a miRNA identified herein that is produced by the body to fight a cancer. In some embodiments, methods comprise administering a drug to inhibit a miRNA or decrease the amount of a miRNA identified herein that is part of the cause of or exacerbates a cancer. In some embodiments, methods comprise both administering a drug that increases the amount of a miRNA identified herein that is produced by the body to fight a cancer, and administering a drug to inhibit a miRNA or decrease the amount of a miRNA identified herein that is part of the cause of or exacerbates a cancer. In some embodiments, the subject is treated with the drug and also receives any other standard of care treatment for the cancer. A drug can be any product including, but not limited, to: small molecules; RNAs or vectors encoding RNAs, such as miRNAs (including miRNAs identified herein), snRNAs and antisense RNAs; peptides or polypeptides; and antibody products that penetrate cells.

A further aspect is a method of evaluating the current status of a cancer in an individual comprising obtaining biomarker measurement data and evaluating the current status of a cancer in the individual based on an output from a model, wherein the biomarkers are any biomarker of the invention.

The foregoing paragraphs are not intended to define every aspect of the invention, and additional aspects are described in other sections. This entire document is intended to be related as a unified disclosure, and it should be understood that all combinations of features described herein are contemplated, even if the combination of features are not found together in the same sentence, or paragraph, or section of this document. With respect to aspects of the invention described as a genus, all individual species are individually considered separate aspects of the invention. With respect to aspects described as a range, all sub-ranges and individual values are specifically contemplated.

Aspects and embodiments of the invention are illustrated by the following non-limiting example.

### EXAMPLES

A generalized framework for the inference of regulation by miRNAs (FIRM) was constructed. In Example 1, a compendium of transcriptome profiles was compiled from studies that had interrogated differential expression of genes in response to targeted perturbation of specific miRNAs (Brueckner et al 2007; Ceppi et al. 2009; Tsung-Cheng Chang et al. 2007; Fasanaro et al. 2009; Frankel et al. 2008; Georges et al. 2008; Grimson et al. 2007; Lin He et al. 2007; Hendrickson et al. 2008; Charles D Johnson et al. 2007; Karginov et al. 2007; Lee P Lim et al. 2005; Linsley et al. 2007; Malzkorn et al. 2010; Ozen et al. 2008; Sengupta et al. 2008; Tan et al. 2009; Tsai et al. 2009; Valastyan et al. 2009; Wang-Xia Wang et al. 2010; Frank Weber et al. 2006). In Example 2, using this compendium of miRNA-perturbed transcriptomes it was demonstrated that functional miRNA binding sites (8 bp of complementarity) preferentially reside in the 3′ UTRs. Further, using preferential 3′ UTR localization as a heuristic was demonstrated to significantly increase sensitivity and specificity of miRNA-binding site discovery by Weeder-miRvestigator. In Example 3, using the compendium of miRNA-perturbed transcriptomes the best performing algorithms were identified and integrated into a generalized framework for inference of miRNA regulatory networks. Finally, the utility of this framework was demonstrated by applying it to a set of 2,240 co-expression signatures from 46 different cancers. The original study was able to associate only four signatures to putative regulation by a known miRNA (Goodarzi et al. 2009). In contrast, using the integrated framework 1,324 signatures were explained as potential outcomes of regulation by specific miRNAs in miRBase. By applying functional enrichment and semantic similarity identified within this expansive network specific miRNAs associated with hallmarks of cancer were identified. Further, filtering gene co-expression signatures for specific hallmarks of cancer such as “tissue invasion and metastasis” generated a metastatic cancer-miRNA regulatory network of 33 miRNAs. Importantly, this revealed that a relatively small subset of miRNAs regulate multiple oncogenic processes across different cancers. Through in depth analyses of data from prior studies as well as new data from targeted miRNA-perturbation experiments, the role of miR-29 family members in lung adenocarcinoma was validated and gene targets for regulation by the relatively unknown miR-767-5p were discovered. Example 4 relates to the use of the FIRM approach to identify other miRNAs associated with hallmarks of cancer. The discussion in Example 5 illustrates how these analyses and validations demonstrate how the cancer-miRNA regulatory network can be used to accelerate discovery of miRNA-based biomarkers and therapeutics.

### Methods

### De Novo Identification of 3′ UTR Motifs

Sequences and RefSeq gene definition files were downloaded from the UCSC genome browser FTP site (ftp://hgdownload.cse.ucsc.edulgoldenPath/currentGenomes/Homo_sapiens). Details can be found in the Supplementary Method section below. The Weeder de novo motif detection algoirthm (Pavesi et al. 2006) was then used to identify over-represented miRNA binding sites in the 3′ UTR of putatively miRNA co-regulated genes (Fan et al. 2009; Linhart et al. 2008).

miRvestigator Identification of Complementary miRNA for 3′ UTR Motif

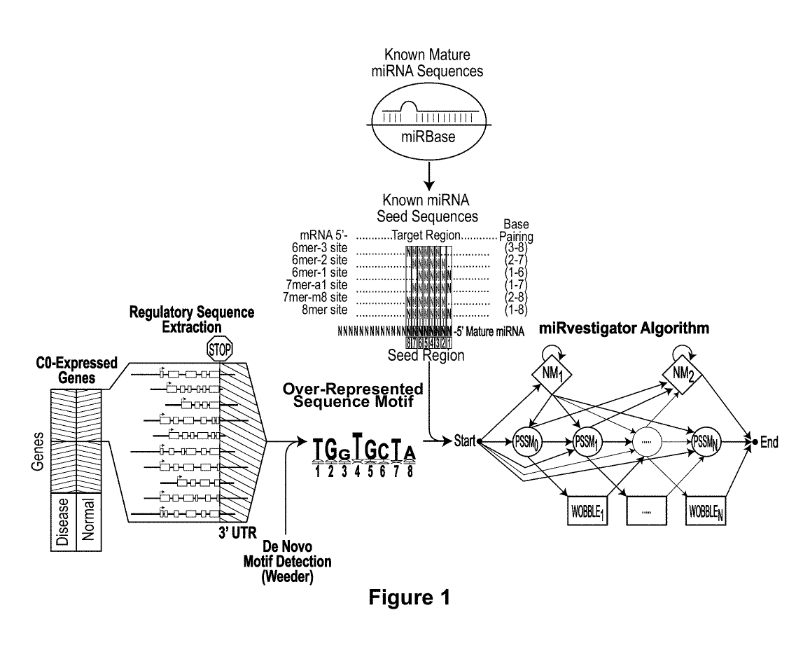

MiRvestigator employs a hidden Markov model (BIMM) to align and compute a probability describing the complementarity of a specific miRNA seed to a 3′ UTR motif (Plaisier et al. 2011). The miRvestigator HIVIM is described in detail in the supplementary methods. The 3′ UTR motif is first converted to a miRvestigator HIVIM and the Viterbi algorithm is used to provide a complementarity p-value by comparing the HIVIM to all potential seed sequences from miRBase. There are different models for the base-pairing of miRNA seeds to the complementary protein coding transcript binding sites as described in FIG. 1 (Bartel 2009; Brennecke et al. 2005). The significance of the complementarity for a given miRNA is then calculated by exhaustively computing the complete distribution of complementarity probabilities for all potential miRNA k-mer seed sequences (where k=6, 7 or 8 bp). The miRNA(s) with the smallest complementarity p-value are considered the most likely to regulate the set of transcripts from which the 3′ UTR motif was derived.

### Simulating Synthetic Motifs and 3′ UTRs Sequences

Motifs were simulated based upon the reverse complement of the 8 bp seed sequence 5′-UGGAAUGU-3′ for miR-1 (MIMAT0000416). The miRNA seed signal determined the percent that the seed nucleotide was given in each column of the PSSM and the remaining signal was distributed randomly to the other three nucleotides. We simulated motifs with different entropies by adding between 10 to 75% noise at a 5 percent interval to each seed nucleotide position. A seed nucleotide signal of 25 percent is the random case as one of the other three nucleotides is likely to have a higher frequency than the seed nucleotide. Thirty sequences were simulated by randomly sampling 8 mers from the distribution 8 mers in 3′ UTRs and inserting an instance of the reverse complement of the miR-1 seed sequence at varying proportions (0 to 100%). The reciever operating characteristic (ROC) area under the curve (AUC) was calculated using the ROCR package (Sing et al. 2005).

Assessing Bias in the Distribution of miRNA Binding Sites

Instances of Weeder motif binding sites from either full transcripts (5′ UTR, coding sequence (CDS), 3′ UTR) or just 3′ UTRs of genes matching to the perturbed miRNA were identified for the compendium of experimentally determined miRNA target gene sets. Significance for the normalized counts per 1 Kbp was calculated for the distribution of matches in each gene region and for each experimentally determined miRNA target gene set by comparison to 1,000,000 randomly sampled gene sets of the same size. A combined p-value was computed by using Stouffer's Z-score method. The ROCR package was again used to compute ROC curves and ROC AUCs for each method. The pROC package was used to calculate the 95% confidence interval and pairwise p-values to determine if there is a significant difference between the ROC curves of the methods (Robin et al. 2011).

Identifying Enriched Predicted miRNA Binding Sites

The PITA, TargetScan, miRanda and miRSVR miRNA target gene prediction databases were downloaded from their respective web sites. The significance for enrichment of genes with a predicted miRNA binding site was calculated using the hypergeometric p-value for each miRNA. The miRNA(s) with the smallest hypergeometric p-value are considered the most likely to regulate the signature. Multiple hypothesis testing correction was applied using the BenjaminHochberg approach for controlling the false discovery rate (FDR) equal to or less than 0.001 (FDR<0.001), and requiring at least 10% of the genes to be targeted by the specific miRNA.

Selecting Optimal Methods to Infer miRNA Regulatory Network

Each inference method was applied to the compendium of 50 miRNA target gene sets (Supplementary Table 2). The ROCR and pROC packages in R were used to compute ROC curves, ROC AUC and p-values between ROC curves.

miR2Disease Overlap

First, we created a mapping between the 46 cancer subtypes and the disease classifications in the manually curated miR2Disease database. Instances were then identified where an inferred miRNA regulator was previously observed to be dysregulated or causal in the same cancer type. Significance of the enrichment of overlap between miR2Disease and the cancer-miRNA regulatory network was calculated using a hypergeometric p-value in R.

### Functional Enrichment and Semantic Similarity to Hallmarks of Cancer

Enrichment of GO biological process terms in each cancer co-expression signature were assessed using the topG0 package in R (Alexa et al. 2006) by computing a hypergeometric pvalue with Benjamini-Hochberg correction (FDR<0.05). All GO terms passing the significance threshold for a co-expression signature were included in downstream analyses. Semantic similarity between a significantly enriched GO term and each hallmark of cancer was assessed by using the Jiang and Conrath similarity measure as implemented in the R package GOSim (Fröhlich et al. 2007). For each co-expression signature the similarity scores between its enriched GO terms and the GO terms for each hallmark of cancer was computed, and the maximum for each hallmark was returned. Similarity scores gyeater than or equal to 0.8 were considered sufficient for inferring a link between the enriched GO terms for a co-expression signature and a hallmark of cancer. Random sampling of 1,000 GO terms and computing the Jiang and Conrath scores demonstrated that a similarity score greater than or equal to 0.8 resulted in a permuted p-value<5.1×10⁻⁴.

miR-29 Family Co-Expression Signature Overlaps

A hypergeometric p-value was used to test for significant overlap between the lung adenocarcinoma signature genes and the genes up-regulated by in vitro due to knock-down of miR-29 family milMAs.

### Luciferase Reporter Assay

The 3′ UTRs for genes of interest were amplified from cDNA (primers in Supplementary Table 12) and cloned into the pmirGLO Dual-Luciferase miRNA target expression vector behind firefly luciferase. The sequence and orientation for all 3′ UTRs inserted into pmirGLO were verified by sequencing. HEK293 cells were plated at a density of 100,000 cells per well and cotransfected in 96 well plates 24 hours after plating. Cells were transfected using DharmaFect DUO (Dharmacon) with 75 ng of the 3′ UTR fused reporter vector and either 50 nM of miR-29a, miR-29b, miR-29c, miR-767-5p or cel-miR-67 (negative control) miRNA mimic (Dharmacon). Twenty-four hours after transfection firefly and renilla luciferase activities were measured using the Dual-Glo assay (Promega) on a Synergy 114 hybrid multi-mode microplate reader (BioTek) per manufacturer recommendations. Experiments were conducted in biological triplicates. Luminescence measurements were first background subtracted using a vehicle only control, and then firefly luminescence was normalized to renilla luminescence. Experimental comparisons are made to vector only controls. Student's T-test and fold-changes were calculated using standard methods. MiRNA binding sites for MMP2 and SPARC were deleted using recombinant PCR (primers in Supplementary Table 12). Dose response curves for COL3A1 and SPARC were conducted using 50 nM, 5 nM, 500 pM, 50 pM and 5 pM miRNA mimic concentrations.

### Supplementary Methods

### De Novo Identification of 3′ UTR Motifs

Sequences and RefSeq gene definition files were downloaded from the UCSC genome browser FTP site (ftp://hgdownload.cse.ucsc.edu/goldenPath/currentGenomes/Homo sapiens). To reduce overlap the set of RefSeq genes that mapped to an Entrez gene were collapsed and the regulatory regions were merged to include all potential regulatory sequences. The RefSeq to Entrez gene mapping was downloaded from NCBI Gene FTP site (ftp://ftp.ncbi.nih.gov/gene/DATA/gene2refseq.gz). To provide a 3′ untranslated region (UTR) for as many genes as possible we set the minimum 3′ UTR length to the median annotated 3′ UTR length of 844 bp (Kertesz et al. 2007). The same approach was used for the 5′ UTR with a minimum 5′ UTR length of 183 bp. The coding sequences were acquired as they were annotated, and were not filtered in anyway. All annotated introns were removed as they are present only transiently in expressed transcripts. The Weeder de novo motif detection algoirthm (Pavesi et al. 2006) was then used to identify over-represented miRNA binding sites in the 3′ UTR of putatively miRNA co-regulated genes (Fan et al. 2009; Linhart et al. 2008).

miRvestigator Hidden Markov Model (HMM) from Position Specific Scoring Matrix

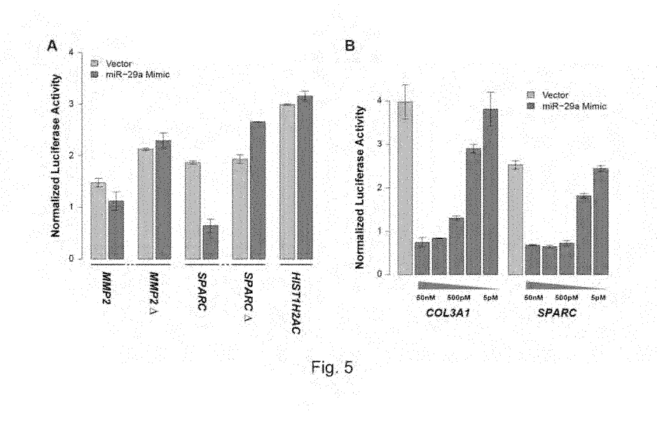

Two general problems are faced when comparing an miRNA seed which is a string of nucleotides 8 base pairs long (and may be complementary for 6, 7 or 8 base pairs) to a PSSM (a matrix of 4 nucleotide probabilities that must sum to 1 in a column by a variable number of columns). First the miRNA seed sequence must be aligned to the PSSM, and second the certainty of the match between the miRNA seed and the PSSM must be computed. The Viterbi algorithm identifies the optimal path through an HMM for an observed sequence of events, and there can solve both of these problems simultaneously by turning the PSSM into an Hidden Markov Model (HMM) and the miRNA seed nucleotide sequence into the observed sequence of events. The overall structure of the miRvestigator HMM is described in FIG. 5. Each column n of the PSSM is converted into a hidden state PSSMₙ which emits the nucleotides A, G, C and T with the probability of each nucleotide in the PSSM column. There are also two non-matching states NM₁ and NM₂, which are used to buffer entry and exit respectively to and from the PSSM. The non-matching states emit nucleotides at a random frequency of 0.25 for each nucleotide, thus not favoring any nucleotide over another. This buffering allows for non-matching states at the start and end of the aligned seed to the PSSM, and do not allow for gapping. From the start state the transmission probability is evenly distributed to each PSSMₙ state and the NM₁ state (1/(length of PSSM+1)). This allows the alignment to start with equal probability at any point in the miRvestigator HMM. If the alignment starts with NM₁ the transition probability back to NMᵢ is 0.01 and the transition to the next PSSM column state is 0.99. The transition between PSSMₙ column state and PSSMₙ₊₁ column state is 0.99, and 0.01 to the end buffering NM₂ non-matching state. The last PSSM_{N} state transitions to the end state with a probability of 1. The NM₂ non-matching state transitions to itself and the end state with a probability of 1, therefore when an alignment transitions to the NM₂ state it stays there till it transitions to the end state. The emitted observations are the miRNA seed sequence being fed into the miRvestigator HMM. The output from the Viterbi algorithm is the optimal state path (a path made up of the PSSMₘ, NM₁, NM₂, WOBBLEₙ states) through the mirvestigator HMM given the miRNA seed nucleotide sequence and a probability for this optimal alignment.

### Significance of the Viterbi Optimal State Path Probability

The significance of a the Viterbi optimal state path probability for a given miRNA is then calculated by exhaustively computing the complete distribution of Viterbi optimal state path probabilities for all potential miRNA k-mer seed sequences (where k=6, 7 or 8 base pairs). Only k-mers which are present in the regulatory regions of the transcripts being investigated are included in the exhaustive computation. The complete distribution of Viterbi probabilities is then used to provide a p-value for each miRBase miRNA seed sequence by counting the number of k-mers with a Viterbi optimal state path probability greater than or equal to the miRNA seed of interest divided by the total number of potential k-mers. This provides a p-value for the alignment and match for each miRNA seed sequences to a PSSM identified from cis-regulatory regions. The miRNAs are then ranked based upon the Viterbi optimal state path p-values and the miRNA(s) with the smallest p-values is the most likely to regulate the set of transcripts.

Modeling Wobble Base-Pairing with miRvestigator HMM

Wobble base-pairing was included in the miRvestigator HMM for the case where a G=U wobble base-pairing defines the miRNA to protein coding transcript complementarity (Baek et al. 2008; Guo et al. 2010; Hendrickson et al. 2009; Selbach et al. 2008). The individual miRNA to protein coding transcript G=U wobble base-pairing is a problem that will need to be solved at the level of de novo motif identification. A wobble base-pairing state is added to the model only if a G and/or U have a nucleotide seed frequency of 25%. For the case where the G seed nucleotide frequency is greater than 25% and the U seed nucleotide frequency is below 25% the wobble state emits the nucleotide A with a probability of 1. For the case where the U seed nucleotide frequency is greater than 25% and the G seed nucleotide frequency is below 25% the wobble state emits the nucleotide C with a probability of 1. For the case where both the G and U seed nucleotide frequencies are greater than 25% the wobble state emits A and C with a probability of 0.5. When a wobble state is added the transition probability from the PSSMₙ state to the WOBBLEₙ₊₁ state is set to 0.19, the transition probability from the PSSMₙ state to the PSSMₙ₊₁ state is set to 0.8, and the transition probability from the PSSMₙ state to the NM₂ state remains at 0.01. The transition probability from the wobble state WOBBLEₙ to PSSMₙ₊₁ is set to 1, which precludes a wobble base-pairing at the terminus of a state path for either transitioning to the NM₂ state or to the end state.

### Example 1

Inferring miRNA Mediated Regulation through Analysis of Co-Expressed Genes

The inference of a miRNA regulatory network can be accomplished in two ways. The first approach requires prior knowledge of genome-wide binding site locations for known miRNAs (Sethupathy et al. 2006). There are many algorithms that utilize this target enrichment strategy for inference of miRNA regulatory networks (Betel et al. 2010; Grimson et al. 2007; Linhart et al. 2008). The second approach performs the de novo discovery of conserved putative miRNA-binding sites within the 3′ UTRs of co-expressed genes. Weeder is one such algorithm that accurately discovers conserved cis-regulatory elements in 3′ UTRs (Fan et al. 2009; Linhart et al. 2008). The information of conserved cis-regulatory sequences can then be utilized for pattern matching to seed sequences of known miRNAs in miRBase. We had previously reported a web framework using the miRvestigator algorithm for performing such pattern matching (Plaisier et al. 2011). Here, we present results on the performance of Weeder and miRvestigator applied to simulated datasets. We then utilize a compendium of experimentally generated data from targeted miRNA perturbation studies to demonstrate that restricting Weeder's search space to 3′ UTRs sequences increases the sensitivity and specificity of Weeder-miRvestigator. Finally, we use the compendium to compare the performance of algorithms for the inference of miRNA regulation and combine the optimal methods into an integrated framework.

### Weeder-miRvestigator

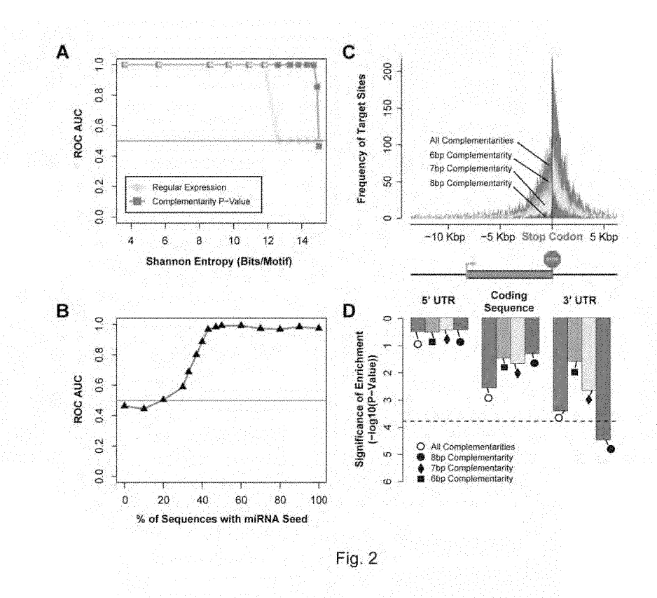

We constructed a framework for accurate inference of miRNA-mediated regulation using as input just the 3′ UTR sequences of co-expressed genes by coupling Weeder de novo motif detection and miRvestigator for subsequent association to known miRNA seeds (FIG. 1). We tested the sensitivity and specificity of miRvestigator independent of Weeder using synthetic 3′ UTR motifs. Starting with the seed sequence of miR-1 we computationally generated a set of synthetic motifs with increasing entropy. Using these synthetic motifs we computed the receiver operating characteristic (ROC) area under the curve (AUC) across a range of motif entropies. The ROC AUC is a standard approach to evaluate the sensitivity and specificity of classification or feature selection by an algorithm. This statistical analysis demonstrated that the miRvestigator scoring function (complementarity p-value metric) outperforms regular expression in both sensitivity and specificity for higher entropies (FIG. 2A, Supplementary Methods). Using the same approach we tested the performance of the integrated Weeder-miRvestigator framework in recovering the miR-1 seed sequence from a set of synthetic sequences into which it was inserted at a known frequency (0 to 100%). The results showed that by integrating the two algorithms we can sensitively and specifically recover the complementary miRNA seed (ROC AUC-0.9) even when it is present in just 40% of the query sequences (FIG. 2B). We conclude from these experiments that the integrated Weeder-miRvestigator approach is a sensitive and specific method for inference of miRNA mediated regulation from 3′ UTRs of coregulated genes.

### Example 2

Restricting Searches to 3′ UTR Increases Sensitivity and Specificity of WeedermiRvestigator

MiRNA target prediction algorithms (including PITA, TargetScan, miRANDA, and miRSVR) improved their performance by restricting searches to the 3′ UTRs of transcripts where it has been demonstrated statistically that functional miRNA binding sites are preferentially located (Grimson et al. 2007). To determine the validity of this heuristic we investigated the distribution of functional miRNA binding sites within co-regulated transcripts by applying Weeder-miRvestigator to full transcript sequences (5′ UTR, coding sequence (CDS) and 3′ UTR). First, we compiled a compendium of miRNA target gene sets from 50 transcriptomes that were generated by perturbing specific miRNAs (22 independent studies, 41 unique mIRNAs, Supplementary Table 2). The analysis was then restricted to target gene sets in the compendium where Weeder-miRvestigator was able to identify the corresponding perturbed miRNA (27 of 50 sets). The 3′ UTRs were significantly enriched for miRNA-binding sites with 8 bp complementarity to the miRNA seed sequence (p-value=3.2×10-5, FIGS. 2C and D). Remarkably, none of the other transcript regions showed significant enrichment of miRNA-binding sites (p-value >1.5×10-4, p-value corrected for 27 miRNAs ×3 transcript regions ×4 instance complementarities to the miRNA seed (All, 8 bp, 7 bp and 6 bp complementarities)). This unbiased analysis has independently confirmed the observation of Grimson, et al. that functional miRNA binding sites preferentially reside in the 3′ UTRs. Next, we compared the sensitivity and specificity of searching full transcripts versus restricting the search space to the 3′ UTRs by computing ROC curves for Weeder-miRvestigator. Restricting the search space to 3′UTRs (ROC AUC=0.96) significantly increased the sensitivity and specificity of miRNA-binding site discovery by Weeder (p-value=1.8×10-2) relative to corresponding searches on full transcript sequences (ROC AUC=0.80). Therefore, all subsequent miRNA-binding site searches with Weeder were restricted to the 3′ UTR of putatively co-regulated gene sets.

### Example 3

Selecting Optimal Methods to Infer a Comprehensive miRNA Regulatory Network

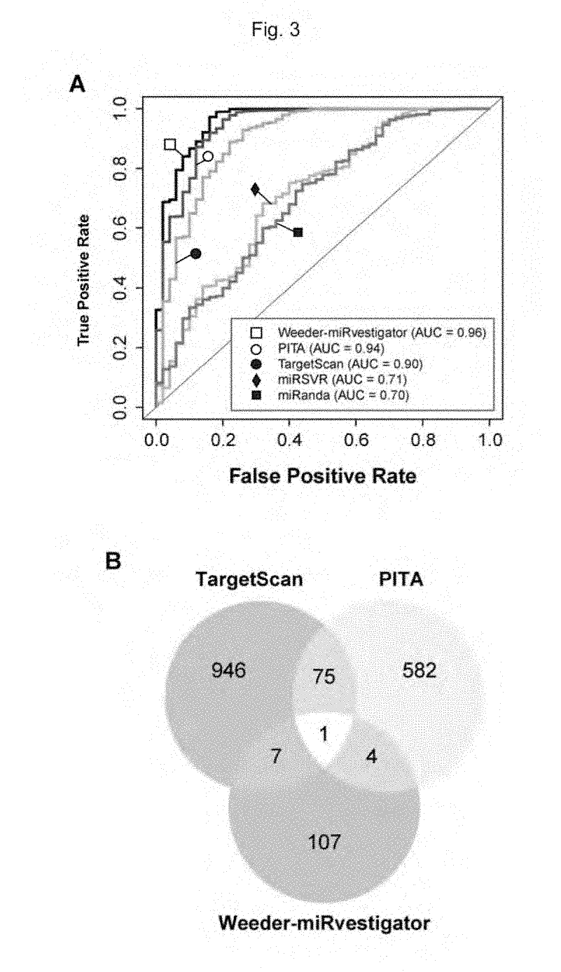

While multiple hypotheses testing correction procedures can reduce the number of false positives (incorrectly inferred regulatory interactions), it also results in a higher false negative rate (i.e. missing regulatory interactions). Therefore, we hypothesized that integrating results from multiple inference methods would construct a more comprehensive cancer-miRNA regulatory network as each method identifies a different subset of the miRNA regulatory network. To assess this we first identified the best performing network inference methods by computing a ROC curve from the predictions of applying each method to the compendium of experimentally determined miRNA target gene sets. In addition to Weeder-miRvestigator, we tested four additional algorithms that infer miRNA regulation through enrichment of predicted binding sites in 3′ UTRs of co-expressed genes: PITA, TargetScan, miRanda and miRSVR. This comparative analysis demonstrated that Weeder-miRvestigator, PITA and TargetScan are the best performing algorithms for inference of miRNA mediated regulation (FIG. 3A; ROC AUC±95% confidence interval=0.96±0.03, 0.94±0.04 and 0.90±0.05, respectively; Supplementary Table 3). Using cancer as an example, we explain in subsequent sections how the integration of these three best performing algorithms provides a generalizable framework for inference of regulation by miRNAs (FIRM) to infer comprehensive miRNA regulatory networks for complex diseases.

### Constructing a Cancer-miRNA Regulatory Network Using FIRM

A previous study published by Goodarzi, et al. analyzed transcriptome profiles from 46 different cancers and identified 2,240 cancer-subtype characteristic co-expression signatures. Interestingly, the authors were able to associate only four of these signatures to regulation by a specific miRNA in miRBase (Goodarzi et al. 2009). We analyzed these co-expression signatures using FIRM with the intent of constructing a comprehensive cancer-miRNA regulatory network. Weeder-miRvestigator, PITA and TargetScan predicted miRNA regulators for 119, 662 and 1,029 co-expression signatures, respectively (Weeder-miRvestigator criteria: perfect 7-mer or 8-mer match, FDR<0.05, Supplementary Table 4; PITA and TargetScan criteria: FDR<0.001 and enrichment>10%, Supplementary Tables 5 and 6, respectively). There was significant overlap in pairwise comparisons of predictions for the same cancer (Weeder-miRvestigator vs. PITA=0.045, Weeder-miRvestigator vs. TargetScan=0.019 and PITA vs. TargetScan=7.4×10⁻²²; FIG. 3B). While this significant overlap demonstrates concordance across the methods, a large fraction of the inferred miRNA regulation was unique to each method. This is not surprising given the high false negative rates of these methods and the different principles they use for identifying miRNA mediated regulation. In other words, predictions made by the three algorithms are mostly complementary. Combining results from all three methods in FIRM resulted in the construction of a comprehensive miRNA regulatory network that links 1,324 co-expression signatures to post-transcriptional regulation mediated by 608 miRNAs (Supplementary Table 7). Within this network 443 co-expression signatures were associated to miRNAs by more than one algorithm. Twenty co-expression signatures were independently associated to the same miRNA by two different algorithms (Supplementary Table 7). Interestingly, the only prediction that was consistent across all algorithms was that the miR-29 family regulates genes whose co-expression is observed in lung adenocarcinoma. In the following sections we investigate which miRNAs regulate oncogenic processes and the degree to which this network recapitulates known dysregulation of miRNAs in miR2Disease.

The Cancer-miRNA Network Recapitulates miR2Disease and Discovers miRNAs that are Causal in Cancers

We investigated whether the cancer-miRNA regulatory network was able to recapitulate miRNAs that are both dysregulated in tumors and causally linked to specific oncogenic processes. We performed this analysis by comparing the cancer-miRNA network to entries in miR2Disease, a manually curated database of miRNAs that are dysregulated and causally associated with 163 human diseases, including the 46 cancers in our study. Remarkably, there was significant enrichment of known dysregulated miRNAs in the cancer-miRNA network. Altogether 191 putative miRNA regulators in our inferred network were previously shown to be dysregulated in patient tumors of the same cancer type (p-value=2.1×10⁻⁹¹, Supplementary Table 7). Importantly, there were significant overlaps with predictions by each of the three algorithms (Weeder-miRvestigator p-value=0.029, PITA p-value=7.4×10⁻²³ and TargetScan p-value=1.1×10⁻³²). This result further demonstrates the value of combining the three algorithms in FIRM to infer a more comprehensive miRNA regulatory network.

Using miR2Disease, we further investigated whether the dysregulated miRNAs predicted by FIRM were also known to causally influence cancer phenotypes. It was striking that over a third of the putative miRNA regulators that were dysregulated were also known to causally affect cancer phenotypes (66 miRNAs, p-value=1.4×10⁻³⁴, Supplementary Table 7). Among these, three of the most highly connected miRNAs (miR-29b, miR-200b and miR-296-5p) were dysregulated in at least 8 cancers and causal in at least 4 cancers. These results demonstrate that the network inferred by FIRM had captured disease-relevant miRNA regulation of cancer. It also suggests that the network contains novel testable hypotheses regarding the role of miRNAs in regulation of cancer beyond what is documented in miR2Disease. A key next step is the prioritization of these novel testable hypotheses by integrating orthogonal information.

Identifying miRNAs Regulating the Hallmarks of Cancer

Associating a miRNA to a co-expression signature in patient tumors does not by itself implicate it in the regulation of key oncogenic processes. However, the network enables the discovery of cancer-relevant miRNAs through analysis of target genes for functional enrichment of one or more hallmarks of cancer (Douglas Hanahan and Robert A Weinberg 2011; D Hanahan and R A Weinberg 2000): 1) “self sufficiency in growth signals”, 2) “insensitivity to antigrowth signals”, 3) “evading apoptosis”, 4) “limitless replicative potential”, 5) “sustained angiogenesis”, 6) “tissue invasion and metastasis”, 7) “genome instability and mutation”, 8) “tumor promoting inflammation”, 9) “reprogramming energy metabolism”, and 10) “evading immune detection”. We analyzed genes within each of the co-expression signatures for hallmarks of cancer through their associations to specific Gene Ontology (GO) biological process terms.

In total 627 of the 2,240 co-expression signatures were significantly enriched for GO terms (FDR<0.05), and 314 were associated with a putative miRNA in the regulatory network (Supplementary Table 8). To further filter this set and discover specific co-expression signatures associated with oncogenesis, we manually curated the lowest level GO terms for each of the 10 hallmarks of cancer (Supplementary Table 9), e.g. the hallmark of cancer “Evading Apoptosis” is associated with the GO term “Positive Regulation of Anti-Apoptosis”. Based on semantic similarity between GO terms we then associated 158 of the 314 putatively miRNA regulated co-expression signatures to one or more hallmarks of cancer (Jiang-Conrath Semantic Similarity Score>0.8, permuted p-value<5.1×10-4, Supplementary Table 8).

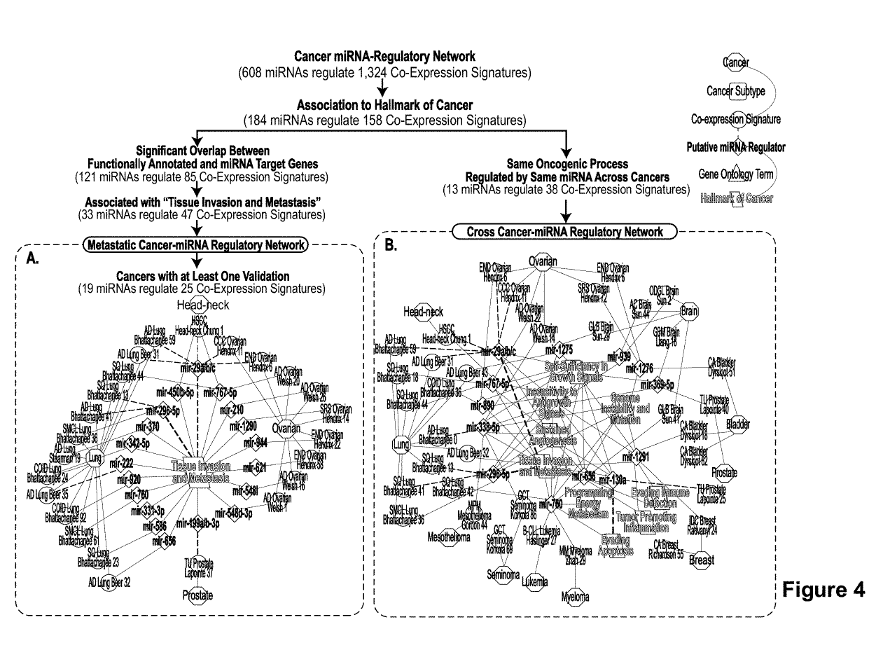

Metastatic potential is one of the defining features of malignant tumors making putative miRNA-regulators of “tissue invasion and metastasis” excellent biomarker candidates. As an initial filter we selected 85 of the 158 “hallmarks of cancer”-associated co-expression signatures that had significant overlap (p-value<0.05) between GO annotated- and putatively miRNAregulated genes. Next, we extracted from these 85 co-expression signatures a subnetwork of 33 miRNAs and their predicted regulatory influences on 47 co-expression signatures associated with “tissue invasion and metastasis”—i.e. the metastatic cancer miRNA-regulatory network (FIG. 4A, Supplementary Table 10). Notably, at least three miRNAs, miR-29a/b/c, miR199a/b-3p and miR-222 are known to be differentially expressed in the cancer type predicted by this subnetwork. While some of these prior studies had independently revealed phenotypic consequences of perturbing the miR-29 family on tumor invasiveness, FIRM proposes a mechanistic explanation by predicting that these miRNAs directly regulate specific genes involved in “tissue invasion and metastasis”. We have performed detailed experimental validations demonstrating the regulation of metastasis associated genes by the miR-29 miRNAs and results of these experiments are presented in a later section.

A Relatively Small Subset of miRNAs Regulate Oncogenic Processes in Diverse Cancers

Regulation of the same oncogenic process by the same miRNA across different cancers reinforces the likelihood that the inferred miRNA regulation is real. In the cancer-miRNA regulatory network the number of co-expression signatures regulated by a miRNA follows a power-law distribution (y=2.1±0.0; goodness of fit p-value<1.0×10-4) with each miRNA predicted to regulate on average 3.3±3.3 co-expression signatures (Barabasi and Albert 1999). This suggests that some miRNAs regulate common biological processes across multiple cancers. Therefore, we filtered the cancer-miRNA regulatory network for miRNAs predicted to regulate genes within two or more co-expression signatures enriched for the same GO term(s). This analysis recovered 24 miRNAs that were predicted to combinatorially regulate 74 non-redundant co-expression signatures. Again, using semantic similarity to the hallmarks of cancer we discovered a subnetwork of 38 co-expression signatures from 30 cancer types that are regulated by 13 highly connected miRNAs (miR-29a/b/c, miR-130a, miR-296-5p, miR-338-5p, miR-369-5p, miR-656, miR-760, miR-767-5p, miR-890, miR-939, miR-1275, miR-1276 and miR-1291)—i.e. a cross-cancer-miRNA regulatory network (FIG. 4B, Supplementary Table 11). Each of the 13 miRNAs putatively regulates the same oncogenic processes across two or more cancers (FIG. 4B). We have already discussed role of miR-29 family in regulation of “tissue invasion and metastasis”. Further, reversing down regulation of miR-130a in metastatic prostate cancer cell lines has been previously demonstrated to increase apoptosis (Boll et al. 2012). This independently validates the cancer-miRNA regulatory network predicted effect of miR-130a on “evading apoptosis”. Finally, the predicted role of miR-296-5p in “activating invasion and metastasis” has also been validated by an independent study that discovered down-regulation of this miRNA in metastases relative to primary tumors (Vaira et al. 2011). Notably, 5 of the 13 miRNAs (hsa-miR-29a/b/c, miR-296-5p, miR-760, miR-767-5p and miR-1276) were inferred for co-expression signatures where a significant fraction of genes are direct miRNA targets and have GO annotated functions in oncogenic processes (FIG. 4A). It is noteworthy that such filtering is too stringent and would have excluded known cancer-related miRNAs such as miR-130a. Therefore, the integration of co-expression, shared miRNA-binding sites, and GO annotations, together overcome the incompleteness and uncertainties across all of these orthogonal datasets to discover novel biologically-meaningful regulation by miRNAs. Thus, we contemplate that all of the 13 miRNAs are useful as general purpose cancer biomarkers.

Extracellular Matrix Genes Co-Regulated by miR-29 Family in Lung Adenocarcinoma

In both the metastatic and cross-cancer-miRNA regulatory network, the miR-29 family (miR-29a, miR-29b and miR-29c) was predicted to be responsible for 8 co-expression signatures, five of which were associated with four hallmarks of cancer, viz. “tissue invasion and metastasis”, “sustained angiogenesis”, “insensitivity to anti-growth signals” and “self sufficiency in growth signals” (FIG. 4A and 4B). Two of these co-expression signatures were from lung adenocarcinoma patient tumors, “AD Lung Beer 31” and “AD Lung Bhattacharjee 59” (Bhattacharjee et al. 2001; David G Beer et al. 2002). The miR-29 family was associated to the co-expression signature from “AD Lung Beer 31” by all three inference methods; on the other hand, only PITA picked miR-29 as the putative regulator responsible for the co-expression signature from “AD Lung Bhattacharjee 59”.

Two independent studies demonstrated that over-expression of miR-29a reduces the invasiveness of lung carcinoma cell lines (Muniyappa et al. 2009) and knock-down of miR-29b increases invasiveness (Rothschild et al. 2012). Serving as independent validation of the network predicted role of miR-29 family as regulators of “activating invasion and metastasis” in lung cancer. The direction of this association is concordant with a different set of studies which independently discovered that miR-29 family members were down-regulated in lung adenocarcinomas relative to normal lung (Landi et al. 2010; Yanaihara et al. 2006). Taken together these orthogonal sets of results strongly suggest that down-regulation of the miR-29 family increases tumor invasiveness thereby decreasing patient survival (Rothschild et al. 2012).

A major strength of the cancer-miRNA regulatory network is that it identifies specific genes that are directly regulated by a specific miRNA. For instance, miR-29 family is implicated in modulating metastatic potential of patient tumors because it is predicted to directly regulate 79 and 64 genes in two co-expression signatures—“AD Lung Beer 31” and “AD Lung Bhatacharjee 59”. Notably, the two co-expression signatures have a significant overlap of 32 genes (p-value=2.1×10⁻⁴⁶). We assessed whether these genes were indeed targets for regulation by the miR-29 family by investigating if they were differentially regulated when endogenous miRNAs of the miR29 family were knocked-down in a fetal lung fibroblast cell line (Cushing et al. 2011). Sixteen genes from “AD Lung Beer 31”, and 9 genes from “AD Lung Bhattacharjee 59” were up-regulated in response to knock-down of the three miR-29 family members (p-values=6.1×10⁻¹⁴ and 1.5×10⁻⁸, respectively). Altogether 17 genes from both co-expression signatures were up-regulated in the Cushing et al. study (Table 1), and notably all of these genes contain one or more miR-29 family binding sites in their 3′ UTRs (Table 1).

Differential regulation of the seventeen genes in the Cushing et al. study does not demonstrate direct regulation by miR29 family miRNAs through physical interaction with predicted binding sites within 3′ UTRs of these genes. However, it is possible to demonstrate direct miRNA regulation by fusing the 3′ UTR of each putative target gene to a luciferase reporter, selectively deleting specific binding sites, and performing luciferase assays in cell lines that are co-transfected with the wildtype or mutated reporter-fusion construct and the synthetic miRNA mimic (at different concentrations) (Lal et al. 2011). We selected a total of 8 genes (COL3A1, COL4A1, COL4A2, FBN1, PDGFRB, SERP1NH1, and SPARC—see Table 1) to investigate using the aforementioned luciferase assay whether they were direct targets for regulation by miR29 family miRNAs (miR-29a, miR-29b and miR-29c). These genes were selected because they were predicted by the FIRM methods to (i) be in co-expression signatures regulated by the miR-29 family, (ii) contain miR-29 family binding sites, (iii) have functional association to “tissue invasion and metastasis” (e.g. collagens, metallo-proteases, etc.), and (iv) be up-regulated by miR-29 family knock-down in lung fibroblasts in the Cushing et al. study.

First, we used qRT-PCR to demonstrate that the miR-29a mimic significantly down regulates transcript levels of luciferase when it is fused to 3′ UTRs of either COL3 A1 or SPARC (COL3A1 p-value=3.2×10⁻², fold-change=−3.9; SPARC p-value=4.2×10⁻², fold-change=−1.7). This validates our central thesis that perturbing a miRNA results in observable changes in transcript levels of the predicted target transcripts with corresponding miRNA-binding sites in the 3′ UTR. We then assayed the effects of all three miR-29 mimics (miR-29a, miR-29b and miR-29c) on normalized luciferase activity relative to a control (i.e. no miRNA mimic). Significant reduction in normalized luciferase expression (p-value<0.05) was observed for 7 of the 8 genes tested (Table 2), and there was no consequence when luciferase was fused to the negative control 3′ UTR from HIST1H2AC (miR-29a: p-value=0.99, fold-change=1.2). Deletion of all the putative miR-29 binding sites from the 3′ UTRs of MMP2 and SPARC abolished down regulation of luciferase activity by the miR-29 family mimics, conclusively demonstrating that miR-29 directly regulates abundance of predicted target transcripts via binding to the predicted 3′ UTR sites (MMP2-deletion: 1 site deleted, fold-change=1.1, p-value=8.6×10⁻¹; SPARC-deletion: 2 sites deleted, fold-change=1.4, p-value=1.0; FIG. 5A).

Finally, titration of the miR-29a mimic demonstrated it down regulates COL3A1 and SPARC in a dose-dependent manner (FIG. 5B).

miR-767-5p Regulates a Collagen-Specific Subset of miR-29 Target Genes

Analysis of predicted regulation by miR-29 demonstrates that the cancer-miRNA regulatory network makes accurate predictions that can be validated experimentally through a combination of miRNA perturbation and targeted mutagenesis of specific binding sites in the 3′ UTRs. We conducted further experimental analysis of predicted regulation by miR-767-5p to assess the specificity of using FIRM inferences to identify genes regulated by a miRNA. We selected miR-767-5p because this miRNA partially shares the miR-29 seed sequence. Specifically, both the metastatic and cross cancer-miRNA regulatory networks contain the PITA predictions that miR-767-5p regulates genes associated with four hallmarks of cancer (“insensitivity to antigrowth signals”, “self sufficiency in growth signals”, “sustained angiogenesis” and “tissue invasion and metastasis”) from four co-expression signatures (AD Ovarian Welsh 20, HSCC Head-Neck Chung 1, and SQ Bhattacharjee 18 and 44) across 3 cancer types (Bhattacharjee et al. 2001; Chung et al. 2004; Welsh et al. 2001).

Unlike the miR-29 family, miR-767-5p has not been previously associated with any oncogenic processes. Therefore, we first evaluated whether there is any evidence for expression of miR-767-5p in head and neck, lung, or ovarian cancers to support the prediction by the cancer-miRNA regulatory network. A scan of miRNA-seq data from The Cancer Genome Atlas (TCGA) shows that miR-767-5p is indeed expressed in lung squamous cell carcinoma, head and neck squamous cell carcinoma, and ovarian serous cystadenocarcinoma (data not shown). Additionally, the MirZ miRNA expression atlas identifies miR-767-5p expression in astrocytoma, osteosarcoma and teratocarcinoma cell lines (Hausser et al. 2009). Future studies with the completed TCGA data will be able to determine whether miR-767-5p is differentially expressed between tumor and normal and whether miR-767-5p is predictive of patient survival. Based on this evidence we proceeded to test the effect of perturbing miR-767-5p on transcript abundance of the PITA predicted targets. Over-expression of miR-767-5p using a miRNA mimic led to significant reduction (p-value<0.05) in the normalized luciferase activity for 3 of the 4 predicted miRNA target genes (COL3A1, COL5A2, COL10A1 and LOX; Table 2).

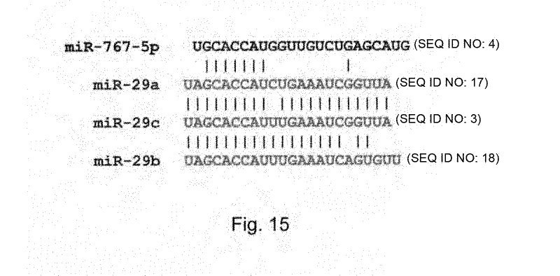

In addition to validating a novel oncogenesis-associated miRNA, the aforementioned rationale for selecting miR-767-5p was that it also shares 6 bp of similarity to the 8 bp seed region of the miR-29 family leading to a significant overlap between their predicted target genes (65% for PITA and 35% for TargetScan). This may explain why miR-767-5p and the miR-29 family are both predicted regulators of the HSCC Head-Neck Chung 1 co-expression signature. However, the two seed sequences have little similarity in the 3′ region (FIG. 15). The partial overlap in the miRNA seeds and their predicted targets provides an opportunity to test the specificity of using FIRM inferences to identify genes regulated by a miRNA. First, we tested all 11 3′ UTR luciferase fusions by over-expressing miR-29a, miR-29b, and miR-29c and miR-767-5p. Of the 22 regulatory interactions tested (Table 2) we observed only 1 false positive (miR-767-5p did not affect LOXtranscript levels) and 2 false negatives (the cancer-miRNA network did not predict the experimentally observed regulation of COL4A2 by miR-767-5p, and regulation of COL10,41 by the miR-29 family). Thus the false discovery rate was 7.1% -a significant improvement over previously published estimates (Sethupathy et al. 2006). Consistent with the cancer-miRNA network predictions, of the 11 genes that were tested only the five collagens were significantly regulated (p-value<0.05) by both miR-767-5p and miR-29 family. Despite sharing 6 bp of similarity in the seed sequence, miR-767-5p had no effect on transcript abundance of the other six bona fide miR-29 family targets to underscore the specificity of the cancer-miRNA regulatory network predictions filtered through FIRM.

### Example 4

The FIRM approach was used to identify miRNAs regulating a number of hallmarks of cancer as described above as well as additional hallmarks of cancer. The miRNAs associated with additional hallmarks of cancer are set out in FIG. 14 along with their particular tissues and cancer types.

### Example 5

### Discussion

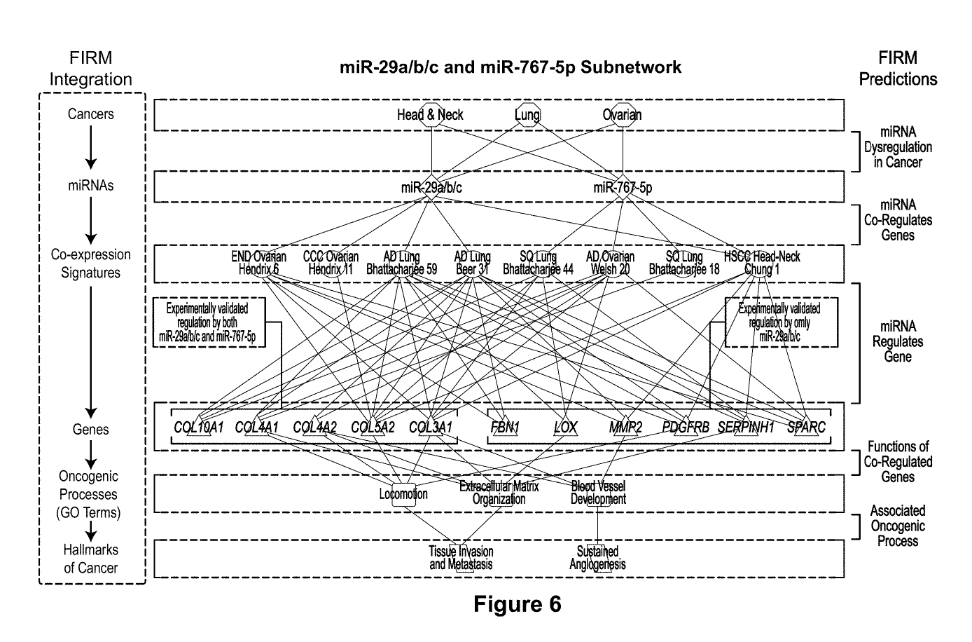

As genome-wide analyses for discovery of molecular signatures of complex disease becomes routine it is imperative that these data are integrated into predictive and actionable models that drive targeted hypothesis-driven discovery of diagnostics, prognostics and, ultimately, therapeutics. The systems integration of disparate kinds of information boosts signal to noise enabling the discovery of biologically meaningful patterns as we have demonstrated here through inference of a cancer miRNA regulatory network. The success of the FIRM approach depended not only on integration of three best performing algorithms that use complementary strategies for inference of miRNA regulatory networks, but also on the integration of disparate data types such as gene co-expression, and distributions of both known and de novo discovered miRNA binding sites (FIG. 6). This is a remarkable achievement given that the information for miRNA binding and regulation exists in a contiguous stretch of merely 6-8 nucleotides located within the expansive 3′ UTRs of >20,000 genes in a genome of 6 billion bps.

Further, we have also demonstrated that by incorporating the mechanistic basis of miRNA regulation, i.e. binding to complementary sequences in the 3′ UTRs of co-expressed genes, the network can be more easily assayed with targeted experimental and functional evaluation. In doing so we were able to demonstrate that the cancer-miRNA regulatory network had captured a significant proportion of known miRNA dysregulation and their causal influence on cancer phenotypes. In fact the network also made specific experimentally testable novel predictions regarding the role of 158 miRNAs in mediating co-expression of genes associated with oncogenic processes. Among these were 33 miRNAs that were predicted to regulate metastatic processes including a core set of 13 miRNAs that were predicted to regulate the same set of oncogenic processes across different cancer types. Our focused investigation of the role of miR-29 family in promoting metastasis in lung adenocarcinoma demonstrates how these network predictions can drive discovery of new biology.

As a generalizable framework for inferring miRNA mediated regulation, FIRM will also benefit from simultaneous measurement of changes in miRNA and mRNA levels in patient tumors. However, negative correlation with gene expression changes alone does not accurately identify bona fide targets for the miRNA (Tsunglin Liu et al. 2007; Ritchie et al. 2009; Liang Wang et al. 2009). Thus clustering of the gene expression data and subsequent analysis with FIRM will be necessary for the inference of accurate miRNA regulatory networks. Correlation with the putative miRNA regulators could be used post hoc as a secondary screen to filter the predicted list of targets, and prioritize miRNAs for further experimental validation. We have demonstrated the power of this approach by performing targeted experiments to test predictions from the cancer-miRNA regulatory network. These experiments have discovered novel regulation of specific oncogenesis-associated genes by miRNAs that are shared across different cancer types. Importantly, in addition to providing mechanistic linkages between a known tumor suppressor miRNA (miR-29) and regulation of specific genes with metastatic potential, we have also discovered a novel oncogenesis associated miRNA (miR-767-5p). The choice of miRNAs for validating network predictions has also helped to highlight the sensitivity and specificity of FIRM performance. As such, we have not only demonstrated the extraordinary value of the cancer-miRNA network in cancer research; but also the power of FIRM to construct from easily generated gene expression data similar miRNA regulatory networks for any disease.

We contemplate integrating inference of miRNA regulation into the clustering procedure. This will act as a constraint for accurate discovery of genes co-regulated by the same miRNA. The cMonkey biclustering algorithm already incorporates de novo discovery of transcription factor binding sites within gene promoters to limit the space of gene-gene associations to accurately discover sets of genes that are regulated by the same transcription factor (Reiss et al. 2006). The incorporation of constraints based on mechanisms of miRNA regulation will greatly improve the ability of cMonkey to model eukaryotic transcriptional regulatory networks. We contemplate that the ability of cMonkey to discover conditional coregulation of genes increases the sensitivity of FIRM and also provides the context (disease type, stage of progression, etc.) for regulatory influence of a miRNA.

Availability of miRvestigator, FIRM and Cancer-miRNA Regulatory Network

MiRvestigator was developed as an open source project using the Python programming language and is available both as a web service (http://mirvestigator.systemsbiology.net) and as source code (http://github.com/cplaisier/miRvestigator) (Plaisier et al. 2011). The FIRM and cancer-miRNA regulatory network are freely available at http://cmrn.systemsbiology.net

### Data Access

To facilitate reader access and usability we have developed and hosted a freely available website (http://cmrn.systemsbiology.net) containing: 1) all data contained within the cancer-miRNA regulatory network, 2) including the compendium of 50 experimentally defined miRNA target gene sets, and 3) the FIRM framework to infer miRNA regulatory networks from gene coexpression information. Our hope is that this will provide cancer researchers with a usable interface to explore the cancer-miRNA regulatory network, computational biologists with a valuable resource to compare methods of inferring miRNA mediated regulation, and researchers with the tools to infer miRNA regulatory networks for their disease of interest.

While the present invention has been described in terms of various embodiments and examples, it is understood that variations and improvements will occur to those skilled in the art. Therefore, only such limitations as appear in the claims should be placed on the invention.

All documents referred to in this application, including priority documents, are hereby incorporated by reference in their entirety with particular attention to the content for which they are referred.

### REFERENCES

Alexa A, Rahnenfiihrer J, and Lengauer T. 2006. Improved scoring of functional groups from gene expression data by decorrelating GO gaph structure. Bioinformatics 22: 1600-1607.

Baek D, Villén J, Shin C, Camargo F D, Gygi S P, and Bartel D P. 2008. The impact of microRNAs on protein output. Nature 455: 64-71.

Barabasi, and Albert. 1999. Emergence of scaling in random networks. Science 286: 509-512.

Bartel D P. 2009. MicroRNAs: target recognition and regulatory functions. Cell 136: 215-233.

Beer D G, Kardia SLR, Huang C-C, Giordano T J, Levin A M, Misek D E, Lin L, Chen G, Gharib T G, Thomas D G, et al. 2002. Gene-expression profiles predict survival of patients with lung adenocarcinoma. Nat. Med. 8: 816-824.

Betel D, Koppal A, Agius P, Sander C, and Leslie C. 2010. Comprehensive modeling of microRNA targets predicts functional non-conserved and non-canonical sites. Genome Biol. 11: R90.

Betel D, Wilson Manda, Gabow A, Marks D S, and Sander C. 2008. The microlMA.org resource: targets and expression. Nucleic Acids Res. 36: D149-153.

Bhattacharjee A, Richards WG, Staunton J, Li C, Monti S, Vasa P, Ladd C, Beheshti J, Bueno R, Gillette M, et al. 2001. Classification of human lung carcinomas by mRNA expressionprofiling reveals distinct adenocarcinoma subclasses. Proc. Natl. Acad. Sci. U.S.A. 98: 13790-13795.

Boll K, Reiche K, Kasack K, Morbt N, Kretzschmar A K, Tomm J M, Verhaegh G, Schalken J, von Bergen M, Horn F, et al. 2012. MiR-130a, miR-203 and miR-205 jointly repress key oncogenic pathways and are downregulated in prostate carcinoma. Oncogene. http://www.ncbi.nlm.nih.gov/pubmed/22391564 (Accessed Apr. 12, 2012).

Brennecke J, Stark A, Russell R B, and Cohen S M. 2005. Principles of microRNA-target recognition. PLoS Biol. 3: e85.

Brueckner B, Stresemann C, Kuner R, Mund C, Musch T, Meister M, Silltmann H, and Lyko F. 2007. The human let-7a-3 locus contains an epigenetically regulated microRNA gene with oncogenic function. Cancer Res. 67: 1419-1423.

Ceppi M, Pereira P M, Dunand-Sauthier I, Bums E, Reith W, Santos M A, and Pierre P. 2009. MicroRNA-155 modulates the interleukin-1 signaling pathway in activated human monocyte-derived dendritic cells. Proc. Natl. Acad Sci. U.S.A. 106: 2735-2740.

Chang T-C, Wentzel E A, Kent O A, Ramachandran K, Mullendore M, Lee K H, Feldmann G, Yamakuchi M, Ferlito M, Lowenstein C J, et al. 2007. Transactivation of miR-34a by p53 broadly influences gene expression and promotes apoptosis. Mol. Cell 26: 745-752.

Chung C H, Parker J S, Karaca G, Wu Junyuan, Funkhouser W K, Moore D, Butterfoss D, Xiang D, Zanation A, Yin X, et al. 2004. Molecular classification of head and neck squamous cell carcinomas using patterns of gene expression. Cancer Cell 5: 489-500.

Cushing L, Kuang P P, Qian J, Shao F, Wu Junjie, Little F, Thannickal V J, Cardoso W V, and Lu J. 2011. miR-29 is a major regulator of genes associated with pulmonary fibrosis. Am. J. Respir. Cell Mol. Biol. 45: 287-294.

Dalmay T, and Edwards D R. 2006. MicroRNAs and the hallmarks of cancer. Oncogene 25: 6170-6175.

Fan D, Bitterman P B, and Larsson 0.2009. Regulatory element identification in subsets of transcripts: comparison and integyation of current computational methods. RNA 15: 1469-1482.

Fasanaro P, Greco S, Lorenzi M, Pescatori M, Brioschi M, Kulshreshtha R, Banfi C, Stubbs A, Cahn George A, Ivan M, et al. 2009. An integyated approach for experimental target identification of hypoxia-induced miR-210. J. Biol. Chem. 284: 35134-35143.

Frankel L B, Christoffersen N R, Jacobsen A, Lindow M, Krogh A, and Lund All. 2008. Programmed cell death 4 (PDCD4) is an important functional target of the microRNA miR-21 in breast cancer cells. J. Biol. Chem. 283: 1026-1033.

Friedman R C, Farh K K-H, Burge C B, and Bartel D P. 2009. Most mammalian mRNAs are conserved targets of microRNAs. Genome Res. 19: 92-105.

Fröhlich 11, Speer N, Poustka A, and Beissbarth T. 2007. GOSim—an R-package for computation of information theoretic GO similarities between terms and gene products. BMC Bioinformatics 8: 166.

Garofalo M, and Croce C M. 2011. microRNAs: Master regulators as potential therapeutics in cancer. Annu. Rev. Pharmacol. Toxicol. 51: 25-43.

Georges S A, Biery M C, Kim S-Y, Schelter J M, Guo J, Chang A N, Jackson A L, Carleton M O, Linsley P S, Cleary M A, et al. 2008. Coordinated regulation of cell cycle transcripts by p53-Inducible microRNAs, miR-192 and miR-215. Cancer Res. 68: 10105-10112.

Goodarzi II, Elemento 0, and Tavazoie S. 2009. Revealing global regulatory perturbations across human cancers. Mol. Cell 36: 900-911.

Grimson A, Farh K K-H, Johnston W K, Garrett-Engele P, Lim L P, and Bartel D P. 2007. MicroRNA targeting specificity in mammals: determinants beyond seed pairing. Mol. Cell 27: 91-105.

Hanahan D, and Weinberg R A. 2000. The hallmarks of cancer. Cell 100: 57-70.

Hausser J, Berninger P, Rodak C, Jantscher Y, Wirth S, and Zavolan M. 2009. MirZ: an integrated microRNA expression atlas and target prediction resource. Nucleic Acids Res. 37: W266-272.

He L, He X, Lim LP, de Stanchina E, Xuan Z, Liang Y, Xue W, Zender L, Magnus J, Ridzon D, et al. 2007. A microRNA component of the p53 tumour suppressor network. Nature 447: 1130-1134.

Hendrickson D G, Hogan D J, Herschlag D, Ferrell J E, and Brown P O. 2008. Systematic identification of mRNAs recruited to argonaute 2 by specific microRNAs and corresponding changes in transcript abundance. PLoS ONE 3: e2126.

Jiang Q, Wang Y, Hao Y, Juan L, Teng M, Zhang X, Li M, Wang G, and Liu Y. 2009. miR2Disease: a manually curated database for microRNA deregulation in human disease. Nucleic Acids Res. 37: D98-104.

Johnson C D, Esquela-Kerscher A, Stefani G, Byrom M, Kelnar K, Ovcharenko D, Wilson Mike, Wang Xiaowei, Shelton J, Shingara J, et al. 2007. The let-7 microRNA represses cell proliferation pathways in human cells. Cancer Res. 67: 7713-7722.

Karginov F V, Conaco C, Xuan Z, Schmidt B H, Parker J S, Mandel G, and Hannon G J. 2007. A biochemical approach to identifying microRNA targets. Proc. Natl. Acad. Sci. U.S.A. 104: 19291-19296.

Kertesz M, lovino N, Unnerstall U, Gaul U, and Segal E. 2007. The role of site accessibility in microRNA target recognition. Nat. Genet. 39: 1278-1284.

Kozomara A, and Griffiths-Jones S. 2011. miRBase: integrating microRNA annotation and deep-sequencing data. Nucleic Acids Res. 39: D152-157.

Lal A, Thomas M P, Altschuler G, Navarro F, O′Day E, Li X L, Concepcion C, Han Y-C, Thiery J, Rajani D K, et al. 2011. Capture of microRNA-bound mRNAs identifies the tumor suppressor miR-34a as a regulator of gyowth factor signaling. PLoS Genet. 7: el 002363.

Landi M T, Zhao Y, Rotunno M, Koshiol J, Liu H, Bergen A W, Rubagotti M, Goldstein A M, Linnoila I, Marincola F M, et al. 2010. MicroRNA expression differentiates histology and predicts survival of lung cancer. Clin. Cancer Res. 16: 430-441.

Lim L P, Lau N C, Garrett-Engele P, Grimson A, Schelter J M, Castle J, Bartel D P, Linsley P S, and Johnson J M. 2005. Microarray analysis shows that some microRNAs downregulate large numbers of target mRNAs. Nature 433: 769-773.

Linhart C, Halperin Y, and Shamir R. 2008. Transcription factor and microRNA motif discovery: the Amadeus platform and a compendium of metazoan target sets. Genome Res. 18: 1180-1189.

Linsley P S, Schelter J, Burchard J, Kibukawa M, Martin M M, Bartz S R, Johnson J M, Cummins J M, Raymond C K, Dai H, et al. 2007. Transcripts targeted by the microRNA-16 family cooperatively regulate cell cycle progression. Mol. Cell. Biol. 27: 2240-2252.

Liu T, Papagiannakopoulos T, Puskar K, Qi S, Santiago F, Clay W, Lao K, Lee Y, Nelson S F, Komblum H I, et al. 2007. Detection of a microRNA signal in an in vivo expression set of mRNAs. PLoS ONE 2: e804.

Malzkorn B, Wolter M, Liesenberg F, Grzendowski M, Stiihler K, Meyer H E, and Reifenberger G. 2010. Identification and functional characterization of microRNAs involved in the malignant progression of gliomas. Brain Pathol. 20: 539-550.

Muniyappa M K, Dowling P, Henry M, Meleady P, Doolan P, Gammell P, Clynes M, and Barron N. 2009. MiRNA-29a regulates the expression of numerous proteins and reduces the invasiveness and proliferation of human carcinoma cell lines. Eur. J. Cancer 45: 3104-3118.

Nana-Sinkam S P, and Croce C M. 2011. MicroRNAs as therapeutic targets in cancer. Transl Res 157: 216-225.

Ozen M, Creighton C J, Ozdemir M, and Ittmann M. 2008. Widespread deregulation of microRNA expression in human prostate cancer. Oncogene 27: 1788-1793.

Pavesi G, Mereghetti P, Zambelli F, Stefani M, Mauri G, and Pesole G. 2006. MoD Tools: regulatory motif discovery in nucleotide sequences from co-regulated or homologous genes. Nucleic Acids Res. 34: W566-570.

Plaisier C L, Bare J C, and Baliga N S. 2011. miRvestigator: web application to identify miRNAs responsible for co-regulated gene expression patterns discovered through transcriptome profiling. Nucleic Acids Res. 39: W125-131.

Reiss D J, Baliga N S, and Bonneau R. 2006. Integrated biclustering of heterogeneous genomewide datasets for the inference of global regulatory networks. BMC Bioinformatics 7: 280.

Ritchie W, Rajasekhar M, Flamant S, and Rasko J E J. 2009. Conserved expression patterns predict microRNA targets. PLoS Comput. Biol. 5: e1000513.

Robin X, Turck N, Hainard A, Tiberti N, Lisacek F, Sanchez J-C, and Muller M. 2011. pROC: an open-source package for R and S+ to analyze and compare ROC curves. BMC Bioinformatics 12: 77.

Rothschild S I, Tschan M P, Federzoni E A, Jaggi R, Fey M F, Gugger M, and Gautschi 0. 2012. MicroRNA-29b is involved in the Src-ID1 signaling pathway and is dysregulated in human lung adenocarcinoma. Oncogene. http://www.ncbi.nlm.nih.gov/pubmed/22249264 (Accessed Apr. 12, 2012).

Ruan K, Fang X, and Ouyang G. 2009. MicroRNAs: novel regulators in the hallmarks of human cancer. Cancer Lett. 285: 116-126.

Selbach M, Schwanhausser B, Thierfelder N, Fang Z, Khanin R, and Rajewsky N. 2008. Widespread changes in protein synthesis induced by microRNAs. Nature 455: 58-63.

Sengupta S, den Boon J A, Chen I-H, Newton M A, Stanhope S A, Cheng Y-J, Chen C-J, Hildesheim A, Sugden B, and Ahlquist P. 2008. MicroRNA 29c is down-regulated innasopharyngeal carcinomas, up-regulating mRNAs encoding extracellular matrix proteins. Proc. Natl. Acad. Sci. U.S.A. 105: 5874-5878.

Sethupathy P, Megraw M, and Hatzigeorgiou AG. 2006. A guide through present computational approaches for the identification of mammalian microRNA targets. Nat. Methods 3: 881-886.

Sing T, Sander 0, Beerenwinkel N, and Lengauer T. 2005. ROCR: visualizing classifier performance in R. Bioinformatics 21: 3940-3941.

Tan L P, Seinen E, Duns G, de Jong D, Sibon O C M, Poppema S, Kroesen B-J, Kok K, and van den Berg A. 2009. A high throughput experimental approach to identify miRNA targets in human cells. Nucleic Acids Res. 37: el 37.

Tsai W-C, Hsu PW-C, Lai T-C, Chau G-Y, Lin C-W, Chen C-M, Lin C-D, Liao Y-L, Wang J-L, Chau Y-P, et al. 2009. MicroRNA-122, a tumor suppressor microRNA that regulates intrahepatic metastasis of hepatocellular carcinoma. Hepatology 49: 1571-1582.

Vaira V, Faversani A, Dohi T, Montorsi M, Augello C, Gatti S, Coggi G, Alfieri D C, and Bosari S. 2011 miR-296 regulation of a cell polarity-cell plasticity module controls tumor progression. Oncogene. http://www.ncbi.nlm.nih.gov/pubmed/21613016 (Accessed Oct. 8, 2011).

Valastyan S, Reinhardt F, Benaich N, Calogrias D, Szasz A M, Wang Z C, Brock J E, Richardson A L, and Weinberg Robert A. 2009. A pleiotropically acting microRNA, miR-31, inhibits breast cancer metastasis. Cell 137: 1032-1046.

Wang L, Oberg A L, Asmann Y W, Sicotte H, McDonnell S K, Riska S M, Liu W, Steer C J, Subramanian S, Cunningham J M, et al. 2009. Genome-wide transcriptional profiling reveals microRNA-correlated genes and biological processes in human lymphoblastoid cell lines. PLoS ONE 4: e5878.

Wang W-X, Wilfred B R, Hu Y, Stromberg A J, and Nelson P T. 2010. Anti-Argonaute RIP-Chip shows that miRNA transfections alter global patterns of mRNA recruitment to microribonucleoprotein complexes. RNA 16: 394-404.

Weber F, Teresi R E, Broelsch C E, Frilling A, and Eng C. 2006. A limited set of human MicroRNA is deregulated in follicular thyroid carcinoma. J. Clin. Endocrinol. Metab. 91: 3584-3591.

Welsh J B, Zarrinkar P P, Sapinoso L M, Kern S G, Behling C A, Monk B J, Lockhart D J, Burger R A, and Hampton G M. 2001. Analysis of gene expression profiles in normal and neoplastic ovarian tissue samples identifies candidate molecular markers of epithelial ovarian cancer. Proc. Natl. Acad. Sci. U.S.A. 98: 1176-1181.

Yanaihara N, Caplen N, Bowman E, Seike M, Kumamoto K, Yi M, Stephens R M, Okamoto A, Yokota J, Tanaka T, et al. 2006. Unique microRNA molecular profiles in lung cancer diagnosis and prognosis. Cancer Cell 9: 189-198.

Zen K, and Zhang C-Y. 2010. Circulating MicroRNAs: a novel class of biomarkers to diagnose and monitor human cancers. Med Res Rev http://www.ncbi.nlm.nih.gov/pubmed/21064190 (Accessed Oct. 8, 2011).

### SUPPLEMENTARY REFERENCES

Baek D, Villén J, Shin C, Camargo F D, Gygi S P, and Bartel D P. 2008. The impact of microRNAs on protein output. Nature 455: 64-71.

Fan D, Bitterman P B, and Larsson O. 2009. Regulatory element identification in subsets of transcripts: comparison and integration of current computational methods. RNA 15: 1469-1482.

Guo H, Ingolia N T, Weissman J S, and Bartel D P. 2010. Mammalian microRNAs predominantly act to decrease target mRNA levels. Nature 466: 835-840.

Hendrickson D G, Hogan D J, McCullough H L, Myers J W, Herschlag D, Ferrell J E, and Brown P O. 2009. Concordant regulation of translation and mRNA abundance for hundreds of targets of a human microRNA. PLoS Biol. 7: e1000238.

Kertesz M, lovino N, Unnerstall U, Gaul U, and Segal E. 2007. The role of site accessibility in microRNA target recognition. Nat. Genet. 39: 1278-1284.

Linhart C, Halperin Y, and Shamir R. 2008. Transcription factor and microRNA motif discovery: the Amadeus platform and a compendium of metazoan target sets. Genome Res. 18: 1180-1189.

Pavesi G, Mereghetti P, Zambelli F, Stefani M, Mauri G, and Pesole G. 2006. MoD Tools: regulatory motif discovery in nucleotide sequences from co-regulated or homologous genes. Nucleic Acids Res. 34: W566-570.

Selbach M, Schwanhäusser B, Thierfelder N, Fang Z, Khanin R, and Rajewsky N. 2008. Widespread changes in protein synthesis induced by microRNAs. Nature 455: 58-63.

TABLE 1
Genes validated to be regulated by miR-29 family.

| | | | | miR-29 Family Target Sites | | |
|---|---|---|---|---|---|---|
| Gene | Entrez | AD Lung | AD Lung | Weeder- | | |
| Symbols | Gene ID | Beer 31 | Bhattacharjee 59 | miRvestigator | PITA | TargetScan |
| COL1A1 | 1277 | Yes | | a/b/c | a/b/c | a |
| COL1A2 | 1278 | | Yes | | a/b/c | a |
| COL3A1 | 1281 | Yes | Yes | a/b/c | a/b/c | b |
| COL4A1 | 1282 | Yes | Yes | a/b/c | a/b/c | b |
| COL4A2 | 1284 | Yes | | a/b/c | a/b/c | a |
| COL5A1 | 1289 | Yes | | a/b/c | a/b/c | a |
| COL5A2 | 1290 | Yes | Yes | a/b/c | a/b/c | a |
| COL15A1 | 1306 | Yes | Yes | a/b/c | a/b/c | b |
| FBN1 | 2200 | Yes | Yes | a/b/c | a/b/c | a |
| FSTL1 | 11167 | Yes | | a/b/c | | a |
| LOXL2 | 4017 | Yes | | a/b/c | | a |
| MMP2 | 4313 | Yes | | a/b/c | a/b/c | a |
| PDGFRB | 5159 | Yes | Yes | a/b/c | a/b/c | a |
| PPIC | 5480 | Yes | | a/b/c | | b |
| SERPINH1 | 871 | Yes | Yes | a/b/c | | b |
| SPARC | 6678 | Yes | Yes | a/b/c | | a |
| TRIB2 | 28951 | Yes | | a/b/c | a/b/c | a |

a = miR-29a, b = miR-29b, c = miR-29c.

SUPPLEMENTARY TABLE 1

| | | | | Free | Gene |
|---|---|---|---|---|---|
| | miRNA | Cross- | | Energy of | Expression |
| | Seed | Species | Free | Secondary | miRNA |
| Inference | Comple- | Conser- | Energy of | mRNA | Perturbation |
| Method | mentarity | vation | Annealing | Structure | Experiments |
| PITA | X | X | X | X | |
| TargetScan | X | X | | | |
| miRanda | X | X | X | | |
| miRSVR | X | X | X | | X |

SUPPLEMENTARY TABLE 2

| PMID | miRNA | Perturbation | System | Biological Model | Environment | Assay | Target Genes |
|---|---|---|---|---|---|---|---|
| 15685193 | hsa-miR-1 | miRNA | HeLa | Ovarian Cancer | in vitro | Transcriptomics | 98 |
| 15685193 | hsa-miR-124 | miRNA | HeLa | Ovarian Cancer | in vitro | Transcriptomics | 165 |
| 15685193 | hsa-miR-hes-3 ^{B} | miRNA | HeLa | Ovarian Cancer | in vitro | Transcriptomics | 61 |
| 16549876 | hsa-miR-124 | miRNA | HepG2 | Liver Cancer | in vitro | Transcriptomics | 13 |
| 16822819 | hsa-miR-192 | Patient GE & | Patients | Follicular Thyroid | in vivo | Transcriptomics | 48 |
| | | Microcosm | | Cancer | | | |
| 16822819 | hsa-miR-197 | Patient GE & | Patients | Follicular Thyroid | in vivo | Transcriptomics | 57 |
| | | Microcosm | | Cancer | | | |
| 16822819 | hsa-miR-346 | Patient GE & | Patients | Follicular Thyroid | in vivo | Transcriptomics | 24 |
| | | Microcosm | | Cancer | | | |
| 17242205 | hsa-miR-103 | miRNA | HCT116 & DLD-1 | Colon Cancer | in vitro | Transcriptomics | 110 |
| 17242205 | hsa-miR-106b | miRNA | HCT116 & DLD-1 | Colon Cancer | in vitro | Transcriptomics | 22 |
| 17242205 | hsa-miR-107 | miRNA | HCT116 & DLD-1 | Colon Cancer | in vitro | Transcriptomics | 81 |
| 17242205 | hsa-miR-141 | miRNA | HCT116 & DLD-1 | Colon Cancer | in vitro | Transcriptomics | 100 |
| 17242205 | hsa-miR-15a | miRNA | HCT116 & DLD-1 | Colon Cancer | in vitro | Transcriptomics | 102 |
| 17242205 | hsa-miR-15b | miRNA | HCT116 & DLD-1 | Colon Cancer | in vitro | Transcriptomics | 186 |
| 17242205 | hsa-miR-16 | miRNA | HCT116 & DLD-1 | Colon Cancer | in vitro | Transcriptomics | 64 |
| 17242205 | hsa-miR-17 | miRNA | HCT116 & DLD-1 | Colon Cancer | in vitro | Transcriptomics | 75 |
| 17242205 | hsa-miR-192 | miRNA | HCT116 & DLD-1 | Colon Cancer | in vitro | Transcriptomics | 483 |
| 17242205 | hsa-miR-195 | miRNA | HCT116 & DLD-1 | Colon Cancer | in vitro | Transcriptomics | 185 |
| 17242205 | hsa-miR-20 | miRNA | HCT116 & DLD-1 | Colon Cancer | in vitro | Transcriptomics | 65 |
| 17242205 | hsa-miR-200a | miRNA | HCT116 & DLD-1 | Colon Cancer | in vitro | Transcriptomics | 89 |
| 17242205 | hsa-miR-200b | miRNA | HCT116 & DLD-1 | Colon Cancer | in vitro | Transcriptomics | 68 |
| 17242205 | hsa-miR-215 | miRNA | HCT116 & DLD-1 | Colon Cancer | in vitro | Transcriptomics | 165 |
| 17242205 | hsa-let-7c | miRNA | HCT116 & DLD-1 | Colon Cancer | in vitro | Transcriptomics | 52 |
| 17308078 | hsa-let-7a | miRNA | A549, HeLa & | Lung, Ovarian | in vitro | Transcriptomics | 195 |
| | | | HepG2 | & Liver Cancer | | | |
| 17540599 | hsa-miR-34a | miRNA | IMR90 | Primary Normal | in vitro | Transcriptomics | 100 |
| | | | | Lung | | | |
| 17554337 | hsa-miR-34a | miRNA | HCT116 | Colon Cancer | in vitro | Transcriptomics | 421 |
| 17612493 | hsa-miR-7 | miRNA | HeLa | Ovarian Cancer | in vitro | Transcriptomics | 49 |
| 17612493 | hsa-miR-9 | miRNA | HeLa | Ovarian Cancer | in vitro | Transcriptomics | 13 |
| 17612493 | hsa-miR-122a | miRNA | HeLa | Ovarian Cancer | in vitro | Transcriptomics | 33 |
| 17612493 | hsa-miR-128 | miRNA | HeLa | Ovarian Cancer | in vitro | Transcriptomics | 34 |
| 17612493 | hsa-miR-132 | miRNA | HeLa | Ovarian Cancer | in vitro | Transcriptomics | 60 |
| 17612493 | hsa-miR-133a | miRNA | HeLa | Ovarian Cancer | in vitro | Transcriptomics | 197 |
| 17612493 | hsa-miR-142-3p | miRNA | HeLa | Ovarian Cancer | in vitro | Transcriptomics | 81 |
| 17612493 | hsa-miR-148b | miRNA | HeLa | Ovarian Cancer | in vitro | Transcriptomics | 56 |
| 17612493 | hsa-miR-181a | miRNA | HeLa | Ovarian Cancer | in vitro | Transcriptomics | 19 |
| 17699775 | hsa-let-7b | miRNA | HCT116 | Colon Cancer | in vitro | Transcriptomics | 197 |
| 17891175 | hsa-miR-125a | Patient GE | Patients | Prostate Cacner | in vivo | Transcriptomics | 25 |
| 17991735 | hsa-miR-21 | Anti-miR | MCF-7 | Breast Cancer | in vitro | Transcriptomics | 309 |
| 18042700 | hsa-miR-124 | RIP-Chip + | HEK293S | Human | in vitro | Transcriptomics | 287 |
| | | miRNA | | Embryonic Kindey | | | |
| 18390668 | hsa-miR-29c | miRNA | HeLa & HepG2 | Ovarian Cancer | in vitro | Transcriptomics | 12 |
| | | | | & Human | | | |
| | | | | Embryonic Kidney | | | |
| 18461144 | hsa-miR-1 | RIP-Chip + | HEK293T | Human | in vitro | Transcriptomics | 68 |
| | | miRNA | | Embryonic Kindey | | | |
| 18461144 | hsa-miR-124 | RIP-Chip + | HEK293T | Human | in vitro | Transcriptomics | 419 |
| | | miRNA | | Embryonic Kindey | | | |
| 19074876 | hsa-miR-192 | miRNA | HCT116 | Colon Cancer | in vitro | Transcriptomics | 18 |
| 19193853 | hsa-miR-155 | Anti-miR | Cultured | Moncyte Derived | in vitro | Transcriptomics | 22 |
| | | | Dendritic Cells | Dendritic Cells | | | |
| 19296470 | hsa-miR-122 | miRNA | Mahlavu, HuH-7 | Hepatocellular | in vitro | Transcriptomics | 31 |
| | | | and SK-HEP-1 | Carcinoma | | | |
| 19524507 | hsa-miR-31 | miRNA | MDA-MB-231 | Breast Cancer | in vitro | Luciferase | 16 |
| 19734348 | hsa-miR-17-5p/ | RIP-Chip + | L428 & L1236 | Hodgkin | in vitro | Transcriptomics | 84 |
| | 20a/93/106a/ | Anti-miR | | Lymphoma | | | |
| | 106b ^{E} | | | | | | |
| 19775293 | hsa-miR-184 | miRNA & | A172 & T98G | Glioma | in vitro | Transcriptomics | 17 |
| | | miRBase | | | | | |
| 19826008 | hsa-miR-210 | Anti-miR | HUVEC | Human | in vitro | Transcriptomics | 32 |
| | | | | Umbilical Vein | | | |
| | | | | Embryonic Cells | | | |
| 20042474 | hsa-miR-124 | RIP-Chip + | H4 & SH-SY5Y | Neuroblastoma | in vitro | Transcriptomics | 10 |
| | | miRNA | | | | | |
| 20042474 | hsa-miR-128 | RIP-Chip + | H4 & SH-SY5Y | Neuroblastoma | in vitro | Transcriptomics | 11 |
| | | miRNA | | | | | |

SUPPLEMENTARY TABLE 4

| | | | Complementarity | | P-value |
|---|---|---|---|---|---|
| Dataset | Cluster | miRNAname | Model | Complementarity | Viterbi |
| AC_Brain_Sun | 4 | hsa-miR-937 | 7mer-a1 | CGCGCGGA | 6.10E−05 |
| | | | | _\|\|\|\|\|\|\| | |
| | | | | -CGCGCCT | |
| AC_Brain_Sun | 5 | hsa-miR-523 | 7mer-a1 | CGCGCGTT | 6.10E−05 |
| | | | | _\|\|\|\|\|\|\| | |
| | | | | -CGCGCAA | |
| AC_Brain_Sun | 17 | hsa-miR-487b | 7mer-a1 | NGTANGAT | 6.10E−05 |
| | | | | _\|\|\|\|\|\|\| | |
| | | | | -CATGCTA | |
| AC_Brain_Sun | 50 | hsa-miR-1234 | 7mer-m8 | TAGGCCNA | 6.10E−05 |
| | | | | _\|\|\|\|\|\|\| | |
| | | | | -TCCGGCT | |
| AD_Lung_Beer | 19 | hsa-miR-487b | 7mer-a1 | GTANNATC | 6.10E−05 |
| | | | | \|\|\|\|\|\|\|_ | |
| | | | | CATGCTA- | |
| AD_Lung_Beer | 31 | hsa-miR-29b | 8mer | TGGTGCTA | 1.53E−05 |
| | | hsa-miR-29c | | \|\|\|\|\|\|\|\| | |
| | | hsa-miR-29a | | ACCACGAT | |
| AD_Lung_Bhattacharjee | 6 | hsa-miR-941 | 8mer | GCCGGGTG | 1.53E−05 |
| | | | | \|\|\|\|\|\|\|\| | |
| | | | | CGGCCCAC | |
| AD_Lung_Bhattacharjee | 27 | hsa-miR-487b | 7mer-a1 | CGTACGAT | 6.10E−05 |
| | | | | _\|\|\|\|\|\|\| | |
| | | | | -CATGCTA | |
| AD_Lung_Bhattacharjee | 44 | hsa-miR-655 | 7mer-a1 | CTGTATTA | 6.10E−05 |
| | | | | _\|\|\|\|\|\|\| | |
| | | | | -ACATAAT | |
| AD_Lung_Bhattacharjee | 64 | hsa-miR-150 | 7mer-a1 | GTTGGGAG | 6.10E−05 |
| | | | | _\|\|\|\|\|\|\| | |
| | | | | -AACCCTC | |
| AD_Lung_Bhattacharjee | 65 | hsa-miR-3177 | 7mer-a1 | CGCCSTGC | 6.10E−05 |
| | | | | _\|\|\|\|\|\|\| | |
| | | | | -CGGCACG | |
| AD_Lung_Stearman | 29 | hsa-miR-598 | 7mer-m8 | TGANNTAT | 6.10E−05 |
| | | | | \|\|\|\|\|\|\|_ | |
| | | | | ACTGCAT- | |
| AD_Ovarian_Welsh | 6 | hsa-let-7e-3p | 8mer | CYNTATAG | 1.53E−05 |
| | | | | \|\|\|\|\|\|\|\| | |
| | | | | GGCATATC | |
| AD_Ovarian_Welsh | 13 | hsa-miR-3178 | 7mer-a1 | TCNCGCCC | 6.10E−05 |
| | | | | _\|\|\|\|\|\|\| | |
| | | | | -GCGCGGG | |
| AD_Ovarian_Welsh | 24 | hsa-miR-1469 | 7mer-a1 | GCGCCGAT | 6.10E−05 |
| | | | | \|\|\|\|\|\|\|_ | |
| | | | | CGCGGCT- | |
| AD_Ovarian_Welsh | 29 | hsa-miR-3194 | 7mer-m8 | GCTGGCCN | 6.10E−05 |
| | | | | \|\|\|\|\|\|\|_ | |
| | | | | CGACCGG- | |
| AD_Pancreas_Logsdon | 3 | hsa-miR-3178 | 7mer-m8 | GCGCCCCG | 6.10E−05 |
| | | | | \|\|\|\|\|\|\|_ | |
| | | | | CGCGGGG- | |
| AD_Pancreas_Logsdon | 10 | hsa-miR-1273 | 7mer-m8 | GGTCKCCC | 6.10E−05 |
| | | | | _\|\|\|\|\|\|\| | |
| | | | | -CAGCGGG | |
| AD_Pancreas_Logsdon | 12 | hsa-let-7f hsa-miR-98 | 7mer-a1 | CTNCCTCN | 6.10E−05 |
| | | hsa-let-7b hsa-let-7c | | \|\|\|\|\|\|\|_ | |
| | | hsa-let-7a hsa-let-7g | | GATGGAG- | |
| | | hsa-let-7e hsa-let-7i | | | |
| | | hsa-let-7d | | | |
| AO_Brain_Bredel | 10 | hsa-miR-4285 | 7mer-m8 | TCNCCNCA | 6.10E−05 |
| | | | | \|\|\|\|\|\|\|_ | |
| | | | | AGCGGCG- | |
| B-CLL_Leukemia_Haslinger | 54 | hsa-miR-937 | 7mer-a1 | CGCGCGGA | 6.10E−05 |
| | | | | _\|\|\|\|\|\|\| | |
| | | | | -CGCGCCT | |
| B-CLL_Leukemia_Haslinger | 58 | hsa-miR-337 | 7mer-m8 | CNCCNTTC | 6.10E−05 |
| | | | | _\|\|\|\|\|\|\| | |
| | | | | -CGGCAAG | |
| B-CLL_Leukemia_Haslinger | 69 | hsa-miR-450a | 7mer-a1 | ATNNCAAA | 6.10E−05 |
| | | | | _\|\|\|\|\|\|\| | |
| | | | | -AGCGTTT | |
| BPH_Prostate_Dhanasekaran | 13 | hsa-miR-423-3p | 7mer-a1 | GACNGAGC | 6.10E−05 |
| | | | | _\|\|\|\|\|\|\| | |
| | | | | -TGGCTCG | |
| CA_Bladder_Dyrskjot | 13 | hsa-miR-548k | 7mer-a1 | NNTACTTT | 6.10E−05 |
| | | | | _\|\|\|\|\|\|\| | |
| | | | | -CATGAAA | |
| CA_Bladder_Dyrskjot | 17 | hsa-miR-885-3p | 7mer-a1 | CNCTGCCN | 6.10E−05 |
| | | | | \|\|\|\|\|\|\|_ | |
| | | | | GCGACGG- | |
| CA_Bladder_Dyrskjot | 26 | hsa-miR-194 | 7mer-m8 | TGTTANAA | 6.10E−05 |
| | | | | \|\|\|\|\|\|\|_ | |
| | | | | ACAATGT- | |
| CA_Bladder_Dyrskjot | 35 | hsa-miR-1469 | 7mer-a1 | GCGCCGAT | 6.10E−05 |
| | | | | \|\|\|\|\|\|\|_ | |
| | | | | CGCGGCT- | |
| CA_Breast_Richardson | 15 | hsa-let-7d-3p | 7mer-a1 | TNNTATAC | 6.10E−05 |
| | | | | \|\|\|\|\|\|\|_ | |
| | | | | AGCATAT- | |
| CA_Breast_Richardson | 17 | hsa-miR-566 | 7mer-m8 | TGGNNCCC | 6.10E−05 |
| | | | | _\|\|\|\|\|\|\| | |
| | | | | -CCGCGGG | |
| CA_Breast_Richardson | 46 | hsa-miR-487b | 7mer-m8 | TANNATTA | 6.10E−05 |
| | | | | \|\|\|\|\|\|\|_ | |
| | | | | ATGCTAA- | |
| CA_Breast_Sorlie | 24 | hsa-miR-1538 | 8mer | CCGGGCCN | 1.53E−05 |
| | | | | \|\|\|\|\|\|\|\| | |
| | | | | GGCCCGGC | |
| CA_Colon_Graudens | 11 | hsa-miR-523 | 8mer | GCGCNTTC | 1.53E−05 |
| | | | | \|\|\|\|\|\|\|\| | |
| | | | | CGCGCAAG | |
| CCC_Ovarian_Hendrix | 11 | hsa-miR-638 | 7mer-a1 | CCNNTCCC | 6.10E−05 |
| | | | | _\|\|\|\|\|\|\| | |
| | | | | -GCTAGGG | |
| CCC_Ovarian_Hendrix | 23 | hsa-miR-523 | 7mer-a1 | GCGCKTTA | 6.10E−05 |
| | | | | \|\|\|\|\|\|\|_ | |
| | | | | CGCGCAA- | |
| CCC_Ovarian_Hendrix | 55 | hsa-miR-1471 | 7mer-a1 | TACGCGGG | 6.10E−05 |
| | | | | _\|\|\|\|\|\|\| | |
| | | | | -TGCGCCC | |
| COID_Lung_Bhattacharjee | 10 | hsa-miR-138-2-3p | 7mer-m8 | AAATAGNN | 6.10E−05 |
| | | | | \|\|\|\|\|\|\|_ | |
| | | | | TTTATCG- | |
| COID_Lung_Bhattacharjee | 48 | hsa-miR-886 | 7mer-a1 | CCNACCCA | 6.10E−05 |
| | | | | \|\|\|\|\|\|\|_ | |
| | | | | GGCTGGG- | |
| COID_Lung_Bhattacharjee | 84 | hsa-miR-615 | 7mer-m8 | CNACCCCC | 6.10E−05 |
| | | | | _\|\|\|\|\|\|\| | |
| | | | | -CTGGGGG | |
| COID_Lung_Bhattacharjee | 87 | hsa-miR-1181 | 7mer-m8 | GNGACGGA | 6.10E−05 |
| | | | | \|\|\|\|\|\|\|_ | |
| | | | | CGCTGCC- | |
| DLBCL_Lymphoma_Alizadeh | 5 | hsa-miR-132-3p | 7mer-m8 | ACCACNGT | 6.10E−05 |
| | | | | _\|\|\|\|\|\|\| | |
| | | | | -GGTGCCA | |
| END_Ovarian_Hendrix | 0 | hsa-miR-598 | 8mer | ATGANNTA | 1.53E−05 |
| | | | | \|\|\|\|\|\|\|\| | |
| | | | | TACTGCAT | |
| END_Ovarian_Hendrix | 18 | hsa-miR-487b | 7mer-a1 | NGTACGAT | 6.10E−05 |
| | | | | _\|\|\|\|\|\|\| | |
| | | | | -CATGCTA | |
| END_Ovarian_Hendrix | 19 | hsa-miR-3195 | 7mer-a1 | ACCGGCGC | 6.10E−05 |
| | | | | _\|\|\|\|\|\|\| | |
| | | | | -GGCCGCG | |
| END_Ovarian_Hendrix | 45 | hsa-miR-1471 | 7mer-a1 | TACGCGGG | 6.10E−05 |
| | | | | _\|\|\|\|\|\|\| | |
| | | | | -TGCGCCC | |
| END_Ovarian_Hendrix | 47 | hsa-miR-1471 | 7mer-m8 | CGCGGGCG | 6.10E−05 |
| | | | | \|\|\|\|\|\|\|_ | |
| | | | | GCGCCCG- | |
| END_Ovarian_Hendrix | 52 | hsa-miR-1538 | 7mer-a1 | CNNGGCCT | 6.10E−05 |
| | | | | \|\|\|\|\|\|\|_ | |
| | | | | GGCCCGG- | |
| END_Ovarian_Hendrix | 58 | hsa-miR-621 | 7mer-m8 | GCTAGCNG | 6.10E−05 |
| | | | | \|\|\|\|\|\|\|_ | |
| | | | | CGATCGG- | |
| END_Ovarian_Hendrix | 82 | hsa-let-7d-3p | 7mer-a1 | GTNNTATA | 6.10E−05 |
| | | | | _\|\|\|\|\|\|\| | |
| | | | | -AGCATAT | |
| FL_Lymphoma_Alizadeh | 2 | hsa-miR-425-3p | 7mer-a1 | TTCCNGAC | 6.10E−05 |
| | | | | \|\|\|\|\|\|\|_ | |
| | | | | AAGGGCT- | |
| FL_Lymphoma_Alizadeh | 4 | hsa-miR-4261 | 7mer-a1 | TGTTTCCC | 6.10E−05 |
| | | | | \|\|\|\|\|\|\|_ | |
| | | | | ACAAAGG- | |
| GBM_Brain_Liang | 14 | hsa-miR-496 | 7mer-a1 | CAATACTC | 6.10E−05 |
| | | | | \|\|\|\|\|\|\|\| | |
| | | | | -TTATGAG | |
| GBM_Brain_Liang | 17 | hsa-miR-361 | 7mer-a1 | TCTGATAG | 6.10E−05 |
| | | | | \|\|\|\|\|\|\|_ | |
| | | | | AGACTAT- | |
| GBM_Brain_Liang | 18 | hsa-miR-369 | 7mer-a1 | GTNGATNG | 6.10E−05 |
| | | | | \|\|\|\|\|\|\|_ | |
| | | | | CAGCTAG- | |
| GCT_Seminoma_Korkola | 3 | hsa-miR-324 | 8mer | GGGATGNG | 1.53E−05 |
| | | | | \|\|\|\|\|\|\|\| | |
| | | | | CCCTACGC | |
| GCT_Seminoma_Korkola | 42 | hsa-miR-1181 | 8mer | GGNGASGG | 1.53E−05 |
| | | | | \|\|\|\|\|\|\|\| | |
| | | | | CCGCTGCC | |
| GCT_Seminoma_Korkola | 75 | hsa-miR-25 | 7mer-a1 | NNTGCAAT | 6.10E−05 |
| | | hsa-miR-32 | | _\|\|\|\|\|\|\| | |
| | | hsa-miR-92a | | -CACGTTA | |
| | | hsa-miR-92b | | | |
| | | hsa-miR-363 | | | |
| | | hsa-miR-367 | | | |
| GL_Brain_Bredel | 15 | hsa-miR-126 | 8mer | CGGTANGA | 1.53E−05 |
| | | | | \|\|\|\|\|\|\|\| | |
| | | | | GCCATGCT | |
| GL_Brain_Bredel | 18 | hsa-miR-187 | 8mer | AGACANGA | 1.53E−05 |
| | | | | \|\|\|\|\|\|\|\| | |
| | | | | TCTGTGCT | |
| GL_Brain_Bredel | 24 | hsa-miR-516b | 7mer-a1 | ACTCCAGA | 6.10E−05 |
| | | | | _\|\|\|\|\|\|\| | |
| | | | | -GAGGTCT | |
| GL_Brain_Bredel | 48 | hsa-miR-2277 | 7mer-a1 | CGCTGTCN | 6.10E−05 |
| | | | | \|\|\|\|\|\|\|_ | |
| | | | | GCGACAG- | |
| GL_Brain_Bredel | 66 | hsa-miR-223 | 7mer-a1 | AACTGACT | 6.10E−05 |
| | | | | \|\|\|\|\|\|\|_ | |
| | | | | TTGACTG- | |
| GL_Brain_Bredel | 78 | hsa-miR-126 | 8mer | CGGTAMGA | 1.53E−05 |
| | | | | \|\|\|\|\|\|\|\| | |
| | | | | GCCATGCT | |
| GL_Brain_Rickman | 2 | hsa-miR-718 | 7mer-a1 | GGNGGAAC | 6.10E−05 |
| | | | | \|\|\|\|\|\|\|_ | |
| | | | | CCGCCTT- | |
| GL_Brain_Rickman | 26 | hsa-miR-1469 | 7mer-a1 | GCGCCSAT | 6.10E−05 |
| | | | | \|\|\|\|\|\|\|_ | |
| | | | | CGCGGCT- | |
| GLB_Brain_Sun | 29 | hsa-miR-1181 | 7mer-m8 | CGCGACGG | 6.10E−05 |
| | | | | _\|\|\|\|\|\|\| | |
| | | | | -CGCTGCC | |
| GLB_Brain_Sun | 64 | hsa-let-7e-3p | 7mer-a1 | CCNTATAC | 6.10E−05 |
| | | | | \|\|\|\|\|\|\|_ | |
| | | | | GGCATAT- | |
| HSCC_Head-Neck_Chung | 1 | hsa-miR-29b | 7mer-m8 | NNGTGCTA | 6.10E−05 |
| | | hsa-miR-29c | | _\|\|\|\|\|\|\| | |
| | | hsa-miR-29a | | -CCACGAT | |
| IDC_Breast_Radvanyi | 8 | hsa-miR-604 | 7mer-a1 | CNCAGCCN | 6.10E−05 |
| | | | | \|\|\|\|\|\|\|_ | |
| | | | | GCGTCGG- | |
| IDC_Breast_Radvanyi | 14 | hsa-miR-590-3p | 7mer-a1 | ATAAAATT | 6.10E−05 |
| | | | | _\|\|\|\|\|\|\| | |
| | | | | -ATTTTAA | |
| IDC_Breast_Radvanyi | 23 | hsa-miR-376a-3p | 7mer-a1 | NNAATCTA | 6.10E−05 |
| | | | | _\|\|\|\|\|\|\| | |
| | | | | -CTTAGAT | |
| IDC_Breast_Radvanyi | 50 | hsa-miR-1247 | 7mer-m8 | CGACGGGT | 6.10E−05 |
| | | | | _\|\|\|\|\|\|\| | |
| | | | | -CTGCCCA | |
| IDC_Breast_Radvanyi | 59 | hsa-miR-1469 | 7mer-a1 | GCGCCGAC | 6.10E−05 |
| | | | | \|\|\|\|\|\|\|_ | |
| | | | | CGCGGCT- | |
| ILC_Breast_Radvanyi | 6 | hsa-let-7d-3p | 7mer-a1 | TNNTATAC | 6.10E−05 |
| | | | | \|\|\|\|\|\|\|_ | |
| | | | | AGCATAT- | |
| ILC_Breast_Radvanyi | 11 | hsa-miR-483 | 7mer-a1 | TCCCNTCT | 6.10E−05 |
| | | | | _\|\|\|\|\|\|\| | |
| | | | | -GGGCAGA | |
| ME_Melanoma_Hoek | 3 | hsa-miR-4285 | 8mer | CTCGCCGC | 1.53E−05 |
| | | | | \|\|\|\|\|\|\|\| | |
| | | | | GAGCGGCG | |
| ML_Melanoma_Talantov | 13 | hsa-miR-337 | 7mer-m8 | GCCNTTCG | 6.10E−05 |
| | | | | \|\|\|\|\|\|\|_ | |
| | | | | CGGCAAG- | |
| ML_Melanoma_Talantov | 19 | hsa-miR-302c-3p | 7mer-m8 | TKTTAAAT | 6.10E−05 |
| | | | | \|\|\|\|\|\|\|_ | |
| | | | | ACAATTT- | |
| ML_Melanoma_Talantov | 28 | hsa-miR-3175 | 7mer-m8 | NCTCCCCN | 6.10E−05 |
| | | | | _\|\|\|\|\|\|\| | |
| | | | | -GAGGGGC | |
| ML_Melanoma_Talantov | 34 | hsa-miR-548c-3p | 7mer-a1 | ANATTTTT | 6.10E−05 |
| | | | | _\|\|\|\|\|\|\| | |
| | | | | -CTAAAAA | |
| MPC_Prostate_Dhanasekaran | 0 | hsa-miR-3074 | 7mer-a1 | GCTGATAT | 6.10E−05 |
| | | | | _\|\|\|\|\|\|\| | |
| | | | | -GACTATA | |
| MPC_Prostate_Dhanasekaran | 42 | hsa-miR-582-3p | 7mer-a1 | TACCAGTT | 6.10E−05 |
| | | | | _\|\|\|\|\|\|\| | |
| | | | | -TGGTCAA | |
| MPM_Mesothelioma_Gordon | 9 | hsa-miR-219 | 7mer-a1 | NNACAATC | 6.10E−05 |
| | | | | _\|\|\|\|\|\|\| | |
| | | | | -CTGTTAG | |
| MPM_Mesothelioma_Gordon | 13 | hsa-miR-1250 | 7mer-m8 | CNCACCGT | 6.10E−05 |
| | | | | _\|\|\|\|\|\|\| | |
| | | | | -CGTGGCA | |
| MPM_Mesothelioma_Gordon | 19 | hsa-miR-615 | 7mer-m8 | CSACCCCC | 6.10E−05 |
| | | | | _\|\|\|\|\|\|\| | |
| | | | | -CTGGGGG | |
| MPM_Mesothelioma_Gordon | 22 | hsa-miR-1284 | 7mer-m8 | NGTATAGA | 6.10E−05 |
| | | | | _\|\|\|\|\|\|\| | |
| | | | | -CATATCT | |
| MPM_Mesothelioma_Gordon | 40 | hsa-miR-3178 | 7mer-a1 | TCNCNCCC | 6.10E−05 |
| | | | | _\|\|\|\|\|\|\| | |
| | | | | -GCGCGGG | |
| MUC_Ovarian_Hendrix | 42 | hsa-miR-1181 | 7mer-m8 | GNGACGGT | 6.10E−05 |
| | | | | \|\|\|\|\|\|\|_ | |
| | | | | CGCTGCC- | |
| MUC_Ovarian_Hendrix | 56 | hsa-miR-508-3p | 7mer-a1 | TACAATNN | 6.10E−05 |
| | | | | \|\|\|\|\|\|\|_ | |
| | | | | ATGTTAG- | |
| OD_Brain_Bredel | 5 | hsa-miR-423-3p | 7mer-a1 | TACCNAGC | 6.10E−05 |
| | | | | _\|\|\|\|\|\|\| | |
| | | | | -TGGCTCG | |
| OD_Brain_Bredel | 16 | hsa-miR-1181 | 8mer | GGNGANGG | 1.53E−05 |
| | | | | \|\|\|\|\|\|\|\| | |
| | | | | CCGCTGCC | |
| OD_Brain_Bredel | 19 | hsa-miR-551a | 7mer-m8 | CGGGTCGC | 6.10E−05 |
| | | hsa-miR-551b | | _\|\|\|\|\|\|\| | |
| | | | | -CCCAGCG | |
| OD_Brain_Bredel | 37 | hsa-miR-29a-3p | 7mer-m8 | AATCAGTA | 6.10E−05 |
| | | | | \|\|\|\|\|\|\|_ | |
| | | | | TTAGTCA- | |
| ODGL_Brain_Sun | 37 | hsa-miR-126 | 8mer | CGGTACGA | 1.53E−05 |
| | | | | \|\|\|\|\|\|\|\| | |
| | | | | GCCATGCT | |
| ODGL_Brain_Sun | 38 | hsa-miR-1469 | 7mer-a1 | GCGCCGAT | 6.10E−05 |
| | | | | \|\|\|\|\|\|\|_ | |
| | | | | CGCGGCT- | |
| PDC_Pancreas_Ishikawa | 3 | hsa-miR-369 | 7mer-a1 | GTNGATCG | 6.10E−05 |
| | | | | \|\|\|\|\|\|\|_ | |
| | | | | CAGCTAG- | |
| PPC_Prostate_Dhanasekaran | 19 | hsa-miR-101-3p | 7mer-a1 | AGATAACT | 6.10E−05 |
| | | | | _\|\|\|\|\|\|\| | |
| | | | | -CTATTGA | |
| RCCC_Renal_Boer | 7 | hsa-miR-32 | 7mer-m8 | TGCAATAC | 6.10E−05 |
| | | hsa-miR-92a | | \|\|\|\|\|\|\|_ | |
| | | hsa-miR-92b | | ACGTTAT- | |
| RCCC_Renal_Lenburg | 2 | hsa-miR-487b | 8mer | GTANNATT | 1.53E−05 |
| | | | | \|\|\|\|\|\|\|\| | |
| | | | | CATGCTAA | |
| RCCC_Renal_Lenburg | 3 | hsa-miR-548f | 7mer-m8 | TANTTTTT | 6.10E−05 |
| | | hsa-miR-548e | | _\|\|\|\|\|\|\| | |
| | | hsa-miR-548x | | -TCAAAAA | |
| SMCL_Lung_Bhattacharjee | 16 | hsa-miR-339-3p | 7mer-a1 | CGGCGCTC | 6.10E−05 |
| | | | | _\|\|\|\|\|\|\| | |
| | | | | -CCGCGAG | |
| SMCL_Lung_Bhattacharjee | 23 | hsa-miR-3183 | 7mer-m8 | GAGAGGCC | 6.10E−05 |
| | | | | \|\|\|\|\|\|\|_ | |
| | | | | CTCTCCG- | |
| SMCL_Lung_Bhattacharjee | 51 | hsa-miR-1273 | 8mer | TGTNNCCC | 1.53E−05 |
| | | | | \|\|\|\|\|\|\|\| | |
| | | | | ACAGCGGG | |
| SMCL_Lung_Bhattacharjee | 60 | hsa-miR-671-3p | 7mer-a1 | GAACCKGC | 6.10E−05 |
| | | | | \|\|\|\|\|\|\|_ | |
| | | | | CTTGGCC- | |
| SQ_Lung_Bhattacharjee | 8 | hsa-miR-1203 | 7mer-a1 | CNCTCCGG | 6.10E−05 |
| | | | | _\|\|\|\|\|\|\| | |
| | | | | -CGAGGCC | |
| SQ_Lung_Bhattacharjee | 34 | hsa-miR-718 | 8mer | GGNGGAAG | 1.53E−05 |
| | | | | \|\|\|\|\|\|\|\| | |
| | | | | CCGCCTTC | |
| SQ_Lung_Bhattacharjee | 35 | hsa-miR-449c-3p | 7mer-m8 | GCTAGCAA | 6.10E−05 |
| | | | | _\|\|\|\|\|\|\| | |
| | | | | -GATCGTT | |
| SQ_Lung_Bhattacharjee | 44 | hsa-miR-4285 | 7mer-m8 | TCNCCGCG | 6.10E−05 |
| | | | | \|\|\|\|\|\|\|_ | |
| | | | | AGCGGCG- | |
| SRS_Ovarian_Hendrix | 67 | hsa-miR-886 | 7mer-a1 | CCNACCCT | 6.10E−05 |
| | | | | \|\|\|\|\|\|\|_ | |
| | | | | GGCTGGG- | |
| SRS_Ovarian_Hendrix | 75 | hsa-miR-937 | 7mer-a1 | CGCGCGGA | 6.10E−05 |
| | | | | _\|\|\|\|\|\|\| | |
| | | | | -CGCGCCT | |
| SRS_Ovarian_Hendrix | 81 | hsa-miR-3135 | 7mer-a1 | CCTAGGNN | 6.10E−05 |
| | | | | \|\|\|\|\|\|\|_ | |
| | | | | GGATCCG- | |
| TU_Prostate_Lapointe | 14 | hsa-miR-138-1-3p | 7mer-a1 | NNAAGTAG | 6.10E−05 |
| | | | | _\|\|\|\|\|\|\| | |
| | | | | -CTTCATC | |
| TU_Prostate_Lapointe | 24 | hsa-miR-487b | 8mer | GTANNATT | 1.53E−05 |
| | | | | \|\|\|\|\|\|\|\| | |
| | | | | CATGCTAA | |
| TU_Prostate_Lapointe | 26 | hsa-miR-566 | 7mer-m8 | GGCGCCCG | 6.10E−05 |
| | | | | \|\|\|\|\|\|\|_ | |
| | | | | CCGCGGG- | |
| TU_Prostate_Lapointe | 40 | hsa-miR-369 | 7mer-a1 | GTNGATCG | 6.10E−05 |
| | | | | \|\|\|\|\|\|\|_ | |
| | | | | CAGCTAG- | |

SUPPLEMENTARY TABLE 5

| | | | Uncorrected | |
|---|---|---|---|---|
| Dataset | Cluster | miRNA | P-value | miR2Disease |
| AC Brain Sun | 4 | hsa-mir-222 | 0.000357084 | glioma |
| AC Brain Sun | 6 | hsa-mir-1284 | 4.83E−06 | — |
| AC Brain Sun | 7 | hsa-mir-582-3p | 1.18E−05 | — |
| AC Brain Sun | 12 | hsa-mir-485-3p | 2.89E−06 | — |
| AC Brain Sun | 19 | hsa-mir-218 | 3.23E−05 | — |
| AC Brain Sun | 32 | hsa-mir-1266 | 0.000659312 | — |
| AC Brain Sun | 39 | hsa-mir-1266 | 0.00017731 | — |
| AC Brain Sun | 46 | hsa-mir-548c-3p | 4.75E−09 | — |
| AC Brain Sun | 50 | hsa-mir-32 | 0.000138143 | — |
| AC Brain Sun | 51 | hsa-mir-922 | 0.000626124 | — |
| AD Lung Beer | 1 | hsa-mir-766 | 2.26E−05 | — |
| AD Lung Beer | 3 | hsa-mir-338 | 3.66E−05 | lung cancer |
| AD Lung Beer | 7 | hsa-mir-548b hsa-mir-548a hsa-mir-548d hsa-mir-548i | 2.53E−05 | — |
| | | hsa-mir-548j hsa-mir-548c hsa-mir-548h | | |
| AD Lung Beer | 8 | hsa-mir-939 | 9.03E−05 | — |
| AD Lung Beer | 9 | hsa-mir-548k | 1.61E−05 | — |
| AD Lung Beer | 11 | hsa-mir-9 | 0.000564437 | lung cancer\|lung cancer\|non-small |
| | | | | cell lung cancer (NSCLC) |
| AD Lung Beer | 12 | hsa-mir-1208 | 7.53E−05 | — |
| AD Lung Beer | 14 | hsa-mir-571 | 4.68E−06 | — |
| AD Lung Beer | 18 | hsa-mir-876-3p | 0.00023225 | — |
| AD Lung Beer | 24 | hsa-mir-770 | 2.97E−05 | — |
| AD Lung Beer | 27 | hsa-mir-320 hsa-mir-320a hsa-mir-320b hsa-mir-320d | 0.000120148 | — |
| AD Lung Beer | 28 | hsa-mir-147 | 0.00058663 | — |
| AD Lung Beer | 29 | hsa-mir-661 | 0.000210827 | — |
| AD Lung Beer | 31 | hsa-mir-29 hsa-mir-29a hsa-mir-29b | 5.80E−06 | lung cancer\|lung cancer |
| AD Lung Beer | 32 | hsa-mir-656 | 0.000276057 | — |
| AD Lung Beer | 33 | hsa-mir-380 | 0.000197824 | — |
| AD Lung Beer | 34 | hsa-mir-193b | 0.000113204 | — |
| AD Lung Beer | 35 | hsa-mir-222 | 0.000175181 | non-small cell lung cancer (NSCLC) |
| AD Lung Beer | 40 | hsa-mir-320 hsa-mir-320a hsa-mir-320b hsa-mir-320d | 4.80E−05 | — |
| AD Lung Beer | 44 | hsa-mir-484 | 3.38E−05 | — |
| AD Lung Beer | 46 | hsa-mir-874 | 2.07E−05 | — |
| AD Lung Beer | 51 | hsa-mir-1202 | 0.00026996 | — |
| AD Lung Beer | 52 | hsa-mir-372 | 0.000455224 | non-small cell lung cancer (NSCLC) |
| AD Lung Beer | 53 | hsa-mir-581 | 3.77E−06 | — |
| AD Lung Bhattacharjee | 2 | hsa-mir-645 | 0.000246382 | — |
| AD Lung Bhattacharjee | 7 | hsa-mir-570 | 3.97E−05 | — |
| AD Lung Bhattacharjee | 14 | hsa-mir-517 hsa-mir-517a | 6.67E−05 | — |
| AD Lung Bhattacharjee | 19 | hsa-mir-1322 | 0.00026893 | — |
| AD Lung Bhattacharjee | 20 | hsa-mir-599 | 8.73E−05 | — |
| AD Lung Bhattacharjee | 22 | hsa-mir-875-3p | 2.47E−05 | — |
| AD Lung Bhattacharjee | 28 | hsa-mir-122 | 0.00016338 | — |
| AD Lung Bhattacharjee | 30 | hsa-mir-607 | 1.31E−05 | — |
| AD Lung Bhattacharjee | 33 | hsa-mir-892a | 0.000442944 | — |
| AD Lung Bhattacharjee | 39 | hsa-mir-525-3p hsa-mir-524-3p | 0.000313314 | — |
| AD Lung Bhattacharjee | 42 | hsa-mir-760 | 0.000234775 | — |
| AD Lung Bhattacharjee | 44 | hsa-mir-633 | 9.95E−06 | — |
| AD Lung Bhattacharjee | 46 | hsa-mir-548a-3p hsa-mir-548e hsa-mir-548f | 4.38E−07 | — |
| AD Lung Bhattacharjee | 47 | hsa-mir-409-3p | 1.02E−05 | — |
| AD Lung Bhattacharjee | 49 | hsa-mir-567 | 0.000411819 | — |
| AD Lung Bhattacharjee | 53 | hsa-mir-146b hsa-mir-146a | 0.000204499 | lung cancer |
| AD Lung Bhattacharjee | 58 | hsa-mir-655 | 2.48E−05 | — |
| AD Lung Bhattacharjee | 59 | hsa-mir-29 hsa-mir-29a hsa-mir-29b | 6.04E−07 | lung cancer\|lung cancer |
| AD Lung Bhattacharjee | 60 | hsa-mir-190b hsa-mir-190 | 9.50E−06 | — |
| AD Lung Bhattacharjee | 67 | hsa-mir-361 | 0.000188291 | — |
| AD Lung Bhattacharjee | 69 | hsa-mir-23b hsa-mir-23a | 2.83E−06 | — |
| AD Lung Bhattacharjee | 72 | hsa-mir-507 hsa-mir-557 | 5.02E−05 | — |
| AD Lung Bhattacharjee | 75 | hsa-mir-1270 hsa-mir-620 | 0.000200137 | — |
| AD Lung Bhattacharjee | 76 | hsa-mir-371-3p | 3.23E−05 | — |
| AD Lung Stearman | 0 | hsa-mir-518a hsa-mir-527 | 7.85E−06 | — |
| AD Lung Stearman | 7 | hsa-mir-1269 | 8.43E−05 | — |
| AD Lung Stearman | 9 | hsa-mir-548g | 0.000161471 | — |
| AD Lung Stearman | 13 | hsa-mir-455 | 0.000102984 | — |
| AD Lung Stearman | 18 | hsa-mir-1257 | 0.000200203 | — |
| AD Lung Stearman | 25 | hsa-mir-200a hsa-mir-141 | 0.000459035 | cancer\|lung cancer |
| AD Lung Stearman | 33 | hsa-mir-196b hsa-mir-196a | 9.42E−05 | — |
| AD Ovarian Welsh | 0 | hsa-mir-510 | 0.000176975 | — |
| AD Ovarian Welsh | 2 | hsa-mir-122 | 0.00027714 | — |
| AD Ovarian Welsh | 3 | hsa-mir-1283 | 2.12E−05 | — |
| AD Ovarian Welsh | 5 | hsa-mir-188-3p | 0.000360695 | — |
| AD Ovarian Welsh | 7 | hsa-mir-650 | 0.00037885 | — |
| AD Ovarian Welsh | 11 | hsa-mir-1323 hsa-mir-548o | 0.00020088 | — |
| AD Ovarian Welsh | 15 | hsa-mir-1282 | 6.41E−06 | — |
| AD Ovarian Welsh | 20 | hsa-mir-767 | 0.000326469 | — |
| AD Ovarian Welsh | 21 | hsa-mir-96 | 0.000127997 | — |
| AD Ovarian Welsh | 25 | hsa-mir-572 | 6.54E−06 | ovarian cancer (OC) |
| AD Ovarian Welsh | 26 | hsa-mir-210 | 8.63E−05 | — |
| AD Ovarian Welsh | 29 | hsa-mir-1280 | 1.97E−06 | — |
| AD Ovarian Welsh | 30 | hsa-mir-1207 | 2.62E−05 | — |
| AD Ovarian Welsh | 31 | hsa-mir-380 | 0.000597339 | — |
| AD Pancreas Logsdon | 13 | hsa-mir-486 | 0.000129758 | — |
| AD Pancreas Logsdon | 14 | hsa-mir-517 hsa-mir-517a | 4.15E−05 | — |
| AD Pancreas Logsdon | 15 | hsa-mir-556-3p | 9.44E−05 | — |
| AD Pancreas Logsdon | 16 | hsa-mir-561 | 0.000136915 | — |
| AO Brain Bredel | 11 | hsa-mir-608 | 1.92E−05 | — |
| AO Brain Bredel | 13 | hsa-mir-506 | 0.000219864 | — |
| AO Brain Bredel | 17 | hsa-mir-191 | 3.22E−05 | — |
| AO Brain Bredel | 20 | hsa-mir-324 | 0.000220545 | — |
| AO Brain Bredel | 30 | hsa-mir-1255b hsa-mir-1255a | 0.000171809 | — |
| AO Brain Bredel | 33 | hsa-mir-450b | 0.000452261 | — |
| AO Brain Bredel | 35 | hsa-mir-939 | 0.000489612 | — |
| B-CLL Leukemia Haslinger | 15 | hsa-mir-338 | 6.09E−05 | — |
| B-CLL Leukemia Haslinger | 21 | hsa-mir-429 hsa-mir-200b hsa-mir-200 | 5.23E−09 | cancer |
| B-CLL Leukemia Haslinger | 25 | hsa-mir-1237 | 9.44E−05 | — |
| B-CLL Leukemia Haslinger | 27 | hsa-mir-760 | 1.19E−07 | — |
| B-CLL Leukemia Haslinger | 29 | hsa-mir-520f | 0.000157234 | — |
| B-CLL Leukemia Haslinger | 30 | hsa-mir-654-3p | 0.000489152 | — |
| B-CLL Leukemia Haslinger | 34 | hsa-mir-492 | 4.95E−05 | — |
| B-CLL Leukemia Haslinger | 35 | hsa-mir-1294 | 2.05E−06 | — |
| B-CLL Leukemia Haslinger | 37 | hsa-mir-545 | 2.30E−05 | — |
| B-CLL Leukemia Haslinger | 40 | hsa-mir-582-3p | 0.000202998 | — |
| B-CLL Leukemia Haslinger | 46 | hsa-mir-1249 | 0.000175139 | — |
| B-CLL Leukemia Haslinger | 48 | hsa-mir-520d hsa-mir-524 | 0.000143885 | — |
| B-CLL Leukemia Haslinger | 49 | hsa-mir-660 | 0.000361137 | — |
| B-CLL Leukemia Haslinger | 52 | hsa-mir-323-3p | 0.000171267 | — |
| B-CLL Leukemia Haslinger | 53 | hsa-mir-513a-3p | 5.06E−05 | — |
| B-CLL Leukemia Haslinger | 55 | hsa-mir-1323 hsa-mir-548o | 2.12E−07 | — |
| B-CLL Leukemia Haslinger | 59 | hsa-mir-636 | 1.65E−05 | — |
| B-CLL Leukemia Haslinger | 62 | hsa-mir-515 | 2.65E−05 | — |
| B-CLL Leukemia Haslinger | 64 | hsa-mir-1278 | 0.000299993 | — |
| B-CLL Leukemia Haslinger | 68 | hsa-mir-377 | 0.000193905 | — |
| B-CLL Leukemia Haslinger | 69 | hsa-mir-532 | 0.000295895 | — |
| BPH Prostate Dhanasekaran | 4 | hsa-mir-543 | 1.06E−05 | — |
| BPH Prostate Dhanasekaran | 5 | hsa-mir-1283 | 0.000141737 | — |
| BPH Prostate Dhanasekaran | 6 | hsa-mir-648 | 0.000281772 | — |
| BPH Prostate Dhanasekaran | 7 | hsa-mir-508 | 0.000256525 | — |
| BPH Prostate Dhanasekaran | 8 | hsa-mir-891b | 7.31E−05 | — |
| BPH Prostate Dhanasekaran | 10 | hsa-mir-1290 | 0.000500185 | — |
| BPH Prostate Dhanasekaran | 11 | hsa-mir-520d hsa-mir-524 | 5.08E−05 | — |
| BPH Prostate Dhanasekaran | 12 | hsa-mir-33b hsa-mir-33a | 0.000395585 | — |
| BPH Prostate Dhanasekaran | 15 | hsa-mir-770 | 2.71E−05 | — |
| BPH Prostate Dhanasekaran | 16 | hsa-mir-548e | 4.60E−05 | — |
| CA Bladder Dyrskjot | 0 | hsa-mir-607 | 6.23E−05 | — |
| CA Bladder Dyrskjot | 1 | hsa-mir-940 | 1.95E−05 | — |
| CA Bladder Dyrskjot | 8 | hsa-mir-140-3p | 0.000606179 | — |
| CA Bladder Dyrskjot | 9 | hsa-mir-452 | 6.57E−08 | bladder cancer |
| CA Bladder Dyrskjot | 12 | hsa-mir-607 | 0.000333178 | — |
| CA Bladder Dyrskjot | 16 | hsa-mir-1205 | 4.42E−05 | — |
| CA Bladder Dyrskjot | 19 | hsa-mir-659 | 0.00021994 | — |
| CA Bladder Dyrskjot | 21 | hsa-mir-590-3p | 0.000292988 | — |
| CA Bladder Dyrskjot | 25 | hsa-mir-550 | 0.000110985 | — |
| CA Bladder Dyrskjot | 26 | hsa-mir-1224 | 9.15E−06 | — |
| CA Bladder Dyrskjot | 27 | hsa-mir-365 | 1.19E−05 | — |
| CA Bladder Dyrskjot | 31 | hsa-mir-520d hsa-mir-524 | 0.000672309 | — |
| CA Bladder Dyrskjot | 32 | hsa-mir-320 hsa-mir-320a hsa-mir-320b hsa-mir-320d | 3.19E−06 | — |
| CA Bladder Dyrskjot | 36 | hsa-mir-548n | 2.24E−07 | — |
| CA Bladder Dyrskjot | 42 | hsa-mir-1183 | 4.30E−05 | — |
| CA Bladder Dyrskjot | 47 | hsa-mir-331 | 1.08E−05 | — |
| CA Bladder Dyrskjot | 51 | hsa-mir-1276 | 0.000118306 | — |
| CA Bladder Dyrskjot | 53 | hsa-mir-1206 | 3.97E−05 | — |
| CA Bladder Dyrskjot | 56 | hsa-mir-335 | 8.82E−06 | — |
| CA Bladder Dyrskjot | 64 | hsa-mir-590-3p | 3.58E−12 | — |
| CA Bladder Dyrskjot | 66 | hsa-mir-1827 | 0.00059065 | — |
| CA Bladder Dyrskjot | 71 | hsa-mir-633 | 4.25E−05 | — |
| CA Bladder Dyrskjot | 75 | hsa-mir-487a | 0.000410448 | — |
| CA Bladder Dyrskjot | 79 | hsa-mir-147b | 4.66E−05 | — |
| CA Bladder Dyrskjot | 81 | hsa-mir-455-3p | 0.000167082 | — |
| CA Bladder Dyrskjot | 82 | hsa-mir-519d | 0.000101549 | — |
| CA Breast Richardson | 2 | hsa-mir-590-3p | 9.05E−05 | — |
| CA Breast Richardson | 4 | hsa-mir-433 | 5.10E−05 | — |
| CA Breast Richardson | 13 | hsa-mir-590-3p | 8.34E−05 | — |
| CA Breast Richardson | 32 | hsa-mir-1245 | 3.51E−05 | — |
| CA Breast Richardson | 33 | hsa-mir-876 | 0.000262306 | — |
| CA Breast Richardson | 34 | hsa-mir-410 | 2.79E−07 | — |
| CA Breast Richardson | 38 | hsa-mir-1271 | 5.68E−05 | — |
| CA Breast Richardson | 43 | hsa-mir-1246 | 0.000277995 | — |
| CA Breast Richardson | 45 | hsa-mir-181d hsa-mir-181b | 0.000100856 | breast cancer |
| CA Breast Richardson | 46 | hsa-mir-323-3p | 3.35E−05 | — |
| CA Breast Richardson | 53 | hsa-mir-548m | 1.84E−05 | — |
| CA Breast Richardson | 55 | hsa-mir-130b hsa-mir-301a hsa-mir-301b hsa-mir-130a | 0.000665578 | — |
| | | hsa-mir-454 | | |
| CA Breast Richardson | 56 | hsa-mir-146b hsa-mir-146a | 0.000198109 | breast cancer |
| CA Breast Sorlie | 2 | hsa-mir-653 | 0.000159728 | — |
| CA Breast Sorlie | 5 | hsa-mir-220 | 0.000125447 | — |
| CA Breast Sorlie | 8 | hsa-mir-494 | 0.000325493 | — |
| CA Breast Sorlie | 10 | hsa-mir-23b hsa-mir-23a | 0.000228613 | — |
| CA Breast Sorlie | 11 | hsa-mir-33a | 0.000170146 | — |
| CA Breast Sorlie | 12 | hsa-mir-548c-3p | 0.00053872 | — |
| CA Breast Sorlie | 14 | hsa-mir-632 | 0.000123817 | — |
| CA Breast Sorlie | 18 | hsa-mir-490-3p | 0.000434944 | — |
| CA Breast Sorlie | 19 | hsa-mir-155 | 6.17E−05 | breast cancer\|breast cancer\|breast |
| | | | | cancer\|breast cancer |
| CA Breast Sorlie | 20 | hsa-mir-1279 | 9.67E−07 | — |
| CA Colon Graudens | 2 | hsa-mir-551b hsa-mir-551a | 2.41E−05 | — |
| CA Colon Graudens | 3 | hsa-mir-552 | 0.000397074 | — |
| CA Colon Graudens | 8 | hsa-mir-595 | 0.000150353 | — |
| CA Colon Graudens | 9 | hsa-mir-338-3p | 0.000383018 | — |
| CA Colon Graudens | 14 | hsa-mir-1244 | 0.000207966 | — |
| CA Colon Graudens | 17 | hsa-mir-642 | 0.000254189 | — |
| CA Colon Graudens | 19 | hsa-mir-148a | 6.28E−05 | — |
| CA Colon Graudens | 24 | hsa-mir-381 hsa-mir-300 | 1.03E−05 | — |
| CA Colon Graudens | 25 | hsa-mir-202 | 0.000193429 | — |
| CA Colon Graudens | 28 | hsa-mir-566 | 0.000134232 | — |
| CA Colon Graudens | 29 | hsa-mir-337-3p | 0.00025569 | — |
| CA Colon Graudens | 30 | hsa-mir-125b hsa-mir-125a | 0.000141991 | colorectal cancer\|colorectal cancer |
| CA Colon Graudens | 35 | hsa-mir-325 | 1.27E−06 | — |
| CA Colon Graudens | 36 | hsa-mir-576 | 0.000293879 | — |
| CA Colon Graudens | 38 | hsa-mir-320 hsa-mir-320a hsa-mir-320b hsa-mir-320d | 9.49E−06 | colorectal cancer\|colorectal cancer |
| CA Colon Graudens | 39 | hsa-mir-873 | 1.14E−05 | — |
| CA Colon Graudens | 40 | hsa-mir-507 hsa-mir-557 | 0.000113046 | — |
| CA Colon Graudens | 41 | hsa-mir-1243 | 9.02E−05 | — |
| CA Renal Higgins | 1 | hsa-mir-590-3p | 0.000514794 | — |
| CA Renal Higgins | 10 | hsa-mir-875-3p | 3.13E−05 | — |
| CA Renal Higgins | 13 | hsa-mir-423 | 4.95E−06 | — |
| CA Renal Higgins | 14 | hsa-mir-140 | 0.000244645 | — |
| CCC Ovarian Hendrix | 1 | hsa-mir-1200 | 0.000441527 | — |
| CCC Ovarian Hendrix | 4 | hsa-mir-194 | 2.92E−06 | — |
| CCC Ovarian Hendrix | 7 | hsa-mir-532 | 0.000214675 | — |
| CCC Ovarian Hendrix | 11 | hsa-mir-29 hsa-mir-29a hsa-mir-29b | 3.61E−07 | ovarian cancer (OC) |
| CCC Ovarian Hendrix | 12 | hsa-mir-876 | 1.89E−05 | — |
| CCC Ovarian Hendrix | 13 | hsa-mir-143 | 1.94E−05 | epithelial ovarian cancer (EOC) |
| CCC Ovarian Hendrix | 23 | hsa-mir-105 | 0.00057308 | epithelial ovarian cancer (EOC) |
| CCC Ovarian Hendrix | 24 | hsa-mir-19b hsa-mir-19a | 0.000228998 | — |
| CCC Ovarian Hendrix | 25 | hsa-mir-183 | 2.99E−05 | ovarian cancer (OC) |
| CCC Ovarian Hendrix | 30 | hsa-mir-590 | 6.39E−05 | — |
| CCC Ovarian Hendrix | 33 | hsa-mir-127 | 0.000208615 | cancer\|epithelial ovarian cancer (EOC) |
| CCC Ovarian Hendrix | 35 | hsa-mir-1283 | 0.000100312 | — |
| CCC Ovarian Hendrix | 37 | hsa-mir-1302 | 9.90E−05 | — |
| CCC Ovarian Hendrix | 42 | hsa-mir-590-3p | 3.94E−05 | — |
| CCC Ovarian Hendrix | 45 | hsa-mir-382 | 0.000105777 | — |
| CCC Ovarian Hendrix | 47 | hsa-mir-548p | 4.74E−05 | — |
| CCC Ovarian Hendrix | 51 | hsa-mir-1279 | 0.000323532 | — |
| CCC Ovarian Hendrix | 56 | hsa-mir-409-3p | 0.000166079 | — |
| CCC Ovarian Hendrix | 59 | hsa-mir-552 | 2.65E−05 | — |
| CCC Ovarian Hendrix | 60 | hsa-mir-105 | 1.20E−06 | epithelial ovarian cancer (EOC) |
| CLL Lymphoma Alizadeh | 0 | hsa-mir-1283 | 9.53E−05 | — |
| CLL Lymphoma Alizadeh | 3 | hsa-mir-513b | 1.11E−05 | — |
| CLL Lymphoma Alizadeh | 7 | hsa-mir-1263 | 9.63E−05 | — |
| CLL Lymphoma Alizadeh | 8 | hsa-mir-222 | 0.000378332 | — |
| CLL Lymphoma Alizadeh | 10 | hsa-mir-21 hsa-mir-590 | 6.63E−05 | chronic lymphocytic leukemia (CLL) |
| COID Lung Bhattacharjee | 0 | hsa-mir-663 | 9.57E−05 | — |
| COID Lung Bhattacharjee | 2 | hsa-mir-548d-3p | 0.000222684 | — |
| COID Lung Bhattacharjee | 3 | hsa-mir-940 | 7.85E−05 | — |
| COID Lung Bhattacharjee | 5 | hsa-mir-361 | 0.000204845 | — |
| COID Lung Bhattacharjee | 7 | hsa-mir-431 | 6.94E−05 | — |
| COID Lung Bhattacharjee | 8 | hsa-mir-607 | 0.000109642 | — |
| COID Lung Bhattacharjee | 9 | hsa-mir-622 | 0.000578648 | — |
| COID Lung Bhattacharjee | 10 | hsa-mir-513a-3p | 9.27E−06 | — |
| COID Lung Bhattacharjee | 11 | hsa-mir-501 | 0.000106934 | — |
| COID Lung Bhattacharjee | 12 | hsa-mir-509 hsa-mir-509-3 | 0.00045535 | — |
| COID Lung Bhattacharjee | 17 | hsa-mir-885-3p | 2.71E−07 | — |
| COID Lung Bhattacharjee | 19 | hsa-mir-553 | 2.94E−05 | — |
| COID Lung Bhattacharjee | 21 | hsa-mir-590-3p | 0.000341529 | — |
| COID Lung Bhattacharjee | 25 | hsa-mir-1276 | 1.52E−05 | — |
| COID Lung Bhattacharjee | 28 | hsa-mir-758 | 0.000192537 | — |
| COID Lung Bhattacharjee | 31 | hsa-mir-331-3p | 0.000462838 | — |
| COID Lung Bhattacharjee | 40 | hsa-mir-1181 | 2.31E−05 | — |
| COID Lung Bhattacharjee | 41 | hsa-mir-1303 | 0.000150263 | — |
| COID Lung Bhattacharjee | 44 | hsa-mir-504 | 3.96E−05 | — |
| COID Lung Bhattacharjee | 47 | hsa-mir-574-3p | 2.48E−05 | — |
| COID Lung Bhattacharjee | 60 | hsa-mir-505 | 0.00037429 | — |
| COID Lung Bhattacharjee | 61 | hsa-mir-494 | 1.43E−05 | — |
| COID Lung Bhattacharjee | 67 | hsa-mir-512-3p | 0.000326325 | — |
| COID Lung Bhattacharjee | 71 | hsa-mir-1237 | 0.000255541 | — |
| COID Lung Bhattacharjee | 73 | hsa-mir-1262 | 0.000380974 | — |
| COID Lung Bhattacharjee | 74 | hsa-mir-513a-3p | 4.85E−06 | — |
| COID Lung Bhattacharjee | 75 | hsa-mir-1300 | 0.000178305 | — |
| COID Lung Bhattacharjee | 78 | hsa-mir-920 | 0.000256218 | — |
| COID Lung Bhattacharjee | 81 | hsa-mir-369-3p | 3.12E−05 | — |
| COID Lung Bhattacharjee | 86 | hsa-mir-580 | 5.05E−05 | — |
| COID Lung Bhattacharjee | 89 | hsa-mir-1254 | 0.000134399 | — |
| COID Lung Bhattacharjee | 91 | hsa-mir-1275 | 5.25E−06 | — |
| DLBCL Lymphoma Alizadeh | 1 | hsa-mir-138 | 3.63E−05 | — |
| DLBCL Lymphoma Alizadeh | 5 | hsa-mir-198 | 0.000115292 | — |
| DLBCL Lymphoma Alizadeh | 6 | hsa-mir-542 | 1.20E−05 | — |
| DLBCL Lymphoma Alizadeh | 7 | hsa-mir-532-3p | 3.78E−05 | — |
| DLBCL Lymphoma Alizadeh | 9 | hsa-mir-892b | 0.000231852 | — |
| DLBCL Lymphoma Alizadeh | 10 | hsa-mir-325 | 0.00021668 | — |
| DLBCL Lymphoma Alizadeh | 11 | hsa-mir-1203 | 6.38E−05 | — |
| END Ovarian Hendrix | 3 | hsa-mir-510 | 1.61E−06 | — |
| END Ovarian Hendrix | 4 | hsa-mir-608 | 2.66E−05 | ovarian cancer (OC) |
| END Ovarian Hendrix | 6 | hsa-mir-29 hsa-mir-29a hsa-mir-29b | 6.95E−05 | ovarian cancer (OC) |
| END Ovarian Hendrix | 7 | hsa-mir-1302 | 1.69E−05 | — |
| END Ovarian Hendrix | 8 | hsa-mir-200a hsa-mir-141 | 0.000119842 | cancer\|epithelial ovarian cancer |
| | | | | (EOC)\|ovarian cancer (OC)\|ovarian |
| | | | | cancer (OC)\|ovarian cancer (OC) |
| END Ovarian Hendrix | 13 | hsa-mir-1276 | 0.000413656 | — |
| END Ovarian Hendrix | 18 | hsa-mir-890 | 6.81E−05 | — |
| END Ovarian Hendrix | 22 | hsa-mir-15b hsa-mir-15a hsa-mir-16 hsa-mir-497 | 0.000286656 | ovarian cancer (OC) |
| | | hsa-mir-195 | | |
| END Ovarian Hendrix | 23 | hsa-mir-1259 | 0.000453022 | — |
| END Ovarian Hendrix | 25 | hsa-mir-653 | 0.000151323 | — |
| END Ovarian Hendrix | 26 | hsa-mir-548c-3p | 1.03E−05 | — |
| END Ovarian Hendrix | 32 | hsa-mir-802 | 0.000186055 | — |
| END Ovarian Hendrix | 35 | hsa-mir-1276 | 9.71E−20 | — |
| END Ovarian Hendrix | 38 | hsa-mir-944 | 0.000151186 | — |
| END Ovarian Hendrix | 39 | hsa-mir-508 | 0.000310807 | ovarian cancer (OC) |
| END Ovarian Hendrix | 48 | hsa-mir-125a-3p | 0.000130068 | — |
| END Ovarian Hendrix | 50 | hsa-mir-539 | 3.78E−05 | — |
| END Ovarian Hendrix | 51 | hsa-mir-130b hsa-mir-301a hsa-mir-301b hsa-mir-130a | 6.35E−05 | — |
| | | hsa-mir-454 | | |
| END Ovarian Hendrix | 60 | hsa-mir-580 | 1.92E−06 | — |
| END Ovarian Hendrix | 69 | hsa-mir-607 | 0.000218523 | — |
| END Ovarian Hendrix | 82 | hsa-mir-505 | 8.54E−06 | — |
| FL Lymphoma Alizadeh | 4 | hsa-mir-1295 | 1.54E−05 | — |
| FL Lymphoma Alizadeh | 6 | hsa-mir-338 | 0.000302415 | follicular lymphoma (FL) |
| FL Lymphoma Alizadeh | 9 | hsa-mir-767-3p | 0.000117612 | — |
| FL Lymphoma Alizadeh | 11 | hsa-mir-148a hsa-mir-148b hsa-mir-152 | 0.000429493 | — |
| GBM Brain Liang | 12 | hsa-mir-520g hsa-mir-520h | 0.000557506 | — |
| GBM Brain Liang | 14 | hsa-mir-125a-3p | 0.000424903 | — |
| GBM Brain Liang | 15 | hsa-mir-144 | 0.000662353 | — |
| GBM Brain Liang | 17 | hsa-mir-600 | 0.00022619 | — |
| GCT Seminoma Korkola | 6 | hsa-mir-34b | 0.000166029 | — |
| GCT Seminoma Korkola | 8 | hsa-mir-940 | 1.62E−05 | — |
| GCT Seminoma Korkola | 14 | hsa-mir-1323 hsa-mir-548o | 3.12E−05 | — |
| GCT Seminoma Korkola | 20 | hsa-mir-632 | 4.09E−05 | — |
| GCT Seminoma Korkola | 21 | hsa-mir-548k | 4.81E−06 | — |
| GCT Seminoma Korkola | 25 | hsa-mir-105 | 0.000213709 | — |
| GCT Seminoma Korkola | 26 | hsa-mir-138 | 0.000182637 | — |
| GCT Seminoma Korkola | 31 | hsa-mir-22 | 3.97E−06 | — |
| GCT Seminoma Korkola | 36 | hsa-mir-196b hsa-mir-196a | 0.000488975 | — |
| GCT Seminoma Korkola | 37 | hsa-mir-106b hsa-mir-17 hsa-mir-106a hsa-mir-93 | 7.12E−05 | — |
| | | hsa-mir-20b hsa-mir-20a | | |
| GCT Seminoma Korkola | 38 | hsa-mir-370 | 0.000160824 | — |
| GCT Seminoma Korkola | 39 | hsa-mir-769 | 2.72E−05 | — |
| GCT Seminoma Korkola | 40 | hsa-mir-577 | 0.000614553 | — |
| GCT Seminoma Korkola | 42 | hsa-mir-149 | 0.000282927 | — |
| GCT Seminoma Korkola | 47 | hsa-mir-485 | 0.000532439 | — |
| GCT Seminoma Korkola | 56 | hsa-mir-499-3p | 0.000181753 | — |
| GCT Seminoma Korkola | 62 | hsa-mir-548m | 7.00E−07 | — |
| GCT Seminoma Korkola | 66 | hsa-mir-532-3p | 0.000274953 | — |
| GCT Seminoma Korkola | 72 | hsa-mir-490-3p | 8.74E−07 | — |
| GCT Seminoma Korkola | 74 | hsa-let-7f hsa-let-7g hsa-let-7a hsa-let-7b hsa-let-7d | 1.23E−06 | — |
| | | hsa-let-7i hsa-mir-98 hsa-let-7 | | |
| GCT Seminoma Korkola | 75 | hsa-mir-664 | 0.000114433 | — |
| GCT Seminoma Korkola | 76 | hsa-mir-220b | 5.80E−05 | — |
| GCT Seminoma Korkola | 80 | hsa-mir-623 | 8.85E−05 | — |
| GCT Seminoma Korkola | 87 | hsa-mir-1179 | 4.11E−05 | — |
| GCT Seminoma Korkola | 88 | hsa-mir-129 | 7.94E−05 | — |
| GCT Seminoma Korkola | 89 | hsa-mir-568 | 0.00034329 | — |
| GCT Seminoma Korkola | 90 | hsa-mir-362 | 3.27E−06 | — |
| GCT Seminoma Korkola | 93 | hsa-mir-193a | 0.000207726 | — |
| GCT Seminoma Korkola | 95 | hsa-mir-1283 | 3.81E−07 | — |
| GCT Seminoma Korkola | 98 | hsa-mir-224 | 0.000173618 | — |
| GCT Seminoma Korkola | 100 | hsa-mir-486-3p | 0.000207588 | — |
| GCT Seminoma Korkola | 102 | hsa-mir-125b hsa-mir-125a | 5.68E−06 | — |
| GCT Seminoma Korkola | 105 | hsa-mir-34a hsa-mir-34c hsa-mir-449a hsa-mir-449b | 0.000332836 | — |
| GCT Seminoma Korkola | 107 | hsa-mir-199b-3p hsa-mir-199a-3p | 0.000212588 | cancer |
| GCT Seminoma Korkola | 108 | hsa-mir-570 | 0.000279799 | — |
| GCT Seminoma Korkola | 109 | hsa-mir-548p | 5.74E−06 | — |
| GCT Seminoma Korkola | 110 | hsa-mir-18a hsa-mir-18b | 2.70E−05 | — |
| GCT Seminoma Korkola | 112 | hsa-mir-590-3p | 5.49E−06 | — |
| GL Brain Bredel | 0 | hsa-mir-1208 | 9.31E−05 | — |
| GL Brain Bredel | 9 | hsa-mir-549 | 0.000116643 | — |
| GL Brain Bredel | 11 | hsa-mir-542-3p | 0.000201348 | — |
| GL Brain Bredel | 12 | hsa-mir-384 | 0.000466337 | — |
| GL Brain Bredel | 15 | hsa-mir-654-3p | 6.04E−07 | — |
| GL Brain Bredel | 20 | hsa-mir-487b | 0.000468745 | — |
| GL Brain Bredel | 21 | hsa-mir-633 | 6.38E−05 | — |
| GL Brain Bredel | 24 | hsa-mir-1245 | 0.000261253 | — |
| GL Brain Bredel | 25 | hsa-mir-338 | 0.000377267 | — |
| GL Brain Bredel | 33 | hsa-mir-625 | 0.000450263 | — |
| GL Brain Bredel | 36 | hsa-mir-661 | 0.00010024 | — |
| GL Brain Bredel | 37 | hsa-mir-544 | 0.000666316 | — |
| GL Brain Bredel | 39 | hsa-mir-363 | 0.000215653 | — |
| GL Brain Bredel | 40 | hsa-mir-526b | 0.000108823 | — |
| GL Brain Bredel | 41 | hsa-mir-484 | 0.000147486 | — |
| GL Brain Bredel | 46 | hsa-mir-154 | 0.000413209 | — |
| GL Brain Bredel | 54 | hsa-mir-582-3p | 5.64E−05 | — |
| GL Brain Bredel | 57 | hsa-mir-769-3p hsa-mir-450b-3p | 5.61E−05 | — |
| GL Brain Bredel | 59 | hsa-mir-125a-3p | 2.04E−06 | — |
| GL Brain Bredel | 60 | hsa-mir-513a | 0.000188295 | — |
| GL Brain Bredel | 61 | hsa-mir-572 | 5.05E−06 | — |
| GL Brain Bredel | 64 | hsa-mir-220b | 9.88E−05 | — |
| GL Brain Bredel | 65 | hsa-mir-532 | 0.000243031 | — |
| GL Brain Bredel | 71 | hsa-mir-651 | 0.000504986 | — |
| GL Brain Bredel | 75 | hsa-mir-889 | 0.000677584 | — |
| GL Brain Rickman | 1 | hsa-mir-493 | 3.61E−05 | — |
| GL Brain Rickman | 4 | hsa-mir-302f | 3.04E−05 | — |
| GL Brain Rickman | 9 | hsa-mir-1207 | 0.000100819 | — |
| GL Brain Rickman | 11 | hsa-mir-653 | 8.21E−05 | — |
| GL Brain Rickman | 13 | hsa-mir-486-3p | 0.000165893 | — |
| GL Brain Rickman | 14 | hsa-mir-149 | 0.000224784 | glioblastoma multiforme (GBM) |
| GL Brain Rickman | 21 | hsa-mir-624 | 8.28E−05 | — |
| GL Brain Rickman | 22 | hsa-mir-542-3p | 5.93E−05 | — |
| GL Brain Rickman | 23 | hsa-mir-490 | 1.28E−05 | — |
| GL Brain Rickman | 29 | hsa-mir-544 | 3.71E−06 | — |
| GL Brain Rickman | 31 | hsa-let-7e hsa-let-7f hsa-let-7g hsa-let-7a hsa-let-7b | 1.86E−05 | — |
| | | hsa-let-7d hsa-let-7i hsa-mir-98 hsa-let-7 | | |
| GL Brain Rickman | 34 | hsa-mir-125a-3p | 5.67E−05 | — |
| GLB Brain Sun | 1 | hsa-mir-767 | 9.57E−05 | — |
| GLB Brain Sun | 2 | hsa-mir-577 | 0.000325921 | — |
| GLB Brain Sun | 8 | hsa-mir-140-3p | 6.49E−07 | — |
| GLB Brain Sun | 10 | hsa-mir-588 | 2.76E−05 | — |
| GLB Brain Sun | 12 | hsa-mir-924 | 0.000279414 | — |
| GLB Brain Sun | 14 | hsa-mir-1299 | 0.0006165 | — |
| GLB Brain Sun | 15 | hsa-mir-582-3p | 6.42E−06 | — |
| GLB Brain Sun | 17 | hsa-mir-621 | 4.22E−05 | — |
| GLB Brain Sun | 18 | hsa-mir-655 | 2.74E−05 | — |
| GLB Brain Sun | 20 | hsa-mir-556-3p | 1.56E−05 | — |
| GLB Brain Sun | 21 | hsa-mir-188 | 0.000650779 | — |
| GLB Brain Sun | 24 | hsa-mir-381 hsa-mir-300 | 6.92E−05 | — |
| GLB Brain Sun | 27 | hsa-mir-590-3p | 0.000472328 | — |
| GLB Brain Sun | 28 | hsa-mir-369-3p | 5.05E−05 | — |
| GLB Brain Sun | 31 | hsa-mir-936 | 1.07E−05 | — |
| GLB Brain Sun | 40 | hsa-mir-1274a | 0.000402526 | — |
| GLB Brain Sun | 41 | hsa-mir-760 | 7.71E−09 | — |
| GLB Brain Sun | 43 | hsa-mir-331 | 5.34E−05 | — |
| GLB Brain Sun | 50 | hsa-mir-664 | 6.69E−05 | — |
| GLB Brain Sun | 51 | hsa-mir-28-3p | 0.000446009 | — |
| GLB Brain Sun | 54 | hsa-mir-548m | 5.72E−06 | — |
| GLB Brain Sun | 62 | hsa-mir-216a | 0.000157554 | — |
| GLB Brain Sun | 67 | hsa-mir-544 | 4.07E−07 | — |
| GLB Brain Sun | 68 | hsa-mir-888 | 7.93E−05 | — |
| GLB Brain Sun | 69 | hsa-mir-1288 | 0.000168213 | — |
| GLB Brain Sun | 71 | hsa-mir-1182 | 0.000395608 | — |
| GLB Brain Sun | 72 | hsa-mir-571 | 6.09E−05 | — |
| GLB Brain Sun | 76 | hsa-mir-561 | 3.82E−05 | — |
| HSCC Head-Neck Chung | 1 | hsa-mir-767 | 9.70E−05 | — |
| HSCC Head-Neck Chung | 2 | hsa-mir-148a hsa-mir-148b hsa-mir-152 | 0.000575605 | head and neck squamous cell |
| | | | | carcinoma (HNSCC)\|Oral Squamous |
| | | | | Cell Carcinoma (OSCC) |
| HSCC Head-Neck Chung | 7 | hsa-mir-487a | 4.23E−05 | — |
| HSCC Head-Neck Chung | 10 | hsa-mir-1226 | 0.000450182 | — |
| HSCC Head-Neck Cromer | 3 | hsa-mir-496 | 2.71E−05 | — |
| HSCC Head-Neck Cromer | 11 | hsa-mir-129 | 4.13E−05 | — |
| HSCC Head-Neck Cromer | 12 | hsa-mir-1297 hsa-mir-26a hsa-mir-26b | 3.36E−05 | Oral Squamous Cell Carcinoma (OSCC) |
| HSCC Head-Neck Cromer | 13 | hsa-mir-875-3p | 1.98E−05 | — |
| HSCC Head-Neck Cromer | 18 | hsa-mir-214 | 0.000212009 | head and neck squamous cell |
| | | | | carcinoma (HNSCC) |
| HSCC Head-Neck Cromer | 20 | hsa-mir-543 | 0.000173579 | — |
| HSCC Head-Neck Cromer | 22 | hsa-mir-218 | 0.000659831 | — |
| HSCC Head-Neck Cromer | 23 | hsa-mir-380 | 0.000341454 | — |
| HSCC Head-Neck Cromer | 25 | hsa-mir-1264 | 7.12E−05 | — |
| HSCC Head-Neck Cromer | 26 | hsa-mir-199a hsa-mir-199b | 0.000146104 | cancer\|Oral Squamous Cell Carcinoma |
| | | | | (OSCC)\|Oral Squamous Cell |
| | | | | Carcinoma (OSCC)\|Oral Squamous |
| | | | | Cell Carcinoma (OSCC) |
| IDC Breast Radvanyi | 4 | hsa-mir-661 | 0.00019994 | breast cancer |
| IDC Breast Radvanyi | 5 | hsa-mir-1274a | 9.35E−05 | — |
| IDC Breast Radvanyi | 6 | hsa-mir-652 | 4.44E−06 | — |
| IDC Breast Radvanyi | 8 | hsa-mir-1273 | 1.23E−05 | — |
| IDC Breast Radvanyi | 14 | hsa-mir-556 | 0.000534734 | — |
| IDC Breast Radvanyi | 15 | hsa-mir-182 | 2.40E−05 | breast cancer |
| IDC Breast Radvanyi | 16 | hsa-mir-9 | 0.000460178 | breast cancer\|breast cancer\|breast |
| | | | | cancer |
| IDC Breast Radvanyi | 21 | hsa-mir-493 | 0.000515054 | — |
| IDC Breast Radvanyi | 22 | hsa-mir-1274a | 0.000321613 | — |
| IDC Breast Radvanyi | 34 | hsa-mir-532 | 6.62E−05 | — |
| IDC Breast Radvanyi | 35 | hsa-mir-488 | 0.000481039 | — |
| IDC Breast Radvanyi | 41 | hsa-mir-361-3p | 5.52E−05 | — |
| IDC Breast Radvanyi | 44 | hsa-mir-548a-3p | 0.000104312 | — |
| IDC Breast Radvanyi | 47 | hsa-mir-760 | 0.000124285 | — |
| IDC Breast Radvanyi | 48 | hsa-mir-630 | 0.000196602 | — |
| IDC Breast Radvanyi | 51 | hsa-mir-5481 | 0.000143163 | — |
| IDC Breast Radvanyi | 54 | hsa-mir-1277 | 0.000645264 | — |
| IDC Breast Radvanyi | 56 | hsa-mir-216b | 0.00013594 | — |
| IDC Breast Radvanyi | 60 | hsa-mir-923 | 4.22E−05 | — |
| ILC Breast Radvanyi | 4 | hsa-mir-122 | 1.22E−06 | — |
| ILC Breast Radvanyi | 5 | hsa-mir-501 | 0.000523698 | — |
| ILC Breast Radvanyi | 19 | hsa-mir-663b | 3.73E−05 | — |
| ILC Breast Radvanyi | 22 | hsa-mir-508-3p | 0.000193266 | — |
| MCA Breast Radvanyi | 7 | hsa-mir-595 | 0.000118204 | — |
| MCA Breast Radvanyi | 12 | hsa-mir-489 | 0.000319697 | — |
| MCA Breast Radvanyi | 21 | hsa-mir-626 | 0.000107826 | — |
| ME Melanoma Hoek | 0 | hsa-mir-34b | 9.82E−05 | malignant melanoma |
| ME Melanoma Hoek | 3 | hsa-mir-1308 | 9.97E−05 | — |
| ME Melanoma Hoek | 6 | hsa-mir-410 | 6.65E−05 | — |
| ME Melanoma Hoek | 9 | hsa-mir-106b hsa-mir-17 hsa-mir-106a hsa-mir-93 | 0.000626339 | malignant melanoma |
| | | hsa-mir-20b hsa-mir-20a | | |
| ME Melanoma Hoek | 12 | hsa-mir-892a | 2.65E−05 | — |
| ME Melanoma Hoek | 15 | hsa-mir-217 | 5.33E−05 | — |
| ME Melanoma Hoek | 23 | hsa-mir-32 | 0.000286963 | — |
| ME Melanoma Hoek | 35 | hsa-mir-200b hsa-mir-200 | 4.37E−05 | cancer\|malignant melanoma |
| ME Melanoma Hoek | 39 | hsa-mir-212 hsa-mir-132 | 9.98E−05 | — |
| ME Melanoma Hoek | 41 | hsa-mir-938 | 4.28E−08 | — |
| ME Melanoma Hoek | 44 | hsa-mir-1299 | 3.96E−05 | — |
| ME Melanoma Hoek | 47 | hsa-mir-549 | 5.91E−05 | — |
| ME Melanoma Hoek | 50 | hsa-mir-548c-3p | 0.000144326 | — |
| ML Melanoma Talantov | 0 | hsa-mir-603 | 0.000216878 | — |
| ML Melanoma Talantov | 3 | hsa-mir-181a hsa-mir-181 | 2.83E−06 | malignant melanoma |
| ML Melanoma Talantov | 7 | hsa-mir-125b hsa-mir-125a | 4.64E−05 | malignant melanoma |
| ML Melanoma Talantov | 9 | hsa-mir-151-3p | 0.000153257 | — |
| ML Melanoma Talantov | 10 | hsa-mir-623 | 4.85E−05 | — |
| ML Melanoma Talantov | 12 | hsa-mir-125b hsa-mir-125a | 0.000126841 | malignant melanoma |
| ML Melanoma Talantov | 14 | hsa-mir-519a hsa-mir-519c-3p hsa-mir-519b-3p | 2.70E−07 | — |
| ML Melanoma Talantov | 19 | hsa-mir-421 | 0.000150563 | — |
| ML Melanoma Talantov | 22 | hsa-mir-520d hsa-mir-524 | 3.48E−05 | — |
| ML Melanoma Talantov | 23 | hsa-mir-548p | 7.57E−06 | — |
| ML Melanoma Talantov | 25 | hsa-mir-606 | 0.000376617 | — |
| ML Melanoma Talantov | 26 | hsa-mir-607 | 5.50E−05 | — |
| ML Melanoma Talantov | 34 | hsa-mir-767-3p | 3.81E−06 | — |
| MM Myeloma Zhan | 1 | hsa-mir-519e | 0.000195242 | — |
| MM Myeloma Zhan | 2 | hsa-mir-587 | 0.00038156 | — |
| MM Myeloma Zhan | 3 | hsa-mir-590-3p | 2.57E−08 | — |
| MM Myeloma Zhan | 4 | hsa-mir-361-3p | 0.00038289 | — |
| MM Myeloma Zhan | 6 | hsa-mir-516b | 5.95E−05 | — |
| MM Myeloma Zhan | 7 | hsa-mir-510 | 0.000138587 | — |
| MM Myeloma Zhan | 9 | hsa-mir-9 | 0.000100248 | — |
| MM Myeloma Zhan | 13 | hsa-mir-655 | 7.50E−05 | — |
| MM Myeloma Zhan | 17 | hsa-mir-320 hsa-mir-320a hsa-mir-320b hsa-mir-320d | 8.23E−06 | — |
| MM Myeloma Zhan | 20 | hsa-mir-1226 | 3.53E−05 | — |
| MM Myeloma Zhan | 21 | hsa-mir-183 | 0.000548746 | — |
| MM Myeloma Zhan | 22 | hsa-mir-1236 | 0.000330323 | — |
| MM Myeloma Zhan | 24 | hsa-mir-140-3p | 4.31E−05 | multiple myeloma (MM) |
| MM Myeloma Zhan | 25 | hsa-mir-188 | 0.000241687 | — |
| MM Myeloma Zhan | 29 | hsa-mir-760 | 1.88E−08 | — |
| MM Myeloma Zhan | 30 | hsa-mir-588 | 0.000131928 | — |
| MM Myeloma Zhan | 31 | hsa-mir-597 | 0.000240387 | — |
| MM Myeloma Zhan | 32 | hsa-mir-212 hsa-mir-132 | 0.000138448 | — |
| MM Myeloma Zhan | 35 | hsa-mir-650 | 0.000432285 | — |
| MM Myeloma Zhan | 40 | hsa-mir-382 | 0.000167046 | — |
| MM Myeloma Zhan | 43 | hsa-mir-23b hsa-mir-23a | 1.74E−05 | — |
| MM Myeloma Zhan | 45 | hsa-mir-595 | 0.00024781 | — |
| MM Myeloma Zhan | 46 | hsa-mir-425 | 5.54E−05 | — |
| MM Myeloma Zhan | 47 | hsa-mir-875-3p | 0.000422238 | — |
| MPC Prostate Dhanasekaran | 0 | hsa-mir-1323 hsa-mir-548o | 6.62E−07 | — |
| MPC Prostate Dhanasekaran | 7 | hsa-mir-330 hsa-mir-326 | 6.20E−05 | prostate cancer |
| MPC Prostate Dhanasekaran | 9 | hsa-mir-190b hsa-mir-190 | 2.92E−06 | — |
| MPC Prostate Dhanasekaran | 12 | hsa-mir-139 | 7.63E−07 | — |
| MPC Prostate Dhanasekaran | 13 | hsa-mir-1257 | 8.37E−06 | — |
| MPC Prostate Dhanasekaran | 14 | hsa-mir-133b hsa-mir-133a | 0.00010216 | — |
| MPC Prostate Dhanasekaran | 16 | hsa-mir-944 | 7.22E−07 | — |
| MPC Prostate Dhanasekaran | 19 | hsa-mir-661 | 1.85E−05 | — |
| MPC Prostate Dhanasekaran | 21 | hsa-mir-498 | 0.000467932 | prostate cancer |
| MPC Prostate Dhanasekaran | 22 | hsa-mir-105 | 2.03E−05 | — |
| MPC Prostate Dhanasekaran | 26 | hsa-mir-935 | 0.000148827 | — |
| MPC Prostate Dhanasekaran | 30 | hsa-mir-659 | 0.000152617 | — |
| MPC Prostate Dhanasekaran | 34 | hsa-mir-544 | 6.81E−07 | — |
| MPC Prostate Dhanasekaran | 35 | hsa-mir-203 | 0.000205357 | — |
| MPC Prostate Dhanasekaran | 44 | hsa-mir-1323 hsa-mir-548o | 1.10E−06 | — |
| MPC Prostate Dhanasekaran | 45 | hsa-mir-490-3p | 1.70E−06 | — |
| MPM Mesothelioma Gordon | 7 | hsa-mir-526b | 7.16E−05 | — |
| MPM Mesothelioma Gordon | 11 | hsa-mir-491-3p | 3.42E−05 | — |
| MPM Mesothelioma Gordon | 14 | hsa-mir-361-3p | 1.00E−04 | — |
| MPM Mesothelioma Gordon | 16 | hsa-mir-548m | 3.90E−08 | — |
| MPM Mesothelioma Gordon | 27 | hsa-mir-1301 | 4.84E−05 | — |
| MPM Mesothelioma Gordon | 35 | hsa-mir-371 | 0.000204415 | — |
| MPM Mesothelioma Gordon | 38 | hsa-mir-1276 | 8.53E−06 | — |
| MPM Mesothelioma Gordon | 41 | hsa-mir-519e | 0.000107989 | — |
| MPM Mesothelioma Gordon | 42 | hsa-mir-183 | 0.00048608 | — |
| MPM Mesothelioma Gordon | 48 | hsa-mir-561 | 0.000418507 | — |
| MPM Mesothelioma Gordon | 51 | hsa-mir-605 | 6.36E−05 | — |
| MPM Mesothelioma Gordon | 53 | hsa-mir-1182 | 0.00019028 | — |
| MPM Mesothelioma Gordon | 56 | hsa-mir-595 | 4.65E−05 | Malignant mesothelioma (MM) |
| MPM Mesothelioma Gordon | 61 | hsa-mir-545 | 5.77E−05 | — |
| MPM Mesothelioma Gordon | 62 | hsa-mir-1279 | 0.000304825 | — |
| MPM Mesothelioma Gordon | 63 | hsa-mir-548b hsa-mir-548a hsa-mir-548d hsa-mir-548i | 3.47E−06 | — |
| | | hsa-mir-548j hsa-mir-548c hsa-mir-548h | | |
| MPM Mesothelioma Gordon | 65 | hsa-mir-590-3p | 0.00046845 | — |
| MUC Ovarian Hendrix | 0 | hsa-mir-556-3p | 1.09E−05 | — |
| MUC Ovarian Hendrix | 2 | hsa-mir-22 | 0.00025469 | — |
| MUC Ovarian Hendrix | 7 | hsa-mir-607 | 0.000283493 | — |
| MUC Ovarian Hendrix | 8 | hsa-mir-1226 | 4.84E−05 | — |
| MUC Ovarian Hendrix | 14 | hsa-mir-148a hsa-mir-148b hsa-mir-152 | 8.43E−05 | epithelial ovarian cancer (EOC) |
| MUC Ovarian Hendrix | 15 | hsa-mir-340 | 1.89E−05 | — |
| MUC Ovarian Hendrix | 18 | hsa-mir-548n | 0.000222366 | — |
| MUC Ovarian Hendrix | 21 | hsa-mir-216b | 0.000279976 | — |
| MUC Ovarian Hendrix | 25 | hsa-mir-548c-3p | 1.02E−05 | — |
| MUC Ovarian Hendrix | 41 | hsa-mir-34a hsa-mir-34c hsa-mir-449a hsa-mir-449b | 0.000165758 | ovarian cancer (OC)\|ovarian cancer |
| | | | | (OC) |
| MUC Ovarian Hendrix | 44 | hsa-mir-217 | 0.000155736 | — |
| MUC Ovarian Hendrix | 45 | hsa-mir-765 | 8.97E−05 | — |
| MUC Ovarian Hendrix | 57 | hsa-mir-766 | 0.000194385 | — |
| MUC Ovarian Hendrix | 60 | hsa-mir-369-3p | 0.000117663 | — |
| MUC Ovarian Hendrix | 61 | hsa-mir-940 | 2.22E−05 | — |
| MUC Ovarian Hendrix | 63 | hsa-mir-184 | 3.31E−05 | epithelial ovarian cancer (EOC) |
| MUC Ovarian Hendrix | 67 | hsa-mir-1224-3p | 0.000283759 | — |
| MUC Ovarian Hendrix | 68 | hsa-mir-606 | 8.68E−06 | — |
| MUC Ovarian Hendrix | 71 | hsa-mir-361-3p | 0.00031521 | — |
| MUC Ovarian Hendrix | 74 | hsa-mir-27a hsa-mir-27b | 0.000125726 | — |
| MUC Ovarian Hendrix | 76 | hsa-mir-144 | 9.98E−05 | — |
| OD Brain Bredel | 2 | hsa-mir-768 | 2.72E−05 | — |
| OD Brain Bredel | 3 | hsa-mir-615-3p | 0.000123587 | — |
| OD Brain Bredel | 8 | hsa-mir-651 | 0.000193833 | — |
| OD Brain Bredel | 9 | hsa-mir-548e | 0.000425304 | — |
| OD Brain Bredel | 12 | hsa-mir-588 | 9.48E−05 | — |
| OD Brain Bredel | 18 | hsa-mir-1261 | 0.00060741 | — |
| OD Brain Bredel | 19 | hsa-mir-487a | 0.000532445 | — |
| OD Brain Bredel | 21 | hsa-mir-369-3p | 6.82E−05 | — |
| OD Brain Bredel | 24 | hsa-mir-193a-3p | 0.000341762 | — |
| OD Brain Bredel | 25 | hsa-mir-512-3p | 0.000343389 | — |
| OD Brain Bredel | 33 | hsa-mir-1231 | 4.51E−05 | — |
| OD Brain Bredel | 41 | hsa-mir-1276 | 5.88E−05 | — |
| OD Brain Bredel | 43 | hsa-mir-412 | 0.000207539 | — |
| OD Brain Bredel | 45 | hsa-mir-324 | 0.000220545 | — |
| OD Brain Bredel | 52 | hsa-mir-325 | 0.000190267 | — |
| OD Brain Bredel | 56 | hsa-mir-126 | 1.37E−05 | — |
| OD Brain Bredel | 57 | hsa-mir-148b hsa-mir-152 | 1.28E−05 | — |
| ODGL Brain Sun | 9 | hsa-mir-142-3p | 2.46E−05 | — |
| ODGL Brain Sun | 18 | hsa-mir-1274b | 4.99E−05 | — |
| ODGL Brain Sun | 20 | hsa-mir-570 | 1.84E−05 | — |
| ODGL Brain Sun | 25 | hsa-mir-548p | 2.43E−05 | — |
| ODGL Brain Sun | 26 | hsa-mir-622 | 0.000200859 | — |
| ODGL Brain Sun | 31 | hsa-mir-490-3p | 0.00051022 | — |
| ODGL Brain Sun | 33 | hsa-mir-335 | 4.85E−05 | — |
| ODGL Brain Sun | 36 | hsa-mir-361-3p | 5.23E−06 | — |
| ODGL Brain Sun | 39 | hsa-mir-218 | 0.000131483 | — |
| ODGL Brain Sun | 41 | hsa-mir-374b hsa-mir-374a | 1.52E−05 | — |
| ODGL Brain Sun | 44 | hsa-mir-1276 | 0.00049542 | — |
| ODGL Brain Sun | 50 | hsa-mir-548c-3p | 3.15E−05 | — |
| ODGL Brain Sun | 52 | hsa-mir-1270 hsa-mir-620 | 6.91E−05 | — |
| ODGL Brain Sun | 53 | hsa-mir-548m | 9.39E−05 | — |
| ODGL Brain Sun | 58 | hsa-mir-181d hsa-mir-181b | 2.72E−05 | glioma |
| ODGL Brain Sun | 60 | hsa-mir-556-3p | 0.000290266 | — |
| ODGL Brain Sun | 64 | hsa-mir-539 | 0.00028035 | — |
| ODGL Brain Sun | 65 | hsa-mir-340 | 3.02E−06 | — |
| PPC Prostate Dhanasekaran | 0 | hsa-mir-558 | 0.000302978 | — |
| PPC Prostate Dhanasekaran | 1 | hsa-mir-520d hsa-mir-524 | 0.000129964 | — |
| PPC Prostate Dhanasekaran | 2 | hsa-mir-217 | 0.000339362 | — |
| PPC Prostate Dhanasekaran | 3 | hsa-mir-624 | 0.000227035 | — |
| PPC Prostate Dhanasekaran | 4 | hsa-mir-760 | 0.000111326 | — |
| PPC Prostate Dhanasekaran | 9 | hsa-mir-153 | 1.70E−06 | — |
| PPC Prostate Dhanasekaran | 10 | hsa-mir-421 | 0.000113825 | — |
| PPC Prostate Dhanasekaran | 11 | hsa-mir-628-3p | 4.46E−05 | — |
| PPC Prostate Dhanasekaran | 14 | hsa-mir-34a hsa-mir-34c hsa-mir-449a hsa-mir-449b | 0.000448451 | prostate cancer |
| PPC Prostate Dhanasekaran | 19 | hsa-mir-548e | 4.34E−07 | — |
| PPC Prostate Dhanasekaran | 22 | hsa-mir-802 | 1.01E−05 | — |
| PPC Prostate Dhanasekaran | 23 | hsa-mir-376 | 8.08E−05 | — |
| PPC Prostate Dhanasekaran | 27 | hsa-mir-654-3p | 0.000130064 | — |
| PPC Prostate Dhanasekaran | 28 | hsa-mir-544 | 1.12E−10 | — |
| PPC Prostate Dhanasekaran | 41 | hsa-mir-18a hsa-mir-18b | 1.28E−05 | — |
| RCCC Renal Boer | 0 | hsa-mir-140-3p | 0.000185774 | — |
| RCCC Renal Boer | 3 | hsa-mir-217 | 0.000238884 | — |
| RCCC Renal Boer | 4 | hsa-mir-1262 | 0.0004252 | — |
| RCCC Renal Boer | 8 | hsa-mir-1206 | 0.000348303 | — |
| RCCC Renal Boer | 9 | hsa-mir-1256 | 0.000206072 | — |
| RCCC Renal Boer | 12 | hsa-mir-760 | 0.000652786 | — |
| RCCC Renal Boer | 13 | hsa-mir-200a hsa-mir-141 | 6.74E−05 | cancer |
| RCCC Renal Boer | 14 | hsa-mir-590-3p | 0.000647061 | — |
| RCCC Renal Boer | 15 | hsa-mir-548k | 0.000242163 | — |
| RCCC Renal Lenburg | 6 | hsa-mir-651 | 0.000604766 | — |
| RCCC Renal Lenburg | 8 | hsa-mir-660 | 3.63E−05 | — |
| SMCL Lung Bhattacharjee | 1 | hsa-mir-1237 | 0.00015325 | — |
| SMCL Lung Bhattacharjee | 2 | hsa-mir-1266 | 0.000383019 | — |
| SMCL Lung Bhattacharjee | 9 | hsa-mir-362-3p hsa-mir-329 | 0.000160237 | — |
| SMCL Lung Bhattacharjee | 10 | hsa-mir-720 | 0.000354772 | — |
| SMCL Lung Bhattacharjee | 15 | hsa-mir-216b | 0.000194459 | — |
| SMCL Lung Bhattacharjee | 16 | hsa-mir-1274a | 0.000233435 | — |
| SMCL Lung Bhattacharjee | 17 | hsa-mir-888 | 0.000289329 | — |
| SMCL Lung Bhattacharjee | 18 | hsa-mir-624 | 6.67E−06 | — |
| SMCL Lung Bhattacharjee | 21 | hsa-mir-570 | 0.000277837 | — |
| SMCL Lung Bhattacharjee | 22 | hsa-mir-548n | 0.000215679 | — |
| SMCL Lung Bhattacharjee | 25 | hsa-mir-101 | 0.000395032 | lung cancer\|lung cancer |
| SMCL Lung Bhattacharjee | 29 | hsa-mir-513a-3p | 1.00E−05 | — |
| SMCL Lung Bhattacharjee | 30 | hsa-mir-548n | 4.80E−06 | — |
| SMCL Lung Bhattacharjee | 34 | hsa-mir-1207 | 3.88E−05 | — |
| SMCL Lung Bhattacharjee | 41 | hsa-mir-512-3p | 0.000567462 | — |
| SMCL Lung Bhattacharjee | 45 | hsa-mir-432 | 0.000596485 | — |
| SMCL Lung Bhattacharjee | 46 | hsa-mir-499 | 8.39E−05 | — |
| SMCL Lung Bhattacharjee | 52 | hsa-mir-483-3p | 3.55E−05 | — |
| SMCL Lung Bhattacharjee | 55 | hsa-mir-590-3p | 0.000205653 | — |
| SMCL Lung Bhattacharjee | 56 | hsa-mir-888 | 0.00034326 | — |
| SQ Lung Bhattacharjee | 4 | hsa-mir-423 | 0.000143527 | lung cancer |
| SQ Lung Bhattacharjee | 15 | hsa-mir-96 | 4.75E−05 | non-small cell lung cancer (NSCLC) |
| SQ Lung Bhattacharjee | 16 | hsa-mir-802 | 0.000178602 | — |
| SQ Lung Bhattacharjee | 18 | hsa-mir-767 | 2.17E−06 | — |
| SQ Lung Bhattacharjee | 19 | hsa-mir-423 | 3.84E−05 | lung cancer |
| SQ Lung Bhattacharjee | 22 | hsa-mir-548n | 0.000156966 | — |
| SQ Lung Bhattacharjee | 28 | hsa-mir-616 | 0.000166731 | — |
| SQ Lung Bhattacharjee | 32 | hsa-mir-532-3p | 0.000103573 | — |
| SQ Lung Bhattacharjee | 33 | hsa-mir-339 | 3.36E−06 | lung cancer |
| SQ Lung Bhattacharjee | 37 | hsa-mir-770 | 0.000210707 | — |
| SQ Lung Bhattacharjee | 41 | hsa-mir-885-3p | 5.21E−05 | — |
| SQ Lung Bhattacharjee | 42 | hsa-mir-637 | 8.30E−06 | — |
| SQ Lung Bhattacharjee | 43 | hsa-mir-597 | 4.56E−05 | — |
| SQ Lung Bhattacharjee | 44 | hsa-mir-767 | 0.000168025 | — |
| SQ Lung Bhattacharjee | 46 | hsa-mir-650 | 0.0001481 | — |
| SQ Lung Bhattacharjee | 52 | hsa-mir-411 | 0.000356853 | — |
| SRS Ovarian Hendrix | 1 | hsa-mir-641 | 5.26E−05 | — |
| SRS Ovarian Hendrix | 2 | hsa-mir-505 | 8.02E−06 | — |
| SRS Ovarian Hendrix | 11 | hsa-mir-361 | 1.61E−05 | ovarian cancer (OC) |
| SRS Ovarian Hendrix | 15 | hsa-mir-1293 | 1.58E−05 | — |
| SRS Ovarian Hendrix | 22 | hsa-mir-624 | 0.000609064 | — |
| SRS Ovarian Hendrix | 27 | hsa-mir-362 | 3.10E−05 | — |
| SRS Ovarian Hendrix | 30 | hsa-mir-424 | 5.59E−07 | ovarian cancer (OC) |
| SRS Ovarian Hendrix | 41 | hsa-mir-650 | 7.93E−06 | — |
| SRS Ovarian Hendrix | 45 | hsa-mir-146b-3p | 0.000287347 | — |
| SRS Ovarian Hendrix | 52 | hsa-mir-626 | 0.000199193 | — |
| SRS Ovarian Hendrix | 53 | hsa-mir-376b hsa-mir-376a | 9.72E−08 | epithelial ovarian cancer (EOC) |
| SRS Ovarian Hendrix | 54 | hsa-mir-448 | 0.00035465 | — |
| SRS Ovarian Hendrix | 55 | hsa-mir-548l | 4.93E−05 | — |
| SRS Ovarian Hendrix | 57 | hsa-mir-561 | 5.83E−05 | — |
| SRS Ovarian Hendrix | 60 | hsa-mir-188-3p | 0.000485917 | — |
| SRS Ovarian Hendrix | 66 | hsa-mir-892b | 6.69E−06 | — |
| SRS Ovarian Hendrix | 69 | hsa-mir-330-3p | 9.80E−09 | — |
| SRS Ovarian Hendrix | 70 | hsa-mir-641 | 3.14E−07 | — |
| SRS Ovarian Hendrix | 72 | hsa-mir-1276 | 2.59E−10 | — |
| SRS Ovarian Hendrix | 73 | hsa-mir-340 | 3.32E−06 | — |
| TU Prostate Lapointe | 0 | hsa-mir-874 | 5.58E−05 | — |
| TU Prostate Lapointe | 1 | hsa-mir-1297 hsa-mir-26a hsa-mir-26b | 0.000505563 | prostate cancer\|prostate cancer |
| TU Prostate Lapointe | 4 | hsa-mir-532-3p | 3.70E−07 | — |
| TU Prostate Lapointe | 9 | hsa-mir-208a hsa-mir-208b | 1.34E−05 | — |
| TU Prostate Lapointe | 17 | hsa-mir-96 | 0.000125874 | prostate cancer |
| TU Prostate Lapointe | 18 | hsa-mir-760 | 6.17E−05 | — |
| TU Prostate Lapointe | 24 | hsa-mir-496 | 3.93E−06 | — |
| TU Prostate Lapointe | 31 | hsa-mir-631 | 0.000651307 | — |
| TU Prostate Lapointe | 33 | hsa-mir-802 | 9.41E−05 | — |
| TU Prostate Lapointe | 35 | hsa-mir-758 | 1.84E−05 | — |
| TU Prostate Lapointe | 37 | hsa-mir-199b-3p hsa-mir-199a-3p | 0.000280073 | cancer\|prostate cancer |
| TU Prostate Lapointe | 38 | hsa-mir-1264 | 0.000492613 | — |
| TU Prostate Lapointe | 39 | hsa-mir-590-3p | 1.58E−05 | — |

SUPPLEMENTRY TABLE 6

| | | | Uncorrected | |
|---|---|---|---|---|
| Dataset | Cluster | miRNA | P-value | miR2Disease |
| IDC Breast Radvanyi | 9 | hsa-mir-661 | 1.62E−05 | breast cancer |
| CA Breast Richardson | 16 | hsa-mir-18a | 3.62E−06 | breast cancer |
| IDC Breast Radvanyi | 28 | hsa-mir-149 | 2.94E−05 | breast cancer |
| ILC Breast Radvanyi | 20 | hsa-mir-328 | 0.000540368 | breast cancer |
| MCA Breast Radvanyi | 18 | hsa-mir-663 | 3.07E−05 | breast cancer\|breast cancer |
| CA Breast Richardson | 27 | hsa-mir-663 | 2.76E−06 | breast cancer\|breast cancer |
| MCA Breast Radvanyi | 2 | hsa-mir-204 | 0.000170357 | breast cancer\|breast cancer |
| CA Breast Richardson | 68 | hsa-mir-663 | 1.21E−06 | breast cancer\|breast cancer |
| IDC Breast Radvanyi | 15 | hsa-mir-205 | 6.43E−06 | breast cancer\|breast cancer\|breast cancer |
| MCA Breast Radvanyi | 19 | hsa-mir-210 | 0.00017588 | breast cancer\|breast cancer\|breast cancer |
| B-CLL Leukemia Haslinger | 44 | hsa-mir-200b | 0.000562281 | cancer |
| OD Brain Bredel | 12 | hsa-mir-200a | 1.77E−05 | cancer |
| MPM Mesothelioma Gordon | 31 | hsa-mir-127 | 0.000264768 | cancer |
| MPC Prostate Dhanasekaran | 0 | hsa-mir-200b | 4.80E−05 | cancer |
| B-CLL Leukemia Haslinger | 55 | hsa-mir-200b | 3.75E−07 | cancer |
| CA Renal Higgins | 3 | hsa-mir-199a-3p | 0.000230566 | cancer |
| AD Ovarian Welsh | 0 | hsa-mir-200b | 1.65E−06 | cancer\|epithelial ovarian cancer (EOC)\|ovarian cancer (OC)\|ovarian |
| | | | | cancer (OC) |
| MUC Ovarian Hendrix | 49 | hsa-mir-200b | 3.93E−06 | cancer\|epithelial ovarian cancer (EOC)\|ovarian cancer (OC)\|ovarian |
| | | | | cancer (OC) |
| SRS Ovarian Hendrix | 68 | hsa-mir-200b | 4.92E−06 | cancer\|epithelial ovarian cancer (EOC)\|ovarian cancer (OC)\|ovarian cancer |
| | | | | (OC)\|serous ovarian cancer |
| ME Melanoma Hoek | 35 | hsa-mir-200b | 1.13E−06 | cancer\|malignant melanoma |
| B-CLL Leukemia Haslinger | 69 | hsa-mir-146a | 0.000450962 | chronic lymphocytic leukemia (CLL) |
| CLL Lymphoma Alizadeh | 4 | hsa-mir-29b | 0.000197019 | chronic lymphocytic leukemia (CLL)\|chronic lymphocytic leukemia |
| | | | | (CLL)\|chronic lymphocytic leukemia (CLL) |
| CA Colon Graudens | 32 | hsa-mir-296 | 1.18E−06 | colorectal cancer |
| CA Colon Graudens | 2 | hsa-mir-451 | 1.16E−05 | colorectal cancer |
| CA Colon Graudens | 37 | hsa-mir-32 | 0.000107611 | colorectal cancer |
| CA Colon Graudens | 15 | hsa-mir-140 | 0.00034922 | colorectal cancer |
| CA Colon Graudens | 38 | hsa-mir-96 | 1.79E−05 | colorectal cancer\|colorectal cancer |
| SRS Ovarian Hendrix | 60 | hsa-mir-184 | 0.000490759 | epithelial ovarian cancer (EOC) |
| SRS Ovarian Hendrix | 64 | hsa-mir-377 | 0.000266561 | epithelial ovarian cancer (EOC) |
| END Ovarian Hendrix | 66 | hsa-mir-101 | 4.40E−06 | epithelial ovarian cancer (EOC) |
| CCC Ovarian Hendrix | 20 | hsa-mir-495 | 2.23E−05 | epithelial ovarian cancer (EOC) |
| END Ovarian Hendrix | 28 | hsa-mir-495 | 1.97E−05 | epithelial ovarian cancer (EOC) |
| CCC Ovarian Hendrix | 61 | hsa-mir-145 | 0.000153611 | epithelial ovarian cancer (EOC)\|epithelial ovarian cancer (EOC) |
| END Ovarian Hendrix | 49 | hsa-mir-145 | 0.000588653 | epithelial ovarian cancer (EOC)\|epithelial ovarian cancer (EOC) |
| SRS Ovarian Hendrix | 15 | hsa-let-7d | 0.000397689 | epithelial ovarian cancer (EOC)\|epithelial ovarian cancer (EOC)\|ovarian |
| | | | | cancer (OC) |
| END Ovarian Hendrix | 37 | hsa-mir-125b | 0.000475101 | epithelial ovarian cancer (EOC)\|ovarian cancer (OC) |
| END Ovarian Hendrix | 78 | hsa-mir-125a | 0.000362188 | epithelial ovarian cancer (EOC)\|ovarian cancer (OC) |
| FL Lymphoma Alizadeh | 2 | hsa-mir-149 | 2.16E−05 | follicular lymphoma (FL) |
| GBM Brain Liang | 7 | hsa-mir-323 | 5.85E−05 | glioblastoma multiforme (GBM) |
| GL Brain Bredel | 60 | hsa-mir-25 | 9.54E−05 | glioblastoma\|glioma |
| ODGL Brain Sun | 57 | hsa-mir-296 | 9.53E−06 | glioma |
| GL Brain Bredel | 32 | hsa-mir-15a | 0.000311239 | glioma |
| OD Brain Bredel | 14 | hsa-mir-296 | 7.12E−06 | glioma |
| AO Brain Bredel | 1 | hsa-mir-181a | 0.000168423 | glioma |
| AO Brain Bredel | 28 | hsa-mir-296 | 7.75E−06 | glioma |
| AC Brain Sun | 12 | hsa-mir-210 | 6.54E−05 | glioma |
| HSCC Head-Neck Cromer | 22 | hsa-mir-30b | 3.14E−05 | head and neck squamous cell carcinoma (HNSCC) |
| RCCC Renal Lenburg | 0 | hsa-mir-489 | 2.02E−05 | kidney cancer |
| RCCC Renal Boer | 7 | hsa-mir-106b | 0.000251281 | kidney cancer |
| SMCL Lung Bhattacharjee | 58 | hsa-mir-423 | 0.000179756 | lung cancer |
| AD Lung Beer | 18 | hsa-mir-19a | 5.85E−07 | lung cancer |
| AO Lung Bhattacharjee | 0 | hsa-mir-338 | 1.12E−06 | lung cancer |
| SMCL Lung Bhattacharjee | 20 | hsa-mir-423 | 7.09E−06 | lung cancer |
| AD Lung Stearman | 29 | hsa-mir-425 | 3.76E−05 | lung cancer |
| SQ Lung Bhattacharjee | 4 | hsa-mir-214 | 7.88E−05 | lung cancer |
| COID Lung Bhattacharjee | 78 | hsa-mir-27b | 0.000262632 | lung cancer |
| AD Lung Bhattacharjee | 27 | hsa-mir-451 | 0.000277652 | lung cancer |
| COID Lung Bhattacharjee | 30 | hsa-mir-423 | 0.000201932 | lung cancer |
| COID Lung Bhattacharjee | 40 | hsa-mir-93 | 0.000170538 | lung cancer |
| SMCL Lung Bhattacharjee | 35 | hsa-mir-212 | 0.000352999 | lung cancer |
| SMCL Lung Bhattacharjee | 45 | hsa-mir-125a | 0.000506289 | lung cancer\|lung cancer |
| SQ Lung Bhattacharjee | 32 | hsa-mir-210 | 0.000299713 | lung cancer\|lung cancer |
| AD Lung Beer | 31 | hsa-mir-29b | 1.59E−06 | lung cancer\|lung cancer |
| COID Lung Bhattacharjee | 10 | hsa-mir-101 | 7.34E−07 | lung cancer\|lung cancer |
| SMCL Lung Bhattacharjee | 53 | hsa-mir-20a | 2.42E−05 | lung cancer\|lung cancer\|lung cancer |
| AD Lung Bhattacharjee | 52 | hsa-let-7a | 0.000427939 | lung cancer\|lung cancer\|lung cancer\|lung cancer\|lung cancer\|lung |
| | | | | cancer\|non-small cell lung cancer (NSCLC)\|non-small cell lung cancer |
| | | | | (NSCLC) |
| AD Lung Beer | 0 | hsa-let-7d | 8.49E−05 | lung cancer\|non-small cell lung cancer (NSCLC) |
| AD Lung Stearman | 7 | hsa-mir-16 | 6.72E−05 | lung cancer\|non-small cell lung cancer (NSCLC) |
| ME Melanoma Hoek | 20 | hsa-mir-331 | 0.000508693 | malignant melanoma |
| ML Melanoma Talantov | 8 | hsa-mir-199b | 3.48E−05 | malignant melanoma |
| ML Melanoma Talantov | 27 | hsa-mir-96 | 0.000474947 | malignant melanoma |
| MPM Mesothelioma Gordon | 59 | hsa-mir-423 | 7.33E−06 | Malignant mesothelioma (MM) |
| MPM Mesothelioma Gordon | 19 | hsa-mir-423 | 0.000166269 | Malignant mesothelioma (MM) |
| SQ Lung Bhattacharjee | 49 | hsa-mir-34a | 0.000184016 | non-small cell lung cancer (NSCLC) |
| SRS Ovarian Hendrix | 24 | hsa-mir-635 | 0.000119101 | ovarian cancer (OC) |
| MUC Ovarian Hendrix | 28 | hsa-mir-296 | 8.33E−06 | ovarian cancer (OC) |
| AD Ovarian Welsh | 25 | hsa-mir-572 | 0.000107609 | ovarian cancer (OC) |
| MUC Ovarian Hendrix | 30 | hsa-mir-635 | 0.000153945 | ovarian cancer (OC) |
| SRS Ovarian Hendrix | 83 | hsa-mir-637 | 1.65E−06 | ovarian cancer (OC) |
| SRS Ovarian Hendrix | 20 | hsa-mir-296 | 0.000493587 | ovarian cancer (OC) |
| SRS Ovarian Hendrix | 67 | hsa-mir-608 | 6.23E−07 | ovarian cancer (OC) |
| MUC Ovarian Hendrix | 26 | hsa-mir-296 | 6.09E−05 | ovarian cancer (OC) |
| MUC Ovarian Hendrix | 48 | hsa-mir-608 | 8.32E−05 | ovarian cancer (OC) |
| SRS Ovarian Hendrix | 78 | hsa-mir-206 | 0.000254763 | ovarian cancer (OC) |
| END Ovarian Hendrix | 15 | hsa-mir-542-3p | 3.11E−05 | ovarian cancer (OC) |
| END Ovarian Hendrix | 17 | hsa-mir-30e | 0.000158264 | ovarian cancer (OC) |
| AD Ovarian Welsh | 4 | hsa-mir-424 | 9.76E−06 | ovarian cancer (OC) |
| TU Prostate Lapointe | 10 | hsa-mir-32 | 9.82E−06 | prostate cancer |
| PPC Prostate Dhanasekaran | 34 | hsa-mir-149 | 0.000124363 | prostate cancer |
| TU Prostate Lapointe | 13 | hsa-mir-497 | 0.000100169 | prostate cancer |
| TU Prostate Lapointe | 23 | hsa-mir-25 | 0.00024991 | prostate cancer |
| TU Prostate Lapointe | 19 | hsa-mir-296 | 6.89E−07 | prostate cancer |
| MPC Prostate Dhanasekaran | 11 | hsa-mir-34a | 0.000291101 | prostate cancer |
| MPC Prostate Dhanasekaran | 7 | hsa-mir-296 | 4.29E−06 | prostate cancer |
| PPC Prostate Dhanasekaran | 17 | hsa-mir-34a | 0.000201155 | prostate cancer |
| PPC Prostate Dhanasekaran | 32 | hsa-mir-296 | 1.77E−06 | prostate cancer |
| MPC Prostate Dhanasekaran | 16 | hsa-mir-101 | 2.53E−06 | prostate cancer\|prostate cancer\|prostate cancer |
| RCCC Renal Boer | 0 | hsa-mir-494 | 4.34E−06 | renal clear cell carcinoma |
| GCT Seminoma Korkola | 87 | hsa-mir-371 | 8.90E−07 | testicular germ cell tumor |
| GCT Seminoma Korkola | 101 | hsa-mir-372 | 3.92E−07 | testicular germ cell tumor\|testicular germ cell tumor |
| MUC Ovarian Hendrix | 21 | hsa-mir-520d | 3.10E−05 | — |
| MUC Ovarian Hendrix | 19 | hsa-mir-548g | 1.81E−05 | — |
| AD Lung Bhattacharjee | 55 | hsa-mir-1286 | 5.31E−05 | — |
| GL Brain Bredel | 22 | hsa-mir-657 | 0.000105499 | — |
| GLB Brain Sun | 36 | hsa-mir-423 | 2.03E−07 | — |
| COID Lung Bhattacharjee | 58 | hsa-mir-23a | 0.000106957 | — |
| AD Ovarian Welsh | 1 | hsa-mir-548d-3p | 6.48E−07 | — |
| CA Colon Graudens | 24 | hsa-mir-522 | 1.62E−06 | — |
| SMCL Lung Bhattacharjee | 18 | hsa-mir-513 | 4.74E−05 | — |
| ME Melanoma Hoek | 26 | hsa-mir-577 | 0.00015717 | — |
| B-CLL Leukemia Haslinger | 38 | hsa-mir-548g | 8.13E−05 | — |
| SMCL Lung Bhattacharjee | 6 | hsa-mir-1201 | 0.000464237 | — |
| AD Lung Beer | 38 | hsa-mir-933 | 2.88E−05 | — |
| BPH Prostate Dhanasekaran | 18 | hsa-mir-513a-3p | 1.76E−05 | — |
| GCT Seminoma Korkola | 89 | hsa-mir-1257 | 5.15E−06 | — |
| MPM Mesothelioma Gordon | 64 | hsa-mir-655 | 6.39E−06 | — |
| GCT Seminoma Korkola | 28 | hsa-mir-146a | 0.000148787 | — |
| HSCC Head-Neck Chung | 9 | hsa-mir-708 | 0.000132985 | — |
| GLB Brain Sun | 72 | hsa-mir-1294 | 6.36E−05 | — |
| B-CLL Leukemia Haslinger | 36 | hsa-mir-587 | 0.000191245 | — |
| CA Breast Sorlie | 20 | hsa-mir-590-3p | 3.24E−07 | — |
| GCT Seminoma Korkola | 112 | hsa-mir-548n | 7.79E−15 | — |
| AD Lung Stearman | 13 | hsa-mir-23a | 5.53E−05 | — |
| COID Lung Bhattacharjee | 51 | hsa-mir-656 | 3.16E−09 | — |
| CA Breast Richardson | 28 | hsa-mir-765 | 0.000106308 | — |
| AD Lung Bhattacharjee | 30 | hsa-mir-548c-3p | 9.03E−14 | — |
| ILC Breast Radvanyi | 2 | hsa-mir-423-3p | 0.000203181 | — |
| MPM Mesotheiioma Gordon | 29 | hsa-mir-1265 | 0.000313362 | — |
| ML Melanoma Talantov | 26 | hsa-mir-664 | 2.21E−05 | — |
| CA Bladder Dyrskjot | 39 | hsa-mir-486-3p | 0.000214753 | — |
| GLB Brain Sun | 18 | hsa-mir-498 | 6.03E−09 | — |
| PPC Prostate Dhanasekaran | 29 | hsa-mir-509-3p | 0.000145973 | — |
| CA Breast Richardson | 33 | hsa-mir-1251 | 0.000477316 | — |
| CA Breast Richardson | 3 | hsa-mir-578 | 8.26E−06 | — |
| GCT Seminoma Korkola | 37 | hsa-mir-519a | 1.33E−09 | — |
| GL Brain Rickman | 22 | hsa-mir-374b | 4.01E−07 | — |
| CA Bladder Dyrskjot | 1 | hsa-mir-637 | 6.92E−11 | — |
| SRS Ovarian Hendrix | 16 | hsa-mir-548k | 1.82E−06 | — |
| AD Lung Bhattacharjee | 81 | hsa-mir-516b | 1.26E−05 | — |
| FL Lymphoma Alizadeh | 0 | hsa-mir-92a | 1.01E−07 | — |
| MPC Prostate Dhanasekaran | 29 | hsa-mir-663 | 6.65E−05 | — |
| AD Lung Beer | 43 | hsa-mir-1275 | 0.000105766 | — |
| CA Bladder Dyrskjot | 3 | hsa-mir-1308 | 8.73E−07 | — |
| SRS Ovarian Hendrix | 10 | hsa-mir-34c-3p | 4.69E−07 | — |
| MPC Prostate Dhanasekaran | 26 | hsa-mir-548j | 0.000385787 | — |
| CCC Ovarian Hendrix | 44 | hsa-mir-548m | 1.07E−05 | — |
| CA Bladder Dyrskjot | 53 | hsa-mir-944 | 1.08E−06 | — |
| GCT Seminoma Korkola | 31 | hsa-mir-891a | 0.000233697 | — |
| COID Lung Bhattacharjee | 25 | hsa-mir-361 | 6.17E−05 | — |
| MM Myeloma Zhan | 50 | hsa-mir-28 | 7.86E−05 | — |
| AD Ovarian Welsh | 10 | hsa-mir-921 | 6.01E−06 | — |
| SMCL Lung Bhattacharjee | 46 | hsa-mir-891b | 0.000383258 | — |
| RCCC Renal Boer | 1 | hsa-mir-371 | 0.000530325 | — |
| PPC Prostate Dhanasekaran | 1 | hsa-mir-561 | 1.34E−05 | — |
| GCT Seminoma Korkola | 80 | hsa-mir-573 | 3.65E−06 | — |
| CCC Ovarian Hendrix | 14 | hsa-mir-1276 | 1.06E−05 | — |
| GL Brain Bredel | 17 | hsa-mir-877 | 0.000266194 | — |
| END Ovarian Hendrix | 32 | hsa-mir-548d-3p | 6.61E−11 | — |
| COID Lung Bhattacharjee | 48 | hsa-mir-886 | 0.00018962 | — |
| CA Bladder Dyrskjot | 23 | hsa-mir-211 | 8.07E−07 | — |
| MPM Mesothelioma Gordon | 56 | hsa-mir-590-3p | 9.29E−06 | — |
| BPH Prostate Dhanasekaran | 7 | hsa-mir-380 | 4.14E−06 | — |
| SRS Ovarian Hendrix | 75 | hsa-mir-372 | 1.97E−08 | — |
| COID Lung Bhattacharjee | 5 | hsa-mir-548o | 2.41E−05 | — |
| GL Brain Bredel | 78 | hsa-mir-424 | 0.000301626 | — |
| BPH Prostate Dhanasekaran | 9 | hsa-mir-574 | 0.000269626 | — |
| FL Lymphoma Alizadeh | 11 | hsa-mir-614 | 0.000529183 | — |
| GCT Seminoma Korkola | 63 | hsa-mir-889 | 3.04E−06 | — |
| END Ovarian Hendrix | 77 | hsa-mir-590-3p | 4.95E−09 | — |
| DLBCL Lymphoma Alizadeh | 3 | hsa-mir-744 | 1.82E−05 | — |
| MPM Mesothelioma Gordon | 52 | hsa-mir-606 | 8.63E−08 | — |
| SQ Lung Bhattacharjee | 30 | hsa-mir-1233 | 0.000445813 | — |
| CA Bladder Dyrskjot | 25 | hsa-mir-1204 | 2.52E−05 | — |
| GCT Seminoma Korkola | 73 | hsa-mir-675 | 0.000190634 | — |
| MUC Ovarian Hendrix | 55 | hsa-mir-486-3p | 1.46E−05 | — |
| SMCL Lung Bhattacharjee | 2 | hsa-mir-940 | 1.23E−05 | — |
| GCT Seminoma Korkola | 3 | hsa-mir-296 | 2.40E−07 | — |
| CCC Ovarian Hendrix | 37 | hsa-mir-1237 | 0.000328049 | — |
| AC Brain Sun | 46 | hsa-mir-590-3p | 9.16E−09 | — |
| PPC Prostate Dhanasekaran | 28 | hsa-mir-944 | 3.15E−12 | — |
| HSCC Head-Neck Cromer | 20 | hsa-mir-567 | 4.16E−07 | — |
| MUC Ovarian Hendrix | 23 | hsa-mir-486-3p | 2.97E−07 | — |
| GLB Brain Sun | 73 | hsa-mir-187 | 5.00E−06 | — |
| ME Melanoma Hoek | 16 | hsa-mir-631 | 0.000590804 | — |
| COID Lung Bhattacharjee | 64 | hsa-mir-661 | 6.54E−07 | — |
| COID Lung Bhattacharjee | 75 | hsa-mir-874 | 7.97E−05 | — |
| CA Breast Richardson | 58 | hsa-mir-889 | 6.58E−05 | — |
| CA Breast Richardson | 53 | hsa-mir-486 | 3.46E−05 | — |
| GCT Seminoma Korkola | 85 | hsa-mir-181 | 0.000115903 | — |
| ODGL Brain Sun | 11 | hsa-mir-219-1-3p | 0.000228608 | — |
| CA Breast Richardson | 65 | hsa-mir-1254 | 4.75E−05 | — |
| AD Ovarian Welsh | 21 | hsa-mir-606 | 8.71E−05 | — |
| MPC Prostate Dhanasekaran | 48 | hsa-mir-625 | 0.000326443 | — |
| SMCL Lung Bhattacharjee | 11 | hsa-mir-1321 | 0.000308592 | — |
| HSCC Head-Neck Chung | 10 | hsa-mir-607 | 3.02E−05 | — |
| GBM Brain Liang | 13 | hsa-mir-1278 | 0.000208092 | — |
| GL Brain Rickman | 23 | hsa-mir-935 | 9.26E−06 | — |
| AC Brain Sun | 28 | hsa-mir-539 | 4.32E−05 | — |
| GLB Brain Sun | 1 | hsa-mir-324 | 3.16E−05 | — |
| DLBCL Lymphoma Alizadeh | 7 | hsa-mir-1301 | 0.000173451 | — |
| SRS Ovarian Hendrix | 21 | hsa-mir-944 | 4.95E−07 | — |
| ODGL Brain Sun | 63 | hsa-mir-410 | 0.000134983 | — |
| AD Lung Beer | 15 | hsa-mir-586 | 0.000587308 | — |
| MUC Ovarian Hendrix | 72 | hsa-mir-409 | 0.00020328 | — |
| CA Breast Richardson | 51 | hsa-mir-633 | 1.08E−05 | — |
| GCT Seminoma Korkola | 83 | hsa-mir-1254 | 0.000197016 | — |
| CCC Ovarian Hendrix | 43 | hsa-mir-1247 | 2.38E−07 | — |
| B-CLL Leukemia Haslinger | 31 | hsa-mir-1321 | 0.000537156 | — |
| MM Myeloma Zhan | 39 | hsa-mir-647 | 0.000511455 | — |
| CA Breast Richardson | 25 | hsa-mir-1252 | 0.000611632 | — |
| BPH Prostate Dhanasekaran | 20 | hsa-mir-1224-3p | 9.26E−06 | — |
| GCT Seminoma Korkola | 72 | hsa-mir-760 | 4.02E−05 | — |
| SRS Ovarian Hendrix | 55 | hsa-mir-338 | 1.40E−06 | — |
| IDC Breast Radvanyi | 7 | hsa-mir-450b-3p | 0.000288602 | — |
| MUC Ovarian Hendrix | 34 | hsa-mir-1291 | 0.000213025 | — |
| MUC Ovarian Hendrix | 56 | hsa-mir-548c-3p | 2.02E−10 | — |
| OD Brain Bredel | 57 | hsa-mir-423 | 2.25E−06 | — |
| TU Prostate Lapointe | 0 | hsa-mir-219-2-3p | 7.92E−05 | — |
| SMCL Lung Bhattacharjee | 28 | hsa-mir-744 | 9.04E−05 | — |
| TU Prostate Lapointe | 17 | hsa-mir-219 | 0.000106525 | — |
| END Ovarian Hendrix | 20 | hsa-mir-663b | 9.77E−05 | — |
| GCT Seminoma Korkola | 96 | hsa-mir-29a | 8.00E−05 | — |
| B-CLL Leukemia Haslinger | 53 | hsa-mir-513a-3p | 1.99E−05 | — |
| CCC Ovarian Hendrix | 7 | hsa-mir-382 | 0.000180447 | — |
| CA Bladder Dyrskjot | 9 | hsa-mir-548c-3p | 1.50E−14 | — |
| CA Bladder Dyrskjot | 65 | hsa-mir-888 | 1.94E−05 | — |
| END Ovarian Hendrix | 76 | hsa-mir-888 | 8.60E−06 | — |
| GLB Brain Sun | 54 | hsa-mir-921 | 2.44E−08 | — |
| GCT Seminoma Korkola | 40 | hsa-mir-548n | 0.000170776 | — |
| GLB Brain Sun | 0 | hsa-mir-637 | 3.57E−05 | — |
| RCCC Renal Boer | 13 | hsa-mir-1279 | 1.39E−06 | — |
| MUC Ovarian Hendrix | 76 | hsa-mir-573 | 7.31E−08 | — |
| AD Lung Bhattacharjee | 22 | hsa-mir-619 | 0.000399164 | — |
| END Ovarian Hendrix | 9 | hsa-mir-486-3p | 2.89E−06 | — |
| AD Lung Bhattacharjee | 75 | hsa-mir-637 | 2.83E−06 | — |
| ME Melanoma Hoek | 0 | hsa-mir-582 | 4.80E−05 | — |
| MUC Ovarian Hendrix | 73 | hsa-mir-361-3p | 4.66E−10 | — |
| CA Breast Richardson | 61 | hsa-mir-1271 | 0.000378242 | — |
| AC Brain Sun | 17 | hsa-mir-617 | 0.000105979 | — |
| TU Prostate Lapointe | 34 | hsa-mir-886 | 0.000346316 | — |
| MUC Ovarian Hendrix | 13 | hsa-mir-1280 | 0.000351255 | — |
| DLBCL Lymphoma Alizadeh | 2 | hsa-mir-660 | 0.000277854 | — |
| GLB Brain Sun | 31 | hsa-mir-501-3p | 0.000148691 | — |
| MUC Ovarian Hendrix | 15 | hsa-mir-944 | 7.48E−12 | — |
| GCT Seminoma Korkola | 102 | hsa-mir-34a | 9.24E−05 | — |
| HSCC Head-Neck Cromer | 10 | hsa-mir-520a | 0.000571699 | — |
| ODGL Brain Sun | 0 | hsa-mir-637 | 0.000354231 | — |
| MPM Mesothelioma Gordon | 27 | hsa-mir-128 | 0.000309287 | — |
| GL Brain Bredel | 57 | hsa-mir-193a-3p | 0.000180522 | — |
| CA Bladder Dyrskjot | 42 | hsa-mir-362 | 9.61E−05 | — |
| MPC Prostate Dhanasekaran | 22 | hsa-mir-382 | 5.46E−06 | — |
| ILC Breast Radvanyi | 4 | hsa-mir-548a-3p | 0.000525313 | — |
| CA Bladder Dyrskjot | 13 | hsa-mir-548c-3p | 6.28E−07 | — |
| AD Lung Bhattacharjee | 35 | hsa-mir-133a | 1.57E−05 | — |
| MUC Ovarian Hendrix | 27 | hsa-mir-423 | 0.000146228 | — |
| MUC Ovarian Hendrix | 18 | hsa-mir-606 | 5.19E−05 | — |
| GCT Seminoma Korkola | 25 | hsa-mir-607 | 1.21E−08 | — |
| MPM Mesothelioma Gordon | 51 | hsa-mir-376a | 0.000159942 | — |
| GLB Brain Sun | 20 | hsa-mir-331 | 3.35E−05 | — |
| AD Lung Bhattacharjee | 44 | hsa-mir-1284 | 6.45E−06 | — |
| GCT Seminoma Korkola | 21 | hsa-mir-520f | 2.04E−08 | — |
| SRS Ovarian Hendrix | 66 | hsa-mir-548c-3p | 8.80E−09 | — |
| CA Breast Richardson | 59 | hsa-mir-608 | 1.67E−05 | — |
| AD Lung Bhattacharjee | 10 | hsa-mir-1321 | 6.35E−06 | — |
| DLBCL Lymphoma Alizadeh | 6 | hsa-mir-483-3p | 0.000603016 | — |
| END Ovarian Hendrix | 38 | hsa-mir-944 | 3.49E−09 | — |
| CCC Ovarian Hendrix | 51 | hsa-mir-499-3p | 0.000444328 | — |
| MPC Prostate Dhanasekaran | 17 | hsa-mir-1266 | 3.02E−06 | — |
| END Ovarian Hendrix | 34 | hsa-mir-1826 | 3.03E−05 | — |
| RCCC Renal Lenburg | 8 | hsa-mir-889 | 6.31E−06 | — |
| END Ovarian Hendrix | 11 | hsa-mir-940 | 9.79E−05 | — |
| GL Brain Bredel | 72 | hsa-mir-760 | 0.000219694 | — |
| AD Lung Bhattacharjee | 17 | hsa-mir-491 | 4.14E−05 | — |
| COID Lung Bhattacharjee | 52 | hsa-mir-154 | 0.000584551 | — |
| GCT Seminoma Korkola | 75 | hsa-mir-708 | 3.60E−05 | — |
| ML Melanoma Talantov | 23 | hsa-mir-1259 | 2.73E−07 | — |
| ME Melanoma Hoek | 9 | hsa-mir-494 | 4.40E−05 | — |
| ML Melanoma Talantov | 14 | hsa-mir-548c-3p | 4.53E−14 | — |
| AD Lung Stearman | 36 | hsa-mir-1269 | 1.34E−05 | — |
| ILC Breast Radvanyi | 24 | hsa-mir-340 | 0.000381565 | — |
| ODGL Brain Sun | 47 | hsa-mir-1204 | 1.23E−05 | — |
| CCC Ovarian Hendrix | 9 | hsa-mir-1291 | 5.79E−05 | — |
| B-CLL Leukemia Haslinger | 68 | hsa-mir-520g | 0.000419055 | — |
| GLB Brain Sun | 68 | hsa-mir-299 | 0.000302475 | — |
| BPH Prostate Dhanasekaran | 13 | hsa-mir-922 | 3.12E−05 | — |
| OD Brain Bredel | 3 | hsa-mir-195 | 0.00018012 | — |
| MUC Ovarian Hendrix | 3 | hsa-mir-1205 | 0.000111582 | — |
| SQ Lung Bhattacharjee | 15 | hsa-mir-569 | 3.55E−07 | — |
| AD Lung Beer | 6 | hsa-mir-548c-3p | 1.66E−05 | — |
| CA Renal Higgins | 17 | hsa-mir-663 | 3.57E−07 | — |
| CCC Ovarian Hendrix | 10 | hsa-mir-615 | 2.06E−05 | — |
| SRS Ovarian Hendrix | 30 | hsa-mir-582 | 2.24E−07 | — |
| CA Renal Higgins | 13 | hsa-mir-663 | 4.59E−05 | — |
| CA Breast Richardson | 50 | hsa-mir-380 | 1.42E−05 | — |
| CA Bladder Dyrskjot | 71 | hsa-mir-662 | 0.000356281 | — |
| SMCL Lung Bhattacharjee | 5 | hsa-mir-1321 | 0.000105392 | — |
| SRS Ovarian Hendrix | 17 | hsa-mir-193b | 0.000508028 | — |
| RCCC Renal Boer | 9 | hsa-mir-502-3p | 0.000234434 | — |
| SQ Lung Bhattacharjee | 19 | hsa-mir-647 | 9.65E−06 | — |
| END Ovarian Hendrix | 31 | hsa-mir-1253 | 0.000484638 | — |
| MPM Mesothelioma Gordon | 36 | hsa-mir-487a | 3.08E−06 | — |
| CA Breast Richardson | 15 | hsa-mir-494 | 1.40E−06 | — |
| MM Myeloma Zhan | 4 | hsa-mir-185 | 6.20E−06 | — |
| CA Colon Graudens | 40 | hsa-mir-369-3p | 4.96E−06 | — |
| CA Breast Richardson | 18 | hsa-mir-135a | 3.32E−06 | — |
| ILC Breast Radvanyi | 10 | hsa-mir-548c-3p | 0.000529657 | — |
| MUC Ovarian Hendrix | 8 | hsa-mir-486-3p | 2.29E−05 | — |
| GL Brain Rickman | 32 | hsa-mir-889 | 0.000162082 | — |
| AD Lung Bhattacharjee | 34 | hsa-mir-296 | 1.13E−05 | — |
| GCT Seminoma Korkola | 52 | hsa-mir-508 | 1.79E−05 | — |
| GLB Brain Sun | 3 | hsa-mir-320a | 0.000334921 | — |
| CA Breast Richardson | 46 | hsa-mir-607 | 8.39E−07 | — |
| MPM Mesothelioma Gordon | 15 | hsa-mir-1229 | 0.000201101 | — |
| CA Colon Graudens | 31 | hsa-mir-548o | 4.28E−06 | — |
| PPC Prostate Dhanasekaran | 2 | hsa-mir-641 | 0.000166542 | — |
| END Ovarian Hendrix | 65 | hsa-mir-331-3p | 0.000108718 | — |
| SRS Ovarian Hendrix | 59 | hsa-mir-656 | 0.000449134 | — |
| MM Myeloma Zhan | 34 | hsa-mir-147b | 0.000550706 | — |
| CA Bladder Dyrskjot | 22 | hsa-mir-663 | 9.26E−07 | — |
| CCC Ovarian Hendrix | 13 | hsa-mir-500 | 0.000239274 | — |
| ME Melanoma Hoek | 15 | hsa-mir-1292 | 0.000135688 | — |
| CA Bladder Dyrskjot | 10 | hsa-mir-597 | 7.62E−05 | — |
| AC Brain Sun | 31 | hsa-mir-1280 | 5.32E−05 | — |
| GCT Seminoma Korkola | 16 | hsa-mir-1183 | 4.57E−05 | — |
| SMCL Lung Bhattacharjee | 47 | hsa-mir-302e | 0.000548428 | — |
| GL Brain Bredel | 47 | hsa-mir-1236 | 0.000346282 | — |
| GCT Seminoma Korkola | 32 | hsa-mir-608 | 0.000145012 | — |
| CCC Ovarian Hendrix | 0 | hsa-mir-194 | 6.00E−06 | — |
| MUC Ovarian Hendrix | 65 | hsa-mir-380 | 2.65E−05 | — |
| GLB Brain Sun | 59 | hsa-mir-1252 | 3.13E−05 | — |
| AC Brain Sun | 18 | hsa-mir-1305 | 0.000570172 | — |
| GCT Seminoma Korkola | 69 | hsa-mir-296 | 0.000405059 | — |
| END Ovarian Hendrix | 43 | hsa-mir-590-3p | 1.16E−05 | — |
| MM Myeloma Zhan | 26 | hsa-mir-1321 | 5.01E−06 | — |
| MPM Mesothelioma Gordon | 2 | hsa-mir-1207 | 0.000107599 | — |
| HSCC Head-Neck Chung | 4 | hsa-mir-548l | 5.28E−05 | — |
| CA Bladder Dyrskjot | 36 | hsa-mir-548c-3p | 5.69E−13 | — |
| AD Lung Bhattacharjee | 19 | hsa-mir-1247 | 0.000325418 | — |
| CA Bladder Dyrskjot | 16 | hsa-mir-939 | 9.40E−07 | — |
| SRS Ovarian Hendrix | 11 | hsa-mir-590-3p | 0.000114458 | — |
| MUC Ovarian Hendrix | 66 | hsa-mir-889 | 0.000101883 | — |
| AD Lung Stearman | 6 | hsa-mir-380 | 9.21E−05 | — |
| BPH Prostate Dhanasekaran | 4 | hsa-mir-1278 | 0.000153909 | — |
| CA Breast Richardson | 29 | hsa-mir-1302 | 5.51E−05 | — |
| AD Ovarian Welsh | 11 | hsa-mir-1225 | 0.000597055 | — |
| COID Lung Bhattacharjee | 20 | hsa-mir-615-3p | 0.000534605 | — |
| MPM Mesothelioma Gordon | 54 | hsa-mir-548h | 0.000153669 | — |
| GCT Seminoma Korkola | 34 | hsa-mir-136 | 9.69E−10 | — |
| CA Colon Graudens | 5 | hsa-mir-548h | 0.000122758 | — |
| CA Breast Richardson | 45 | hsa-mir-513a-3p | 7.34E−10 | — |
| TU Prostate Lapointe | 11 | hsa-mir-152 | 1.30E−05 | — |
| OD Brain Bredel | 47 | hsa-mir-219-1-3p | 0.000375423 | — |
| SMCL Lung Bhattacharjee | 50 | hsa-mir-664 | 5.89E−14 | — |
| MPM Mesothelioma Gordon | 33 | hsa-mir-768 | 0.000220019 | — |
| MPC Prostate Dhanasekaran | 8 | hsa-mir-219-1-3p | 0.000270967 | — |
| CCC Ovarian Hendrix | 33 | hsa-mir-770 | 0.000104352 | — |
| GLB Brain Sun | 49 | hsa-mir-340 | 5.60E−05 | — |
| END Ovarian Hendrix | 48 | hsa-mir-1304 | 1.80E−05 | — |
| CA Bladder Dyrskjot | 72 | hsa-mir-296 | 1.00E−10 | — |
| AD Lung Beer | 42 | hsa-mir-651 | 0.000137443 | — |
| MM Myeloma Zhan | 15 | hsa-mir-922 | 2.66E−05 | — |
| SRS Ovarian Hendrix | 26 | hsa-mir-325 | 9.20E−05 | — |
| CA Colon Graudens | 14 | hsa-mir-886 | 0.00042904 | — |
| MPC Prostate Dhanasekaran | 36 | hsa-mir-942 | 4.86E−05 | — |
| GL Brain Bredel | 66 | hsa-mir-27a | 0.000131506 | — |
| ILC Breast Radvanyi | 26 | hsa-mir-486-3p | 1.82E−06 | — |
| SRS Ovarian Hendrix | 35 | hsa-mir-220a | 6.69E−06 | — |
| PPC Prostate Dhanasekaran | 27 | hsa-mir-1279 | 2.59E−05 | — |
| COID Lung Bhattacharjee | 11 | hsa-mir-513b | 1.56E−05 | — |
| MUC Ovarian Hendrix | 50 | hsa-mir-889 | 4.39E−06 | — |
| ME Melanoma Hoek | 12 | hsa-mir-935 | 8.40E−06 | — |
| SQ Lung Bhattacharjee | 24 | hsa-mir-889 | 9.55E−06 | — |
| ODGL Brain Sun | 20 | hsa-mir-548e | 2.16E−06 | — |
| ODGL Brain Sun | 25 | hsa-mir-548l | 3.21E−08 | — |
| ODGL Brain Sun | 2 | hsa-mir-939 | 1.10E−11 | — |
| COID Lung Bhattacharjee | 69 | hsa-mir-765 | 0.000160635 | — |
| END Ovarian Hendrix | 3 | hsa-mir-1279 | 0.000316131 | — |
| END Ovarian Hendrix | 61 | hsa-mir-342 | 4.60E−05 | — |
| SQ Lung Bhattacharjee | 45 | hsa-mir-654 | 9.06E−05 | — |
| MPC Prostate Dhanasekaran | 34 | hsa-mir-1279 | 3.65E−08 | — |
| AD Lung Bhattacharjee | 12 | hsa-mir-590-3p | 0.000565441 | — |
| AD Pancreas Logsdon | 18 | hsa-mir-939 | 9.11E−05 | — |
| B-CLL Leukemia Haslinger | 41 | hsa-mir-483 | 0.000426084 | — |
| ML Melanoma Talantov | 36 | hsa-mir-663 | 7.10E−09 | — |
| GCT Seminoma Korkola | 106 | hsa-mir-513b | 0.000341133 | — |
| SQ Lung Bhattacharjee | 50 | hsa-mir-607 | 6.96E−05 | — |
| GCT Seminoma Korkola | 111 | hsa-mir-296 | 0.000178861 | — |
| SQ Lung Bhattacharjee | 27 | hsa-mir-940 | 8.78E−05 | — |
| MM Myeloma Zhan | 47 | hsa-mir-662 | 7.87E−05 | — |
| END Ovarian Hendrix | 40 | hsa-mir-944 | 8.92E−07 | — |
| IDC Breast Radvanyi | 4 | hsa-mir-608 | 0.000192757 | — |
| SRS Ovarian Hendrix | 6 | hsa-mir-185 | 0.000528828 | — |
| GL Brain Rickman | 9 | hsa-mir-1207 | 4.71E−06 | — |
| GL Brain Bredel | 0 | hsa-mir-548i | 0.000241782 | — |
| B-CLL Leukemia Haslinger | 65 | hsa-mir-1247 | 0.000436296 | — |
| AD Lung Bhattacharjee | 6 | hsa-mir-602 | 0.000348747 | — |
| SRS Ovarian Hendrix | 32 | hsa-mir-331-3p | 1.37E−05 | — |
| END Ovarian Hendrix | 72 | hsa-mir-548d-3p | 8.31E−06 | — |
| CA Bladder Dyrskjot | 40 | hsa-mir-548d-3p | 0.000121089 | — |
| ME Melanoma Hoek | 39 | hsa-mir-944 | 3.30E−06 | — |
| GCT Seminoma Korkola | 39 | hsa-mir-639 | 9.46E−05 | — |
| GCT Seminoma Korkola | 98 | hsa-mir-203 | 5.28E−08 | — |
| SRS Ovarian Hendrix | 48 | hsa-mir-1278 | 7.16E−05 | — |
| END Ovarian Hendrix | 60 | hsa-mir-570 | 2.61E−09 | — |
| GL Brain Rickman | 15 | hsa-mir-487a | 0.000117352 | — |
| AD Lung Beer | 53 | hsa-mir-548d-3p | 1.04E−06 | — |
| MUC Ovarian Hendrix | 63 | hsa-mir-1247 | 6.75E−05 | — |
| COID Lung Bhattacharjee | 23 | hsa-mir-1228 | 0.000222914 | — |
| SMCL Lung Bhattacharjee | 61 | hsa-mir-331-3p | 3.73E−06 | — |
| MUC Ovarian Hendrix | 0 | hsa-mir-410 | 5.36E−08 | — |
| COID Lung Bhattacharjee | 12 | hsa-mir-494 | 8.53E−06 | — |
| ME Melanoma Hoek | 41 | hsa-mir-451 | 0.000175004 | — |
| MUC Ovarian Hendrix | 69 | hsa-mir-579 | 5.8SE−05 | — |
| MUC Ovarian Hendrix | 60 | hsa-mir-574-3p | 3.68E−05 | — |
| END Ovarian Hendrix | 50 | hsa-mir-548c-3p | 2.18E−10 | — |
| PPC Prostate Dhanasekaran | 33 | hsa-mir-548e | 9.16E−06 | — |
| GLB Brain Sun | 25 | hsa-mir-600 | 6.06E−06 | — |
| MM Myeloma Zhan | 11 | hsa-mir-658 | 0.00033622 | — |
| IDC Breast Radvanyi | 41 | hsa-mir-939 | 6.12E−06 | — |
| CA Breast Richardson | 37 | hsa-mir-1289 | 0.000579338 | — |
| SRS Ovarian Hendrix | 79 | hsa-mir-944 | 5.09E−05 | — |
| AD Pancreas Logsdon | 12 | hsa-mir-933 | 0.000128551 | — |
| SRS Ovarian Hendrix | 9 | hsa-mir-1297 | 0.000150331 | — |
| RCCC Renal Lenburg | 9 | hsa-mir-1238 | 0.000381766 | — |
| AC Brain Sun | 37 | hsa-mir-569 | 0.000257268 | — |
| MPC Prostate Dhanasekaran | 12 | hsa-mir-340 | 5.22E−11 | — |
| IDC Breast Radvanyi | 14 | hsa-mir-323-3p | 0.000103181 | — |
| PPC Prostate Dhanasekaran | 37 | hsa-mir-553 | 0.000599727 | — |
| CA Bladder Dyrskjot | 15 | hsa-mir-1323 | 2.35E−07 | — |
| END Ovarian Hendrix | 82 | hsa-mir-320b | 1.53E−05 | — |
| HSCC Head-Neck Chung | 7 | hsa-mir-573 | 4.02E−05 | — |
| MM Myeloma Zhan | 13 | hsa-mir-770 | 3.53E−05 | — |
| B-CLL Leukemia Haslinger | 35 | hsa-mir-486-3p | 5.55E−09 | — |
| CA Bladder Dyrskjot | 54 | hsa-mir-491 | 6.59E−06 | — |
| SRS Ovarian Hendrix | 61 | hsa-mir-1224-3p | 0.000355435 | — |
| AO Brain Bredel | 22 | hsa-mir-663 | 3.41E−06 | — |
| IDC Breast Radvanyi | 56 | hsa-mir-576 | 0.000441942 | — |
| B-CLL Leukemia Haslinger | 21 | hsa-mir-548c-3p | 1.93E−14 | — |
| MUC Ovarian Hendrix | 7 | hsa-mir-590-3p | 1.46E−10 | — |
| B-CLL Leukemia Haslinger | 52 | hsa-mir-302a | 0.000364911 | — |
| GL Brain Rickman | 34 | hsa-mir-1274a | 4.31E−05 | — |
| ML Melanoma Talantov | 9 | hsa-mir-493 | 0.000491063 | — |
| MPC Prostate Dhanasekaran | 40 | hsa-mir-220 | 9.77E−06 | — |
| CA Breast Sorlie | 23 | hsa-mir-423 | 0.000106954 | — |
| AD Lung Bhattacharjee | 82 | hsa-mir-647 | 0.000162289 | — |
| AD Ovarian Welsh | 9 | hsa-mir-524 | 3.48E−05 | — |
| GL Brain Rickman | 27 | hsa-mir-182 | 7.81E−05 | — |
| CA Bladder Dyrskjot | 80 | hsa-mir-616 | 7.12E−05 | — |
| FL Lymphoma Alizadeh | 5 | hsa-mir-581 | 0.000111755 | — |
| TU Prostate Lapointe | 32 | hsa-mir-608 | 6.56E−07 | — |
| SRS Ovarian Hendrix | 73 | hsa-mir-340 | 1.71E−08 | — |
| B-CLL Leukemia Haslinger | 51 | hsa-mir-448 | 0.000227269 | — |
| CA Bladder Dyrskjot | 24 | hsa-mir-663 | 2.33E−05 | — |
| ODGL Brain Sun | 7 | hsa-mir-548k | 0.000124326 | — |
| ODGL Brain Sun | 41 | hsa-mir-26b | 5.24E−05 | — |
| AD Lung Beer | 28 | hsa-mir-1245 | 0.000608297 | — |
| CA Breast Sorlie | 17 | hsa-mir-640 | 0.000265634 | — |
| SMCL Lung Bhattacharjee | 7 | hsa-mir-615 | 0.00025691 | — |
| CA Bladder Dyrskjot | 29 | hsa-mir-548c-3p | 1.79E−06 | — |
| CA Breast Richardson | 2 | hsa-mir-590-3p | 8.86E−05 | — |
| PPC Prostate Dhanasekaran | 20 | hsa-mir-1207 | 0.000191478 | — |
| SRS Ovarian Hendrix | 57 | hsa-mir-548d-3p | 5.11E−06 | — |
| OD Brain Bredel | 54 | hsa-mir-1275 | 7.51E−05 | — |
| MUC Ovarian Hendrix | 68 | hsa-mir-1305 | 1.03E−05 | — |
| MPM Mesothelioma Gordon | 32 | hsa-mir-541 | 0.000130877 | — |
| AD Lung Stearman | 27 | hsa-mir-300 | 5.00E−06 | — |
| RCCC Renal Boer | 15 | hsa-mir-300 | 0.000145597 | — |
| MPC Prostate Dhanasekaran | 2 | hsa-mir-548c-3p | 2.35E−06 | — |
| CA Bladder Dyrskjot | 64 | hsa-mir-590-3p | 1.06E−25 | — |
| AC Brain Sun | 3 | hsa-mir-1205 | 2.84E−05 | — |
| CA Breast Richardson | 54 | hsa-mir-361-3p | 2.22E−12 | — |
| B-CLL Leukemia Haslinger | 28 | hsa-mir-376 | 0.000318075 | — |
| B-CLL Leukemia Haslinger | 54 | hsa-mir-1321 | 3.27E−05 | — |
| SMCL Lung Bhattacharjee | 31 | hsa-mir-513a-3p | 1.28E−08 | — |
| AD Lung Bhattacharjee | 41 | hsa-mir-296 | 6.11E−05 | — |
| AD Lung Stearman | 32 | hsa-mir-588 | 0.00048611 | — |
| GCT Seminoma Korkola | 0 | hsa-mir-1251 | 3.03E−05 | — |
| AD Lung Bhattacharjee | 14 | hsa-mir-938 | 0.000464954 | — |
| GCT Seminoma Korkola | 108 | hsa-mir-590 | 0.000599626 | — |
| ODGL Brain Sun | 65 | hsa-mir-495 | 3.90E−08 | — |
| B-CLL Leukemia Haslinger | 16 | hsa-mir-1285 | 8.55E−08 | — |
| AD Ovarian Welsh | 31 | hsa-mir-548c-3p | 1.17E−06 | — |
| GLB Brain Sun | 37 | hsa-mir-1256 | 0.000394523 | — |
| IDC Breast Radvanyi | 18 | hsa-mir-1207-3p | 0.000171598 | — |
| AD Lung Bhattacharjee | 8 | hsa-mir-1324 | 2.99E−05 | — |
| IDC Breast Radvanyi | 65 | hsa-mir-802 | 3.44E−17 | — |
| CCC Ovarian Hendrix | 56 | hsa-mir-587 | 0.000320898 | — |
| SQ Lung Bhattacharjee | 23 | hsa-mir-586 | 0.000126996 | — |
| GLB Brain Sun | 27 | hsa-mir-889 | 4.14E−06 | — |
| CA Bladder Dyrskjot | 32 | hsa-mir-1270 | 1.86E−06 | — |
| CA Bladder Dyrskjot | 58 | hsa-mir-328 | 0.000324179 | — |
| COID Lung Bhattacharjee | 74 | hsa-mir-513a-3p | 1.46E−07 | — |
| B-CLL Leukemia Haslinger | 43 | hsa-mir-483-3p | 6.86E−05 | — |
| HSCC Head-Neck Cromer | 26 | hsa-mir-885 | 1.44E−05 | — |
| GLB Brain Sun | 35 | hsa-mir-922 | 0.000367795 | — |
| ML Melanoma Talantov | 10 | hsa-mir-1180 | 0.000368404 | — |
| AD Ovarian Welsh | 8 | hsa-mir-1260 | 3.07E−05 | — |
| CCC Ovarian Hendrix | 58 | hsa-mir-586 | 3.47E−08 | — |
| AC Brain Sun | 50 | hsa-mir-483-3p | 0.000171585 | — |
| BPH Prostate Dhanasekaran | 1 | hsa-mir-579 | 0.00020642 | — |
| ODGL Brain Sun | 27 | hsa-mir-136 | 0.000113816 | — |
| COID Lung Bhattacharjee | 43 | hsa-mir-658 | 7.50E−05 | — |
| GL Brain Rickman | 17 | hsa-mir-146b-3p | 0.000528862 | — |
| CA Breast Richardson | 19 | hsa-mir-586 | 0.000229107 | — |
| SRS Ovarian Hendrix | 4 | hsa-mir-1250 | 1.87E−05 | — |
| CA Breast Sorlie | 16 | hsa-mir-579 | 3.19E−05 | — |
| HSCC Head-Neck Cromer | 18 | hsa-mir-615-3p | 0.000195451 | — |
| MPM Mesothelioma Gordon | 62 | hsa-mir-708 | 0.000307699 | — |
| ML Melanoma Talantov | 40 | hsa-mir-296 | 4.56E−06 | — |
| MUC Ovarian Hendrix | 54 | hsa-mir-921 | 1.11E−05 | — |
| CA Bladder Dyrskjot | 12 | hsa-mir-1244 | 0.000260354 | — |
| TU Prostate Lapointe | 33 | hsa-mir-548c-3p | 1.75E−07 | — |
| MPC Prostate Dhanasekaran | 27 | hsa-mir-1271 | 0.000148676 | — |
| CA Renal Higgins | 4 | hsa-mir-34b | 2.00E−07 | — |
| END Ovarian Hendrix | 4 | hsa-mir-612 | 0.00056313 | — |
| MPM Mesothelioma Gordon | 44 | hsa-mir-338 | 8.55E−05 | — |
| B-CLL Leukemia Haslinger | 17 | hsa-mir-590-3p | 1.39E−13 | — |
| CA Bladder Dyrskjot | 48 | hsa-mir-520b | 0.000475673 | — |
| CCC Ovarian Hendrix | 46 | hsa-mir-340 | 7.26E−09 | — |
| HSCC Head-Neck Chung | 1 | hsa-mir-450b | 3.20E−05 | — |
| COID Lung Bhattacharjee | 6 | hsa-mir-874 | 4.15E−05 | — |
| CA Bladder Dyrskjot | 37 | hsa-mir-548n | 9.02E−08 | — |
| CA Bladder Dyrskjot | 14 | hsa-mir-944 | 3.76E−05 | — |
| SRS Ovarian Hendrix | 44 | hsa-mir-584 | 0.000486209 | — |
| ODGL Brain Sun | 22 | hsa-mir-423 | 7.28E−09 | — |
| AD Lung Bhattacharjee | 46 | hsa-mir-548c-3p | 3.06E−06 | — |
| AD Lung Stearman | 19 | hsa-mir-370 | 2.85E−06 | — |
| GL Brain Rickman | 8 | hsa-mir-634 | 0.000383758 | — |
| SRS Ovarian Hendrix | 3 | hsa-mir-146b-3p | 0.000175563 | — |
| CA Bladder Dyrskjot | 56 | hsa-mir-1238 | 2.31E−06 | — |
| IDC Breast Radvanyi | 43 | hsa-mir-1224 | 5.00E−05 | — |
| ILC Breast Radvanyi | 19 | hsa-mir-338-3p | 0.000520796 | — |
| SMCL Lung Bhattacharjee | 22 | hsa-mir-548n | 9.61E−05 | — |
| SRS Ovarian Hendrix | 53 | hsa-mir-576 | 3.87E−05 | — |
| CA Bladder Dyrskjot | 6 | hsa-mir-939 | 1.03E−05 | — |
| AD Lung Bhattacharjee | 7 | hsa-mir-1305 | 2.19E−11 | — |
| AD Lung Beer | 8 | hsa-mir-637 | 3.07E−05 | — |
| CA Bladder Dyrskjot | 49 | hsa-mir-432 | 8.72E−06 | — |
| BPH Prostate Dhanasekaran | 15 | hsa-mir-1259 | 9.49E−05 | — |
| OD Brain Bredel | 21 | hsa-mir-548c-3p | 0.000226458 | — |
| TU Prostate Lapointe | 38 | hsa-mir-633 | 9.66E−05 | — |
| AD Ovarian Welsh | 5 | hsa-mir-455 | 9.98E−05 | — |
| GL Brain Rickman | 29 | hsa-mir-568 | 3.23E−08 | — |
| IDC Breast Radvanyi | 2 | hsa-mir-513a-3p | 3.57E−05 | — |
| GL Brain Bredel | 31 | hsa-mir-615-3p | 2.09E−05 | — |
| AC Brain Sun | 8 | hsa-mir-196a | 0.000415339 | — |
| SRS Ovarian Hendrix | 19 | hsa-mir-135a | 6.95E−06 | — |
| ML Melanoma Talantov | 29 | hsa-mir-1257 | 0.00027138 | — |
| AD Lung Stearman | 0 | hsa-mir-369-3p | 2.30E−06 | — |
| CA Bladder Dyrskjot | 47 | hsa-mir-197 | 7.26E−07 | — |
| COID Lung Bhattacharjee | 72 | hsa-mir-548n | 9.38E−06 | — |
| AD Ovarian Welsh | 30 | hsa-mir-1204 | 0.000439516 | — |
| GLB Brain Sun | 58 | hsa-mir-1287 | 0.000103913 | — |
| SQ Lung Bhattacharjee | 43 | hsa-mir-635 | 0.000124798 | — |
| AC Brain Sun | 34 | hsa-mir-223 | 2.97E−05 | — |
| AD Lung Stearman | 18 | hsa-mir-1257 | 0.000397558 | — |
| GCT Seminoma Korkola | 68 | hsa-mir-1182 | 6.01E−05 | — |
| COID Lung Bhattacharjee | 45 | hsa-mir-615 | 5.02E−06 | — |
| GL Brain Bredel | 25 | hsa-mir-513b | 0.000419468 | — |
| ML Melanoma Talantov | 19 | hsa-mir-548c-3p | 7.99E−10 | — |
| MUC Ovarian Hendrix | 32 | hsa-mir-490-3p | 9.00E−05 | — |
| BPH Prostate Dhanasekaran | 8 | hsa-mir-1246 | 7.27E−06 | — |
| B-CLL Leukemia Haslinger | 5 | hsa-mir-622 | 6.58E−05 | — |
| PPC Prostate Dhanasekaran | 39 | hsa-mir-139-3p | 0.000138404 | — |
| AD Pancreas Logsdon | 1 | hsa-mir-1247 | 1.53E−05 | — |
| CCC Ovarian Hendrix | 2 | hsa-mir-423 | 1.48E−06 | — |
| SQ Lung Bhattacharjee | 40 | hsa-mir-339-3p | 0.000101826 | — |
| GL Brain Bredel | 30 | hsa-mir-342 | 3.60E−07 | — |
| MCA Breast Radvanyi | 7 | hsa-mir-324-3p | 3.23E−06 | — |
| B-CLL Leukemia Haslinger | 0 | hsa-mir-409 | 0.000378892 | — |
| TU Prostate Lapointe | 25 | hsa-mir-130a | 3.79E−05 | — |
| MPM Mesothelioma Gordon | 65 | hsa-mir-548n | 2.71E−09 | — |
| SRS Ovarian Hendrix | 43 | hsa-mir-299-3p | 5.54E−06 | — |
| B-CLL Leukemia Haslinger | 56 | hsa-mir-548b-3p | 0.000133402 | — |
| MM Myeloma Zhan | 14 | hsa-mir-548l | 0.000147742 | — |
| AD Lung Beer | 14 | hsa-mir-542 | 0.000318042 | — |
| SRS Ovarian Hendrix | 80 | hsa-mir-324 | 4.34E−05 | — |
| OD Brain Bredel | 40 | hsa-mir-216b | 9.25E−05 | — |
| END Ovarian Hendrix | 35 | hsa-mir-1276 | 0.000277741 | — |
| CCC Ovarian Hendrix | 12 | hsa-mir-508-3p | 3.68E−05 | — |
| MM Myeloma Zhan | 43 | hsa-mir-124 | 2.37E−05 | — |
| END Ovarian Hendrix | 16 | hsa-mir-588 | 3.82E−06 | — |
| MPM Mesothelioma Gordon | 13 | hsa-mir-585 | 4.85E−05 | — |
| HSCC Head-Neck Cromer | 12 | hsa-mir-513a-3p | 2.34E−05 | — |
| COID Lung Bhattacharjee | 24 | hsa-mir-342 | 7.55E−05 | — |
| IDC Breast Radvanyi | 27 | hsa-mir-323 | 0.000569837 | — |
| ODGL Brain Sun | 50 | hsa-mir-217 | 0.000123791 | — |
| AD Lung Bhattacharjee | 24 | hsa-mir-608 | 1.22E−06 | — |
| GCT Seminoma Korkola | 71 | hsa-mir-361-3p | 4.76E−07 | — |
| AD Lung Beer | 12 | hsa-mir-378 | 0.000355275 | — |
| SQ Lung Bhattacharjee | 29 | hsa-mir-486-3p | 8.12E−06 | — |
| MM Myeloma Zhan | 35 | hsa-mir-129 | 0.000200071 | — |
| CCC Ovarian Hendrix | 4 | hsa-mir-194 | 1.45E−05 | — |
| AD Ovarian Welsh | 16 | hsa-mir-548l | 0.000120211 | — |
| AD Lung Beer | 34 | hsa-mir-646 | 0.00025737 | — |
| GBM Brain Liang | 2 | hsa-mir-548c-3p | 0.000318973 | — |
| OD Brain Bredel | 30 | hsa-mir-1306 | 0.000381469 | — |
| END Ovarian Hendrix | 75 | hsa-mir-489 | 0.000119996 | — |
| AD Lung Beer | 10 | hsa-mir-600 | 1.44E−06 | — |
| MPC Prostate Dhanasekaran | 18 | hsa-mir-1263 | 1.48E−05 | — |
| PPC Prostate Dhanasekaran | 35 | hsa-mir-663b | 0.000144273 | — |
| MPM Mesothelioma Gordon | 61 | hsa-mir-1287 | 2.33E−05 | — |
| AO Brain Bredel | 32 | hsa-mir-564 | 5.13E−06 | — |
| CA Bladder Dyrskjot | 75 | hsa-mir-513 | 7.29E−05 | — |
| CA Colon Graudens | 28 | hsa-mir-508 | 0.000295908 | — |
| ML Melanoma Talantov | 3 | hsa-mir-548n | 4.08E−06 | — |
| GCT Seminoma Korkola | 30 | hsa-mir-588 | 0.000275648 | — |
| PPC Prostate Dhanasekaran | 31 | hsa-mir-515-3p | 0.000197799 | — |
| ODGL Brain Sun | 49 | hsa-mir-423 | 1.08E−05 | — |
| CA Bladder Dyrskjot | 62 | hsa-mir-637 | 1.36E−05 | — |
| CA Bladder Dyrskjot | 46 | hsa-mir-342 | 3.59E−08 | — |
| HSCC Head-Neck Cromer | 19 | hsa-mir-548l | 1.75E−07 | — |
| AD Lung Beer | 40 | hsa-mir-561 | 7.29E−05 | — |
| CCC Ovarian Hendrix | 18 | hsa-mir-326 | 7.22E−06 | — |
| MUC Ovarian Hendrix | 61 | hsa-mir-410 | 4.05E−06 | — |
| AC Brain Sun | 19 | hsa-mir-485 | 1.27E−05 | — |
| COID Lung Bhattacharjee | 14 | hsa-mir-557 | 0.000108663 | — |
| CA Bladder Dyrskjot | 38 | hsa-mir-1281 | 0.000191838 | — |
| CLL Lymphoma Alizadeh | 0 | hsa-mir-548j | 0.000459332 | — |
| SMCL Lung Bhattacharjee | 29 | hsa-mir-548c-3p | 9.77E−08 | — |
| SQ Lung Bhattacharjee | 21 | hsa-mir-455 | 0.000112719 | — |
| MM Myeloma Zhan | 9 | hsa-mir-185 | 7.75E−05 | — |
| HSCC Head-Neck Cromer | 8 | hsa-mir-142 | 1.01E−07 | — |
| CA Bladder Dyrskjot | 18 | hsa-mir-1291 | 2.29E−05 | — |
| PPC Prostate Dhanasekaran | 4 | hsa-mir-939 | 9.40E−06 | — |
| DLBCL Lymphoma Alizadeh | 1 | hsa-mir-16 | 8.17E−05 | — |
| PPC Prostate Dhanasekaran | 14 | hsa-mir-1202 | 0.000130297 | — |
| SRS Ovarian Hendrix | 1 | hsa-mir-409-3p | 9.82E−05 | — |
| COID Lung Bhattacharjee | 66 | hsa-mir-507 | 9.71E−06 | — |
| MM Myeloma Zhan | 10 | hsa-mir-1305 | 6.75E−06 | — |
| ME Melanoma Hoek | 37 | hsa-mir-380 | 1.50E−07 | — |
| OOGL Brain Sun | 62 | hsa-mir-1294 | 6.05E−05 | — |
| CA Bladder Dyrskjot | 21 | hsa-mir-944 | 2.51E−06 | — |
| COID Lung Bhattacharjee | 0 | hsa-mir-518d | 2.57E−05 | — |
| CA Colon Graudens | 6 | hsa-mir-658 | 8.78E−05 | — |
| GCT Seminoma Korkola | 88 | hsa-mir-338 | 3.32E−08 | — |
| AD Lung Stearman | 35 | hsa-mir-668 | 0.000306085 | — |
| B-CLL Leukemia Haslinger | 45 | hsa-mir-889 | 9.32E−06 | — |
| HSCC Head-Neck Cramer | 27 | hsa-mir-615 | 5.07E−06 | — |
| GCT Seminoma Korkola | 66 | hsa-mir-1291 | 1.92E−05 | — |
| AD Lung Beer | 22 | hsa-mir-525 | 0.000251267 | — |
| AO Brain Bredel | 0 | hsa-mir-635 | 9.52E−05 | — |
| MUC Ovarian Hendrix | 75 | hsa-mir-512-3p | 1.28E−06 | — |
| SQ Lung Bhattacharjee | 14 | hsa-mir-486-3p | 5.23E−06 | — |
| MPC Prostate Dhanasekaran | 23 | hsa-mir-302f | 0.000532122 | — |
| IDC Breast Radvanyi | 46 | hsa-mir-675 | 0.000483213 | — |
| CA Bladder Dyrskjot | 35 | hsa-mir-486-3p | 0.000273385 | — |
| CCC Ovarian Hendrix | 45 | hsa-mir-1250 | 2.30E−05 | — |
| ODGL Brain Sun | 1 | hsa-mir-647 | 1.96E−05 | — |
| CA Breast Richardson | 62 | hsa-mir-622 | 0.000211265 | — |
| OD Brain Bredel | 15 | hsa-mir-765 | 0.000578248 | — |
| B-CLL Leukemia Haslinger | 8 | hsa-mir-939 | 9.79E−06 | — |
| CA Breast Sorlie | 8 | hsa-mir-1255a | 3.53E−05 | — |
| GLB Brain Sun | 50 | hsa-mir-548c-3p | 2.66E−06 | — |
| GCT Seminoma Korkola | 82 | hsa-mir-576 | 0.000285312 | — |
| END Ovarian Hendrix | 23 | hsa-mir-22 | 6.91E−05 | — |
| OD Brain Bredel | 62 | hsa-let-7f | 0.000585924 | — |
| MM Myeloma Zhan | 36 | hsa-mir-1273 | 0.000403952 | — |
| CA Colon Graudens | 0 | hsa-mir-138 | 6.29E−05 | — |
| GCT Seminoma Korkola | 29 | hsa-mir-548c-3p | 3.74E−08 | — |
| B-CLL Leukemia Haslinger | 15 | hsa-mir-654-3p | 2.08E−05 | — |
| ILC Breast Radvanyi | 6 | hsa-mir-579 | 8.72E−06 | — |
| MUC Ovarian Hendrix | 12 | hsa-mir-658 | 0.000144043 | — |
| TU Prostate Lapointe | 1 | hsa-mir-590-3p | 3.15E−07 | — |
| HSCC Head-Neck Cromer | 25 | hsa-mir-495 | 1.15E−06 | — |
| CLL Lymphoma Alizadeh | 1 | hsa-mir-1204 | 3.50E−05 | — |
| AO Brain Bredel | 18 | hsa-mir-497 | 0.000198786 | — |
| TU Prostate Lapointe | 31 | hsa-mir-339-3p | 5.79E−05 | — |
| CA Breast Richardson | 55 | hsa-mir-544 | 1.29E−06 | — |
| HSCC Head-Neck Chung | 8 | hsa-mir-410 | 4.98E−05 | — |
| IDC Breast Radvanyi | 26 | hsa-mir-886 | 0.000540903 | — |
| SRS Ovarian Hendrix | 28 | hsa-mir-186 | 1.67E−06 | — |
| BPH Prostate Dhanasekaran | 11 | hsa-mir-369-3p | 0.000568201 | — |
| SQ Lung Bhattacharjee | 7 | hsa-mir-765 | 5.75E−05 | — |
| ML Melanoma Talantov | 7 | hsa-mir-296 | 6.02E−07 | — |
| ME Melanoma Hoek | 33 | hsa-mir-1265 | 0.000457688 | — |
| GCT Seminoma Korkola | 50 | hsa-mir-376 | 2.33E−06 | — |
| END Ovarian Hendrix | 22 | hsa-mir-944 | 1.00E−05 | — |
| MUC Ovarian Hendrix | 22 | hsa-mir-1293 | 5.80E−05 | — |
| AC Brain Sun | 7 | hsa-mir-548l | 1.20E−07 | — |
| AC Brain Sun | 16 | hsa-mir-655 | 1.38E−06 | — |
| HSCC Head-Neck Chung | 5 | hsa-mir-1202 | 0.000335288 | — |
| PPC Prostate Dhanasekaran | 23 | hsa-mir-518a | 7.91E−05 | — |
| GLB Brain Sun | 55 | hsa-mir-32 | 0.000230839 | — |
| MUC Ovarian Hendrix | 31 | hsa-mir-342 | 2.61E−05 | — |
| END Ovarian Hendrix | 69 | hsa-mir-186 | 6.88E−06 | — |
| ODGL Brain Sun | 48 | hsa-mir-384 | 0.000253138 | — |
| TU Prostate Lapointe | 4 | hsa-mir-185 | 0.000290147 | — |
| AD Lung Beer | 5 | hsa-mir-1292 | 9.07E−05 | — |
| PPC Prostate Dhanasekaran | 10 | hsa-mir-889 | 8.82E−06 | — |
| CA Bladder Dyrskjot | 82 | hsa-mir-656 | 1.37E−06 | — |
| AD Lung Bhattacharjee | 4 | hsa-mir-1321 | 3.68E−07 | — |
| IDC Breast Radvanyi | 24 | hsa-mir-1291 | 4.64E−05 | — |
| B-CLL Leukemia Haslinger | 37 | hsa-mir-323-3p | 6.95E−10 | — |
| MPM Mesothelioma Gordon | 17 | hsa-mir-1296 | 0.000161062 | — |
| AC Brain Sun | 6 | hsa-mir-139 | 0.000306314 | — |
| GLB Brain Sun | 4 | hsa-mir-542 | 4.28E−07 | — |
| ODGL Brain Sun | 30 | hsa-mir-483 | 0.000412141 | — |
| ODGL Brain Sun | 60 | hsa-let-7d | 0.000130896 | — |
| COID Lung Bhattacharjee | 80 | hsa-mir-548l | 6.43E−13 | — |
| ODGL Brain Sun | 32 | hsa-mir-153 | 2.80E−06 | — |
| GCT Seminoma Korkola | 17 | hsa-mir-615 | 2.79E−07 | — |
| B-CLL Leukemia Haslinger | 25 | hsa-mir-548c-3p | 7.95E−06 | — |
| AC Brain Sun | 22 | hsa-mir-185 | 3.82E−05 | — |
| BPH Prostate Dhanasekaran | 12 | hsa-mir-939 | 8.80E−05 | — |
| CA Bladder Dyrskjot | 63 | hsa-mir-372 | 1.77E−07 | — |
| B-CLL Leukemia Haslinger | 13 | hsa-mir-892b | 0.000527397 | — |
| GL Brain Bredel | 46 | hsa-mir-548l | 1.16E−05 | — |
| MPM Mesothelioma Gordon | 41 | hsa-mir-381 | 0.000443852 | — |
| ODGL Brain Sun | 10 | hsa-mir-208a | 0.000136267 | — |
| MPC Prostate Dhanasekaran | 44 | hsa-mir-576-3p | 9.02E−05 | — |
| CA Colon Graudens | 10 | hsa-mir-1225 | 0.000267591 | — |
| PPC Prostate Dhanasekaran | 38 | hsa-mir-607 | 2.03E−05 | — |
| GLB Brain Sun | 29 | hsa-mir-1275 | 9.58E−09 | — |
| SQ Lung Bhattacharjee | 13 | hsa-mir-296 | 2.05E−08 | — |
| END Ovarian Hendrix | 62 | hsa-mir-571 | 6.37E−05 | — |
| AC Brain Sun | 23 | hsa-mir-1207-3p | 0.000547066 | — |
| CA Bladder Dyrskjot | 7 | hsa-mir-361 | 0.000142136 | — |
| ODGL Brain Sun | 39 | hsa-mir-1283 | 3.48E−05 | — |
| CLL Lymphoma Alizadeh | 13 | hsa-mir-423 | 0.000349636 | — |
| CCC Ovarian Hendrix | 25 | hsa-mir-744 | 0.000564266 | — |
| SQ Lung Bhattacharjee | 18 | hsa-mir-410 | 2.12E−05 | — |
| TU Prostate Lapointe | 39 | hsa-mir-656 | 3.01E−07 | — |
| GL Brain Bredel | 79 | hsa-mir-340 | 2.04E−18 | — |
| PPC Prostate Dhanasekaran | 41 | hsa-mir-369-3p | 3.86E−06 | — |
| MUC Ovarian Hendrix | 20 | hsa-mir-640 | 0.000235199 | — |
| PPC Prostate Dhanasekaran | 9 | hsa-mir-661 | 1.10E−05 | — |
| MUC Ovarian Hendrix | 52 | hsa-mir-541 | 0.00046487 | — |
| GBM Brain Liang | 5 | hsa-mir-939 | 0.00056305 | — |
| FL Lymphoma Alizadeh | 7 | hsa-mir-1282 | 0.000138941 | — |
| AD Lung Bhattacharjee | 47 | hsa-mir-1245 | 2.22E−06 | — |
| B-CLL Leukemia Haslinger | 62 | hsa-mir-548c-3p | 8.00E−10 | — |
| CCC Ovarian Hendrix | 60 | hsa-mir-1305 | 3.35E−05 | — |
| B-CLL Leukemia Haslinger | 29 | hsa-mir-1270 | 0.000171591 | — |
| HSCC Head-Neck Chung | 3 | hsa-mir-1286 | 9.91E−06 | — |
| MM Myeloma Zhan | 16 | hsa-mir-134 | 8.11E−05 | — |
| GCT Seminoma Korkola | 60 | hsa-mir-551a | 6.95E−05 | — |
| COID Lung Bhattacharjee | 21 | hsa-mir-944 | 8.19E−10 | — |
| IDC Breast Radvanyi | 8 | hsa-mir-631 | 0.000564379 | — |
| CA Bladder Dyrskjot | 70 | hsa-mir-502 | 2.29E−05 | — |
| CA Breast Richardson | 23 | hsa-mir-544 | 0.000153218 | — |
| AO Brain Bredel | 38 | hsa-mir-1282 | 0.000299426 | — |
| ODGL Brain Sun | 53 | hsa-mir-548n | 5.14E−09 | — |
| MUC Ovarian Hendrix | 24 | hsa-mir-361-3p | 0.000241059 | — |
| GLB Brain Sun | 5 | hsa-mir-515 | 0.000373578 | — |
| MPM Mesothelioma Gordon | 24 | hsa-mir-361-3p | 0.000101355 | — |
| AD Ovarian Welsh | 17 | hsa-mir-1321 | 1.68E−05 | — |
| HSCC Head-Neck Chung | 6 | hsa-mir-590-3p | 1.29E−05 | — |
| MM Myeloma Zhan | 3 | hsa-mir-548c-3p | 2.85E−14 | — |
| CA Colon Graudens | 17 | hsa-mir-597 | 7.44E−05 | — |
| B-CLL Leukemia Haslinger | 46 | hsa-mir-1247 | 0.000479165 | — |
| CA Bladder Dyrskjot | 79 | hsa-mir-508-3p | 0.00023817 | — |
| CCC Ovarian Hendrix | 47 | hsa-mir-122 | 7.65E−06 | — |
| RCCC Renal Lenburg | 3 | hsa-mir-888 | 0.00018121 | — |
| AD Ovarian Welsh | 29 | hsa-mir-1225-3p | 0.000495059 | — |
| ML Melanoma Talantov | 18 | hsa-mir-513a-3p | 1.30E−08 | — |
| MPM Mesothelioma Gordon | 38 | hsa-mir-145 | 8.33E−05 | — |
| CA Breast Sorlie | 19 | hsa-mir-129 | 8.52E−06 | — |
| CA Renal Higgins | 10 | hsa-mir-483-3p | 0.000138161 | — |
| MPM Mesothelioma Gordon | 42 | hsa-mir-539 | 3.04E−05 | — |
| ME Melanoma Hoek | 25 | hsa-mir-26a | 2.11E−05 | — |
| GL Brain Rickman | 4 | hsa-mir-361 | 3.19E−05 | — |
| GLB Brain Sun | 28 | hsa-mir-323-3p | 4.84E−05 | — |
| GL Brain Rickman | 10 | hsa-mir-648 | 3.05E−06 | — |
| AD Lung Beer | 33 | hsa-mir-379 | 6.19E−05 | — |
| MPC Prostate Dhanasekaran | 5 | hsa-mir-211 | 0.000182393 | — |
| TU Prostate Lapointe | 29 | hsa-mir-522 | 2.97E−11 | — |
| END Ovarian Hendrix | 13 | hsa-mir-23a | 6.11E−07 | — |
| CA Bladder Dyrskjot | 31 | hsa-mir-656 | 1.34E−05 | — |
| AD Lung Stearman | 25 | hsa-mir-519a | 8.68E−05 | — |
| GL Brain Bredel | 77 | hsa-mir-744 | 1.44E−05 | — |
| COID Lung Bhattacharjee | 16 | hsa-mir-376a | 0.000262419 | — |
| MPM Mesothelioma Gordon | 1 | hsa-mir-1224 | 2.48E−05 | — |
| RCCC Renal Lenburg | 10 | hsa-mir-1273 | 0.000324812 | — |
| ILC Breast Radvanyi | 3 | hsa-mir-383 | 0.000402616 | — |
| ML Melanoma Talantov | 12 | hsa-mir-324-3p | 6.94E−05 | — |
| MCA Breast Radvanyi | 17 | hsa-mir-219 | 1.57E−07 | — |
| B-CLL Leukemia Haslinger | 66 | hsa-mir-939 | 3.01E−10 | — |
| GL Brain Bredel | 27 | hsa-mir-181 | 0.000133619 | — |
| B-CLL Leukemia Haslinger | 10 | hsa-mir-454 | 1.68E−05 | — |
| OD Brain Bredel | 52 | hsa-mir-411 | 0.000409711 | — |
| MPM Mesothelioma Gordon | 16 | hsa-mir-590-3p | 9.51E−17 | — |
| CA Bladder Dyrskjot | 45 | hsa-mir-608 | 4.60E−07 | — |
| CA Bladder Dyrskjot | 59 | hsa-mir-374a | 8.54E−06 | — |
| SQ Lung Bhattacharjee | 22 | hsa-mir-607 | 1.71E−06 | — |
| MPC Prostate Dhanasekaran | 14 | hsa-mir-502 | 0.000300801 | — |
| B-CLL Leukemia Haslinger | 49 | hsa-mir-1227 | 0.00034531 | — |
| B-CLL Leukemia Haslinger | 9 | hsa-mir-1245 | 0.000110184 | — |
| PPC Prostate Dhanasekaran | 19 | hsa-mir-144 | 3.07E−06 | — |
| GBM Brain Liang | 1 | hsa-mir-423 | 7.21E−07 | — |
| CA Bladder Dyrskjot | 67 | hsa-mir-630 | 0.000123706 | — |
| MPC Prostate Dhanasekaran | 35 | hsa-mir-203 | 1.86E−06 | — |
| GLB Brain Sun | 8 | hsa-mir-501 | 3.42E−06 | — |
| MM Myeloma Zhan | 0 | hsa-mir-1207 | 3.32E−06 | — |
| AC Brain Sun | 4 | hsa-mir-371-3p | 8.52E−05 | — |
| CCC Ovarian Hendrix | 27 | hsa-mir-556 | 0.000258408 | — |
| SQ Lung Bhattacharjee | 5 | hsa-mir-595 | 5.58E−05 | — |
| COID Lung Bhattacharjee | 60 | hsa-mir-516a-3p | 5.94E−05 | — |
| MPC Prostate Dhanasekaran | 42 | hsa-mir-331 | 2.97E−05 | — |
| COID Lung Bhattacharjee | 8 | hsa-mir-340 | 7.24E−07 | — |
| MUC Ovarian Hendrix | 25 | hsa-mir-582 | 3.54E−06 | — |
| AD Lung Bhattacharjee | 28 | hsa-mir-548a-3p | 5.98E−06 | — |
| AD Lung Bhattacharjee | 64 | hsa-mir-765 | 2.85E−06 | — |
| MPM Mesothelioma Gordon | 28 | hsa-mir-590-3p | 0.000287627 | — |
| MPM Mesothelioma Gordon | 63 | hsa-mir-548c-3p | 2.51E−09 | — |
| B-CLL Leukemia Haslinger | 23 | hsa-mir-548o | 0.000480335 | — |
| MPM Mesothelioma Gordon | 6 | hsa-mir-192 | 0.000120946 | — |
| TU Prostate Lapointe | 24 | hsa-mir-548g | 1.22E−06 | — |
| OD Brain Bredel | 7 | hsa-mir-23b | 2.03E−06 | — |
| COID Lung Bhattacharjee | 68 | hsa-mir-1272 | 0.00018659 | — |
| PDC Pancreas Ishikawa | 2 | hsa-mir-448 | 8.41E−05 | — |
| GLB Brain Sun | 62 | hsa-mir-495 | 0.000134046 | — |
| MM Myeloma Zhan | 40 | hsa-mir-612 | 0.000176116 | — |
| CA Breast Richardson | 17 | hsa-mir-423 | 2.85E−12 | — |
| ML Melanoma Talantov | 28 | hsa-mir-1207 | 1.66E−07 | — |
| CA Renal Higgins | 20 | hsa-mir-1261 | 0.000372363 | — |
| ML Melanoma Talantov | 4 | hsa-mir-146b-3p | 0.000174949 | — |
| ODGL Brain Sun | 17 | hsa-mir-548l | 1.04E−07 | — |
| GLB Brain Sun | 10 | hsa-mir-409 | 6.77E−05 | — |
| MM Myeloma Zhan | 33 | hsa-mir-507 | 4.95E−06 | — |
| TU Prostate Lapointe | 21 | hsa-mir-608 | 2.73E−06 | — |
| RCCC Renal Lenburg | 2 | hsa-mir-499 | 4.25E−06 | — |
| ME Melanoma Hoek | 6 | hsa-mir-186 | 1.22E−05 | — |
| SMCL Lung Bhattacharjee | 52 | hsa-mir-939 | 8.05E−05 | — |
| GLB Brain Sun | 63 | hsa-mir-939 | 0.000398514 | — |
| MM Myeloma Zhan | 32 | hsa-mir-379 | 0.000580382 | — |
| AD Lung Stearman | 33 | hsa-mir-1293 | 1.13E−05 | — |
| CCC Ovarian Hendrix | 42 | hsa-mir-590-3p | 4.63E−06 | — |
| GL Brain Bredel | 39 | hsa-mir-572 | 0.000290782 | — |
| HSCC Head-Neck Chung | 2 | hsa-mir-944 | 0.00016901 | — |
| ODGL Brain Sun | 28 | hsa-mir-219-2-3p | 0.000579468 | — |
| COID Lung Bhattacharjee | 19 | hsa-mir-615 | 0.000145391 | — |
| SQ Lung Bhattacharjee | 0 | hsa-mir-662 | 5.74E−05 | — |
| MPM Mesothelioma Gordon | 5 | hsa-mir-603 | 0.000121912 | — |
| AC Brain Sun | 2 | hsa-mir-548c-3p | 3.42E−07 | — |
| GCT Seminoma Korkola | 104 | hsa-mir-557 | 0.000174051 | — |
| CA Bladder Dyrskjot | 43 | hsa-mir-939 | 6.16E−05 | — |
| FL Lymphoma Alizadeh | 9 | hsa-mir-942 | 0.000136728 | — |
| GL Brain Rickman | 1 | hsa-mir-1283 | 0.000424966 | — |
| PPC Prostate Dhanasekaran | 8 | hsa-mir-340 | 0.000508247 | — |
| CA Breast Richardson | 67 | hsa-mir-889 | 8.08E−06 | — |
| HSCC Head-Neck Cromer | 2 | hsa-mir-374b | 9.21E−06 | — |
| ODGL Brain Sun | 61 | hsa-mir-142 | 0.000296262 | — |
| BPH Prostate Dhanasekaran | 16 | hsa-mir-302f | 1.09E−05 | — |
| CA Bladder Dyrskjot | 27 | hsa-mir-129 | 6.69E−05 | — |
| HSCC Head-Neck Cromer | 5 | hsa-mir-486-3p | 4.46E−05 | — |
| MM Myeloma Zhan | 17 | hsa-mir-495 | 3.10E−07 | — |
| ODGL Brain Sun | 58 | hsa-mir-889 | 2.02E−06 | — |
| COID Lung Bhattacharjee | 86 | hsa-mir-410 | 1.85E−06 | — |
| HSCC Head-Neck Cromer | 13 | hsa-mir-622 | 0.000123772 | — |
| COID Lung Bhattacharjee | 92 | hsa-mir-920 | 0.000526041 | — |
| AD Lung Bhattacharjee | 60 | hsa-mir-495 | 2.46E−06 | — |
| END Ovarian Hendrix | 6 | hsa-mir-890 | 2.65E−06 | — |
| SRS Ovarian Hendrix | 70 | hsa-mir-433 | 1.13E−06 | — |
| SMCL Lung Bhattacharjee | 32 | hsa-mir-582 | 1.16E−05 | — |
| ILC Breast Radvanyi | 15 | hsa-mir-518b | 0.000273711 | — |
| ML Melanoma Talantov | 34 | hsa-mir-548n | 6.91E−09 | — |
| AD Lung Bhattacharjee | 33 | hsa-mir-490 | 5.05E−05 | — |
| CCC Ovarian Hendrix | 57 | hsa-mir-590 | 0.000391567 | — |
| GCT Seminoma Korkola | 4 | hsa-mir-376 | 0.000177892 | — |
| MM Myeloma Zhan | 6 | hsa-mir-600 | 0.000129022 | — |
| GCT Seminoma Korkola | 42 | hsa-mir-637 | 0.000565519 | — |
| AC Brain Sun | 44 | hsa-mir-939 | 4.50E−09 | — |
| GL Brain Bredel | 38 | hsa-mir-486-3p | 2.00E−07 | — |
| CA Renal Higgins | 14 | hsa-mir-549 | 2.84E−05 | — |
| CA Breast Richardson | 34 | hsa-mir-186 | 1.78E−13 | — |
| PPC Prostate Dhanasekaran | 22 | hsa-mir-561 | 5.02E−08 | — |
| CCC Ovarian Hendrix | 11 | hsa-mir-602 | 0.000244248 | — |
| AD Lung Bhattacharjee | 9 | hsa-mir-154 | 0.000580128 | — |
| SRS Ovarian Hendrix | 36 | hsa-mir-890 | 0.000614411 | — |
| COID Lung Bhattacharjee | 7 | hsa-mir-608 | 0.000285439 | — |
| AD Lung Beer | 44 | hsa-mir-452 | 0.000208474 | — |
| AD Lung Beer | 46 | hsa-mir-606 | 1.91E−05 | — |
| CA Bladder Dyrskjot | 28 | hsa-mir-296 | 2.19E−05 | — |
| AC Brain Sun | 11 | hsa-mir-574-3p | 6.73E−05 | — |
| AD Lung Bhattacharjee | 65 | hsa-mir-486-3p | 0.000152303 | — |
| COID Lung Bhattacharjee | 36 | hsa-mir-890 | 0.000161298 | — |
| GLB Brain Sun | 76 | hsa-mir-139 | 3.32E−05 | — |
| COID Lung Bhattacharjee | 85 | hsa-mir-1180 | 0.000353754 | — |
| CA Breast Richardson | 38 | hsa-mir-103 | 1.94E−07 | — |
| PPC Prostate Dhanasekaran | 7 | hsa-mir-1202 | 0.000547775 | — |
| GCT Seminoma Korkola | 6 | hsa-mir-448 | 5.34E−05 | — |
| CA Bladder Dyrskjot | 68 | hsa-mir-550 | 0.000398159 | — |
| COID Lung Bhattacharjee | 18 | hsa-mir-663b | 0.000354525 | — |
| ME Melanoma Hoek | 44 | hsa-mir-1183 | 5.26E−08 | — |
| DLBCL Lymphoma Alizadeh | 4 | hsa-mir-297 | 0.000102804 | — |
| SRS Ovarian Hendrix | 38 | hsa-mir-17 | 8.61E−05 | — |
| GCT Seminoma Korkola | 62 | hsa-mir-144 | 9.85E−06 | — |
| SMCL Lung Bhattacharjee | 36 | hsa-mir-296 | 1.79E−06 | — |
| AD Lung Bhattacharjee | 16 | hsa-mir-361-3p | 6.21E−06 | — |
| CCC Ovarian Hendrix | 29 | hsa-mir-636 | 0.000218326 | — |
| AD Lung Bhattacharjee | 20 | hsa-mir-1245 | 4.94E−07 | — |
| GLB Brain Sun | 15 | hsa-mir-219-1-3p | 6.29E−06 | — |
| GLB Brain Sun | 57 | hsa-mir-921 | 7.38E−05 | — |
| AD Lung Beer | 4 | hsa-mir-1183 | 1.54E−05 | — |
| GLB Brain Sun | 67 | hsa-mir-544 | 3.10E−07 | — |
| B-CLL Leukemia Haslinger | 61 | hsa-mir-588 | 0.000290466 | — |
| COID Lung Bhattacharjee | 67 | hsa-mir-181d | 0.000603461 | — |
| MUC Ovarian Hendrix | 35 | hsa-mir-340 | 1.91E−08 | — |
| CA Colon Graudens | 4 | hsa-mir-144 | 2.99E−06 | — |
| AD Lung Beer | 9 | hsa-mir-548k | 1.83E−07 | — |
| GCT Seminoma Korkola | 48 | hsa-mir-455 | 0.000192751 | — |
| GCT Seminoma Korkola | 14 | hsa-mir-340 | 1.72E−06 | — |
| MPC Prostate Dhanasekaran | 10 | hsa-mir-554 | 0.000168264 | — |
| ODGL Brain Sun | 23 | hsa-mir-1225 | 0.000182529 | — |
| AC Brain Sun | 1 | hsa-mir-642 | 4.72E−05 | — |
| MPM Mesothelioma Gordon | 21 | hsa-mir-548c-3p | 5.39E−10 | — |
| TU Prostate Lapointe | 7 | hsa-mir-579 | 2.35E−09 | — |
| CA Breast Richardson | 31 | hsa-mir-376a | 0.000581498 | — |
| IDC Breast Radvanyi | 51 | hsa-mir-548n | 2.37E−07 | — |
| GLB Brain Sun | 60 | hsa-mir-548f | 3.12E−07 | — |
| CA Breast Richardson | 7 | hsa-mir-590-3p | 2.62E−05 | — |
| GCT Seminoma Korkola | 92 | hsa-mir-564 | 1.88E−05 | — |
| MM Myeloma Zhan | 38 | hsa-mir-652 | 0.000174084 | — |
| OD Brain Bredel | 5 | hsa-mir-1180 | 0.000184744 | — |
| AD Pancreas Logsdon | 4 | hsa-mir-22 | 0.000277647 | — |
| PDC Pancreas Ishikawa | 1 | hsa-mir-106b | 0.000224128 | — |
| SRS Ovarian Hendrix | 69 | hsa-mir-29b | 5.50E−06 | — |
| END Ovarian Hendrix | 70 | hsa-mir-939 | 1.49E−07 | — |
| CA Bladder Dyrskjot | 26 | hsa-mir-486 | 7.83E−07 | — |
| GLB Brain Sun | 13 | hsa-mir-744 | 0.000603573 | — |
| AD Pancreas Logsdon | 15 | hsa-mir-130b | 0.000605398 | — |
| AD Ovarian Welsh | 14 | hsa-mir-338 | 2.13E−06 | — |
| ML Melanoma Talantov | 33 | hsa-mir-605 | 1.62E−05 | — |
| SRS Ovarian Hendrix | 42 | hsa-mir-1298 | 9.78E−06 | — |
| GCT Seminoma Korkola | 10 | hsa-mir-153 | 0.00019375 | — |
| MPM Mesothelioma Gordon | 11 | hsa-mir-889 | 9.17E−07 | — |
| RCCC Renal Boer | 11 | hsa-mir-659 | 0.000194706 | — |
| MM Myeloma Zhan | 7 | hsa-mir-645 | 0.000256879 | — |
| PPC Prostate Dhanasekaran | 12 | hsa-mir-342-3p | 6.39E−05 | — |
| GL Brain Bredel | 52 | hsa-mir-516a-3p | 1.62E−06 | — |
| CA Breast Richardson | 63 | hsa-mir-561 | 7.97E−06 | — |
| CA Colon Graudens | 36 | hsa-mir-512-3p | 3.64E−07 | — |
| TU Prostate Lapointe | 35 | hsa-mir-548a | 5.28E−05 | — |
| SRS Ovarian Hendrix | 12 | hsa-mir-125a-3p | 5.25E−05 | — |
| ML Melanoma Talantov | 22 | hsa-mir-570 | 1.67E−05 | — |
| MM Myeloma Zhan | 49 | hsa-mir-138 | 0.000244706 | — |
| GBM Brain Liang | 16 | hsa-mir-663 | 2.49E−05 | — |
| GCT Seminoma Korkola | 15 | hsa-mir-1323 | 0.000155769 | — |
| SMCL Lung Bhattacharjee | 30 | hsa-mir-655 | 4.15E−08 | — |
| GCT Seminoma Korkola | 110 | hsa-mir-1306 | 3.03E−05 | — |
| BPH Prostate Dhanasekaran | 5 | hsa-mir-1290 | 4.31E−05 | — |
| SQ Lung Bhattacharjee | 9 | hsa-mir-891a | 2.16E−05 | — |
| CA Bladder Dyrskjot | 8 | hsa-mir-331 | 5.47E−05 | — |
| IDC Breast Radvanyi | 12 | hsa-mir-590-3p | 4.83E−05 | — |
| SMCL Lung Bhattacharjee | 57 | hsa-mir-448 | 1.38E−09 | — |
| OD Brain Bredel | 50 | hsa-mir-1254 | 0.00026912 | — |
| TU Prostate Lapointe | 26 | hsa-mir-663 | 6.26E−07 | — |
| GCT Seminoma Korkola | 26 | hsa-mir-374a | 2.65E−05 | — |
| OD Brain Bredel | 36 | hsa-mir-326 | 0.000315988 | — |
| GL Brain Rickman | 16 | hsa-mir-586 | 4.77E−05 | — |
| AC Brain Sun | 27 | hsa-mir-612 | 0.000162631 | — |
| GCT Seminoma Korkola | 2 | hsa-mir-661 | 3.65E−05 | — |
| B-CLL Leukemia Haslinger | 59 | hsa-mir-1228 | 0.000144933 | — |
| MM Myeloma Zhan | 41 | hsa-mir-339 | 1.98E−05 | — |
| GCT Seminoma Korkola | 95 | hsa-mir-548c-3p | 6.76E−11 | — |
| COID Lung Bhattacharjee | 61 | hsa-mir-34c-3p | 2.25E−05 | — |
| END Ovarian Hendrix | 29 | hsa-mir-944 | 0.000347953 | — |
| GLB Brain Sun | 42 | hsa-mir-602 | 0.000127309 | — |
| CA Breast Sorlie | 4 | hsa-mir-611 | 4.29E−05 | — |
| GCT Seminoma Korkola | 35 | hsa-mir-18a | 0.000590569 | — |
| AD Ovarian Welsh | 22 | hsa-mir-1207 | 0.000144063 | — |
| B-CLL Leukemia Haslinger | 11 | hsa-mir-34c-3p | 0.000384596 | — |
| GCT Seminoma Korkola | 45 | hsa-mir-183 | 0.000103017 | — |
| DLBCL Lymphoma Alizadeh | 9 | hsa-mir-1277 | 0.000225187 | — |
| CA Bladder Dyrskjot | 19 | hsa-mir-331-3p | 4.56E−07 | — |
| HSCC Head-Neck Cromer | 6 | hsa-mir-1275 | 0.000221713 | — |
| END Ovarian Hendrix | 26 | hsa-mir-656 | 3.57E−08 | — |
| B-CLL Leukemia Haslinger | 1 | hsa-mir-487a | 5.33E−09 | — |
| RCCC Renal Lenburg | 5 | hsa-mir-515-3p | 9.30E−05 | — |
| SMCL Lung Bhattacharjee | 43 | hsa-mir-671-3p | 0.000437915 | — |
| GCT Seminoma Korkola | 58 | hsa-mir-595 | 1.51E−06 | — |
| B-CLL Leukemia Haslinger | 50 | hsa-mir-296 | 0.000233149 | — |
| TU Prostate Lapointe | 6 | hsa-mir-876-3p | 0.000324998 | — |
| MM Myeloma Zhan | 24 | hsa-mir-1259 | 0.000330827 | — |
| PPC Prostate Dhanasekaran | 3 | hsa-mir-578 | 1.16E−05 | — |
| AD Lung Bhattacharjee | 50 | hsa-mir-642 | 5.69E−05 | — |
| AD Lung Bhattacharjee | 74 | hsa-mir-543 | 7.59E−05 | — |
| GL Brain Rickman | 21 | hsa-mir-495 | 3.72E−05 | — |
| CLL Lymphoma Alizadeh | 10 | hsa-mir-590 | 0.000327927 | — |
| IDC Breast Radvanyi | 42 | hsa-mir-766 | 0.000456161 | — |
| AD Lung Bhattacharjee | 69 | hsa-mir-522 | 4.93E−10 | — |
| OD Brain Bredel | 61 | hsa-mir-937 | 0.000101996 | — |
| MM Myeloma Zhan | 31 | hsa-mir-512-3p | 8.13E−05 | — |
| IDC Breast Radvanyi | 39 | hsa-mir-125a-3p | 0.000512311 | — |
| AD Lung Bhattacharjee | 45 | hsa-mir-340 | 6.92E−08 | — |
| AD Lung Bhattacharjee | 58 | hsa-mir-655 | 7.25E−09 | — |
| GCT Seminoma Korkola | 93 | hsa-mir-640 | 5.11E−06 | — |
| HSCC Head-Neck Cromer | 4 | hsa-mir-662 | 0.000280826 | — |
| MPM Mesothelioma Gordon | 8 | hsa-mir-1180 | 1.21E−06 | — |
| AD Lung Stearman | 5 | hsa-mir-342-3p | 1.18E−06 | — |
| GL Brain Rickman | 3 | hsa-mir-182 | 1.60E−05 | — |
| GCT Seminoma Korkola | 67 | hsa-mir-1266 | 2.88E−05 | — |
| COID Lung Bhattacharjee | 39 | hsa-mir-608 | 0.000130832 | — |
| GCT Seminoma Korkola | 1 | hsa-mir-1234 | 4.72E−05 | — |
| GCT Seminoma Korkola | 5 | hsa-mir-1204 | 0.000522965 | — |
| CA Bladder Dyrskjot | 77 | hsa-mir-620 | 0.00055255 | — |
| ODGL Brain Sun | 29 | hsa-mir-509-3 | 0.000305758 | — |
| B-CLL Leukemia Haslinger | 12 | hsa-mir-1302 | 0.000390653 | — |
| AC Brain Sun | 5 | hsa-mir-637 | 0.000149584 | — |
| GLB Brain Sun | 74 | hsa-mir-369-3p | 7.86E−05 | — |
| COID Lung Bhattacharjee | 42 | hsa-mir-671 | 4.57E−05 | — |
| CA Bladder Dyrskjot | 0 | hsa-mir-643 | 0.000314026 | — |
| GLB Brain Sun | 2 | hsa-mir-577 | 4.84E−05 | — |
| MPC Prostate Dhanasekaran | 33 | hsa-mir-886 | 0.000278531 | — |
| ODGL Brain Sun | 9 | hsa-mir-186 | 3.31E−06 | — |
| ODGL Brain Sun | 3 | hsa-mir-889 | 6.19E−07 | — |
| GCT Seminoma Korkola | 20 | hsa-mir-495 | 5.27E−07 | — |
| MUC Ovarian Hendrix | 47 | hsa-mir-654 | 0.000586378 | — |
| CCC Ovarian Hendrix | 59 | hsa-mir-1258 | 7.38E−05 | — |
| IDC Breast Radvanyi | 63 | hsa-mir-548h | 0.000316845 | — |
| GCT Seminoma Korkola | 9 | hsa-mir-296 | 4.54E−11 | — |
| GLB Brain Sun | 24 | hsa-mir-494 | 7.74E−06 | — |
| COID Lung Bhattacharjee | 79 | hsa-mir-508-3p | 0.000523991 | — |
| GCT Seminoma Korkola | 56 | hsa-mir-548m | 7.53E−06 | — |
| MUC Ovarian Hendrix | 40 | hsa-mir-618 | 2.30E−05 | — |
| TU Prostate Lapointe | 37 | hsa-mir-509-3p | 1.50E−05 | — |
| CCC Ovarian Hendrix | 35 | hsa-mir-607 | 4.23E−05 | — |
| RCCC Renal Boer | 5 | hsa-mir-374a | 0.000225536 | — |
| END Ovarian Hendrix | 41 | hsa-mir-338 | 3.20E−06 | — |
| SMCL Lung Bhattacharjee | 59 | hsa-mir-340 | 8.26E−06 | — |
| GCT Seminoma Korkola | 70 | hsa-mir-939 | 5.97E−09 | — |
| CA Breast Richardson | 56 | hsa-mir-34b | 9.69E−06 | — |
| MPM Mesothelioma Gordon | 48 | hsa-mir-606 | 4.93E−05 | — |
| AO Brain Bredel | 21 | hsa-mir-1203 | 0.000359483 | — |
| COID Lung Bhattacharjee | 28 | hsa-mir-608 | 0.000125912 | — |
| SRS Ovarian Hendrix | 14 | hsa-mir-1290 | 0.000463364 | — |
| ML Melanoma Talantov | 21 | hsa-mir-450b | 7.89E−05 | — |
| BPH Prostate Dhanasekaran | 10 | hsa-mir-624 | 2.78E−05 | — |

SUPPLEMENTARY TABLE 7

| | | |
|---|---|---|
| miRvestigator | PITA | TargetScan |

| | | | miR2Disease | | miR2Disease | | miR2Disease | |
|---|---|---|---|---|---|---|---|---|
| Dataset | Signature | miRNA | Validation | miRNA | Validation | miRNA | Validation | Overlap |
| GL Brain Rickman | 9 | | | hsa-mir-1207-5p | | hsa-mir-1207-5p | | 1 |
| CA Bladder Dyrskjot | 64 | | | hsa-mir-590-3p | | hsa-mir-590-3p | | 1 |
| GLB Brain Sun | 67 | | | hsa-mir-544 | | hsa-mir-544 | | 1 |
| | | | | hsa-mir-544b | | hsa-mir-544b | | |
| GLB Brain Sun | 2 | | | hsa-mir-577 | | hsa-mir-577 | | 1 |
| CA Breast Richardson | 2 | | | hsa-mir-590-3p | | hsa-mir-590-3p | | 1 |
| B-CLL Leukemia Haslinger | 53 | | | hsa-mir-513a-3p | | hsa-mir-513a-3p | | 1 |
| AD Lung Beer | 9 | | | hsa-mir-548k | | hsa-mir-548k | | 1 |
| AD Lung Beer | 31 | hsa-mir-29b | Causal | hsa-mir-29b | Causal | hsa-mir-29b | Causal | 1 |
| | | hsa-mir-29c | | hsa-mir-29c | | | | |
| | | hsa-mir-29a | | hsa-mir-29a | | | | |
| AD Lung Bhattacharjee | 58 | | | hsa-mir-655 | | hsa-mir-655 | | 1 |
| COID Lung Bhattacharjee | 74 | | | hsa-mir-513a-3p | | hsa-mir-513a-3p | | 1 |
| SMCL Lung Bhattacharjee | 22 | | | hsa-mir-548n | | hsa-mir-548n | | 1 |
| AD Lung Stearman | 18 | | | hsa-mir-1257 | | hsa-mir-1257 | | 1 |
| CLL Lymphoma Alizadeh | 10 | | | hsa-mir-21 | Dysregulated | hsa-mir-590-5p | | 1 |
| | | | | hsa-mir-590-5p | | | | |
| ME Melanoma Hoek | 35 | | | hsa-mir-200b | Causal | hsa-mir-200b | Causal | 1 |
| | | | | hsa-mir-200c | | | | |
| | | | | hsa-mir-200a | | | | |
| AD Ovarian Welsh | 25 | | | hsa-mir-572 | Dysregulated | hsa-mir-572 | Dysregulated | 1 |
| CCC Ovarian Hendrix | 42 | | | hsa-mir-590-3p | | hsa-mir-590-3p | | 1 |
| CCC Ovarian Hendrix | 4 | | | hsa-mir-194 | | hsa-mir-194 | | 1 |
| SRS Ovarian Hendrix | 73 | | | hsa-mir-340 | | hsa-mir-340 | | 1 |
| END Ovarian Hendrix | 35 | | | hsa-mir-1276 | | hsa-mir-1276 | | 1 |
| MPC Prostate Dhanasekaran | 35 | | | hsa-mir-203 | | hsa-mir-203 | | 1 |
| TU Prostate Lapointe | 39 | | | hsa-mir-590-3p | | hsa-mir-656 | | 0 |
| CA Bladder Dyrskjot | 56 | | | hsa-mir-335 | | hsa-mir-1238 | | 0 |
| CA Bladder Dyrskjot | 36 | | | hsa-mir-548n | | hsa-mir-548c-3p | | 0 |
| CA Bladder Dyrskjot | 51 | | | hsa-mir-1276 | | | | 0 |
| TU Prostate Lapointe | 13 | | | | | hsa-mir-497 | Dysregulated | 0 |
| CA Bladder Dyrskjot | 22 | | | | | hsa-mir-663b | | 0 |
| | | | | | | hsa-mir-663 | | |
| CA Bladder Dyrskjot | 45 | | | | | hsa-mir-608 | | 0 |
| TU Prostate Lapointe | 14 | hsa-mir-138-1* | | | | | | 0 |
| TU Prostate Lapointe | 17 | | | hsa-mir-96 | Dysregulated | hsa-mir-219-5p | | 0 |
| TU Prostate Lapointe | 33 | | | hsa-mir-802 | | hsa-mir-548c-3p | | 0 |
| TU Prostate Lapointe | 18 | | | hsa-mir-760 | | | | 0 |
| TU Prostate Lapointe | 31 | | | hsa-mir-631 | | hsa-mir-339-3p | | 0 |
| CA Bladder Dyrskjot | 53 | | | hsa-mir-1206 | | hsa-mir-944 | | 0 |
| TU Prostate Lapointe | 37 | | | hsa-mir-199b-3p | Causal | hsa-mir-509-3p | | 0 |
| | | | | hsa-mir-199a-3p | | hsa-mir-509-3-5p | | |
| CA Bladder Dyrskjot | 67 | | | | | hsa-mir-630 | | 0 |
| CA Bladder Dyrskjot | 82 | | | hsa-mir-519d | | hsa-mir-656 | | 0 |
| TU Prostate Lapointe | 19 | | | | | hsa-mir-296-5p | Dysregulated | 0 |
| CA Bladder Dyrskjot | 81 | | | hsa-mir-455-3p | | | | 0 |
| CA Bladder Dyrskjot | 14 | | | | | hsa-mir-944 | | 0 |
| CA Bladder Dyrskjot | 24 | | | | | hsa-mir-663b | | 0 |
| | | | | | | hsa-mir-663 | | |
| CA Bladder Dyrskjot | 25 | | | hsa-mir-550b | | hsa-mir-1204 | | 0 |
| | | | | hsa-mir-550a | | | | |
| CA Bladder Dyrskjot | 26 | hsa-mir-194 | | hsa-mir-1224-5p | | hsa-mir-486-5p | | 0 |
| CA Bladder Dyrskjot | 27 | | | hsa-mir-365 | | hsa-mir-129-5p | | 0 |
| CA Bladder Dyrskjot | 21 | | | hsa-mir-590-3p | | hsa-mir-944 | | 0 |
| CA Bladder Dyrskjot | 48 | | | | | hsa-mir-520b | | 0 |
| CA Bladder Dyrskjot | 49 | | | | | hsa-mir-432 | | 0 |
| CA Bladder Dyrskjot | 46 | | | | | hsa-mir-342-5p | | 0 |
| CA Bladder Dyrskjot | 47 | | | hsa-mir-331-5p | | hsa-mir-197 | | 0 |
| CA Bladder Dyrskjot | 42 | | | hsa-mir-1183 | | hsa-mir-362-5p | | 0 |
| CA Bladder Dyrskjot | 28 | | | | | hsa-mir-296-5p | | 0 |
| CA Bladder Dyrskjot | 43 | | | | | hsa-mir-939 | | 0 |
| CA Bladder Dyrskjot | 40 | | | | | hsa-mir-548d-3p | | 0 |
| CA Bladder Dyrskjot | 1 | | | hsa-mir-940 | | hsa-mir-637 | | 0 |
| CA Bladder Dyrskjot | 0 | | | hsa-mir-607 | | hsa-mir-643 | | 0 |
| CA Renal Higgins | 10 | | | hsa-mir-875-3p | | hsa-mir-483-3p | | 0 |
| CA Renal Higgins | 13 | | | hsa-mir-423-5p | | hsa-mir-663b | | 0 |
| | | | | | | hsa-mir-663 | | |
| CA Bladder Dyrskjot | 7 | | | | | hsa-mir-361-5p | | 0 |
| CA Bladder Dyrskjot | 6 | | | | | hsa-mir-939 | | 0 |
| CA Renal Higgins | 14 | | | hsa-mir-140-5p | | hsa-mir-549 | | 0 |
| CA Bladder Dyrskjot | 8 | | | hsa-mir-140-3p | | hsa-mir-331-5p | | 0 |
| CA Bladder Dyrskjot | 68 | | | | | hsa-mir-550b | | 0 |
| | | | | | | hsa-mir-550a | | |
| CA Bladder Dyrskjot | 79 | | | hsa-mir-147 | | hsa-mir-508-3p | | 0 |
| | | | | hsa-mir-147b | | | | |
| RCCC Renal Boer | 11 | | | | | hsa-mir-659 | | 0 |
| CA Bladder Dyrskjot | 13 | hsa-mir-548k | | | | hsa-mir-548c-3p | | 0 |
| RCCC Renal Boer | 13 | | | hsa-mir-200a | Causal | hsa-mir-1279 | | 0 |
| | | | | hsa-mir-141 | | | | |
| RCCC Renal Boer | 12 | | | hsa-mir-760 | | | | 0 |
| CA Bladder Dyrskjot | 75 | | | hsa-mir-487a | | hsa-mir-513c | | 0 |
| | | | | | | hsa-mir-513b | | |
| | | | | | | hsa-mir-513a-5p | | |
| RCCC Renal Boer | 14 | | | hsa-mir-590-3p | | | | 0 |
| CA Bladder Dyrskjot | 72 | | | | | hsa-mir-296-5p | | 0 |
| CA Bladder Dyrskjot | 71 | | | hsa-mir-633 | | hsa-mir-662 | | 0 |
| RCCC Renal Boer | 3 | | | hsa-mir-217 | | | | 0 |
| CA Bladder Dyrskjot | 15 | | | | | hsa-mir-1323 | | 0 |
| CA Bladder Dyrskjot | 32 | | | hsa-mir-320e | | hsa-mir-1270 | | 0 |
| | | | | hsa-mir-320d | | | | |
| | | | | hsa-mir-320c | | | | |
| | | | | hsa-mir-320b | | | | |
| | | | | hsa-mir-320a | | | | |
| RCCC Renal Boer | 4 | | | hsa-mir-1262 | | | | 0 |
| RCCC Renal Boer | 7 | hsa-mir-32 | | | | hsa-mir-106b | Dysregulated | 0 |
| | | hsa-mir-92a | | | | | | |
| | | hsa-mir-92b | | | | | | |
| CA Bladder Dyrskjot | 10 | | | | | hsa-mir-597 | | 0 |
| RCCC Renal Boer | 9 | | | hsa-mir-1256 | | hsa-mir-502-3p | | 0 |
| RCCC Renal Boer | 8 | | | hsa-mir-1206 | | | | 0 |
| CA Bladder Dyrskjot | 63 | | | | | hsa-mir-372 | | 0 |
| CA Bladder Dyrskjot | 58 | | | | | hsa-mir-328 | | 0 |
| CA Bladder Dyrskjot | 17 | hsa-mir-885-3p | | | | | | 0 |
| CA Bladder Dyrskjot | 16 | | | hsa-mir-1205 | | hsa-mir-939 | | 0 |
| CA Bladder Dyrskjot | 19 | | | hsa-mir-659 | | hsa-mir-331-3p | | 0 |
| RCCC Renal Lenburg | 3 | hsa-mir-548f | | | | hsa-mir-888 | | 0 |
| | | hsa-mir-548e | | | | | | |
| | | hsa-mir-548x | | | | | | |
| RCCC Renal Lenburg | 2 | hsa-mir-487b | | | | hsa-mir-499a-5p | | 0 |
| | | | | | | hsa-mir-499-5p | | |
| CA Bladder Dyrskjot | 23 | | | | | hsa-mir-211 | | 0 |
| CA Bladder Dyrskjot | 37 | | | | | hsa-mir-548n | | 0 |
| CA Bladder Dyrskjot | 35 | hsa-mir-1469 | | | | hsa-mir-486-3p | | 0 |
| RCCC Renal Lenburg | 9 | | | | | hsa-mir-1238 | | 0 |
| RCCC Renal Lenburg | 8 | | | hsa-mir-660 | | hsa-mir-889 | | 0 |
| CA Bladder Dyrskjot | 54 | | | | | hsa-mir-491-5p | | 0 |
| GCT Seminoma Korkola | 25 | | | hsa-mir-105 | | hsa-mir-607 | | 0 |
| CA Bladder Dyrskjot | 31 | | | hsa-mir-520d-5p | | hsa-mir-656 | | 0 |
| | | | | hsa-mir-524-5p | | | | |
| ODGL Brain Sun | 0 | | | | | hsa-mir-637 | | 0 |
| GCT Seminoma Korkola | 20 | | | hsa-mir-632 | | hsa-mir-495 | | 0 |
| GCT Seminoma Korkola | 21 | | | hsa-mir-548k | | hsa-mir-520f | | 0 |
| GBM Brain Liang | 13 | | | | | hsa-mir-1278 | | 0 |
| GBM Brain Liang | 12 | | | hsa-mir-520g | | | | 0 |
| | | | | hsa-mir-520h | | | | |
| GBM Brain Liang | 15 | | | hsa-mir-144 | | | | 0 |
| GBM Brain Liang | 14 | hsa-mir-496 | | hsa-mir-125a-3p | | | | 0 |
| GBM Brain Liang | 17 | hsa-mir-361-5p | | hsa-mir-600 | | | | 0 |
| GCT Seminoma Korkola | 4 | | | | | hsa-mir-376a | | 0 |
| | | | | | | hsa-mir-376b | | |
| | | | | | | hsa-mir-376c | | |
| GBM Brain Liang | 18 | hsa-mir-369-5p | | | | | | 0 |
| GBM Brain Liang | 1 | | | | | hsa-mir-423-5p | | 0 |
| GCT Seminoma Korkola | 58 | | | | | hsa-mir-595 | | 0 |
| GBM Brain Liang | 2 | | | | | hsa-mir-548c-3p | | 0 |
| TU Prostate Lapointe | 35 | | | hsa-mir-758 | | hsa-mir-548am | | 0 |
| | | | | | | hsa-mir-548ab | | |
| | | | | | | hsa-mir-548aa | | |
| | | | | | | hsa-mir-548ag | | |
| | | | | | | hsa-mir-548ak | | |
| | | | | | | hsa-mir-548aj | | |
| | | | | | | hsa-mir-548ai | | |
| | | | | | | hsa-mir-548ah | | |
| | | | | | | hsa-mir-548an | | |
| | | | | | | hsa-mir-548al | | |
| | | | | | | hsa-mir-548ae | | |
| | | | | | | hsa-mir-548ad | | |
| | | | | | | hsa-mir-548a-5p | | |
| | | | | | | hsa-mir-548ac | | |
| GCT Seminoma Korkola | 56 | | | hsa-mir-499-3p | | hsa-mir-548m | | 0 |
| GBM Brain Liang | 7 | | | | | hsa-mir-323-5p | Dysregulated | 0 |
| | | | | | | hsa-mir-323b-5p | | |
| TU Prostate Lapointe | 38 | | | hsa-mir-1264 | | hsa-mir-633 | | 0 |
| GCT Seminoma Korkola | 52 | | | | | hsa-mir-508-5p | | 0 |
| GCT Seminoma Korkola | 88 | | | hsa-mir-129-5p | | hsa-mir-338-5p | | 0 |
| GCT Seminoma Korkola | 89 | | | hsa-mir-568 | | hsa-mir-1257 | | 0 |
| GCT Seminoma Korkola | 111 | | | | | hsa-mir-296-5p | | 0 |
| GCT Seminoma Korkola | 110 | | | hsa-mir-18a | | hsa-mir-1306 | | 0 |
| | | | | hsa-mir-18b | | | | |
| GCT Seminoma Korkola | 112 | | | hsa-mir-590-3p | | hsa-mir-548n | | 0 |
| OD Brain Bredel | 19 | hsa-mir-551a | | hsa-mir-487a | | | | 0 |
| | | hsa-mir-551b | | | | | | |
| OD Brain Bredel | 62 | | | | | hsa-let-7f | | 0 |
| GCT Seminoma Korkola | 80 | | | hsa-mir-623 | | hsa-mir-573 | | 0 |
| OD Brain Bredel | 52 | | | hsa-mir-325 | | hsa-mir-411 | Dysregulated | 0 |
| OD Brain Bredel | 24 | | | hsa-mir-193a-3p | | | | 0 |
| OD Brain Bredel | 25 | | | hsa-mir-512-3p | | | | 0 |
| GCT Seminoma Korkola | 85 | | | | | hsa-mir-181d | | 0 |
| | | | | | | hsa-mir-181a | | |
| | | | | | | hsa-mir-181b | | |
| | | | | | | hsa-mir-181c | | |
| GCT Seminoma Korkola | 3 | hsa-mir-324-5p | | | | hsa-mir-296-5p | | 0 |
| OD Brain Bredel | 21 | | | hsa-mir-369-3p | | hsa-mir-548c-3p | | 0 |
| GCT Seminoma Korkola | 109 | | | hsa-mir-548p | | | | 0 |
| GCT Seminoma Korkola | 102 | | | hsa-mir-125b | | hsa-mir-34a | | 0 |
| | | | | hsa-mir-125a-5p | | | | |
| GCT Seminoma Korkola | 67 | | | | | hsa-mir-1266 | | 0 |
| GCT Seminoma Korkola | 100 | | | hsa-mir-486-3p | | | | 0 |
| GCT Seminoma Korkola | 101 | | | | | hsa-mir-372 | Causal | 0 |
| OD Brain Bredel | 45 | | | hsa-mir-324-5p | | | | 0 |
| GCT Seminoma Korkola | 107 | | | hsa-mir-199b-3p | Causal | | | 0 |
| | | | | hsa-mir-199a-3p | | | | |
| OD Brain Bredel | 43 | | | hsa-mir-412 | | | | 0 |
| GCT Seminoma Korkola | 105 | | | hsa-mir-34a | | | | 0 |
| | | | | hsa-mir-34c-5p | | | | |
| | | | | hsa-mir-449a | | | | |
| | | | | hsa-mir-449b | | | | |
| GCT Seminoma Korkola | 39 | | | hsa-mir-769-5p | | hsa-mir-639 | | 0 |
| GCT Seminoma Korkola | 38 | | | hsa-mir-370 | | | | 0 |
| TU Prostate Lapointe | 11 | | | | | hsa-mir-152 | | 0 |
| OD Brain Bredel | 3 | | | hsa-mir-615-3p | | hsa-mir-195 | Dysregulated | 0 |
| GCT Seminoma Korkola | 31 | | | hsa-mir-22 | | hsa-mir-891a | | 0 |
| OD Brain Bredel | 5 | hsa-mir-423-3p | | | | hsa-mir-1180 | | 0 |
| GCT Seminoma Korkola | 37 | | | hsa-mir-106b | | hsa-mir-519a | | 0 |
| | | | | hsa-mir-17 | | | | |
| | | | | hsa-mir-106a | | | | |
| | | | | hsa-mir-93 | | | | |
| | | | | hsa-mir-20b | | | | |
| | | | | hsa-mir-20a | | | | |
| GCT Seminoma Korkola | 36 | | | hsa-mir-196b | | | | 0 |
| | | | | hsa-mir-196a | | | | |
| GCT Seminoma Korkola | 35 | | | | | hsa-mir-18a | | 0 |
| OD Brain Bredel | 9 | | | hsa-mir-548e | | | | 0 |
| OD Brain Bredel | 8 | | | hsa-mir-651 | | | | 0 |
| OD Brain Bredel | 56 | | | hsa-mir-126 | | | | 0 |
| GCT Seminoma Korkola | 62 | | | hsa-mir-548m | | hsa-mir-144 | | 0 |
| GCT Seminoma Korkola | 63 | | | | | hsa-mir-889 | | 0 |
| OD Brain Bredel | 12 | | | hsa-mir-588 | | hsa-mir-200a | Causal | 0 |
| GCT Seminoma Korkola | 66 | | | hsa-mir-532-3p | | hsa-mir-1291 | | 0 |
| CA Renal Higgins | 20 | | | | | hsa-mir-1261 | | 0 |
| GCT Seminoma Korkola | 68 | | | | | hsa-mir-1182 | | 0 |
| GCT Seminoma Korkola | 69 | | | | | hsa-mir-296-5p | | 0 |
| GCT Seminoma Korkola | 2 | | | | | hsa-mir-661 | | 0 |
| GCT Seminoma Korkola | 6 | | | hsa-mir-34b | | hsa-mir-448 | | 0 |
| GCT Seminoma Korkola | 98 | | | hsa-mir-224 | | hsa-mir-203 | | 0 |
| GCT Seminoma Korkola | 90 | | | hsa-mir-362-5p | | | | 0 |
| OD Brain Bredel | 16 | hsa-mir-1181 | | | | | | 0 |
| OD Brain Bredel | 33 | | | hsa-mir-1231 | | | | 0 |
| GCT Seminoma Korkola | 95 | | | hsa-mir-1283 | | hsa-mir-548c-3p | | 0 |
| OD Brain Bredel | 57 | | | hsa-mir-148b | | hsa-mir-423-5p | | 0 |
| | | | | hsa-mir-152 | | | | |
| OD Brain Bredel | 37 | hsa-mir-29a* | | | | | | 0 |
| TU Prostate Lapointe | 10 | | | | | hsa-mir-32 | Dysregulated | 0 |
| GCT Seminoma Korkola | 10 | | | | | hsa-mir-153 | | 0 |
| GCT Seminoma Korkola | 15 | | | | | hsa-mir-1323 | | 0 |
| GCT Seminoma Korkola | 14 | | | hsa-mir-1323 | | hsa-mir-340 | | 0 |
| | | | | hsa-mir-548o | | | | |
| GCT Seminoma Korkola | 17 | | | | | hsa-mir-615-5p | | 0 |
| GL Brain Bredel | 30 | | | | | hsa-mir-342-5p | Dysregulated | 0 |
| GL Brain Bredel | 54 | | | hsa-mir-582-3p | | | | 0 |
| GL Brain Bredel | 37 | | | hsa-mir-544 | | | | 0 |
| | | | | hsa-mir-544b | | | | |
| GCT Seminoma Korkola | 48 | | | | | hsa-mir-455-5p | | 0 |
| GCT Seminoma Korkola | 47 | | | hsa-mir-485-5p | | | | 0 |
| GL Brain Bredel | 61 | | | hsa-mir-572 | | | | 0 |
| GCT Seminoma Korkola | 45 | | | | | hsa-mir-183 | | 0 |
| GCT Seminoma Korkola | 42 | hsa-mir-1181 | | hsa-mir-149 | | hsa-mir-637 | | 0 |
| GCT Seminoma Korkola | 40 | | | hsa-mir-577 | | hsa-mir-548n | | 0 |
| GL Brain Bredel | 66 | hsa-mir-223 | Dysregulated | | | hsa-mir-27a | Dysregulated | 0 |
| GCT Seminoma Korkola | 1 | | | | | hsa-mir-1234 | | 0 |
| CA Renal Higgins | 1 | | | hsa-mir-590-3p | | | | 0 |
| GCT Seminoma Korkola | 9 | | | | | hsa-mir-296-5p | | 0 |
| GL Brain Bredel | 52 | | | | | hsa-mir-516a-3p | | 0 |
| GL Brain Bredel | 24 | hsa-mir-516b | | hsa-mir-1245b-5p | | | | 0 |
| | | | | hsa-mir-1245 | | | | |
| GCT Seminoma Korkola | 75 | hsa-mir-25 | Dysregulated | hsa-mir-664 | | hsa-mir-708 | | 0 |
| | | hsa-mir-32 | | | | | | |
| | | hsa-mir-92a | | | | | | |
| | | hsa-mir-92b | | | | | | |
| | | hsa-mir-363 | | | | | | |
| | | hsa-mir-367 | | | | | | |
| GCT Seminoma Korkola | 74 | | | hsa-let-7f | | | | 0 |
| | | | | hsa-let-7g | | | | |
| | | | | hsa-let-7a | | | | |
| | | | | hsa-let-7b | | | | |
| | | | | hsa-let-7d | | | | |
| | | | | hsa-let-7i | | | | |
| | | | | hsa-mir-98 | | | | |
| | | | | hsa-let-7c | | | | |
| | | | | hsa-let-7e | | | | |
| GL Brain Bredel | 27 | | | | | hsa-mir-181d | Dysregulated | 0 |
| | | | | | | hsa-mir-181a | | |
| | | | | | | hsa-mir-181b | | |
| | | | | | | hsa-mir-181c | | |
| GL Brain Bredel | 20 | | | hsa-mir-487b | Dysregulated | | | 0 |
| GL Brain Bredel | 21 | | | hsa-mir-633 | | | | 0 |
| GL Brain Bredel | 48 | hsa-mir-2277-5p | | | | | | 0 |
| GCT Seminoma Korkola | 0 | | | | | hsa-mir-1251 | | 0 |
| GL Brain Bredel | 46 | | | hsa-mir-154 | | hsa-mir-548l | | 0 |
| CA Renal Higgins | 3 | | | | | hsa-mir-199a-3p | Causal | 0 |
| GL Brain Bredel | 40 | | | hsa-mir-526b | | | | 0 |
| GL Brain Bredel | 41 | | | hsa-mir-484 | | | | 0 |
| GL Brain Bredel | 0 | | | hsa-mir-1208 | | hsa-mir-548i | | 0 |
| GL Brain Bredel | 9 | | | hsa-mir-549 | | | | 0 |
| GL Brain Bredel | 78 | hsa-mir-126 | | | | hsa-mir-424 | Dysregulated | 0 |
| CA Renal Higgins | 4 | | | | | hsa-mir-34b | | 0 |
| GL Brain Bredel | 33 | | | hsa-mir-625 | | | | 0 |
| GL Brain Bredel | 39 | | | hsa-mir-363 | | hsa-mir-572 | | 0 |
| GL Brain Bredel | 77 | | | | | hsa-mir-744 | | 0 |
| GL Brain Bredel | 75 | | | hsa-mir-889 | | | | 0 |
| GL Brain Bredel | 72 | | | | | hsa-mir-760 | | 0 |
| GL Brain Bredel | 71 | | | hsa-mir-651 | | | | 0 |
| GL Brain Bredel | 59 | | | hsa-mir-125a-3p | | | | 0 |
| GL Brain Bredel | 38 | | | | | hsa-mir-486-3p | | 0 |
| GL Brain Bredel | 79 | | | | | hsa-mir-340 | | 0 |
| GL Brain Bredel | 11 | | | hsa-mir-542-3p | | | | 0 |
| CA Breast Richardson | 43 | | | hsa-mir-1246 | | | | 0 |
| GL Brain Bredel | 12 | | | hsa-mir-384 | | | | 0 |
| GL Brain Bredel | 15 | hsa-mir-126 | | hsa-mir-654-3p | | | | 0 |
| GL Brain Bredel | 17 | | | | | hsa-mir-877 | | 0 |
| GL Brain Bredel | 32 | | | | | hsa-mir-15a | Dysregulated | 0 |
| GL Brain Bredel | 31 | | | | | hsa-mir-615-3p | | 0 |
| GL Brain Bredel | 36 | | | hsa-mir-661 | | | | 0 |
| GL Brain Bredel | 18 | hsa-mir-187 | | | | | | 0 |
| GL Brain Bredel | 57 | | | hsa-mir-769-3p | | hsa-mir-193a-3p | | 0 |
| | | | | hsa-mir-450b-3p | | | | |
| GL Brain Bredel | 65 | | | hsa-mir-532-5p | | | | 0 |
| AO Brain Bredel | 20 | | | hsa-mir-324-5p | | | | 0 |
| AO Brain Bredel | 21 | | | | | hsa-mir-1203 | | 0 |
| AO Brain Bredel | 22 | | | | | hsa-mir-663b | | 0 |
| | | | | | | hsa-mir-663 | | |
| AO Brain Bredel | 28 | | | | | hsa-mir-296-5p | Causal | 0 |
| GCT Seminoma Korkola | 83 | | | | | hsa-mir-1254 | | 0 |
| AO Brain Bredel | 1 | | | | | hsa-mir-181a | Causal | 0 |
| AO Brain Bredel | 0 | | | | | hsa-mir-635 | | 0 |
| GBM Brain Liang | 5 | | | | | hsa-mir-939 | | 0 |
| AO Brain Bredel | 38 | | | | | hsa-mir-1282 | | 0 |
| AO Brain Bredel | 11 | | | hsa-mir-608 | | | | 0 |
| AO Brain Bredel | 10 | hsa-mir-4285 | | | | | | 0 |
| AO Brain Bredel | 13 | | | hsa-mir-506 | | | | 0 |
| AO Brain Bredel | 17 | | | hsa-mir-191 | | | | 0 |
| CA Renal Higgins | 17 | | | | | hsa-mir-663b | | 0 |
| | | | | | | hsa-mir-663 | | |
| AO Brain Bredel | 18 | | | | | hsa-mir-497 | | 0 |
| AO Brain Bredel | 30 | | | hsa-mir-1255b | | | | 0 |
| | | | | hsa-mir-1255a | | | | |
| AO Brain Bredel | 35 | | | hsa-mir-939 | | | | 0 |
| AO Brain Bredel | 33 | | | hsa-mir-450b-5p | | | | 0 |
| AO Brain Bredel | 32 | | | | | hsa-mir-564 | | 0 |
| GL Brain Rickman | 26 | hsa-mir-1469 | | | | | | 0 |
| GL Brain Rickman | 27 | | | | | hsa-mir-182 | | 0 |
| GL Brain Rickman | 21 | | | hsa-mir-624 | | hsa-mir-495 | | 0 |
| GL Brain Rickman | 22 | | | hsa-mir-542-3p | | hsa-mir-374b | | 0 |
| GL Brain Rickman | 23 | | | hsa-mir-490-5p | | hsa-mir-935 | | 0 |
| GL Brain Rickman | 29 | | | hsa-mir-544 | | hsa-mir-568 | | 0 |
| | | | | hsa-mir-544b | | | | |
| GL Brain Rickman | 1 | | | hsa-mir-493 | | hsa-mir-1283 | | 0 |
| GL Brain Rickman | 3 | | | | | hsa-mir-182 | | 0 |
| GL Brain Rickman | 2 | hsa-mir-718 | | | | | | 0 |
| GL Brain Rickman | 4 | | | hsa-mir-302f | | hsa-mir-361-5p | | 0 |
| GL Brain Rickman | 8 | | | | | hsa-mir-634 | | 0 |
| GL Brain Rickman | 11 | | | hsa-mir-653 | | | | 0 |
| GL Brain Rickman | 10 | | | | | hsa-mir-648 | | 0 |
| GL Brain Rickman | 13 | | | hsa-mir-486-3p | | | | 0 |
| GL Brain Rickman | 15 | | | | | hsa-mir-487a | | 0 |
| GL Brain Rickman | 14 | | | hsa-mir-149 | Dysregulated | | | 0 |
| GL Brain Rickman | 17 | | | | | hsa-mir-146b-3p | | 0 |
| GL Brain Rickman | 16 | | | | | hsa-mir-586 | | 0 |
| GL Brain Rickman | 31 | | | hsa-let-7e | Dysregulated | | | 0 |
| | | | | hsa-let-7f | | | | |
| | | | | hsa-let-7g | | | | |
| | | | | hsa-let-7a | | | | |
| | | | | hsa-let-7b | | | | |
| | | | | hsa-let-7d | | | | |
| | | | | hsa-let-7i | | | | |
| | | | | hsa-mir-98 | | | | |
| | | | | hsa-let-7c | | | | |
| GL Brain Rickman | 34 | | | hsa-mir-125a-3p | | | | 0 |
| GL Brain Rickman | 32 | | | | | hsa-mir-889 | | 0 |
| ODGL Brain Sun | 30 | | | | | hsa-mir-483-5p | | 0 |
| RCCC Renal Boer | 15 | | | hsa-mir-548k | | hsa-mir-300 | | 0 |
| ODGL Brain Sun | 22 | | | | | hsa-mir-423-5p | | 0 |
| ODGL Brain Sun | 29 | | | | | hsa-mir-509-3p | | 0 |
| | | | | | | hsa-mir-509-3-5p | | |
| ODGL Brain Sun | 60 | | | hsa-mir-556-3p | | hsa-let-7d | | 0 |
| ODGL Brain Sun | 61 | | | | | hsa-mir-142-5p | | 0 |
| ODGL Brain Sun | 62 | | | | | hsa-mir-1294 | | 0 |
| ODGL Brain Sun | 63 | | | | | hsa-mir-410 | Dysregulated | 0 |
| ODGL Brain Sun | 64 | | | hsa-mir-539 | | | | 0 |
| ODGL Brain Sun | 53 | | | hsa-mir-548m | | hsa-mir-548n | | 0 |
| ODGL Brain Sun | 52 | | | hsa-mir-1270 | | | | 0 |
| | | | | hsa-mir-620 | | | | |
| ODGL Brain Sun | 23 | | | | | hsa-mir-1225-5p | | 0 |
| ODGL Brain Sun | 25 | | | hsa-mir-548p | | hsa-mir-548l | | 0 |
| ODGL Brain Sun | 26 | | | hsa-mir-622 | | | | 0 |
| ODGL Brain Sun | 27 | | | | | hsa-mir-136 | Dysregulated | 0 |
| ODGL Brain Sun | 20 | | | hsa-mir-570 | | hsa-mir-548e | | 0 |
| RCCC Renal Boer | 1 | | | | | hsa-mir-371b-5p | | 0 |
| | | | | | | hsa-mir-371-5p | | |
| ODGL Brain Sun | 48 | | | | | hsa-mir-384 | | 0 |
| ODGL Brain Sun | 49 | | | | | hsa-mir-423-5p | | 0 |
| ODGL Brain Sun | 47 | | | | | hsa-mir-1204 | | 0 |
| ODGL Brain Sun | 44 | | | hsa-mir-1276 | | | | 0 |
| RCCC Renal Boer | 0 | | | hsa-mir-140-3p | | hsa-mir-494 | Dysregulated | 0 |
| ODGL Brain Sun | 28 | | | | | hsa-mir-219-2-3p | | 0 |
| ODGL Brain Sun | 41 | | | hsa-mir-374b | | hsa-mir-26b | | 0 |
| | | | | hsa-mir-374a | | | | |
| ODGL Brain Sun | 1 | | | | | hsa-mir-647 | | 0 |
| CA Bladder Dyrskjot | 70 | | | | | hsa-mir-502-5p | | 0 |
| ODGL Brain Sun | 3 | | | | | hsa-mir-889 | | 0 |
| ODGL Brain Sun | 2 | | | | | hsa-mir-939 | | 0 |
| ODGL Brain Sun | 7 | | | | | hsa-mir-548k | | 0 |
| ODGL Brain Sun | 9 | | | hsa-mir-142-3p | Dysregulated | hsa-mir-186 | | 0 |
| ODGL Brain Sun | 50 | | | hsa-mir-548c-3p | | hsa-mir-217 | | 0 |
| ODGL Brain Sun | 58 | | | hsa-mir-181d | Causal | hsa-mir-889 | | 0 |
| | | | | hsa-mir-181b | | | | |
| ODGL Brain Sun | 11 | | | | | hsa-mir-219-1-3p | | 0 |
| ODGL Brain Sun | 10 | | | | | hsa-mir-208a | | 0 |
| ODGL Brain Sun | 39 | | | hsa-mir-218 | Dysregulated | hsa-mir-1283 | | 0 |
| ODGL Brain Sun | 38 | hsa-mir-1469 | | | | | | 0 |
| ODGL Brain Sun | 17 | | | | | hsa-mir-548l | | 0 |
| ODGL Brain Sun | 33 | | | hsa-mir-335 | | | | 0 |
| ODGL Brain Sun | 57 | | | | | hsa-mir-296-5p | Causal | 0 |
| ODGL Brain Sun | 36 | | | hsa-mir-361-3p | | | | 0 |
| ODGL Brain Sun | 37 | hsa-mir-126 | | | | | | 0 |
| ODGL Brain Sun | 32 | | | | | hsa-mir-153 | | 0 |
| ODGL Brain Sun | 31 | | | hsa-mir-490-3p | | | | 0 |
| ODGL Brain Sun | 65 | | | hsa-mir-340 | | hsa-mir-495 | | 0 |
| AC Brain Sun | 51 | | | hsa-mir-922 | | | | 0 |
| AC Brain Sun | 34 | | | | | hsa-mir-223 | Dysregulated | 0 |
| CA Bladder Dyrskjot | 38 | | | | | hsa-mir-1281 | | 0 |
| AC Brain Sun | 27 | | | | | hsa-mir-612 | | 0 |
| AC Brain Sun | 23 | | | | | hsa-mir-1207-3p | | 0 |
| AC Brain Sun | 46 | | | hsa-mir-548c-3p | | hsa-mir-590-3p | | 0 |
| AC Brain Sun | 44 | | | | | hsa-mir-939 | | 0 |
| OD Brain Bredel | 54 | | | | | hsa-mir-1275 | | 0 |
| RCCC Renal Lenburg | 10 | | | | | hsa-mir-1273 | | 0 |
| | | | | | | hsa-mir-1273c | | |
| | | | | | | hsa-mir-1273d | | |
| | | | | | | hsa-mir-1273e | | |
| | | | | | | hsa-mir-1273f | | |
| | | | | | | hsa-mir-1273g | | |
| AC Brain Sun | 1 | | | | | hsa-mir-642a | | 0 |
| | | | | | | hsa-mir-642b | | |
| AC Brain Sun | 3 | | | | | hsa-mir-1205 | | 0 |
| AC Brain Sun | 2 | | | | | hsa-mir-548c-3p | | 0 |
| AC Brain Sun | 5 | hsa-mir-523 | | | | hsa-mir-637 | | 0 |
| AC Brain Sun | 4 | hsa-mir-937 | | hsa-mir-222 | Causal | hsa-mir-371-3p | | 0 |
| AC Brain Sun | 7 | | | hsa-mir-582-3p | | hsa-mir-548l | | 0 |
| AC Brain Sun | 6 | | | hsa-mir-1284 | | hsa-mir-139-5p | Dysregulated | 0 |
| AC Brain Sun | 8 | | | | | hsa-mir-196a | | 0 |
| AC Brain Sun | 28 | | | | | hsa-mir-539 | | 0 |
| AC Brain Sun | 12 | | | hsa-mir-485-3p | Dysregulated | hsa-mir-210 | Dysregulated | 0 |
| AC Brain Sun | 11 | | | | | hsa-mir-574-3p | | 0 |
| CA Bladder Dyrskjot | 66 | | | hsa-mir-1827 | | | | 0 |
| AC Brain Sun | 39 | | | hsa-mir-1266 | | | | 0 |
| AC Brain Sun | 22 | | | | | hsa-mir-185 | | 0 |
| AC Brain Sun | 17 | hsa-mir-487b | Dysregulated | | | hsa-mir-617 | | 0 |
| AC Brain Sun | 16 | | | | | hsa-mir-655 | | 0 |
| AC Brain Sun | 19 | | | hsa-mir-218 | Dysregulated | hsa-mir-485-5p | | 0 |
| AC Brain Sun | 18 | | | | | hsa-mir-1305 | | 0 |
| AC Brain Sun | 31 | | | | | hsa-mir-1280 | | 0 |
| AC Brain Sun | 37 | | | | | hsa-mir-569 | | 0 |
| AC Brain Sun | 32 | | | hsa-mir-1266 | | | | 0 |
| AC Brain Sun | 50 | hsa-mir-1234 | | hsa-mir-32 | | hsa-mir-483-3p | | 0 |
| GLB Brain Sun | 58 | | | | | hsa-mir-1287 | | 0 |
| RCCC Renal Lenburg | 5 | | | | | hsa-mir-515-3p | | 0 |
| GLB Brain Sun | 36 | | | | | hsa-mir-423-5p | | 0 |
| GLB Brain Sun | 54 | | | hsa-mir-548m | | hsa-mir-921 | | 0 |
| GLB Brain Sun | 51 | | | hsa-mir-28-3p | | | | 0 |
| GLB Brain Sun | 43 | | | hsa-mir-331-5p | | | | 0 |
| GLB Brain Sun | 60 | | | | | hsa-mir-548f | | 0 |
| GLB Brain Sun | 62 | | | hsa-mir-216a | | hsa-mir-495 | | 0 |
| GLB Brain Sun | 63 | | | | | hsa-mir-939 | | 0 |
| GLB Brain Sun | 64 | hsa-let-7e* | | | | | | 0 |
| GLB Brain Sun | 68 | | | hsa-mir-888 | | hsa-mir-299-5p | Dysregulated | 0 |
| GLB Brain Sun | 69 | | | hsa-mir-1288 | | | | 0 |
| RCCC Renal Lenburg | 6 | | | hsa-mir-651 | | | | 0 |
| GLB Brain Sun | 24 | | | hsa-mir-381 | Dysregulated | hsa-mir-494 | | 0 |
| | | | | hsa-mir-300 | | | | |
| GLB Brain Sun | 25 | | | | | hsa-mir-600 | | 0 |
| GLB Brain Sun | 27 | | | hsa-mir-590-3p | | hsa-mir-889 | | 0 |
| GLB Brain Sun | 20 | | | hsa-mir-556-3p | | hsa-mir-331-5p | | 0 |
| GLB Brain Sun | 21 | | | hsa-mir-188-5p | | | | 0 |
| GLB Brain Sun | 49 | | | | | hsa-mir-340 | | 0 |
| GLB Brain Sun | 42 | | | | | hsa-mir-602 | | 0 |
| GLB Brain Sun | 28 | | | hsa-mir-369-3p | | hsa-mir-323-3p | | 0 |
| GLB Brain Sun | 29 | hsa-mir-1181 | | | | hsa-mir-1275 | | 0 |
| GLB Brain Sun | 41 | | | hsa-mir-760 | | | | 0 |
| GLB Brain Sun | 1 | | | hsa-mir-767-5p | | hsa-mir-324-5p | | 0 |
| GLB Brain Sun | 0 | | | | | hsa-mir-637 | | 0 |
| GLB Brain Sun | 3 | | | | | hsa-mir-320a | | 0 |
| GLB Brain Sun | 5 | | | | | hsa-mir-515-5p | | 0 |
| GLB Brain Sun | 4 | | | | | hsa-mir-542-5p | | 0 |
| CA Bladder Dyrskjot | 12 | | | hsa-mir-607 | | hsa-mir-1244 | | 0 |
| GLB Brain Sun | 8 | | | hsa-mir-140-3p | | hsa-mir-501-5p | | 0 |
| GLB Brain Sun | 13 | | | | | hsa-mir-744 | | 0 |
| GCT Seminoma Korkola | 26 | | | hsa-mir-138 | | hsa-mir-374a | | 0 |
| GLB Brain Sun | 12 | | | hsa-mir-924 | | | | 0 |
| GLB Brain Sun | 73 | | | | | hsa-mir-187 | | 0 |
| GLB Brain Sun | 72 | | | hsa-mir-571 | | hsa-mir-1294 | | 0 |
| GLB Brain Sun | 71 | | | hsa-mir-1182 | | | | 0 |
| CA Bladder Dyrskjot | 65 | | | | | hsa-mir-888 | | 0 |
| GLB Brain Sun | 59 | | | | | hsa-mir-1252 | | 0 |
| GLB Brain Sun | 10 | | | hsa-mir-588 | | hsa-mir-409-5p | | 0 |
| GLB Brain Sun | 15 | | | hsa-mir-582-3p | | hsa-mir-219-1-3p | | 0 |
| GLB Brain Sun | 14 | | | hsa-mir-1299 | | | | 0 |
| GLB Brain Sun | 17 | | | hsa-mir-621 | | | | 0 |
| GLB Brain Sun | 31 | | | hsa-mir-936 | | hsa-mir-501-3p | | 0 |
| GLB Brain Sun | 37 | | | | | hsa-mir-1256 | | 0 |
| GLB Brain Sun | 50 | | | hsa-mir-664 | | hsa-mir-548c-3p | | 0 |
| GLB Brain Sun | 35 | | | | | hsa-mir-922 | | 0 |
| CA Colon Graudens | 15 | | | | | hsa-mir-140-5p | Causal | 0 |
| GLB Brain Sun | 55 | | | | | hsa-mir-32 | | 0 |
| GLB Brain Sun | 74 | | | | | hsa-mir-369-3p | | 0 |
| GLB Brain Sun | 18 | | | hsa-mir-655 | | hsa-mir-498 | | 0 |
| GLB Brain Sun | 57 | | | | | hsa-mir-921 | | 0 |
| CA Breast Sorlie | 24 | hsa-mir-1538 | | | | | | 0 |
| CA Breast Sorlie | 20 | | | hsa-mir-1279 | | hsa-mir-590-3p | | 0 |
| GCT Seminoma Korkola | 28 | | | | | hsa-mir-146a | | 0 |
| CA Breast Sorlie | 2 | | | hsa-mir-653 | | | | 0 |
| CA Breast Sorlie | 4 | | | | | hsa-mir-611 | | 0 |
| CA Breast Sorlie | 8 | | | hsa-mir-494 | | hsa-mir-1255a | | 0 |
| CA Breast Sorlie | 11 | | | hsa-mir-33a | | | | 0 |
| CA Breast Sorlie | 10 | | | hsa-mir-23b | | | | 0 |
| | | | | hsa-mir-23a | | | | |
| CA Breast Sorlie | 12 | | | hsa-mir-548c-3p | | | | 0 |
| CA Breast Sorlie | 14 | | | hsa-mir-632 | | | | 0 |
| CA Breast Sorlie | 17 | | | | | hsa-mir-640 | | 0 |
| CA Breast Sorlie | 16 | | | | | hsa-mir-579 | | 0 |
| CA Breast Sorlie | 19 | | | hsa-mir-155 | Causal | hsa-mir-129-5p | | 0 |
| CA Breast Sorlie | 18 | | | hsa-mir-490-3p | | | | 0 |
| GCT Seminoma Korkola | 34 | | | | | hsa-mir-136 | | 0 |
| GCT Seminoma Korkola | 8 | | | hsa-mir-940 | | | | 0 |
| CA Breast Richardson | 61 | | | | | hsa-mir-1271 | | 0 |
| CA Breast Richardson | 62 | | | | | hsa-mir-622 | | 0 |
| CA Breast Richardson | 63 | | | | | hsa-mir-561 | | 0 |
| CA Breast Richardson | 53 | | | hsa-mir-548m | | hsa-mir-486-5p | | 0 |
| CA Breast Richardson | 67 | | | | | hsa-mir-889 | | 0 |
| CA Breast Richardson | 68 | | | | | hsa-mir-663b | Dysregulated | 0 |
| | | | | | | hsa-mir-663 | | |
| CA Breast Richardson | 34 | | | hsa-mir-410 | | hsa-mir-186 | | 0 |
| CA Breast Richardson | 25 | | | | | hsa-mir-1252 | | 0 |
| CA Breast Richardson | 27 | | | | | hsa-mir-663b | Dysregulated | 0 |
| | | | | | | hsa-mir-663 | | |
| CA Breast Richardson | 46 | hsa-mir-487b | | hsa-mir-323-3p | | hsa-mir-607 | | 0 |
| CA Breast Richardson | 45 | | | hsa-mir-181d | Dysregulated | hsa-mir-513a-3p | | 0 |
| | | | | hsa-mir-181b | | | | |
| CA Breast Richardson | 29 | | | | | hsa-mir-1302 | | 0 |
| CA Breast Richardson | 3 | | | | | hsa-mir-578 | | 0 |
| CA Breast Richardson | 4 | | | hsa-mir-433 | | | | 0 |
| CA Breast Richardson | 28 | | | | | hsa-mir-765 | | 0 |
| CA Breast Richardson | 56 | | | hsa-mir-146b-5p | Causal | hsa-mir-34b | | 0 |
| | | | | hsa-mir-146a | | | | |
| CA Breast Richardson | 7 | | | | | hsa-mir-590-3p | | 0 |
| CA Breast Richardson | 33 | | | hsa-mir-876-5p | | hsa-mir-1251 | | 0 |
| CA Breast Richardson | 38 | | | hsa-mir-1271 | | hsa-mir-103b | | 0 |
| | | | | | | hsa-mir-103a | | |
| CA Breast Richardson | 15 | hsa-let-7d* | | | | hsa-mir-494 | | 0 |
| CA Breast Richardson | 32 | | | hsa-mir-1245b-5p | | | | 0 |
| | | | | hsa-mir-1245 | | | | |
| CA Breast Richardson | 58 | | | | | hsa-mir-889 | | 0 |
| CA Breast Richardson | 13 | | | hsa-mir-590-3p | | | | 0 |
| CA Breast Richardson | 59 | | | | | hsa-mir-608 | | 0 |
| CA Breast Richardson | 17 | hsa-mir-566 | | | | hsa-mir-423-5p | | 0 |
| CA Breast Richardson | 16 | | | | | hsa-mir-18a | Dysregulated | 0 |
| CA Breast Richardson | 19 | | | | | hsa-mir-586 | | 0 |
| CA Breast Richardson | 18 | | | | | hsa-mir-135a | | 0 |
| CA Breast Richardson | 23 | | | | | hsa-mir-544 | | 0 |
| | | | | | | hsa-mir-544b | | |
| CA Breast Richardson | 51 | | | | | hsa-mir-633 | | 0 |
| CA Breast Richardson | 50 | | | | | hsa-mir-380 | | 0 |
| CA Breast Richardson | 55 | | | hsa-mir-130b | | hsa-mir-544 | | 0 |
| | | | | hsa-mir-301a | | hsa-mir-544b | | |
| | | | | hsa-mir-301b | | | | |
| | | | | hsa-mir-130a | | | | |
| | | | | hsa-mir-454 | | | | |
| CA Breast Richardson | 54 | | | | | hsa-mir-361-3p | | 0 |
| CA Breast Richardson | 31 | | | | | hsa-mir-376a | | 0 |
| CA Breast Richardson | 65 | | | | | hsa-mir-1254 | | 0 |
| OD Brain Bredel | 30 | | | | | hsa-mir-1306 | | 0 |
| MCA Breast Radvanyi | 21 | | | hsa-mir-626 | | | | 0 |
| MCA Breast Radvanyi | 2 | | | | | hsa-mir-204 | Causal | 0 |
| MCA Breast Radvanyi | 7 | | | hsa-mir-595 | | hsa-mir-324-3p | | 0 |
| COID Lung Bhattacharjee | 71 | | | hsa-mir-1237 | | | | 0 |
| MCA Breast Radvanyi | 12 | | | hsa-mir-489 | | | | 0 |
| OD Brain Bredel | 36 | | | | | hsa-mir-326 | | 0 |
| MCA Breast Radvanyi | 17 | | | | | hsa-mir-219-5p | | 0 |
| MCA Breast Radvanyi | 19 | | | | | hsa-mir-210 | Causal | 0 |
| MCA Breast Radvanyi | 18 | | | | | hsa-mir-663b | Dysregulated | 0 |
| | | | | | | hsa-mir-663 | | |
| ILC Breast Radvanyi | 24 | | | | | hsa-mir-340 | | 0 |
| ILC Breast Radvanyi | 26 | | | | | hsa-mir-486-3p | | 0 |
| ILC Breast Radvanyi | 20 | | | | | hsa-mir-328 | Dysregulated | 0 |
| ILC Breast Radvanyi | 22 | | | hsa-mir-508-3p | | | | 0 |
| GCT Seminoma Korkola | 82 | | | | | hsa-mir-576-5p | | 0 |
| ILC Breast Radvanyi | 3 | | | | | hsa-mir-383 | | 0 |
| ILC Breast Radvanyi | 2 | | | | | hsa-mir-423-3p | | 0 |
| ILC Breast Radvanyi | 5 | | | hsa-mir-501-5p | | | | 0 |
| ILC Breast Radvanyi | 4 | | | hsa-mir-122 | Dysregulated | hsa-mir-548a-3p | | 0 |
| MM Myeloma Zhan | 14 | | | | | hsa-mir-548l | | 0 |
| ILC Breast Radvanyi | 6 | hsa-let-7d* | | | | hsa-mir-579 | | 0 |
| ILC Breast Radvanyi | 11 | hsa-mir-483-5p | | | | | | 0 |
| ILC Breast Radvanyi | 10 | | | | | hsa-mir-548c-3p | | 0 |
| ILC Breast Radvanyi | 15 | | | | | hsa-mir-518b | | 0 |
| ILC Breast Radvanyi | 19 | | | hsa-mir-663b | Dysregulated | hsa-mir-338-3p | | 0 |
| | | | | hsa-mir-663 | | | | |
| IDC Breast Radvanyi | 56 | | | hsa-mir-216b | | hsa-mir-576-5p | | 0 |
| IDC Breast Radvanyi | 54 | | | hsa-mir-1277 | | | | 0 |
| IDC Breast Radvanyi | 48 | | | hsa-mir-630 | | | | 0 |
| IDC Breast Radvanyi | 43 | | | | | hsa-mir-1224-5p | | 0 |
| GCT Seminoma Korkola | 87 | | | hsa-mir-1179 | | hsa-mir-371b-5p | Dysregulated | 0 |
| | | | | | | hsa-mir-371-5p | | |
| IDC Breast Radvanyi | 63 | | | | | hsa-mir-548h | | 0 |
| IDC Breast Radvanyi | 65 | | | | | hsa-mir-802 | | 0 |
| CA Bladder Dyrskjot | 80 | | | | | hsa-mir-616 | | 0 |
| IDC Breast Radvanyi | 42 | | | | | hsa-mir-766 | | 0 |
| IDC Breast Radvanyi | 24 | | | | | hsa-mir-1291 | | 0 |
| IDC Breast Radvanyi | 23 | hsa-mir-376a* | | | | | | 0 |
| IDC Breast Radvanyi | 27 | | | | | hsa-mir-323-5p | | 0 |
| | | | | | | hsa-mir-323b-5p | | |
| GBM Brain Liang | 16 | | | | | hsa-mir-663b | | 0 |
| | | | | | | hsa-mir-663 | | |
| IDC Breast Radvanyi | 21 | | | hsa-mir-493 | | | | 0 |
| GCT Seminoma Korkola | 29 | | | | | hsa-mir-548c-3p | | 0 |
| IDC Breast Radvanyi | 47 | | | hsa-mir-760 | | | | 0 |
| IDC Breast Radvanyi | 44 | | | hsa-mir-548a-3p | | | | 0 |
| IDC Breast Radvanyi | 28 | | | | | hsa-mir-149 | Dysregulated | 0 |
| IDC Breast Radvanyi | 41 | | | hsa-mir-361-3p | | hsa-mir-939 | | 0 |
| IDC Breast Radvanyi | 2 | | | | | hsa-mir-513a-3p | | 0 |
| IDC Breast Radvanyi | 4 | | | hsa-mir-661 | Causal | hsa-mir-608 | | 0 |
| IDC Breast Radvanyi | 7 | | | | | hsa-mir-450b-3p | | 0 |
| GCT Seminoma Korkola | 108 | | | hsa-mir-570 | | hsa-mir-590-5p | | 0 |
| IDC Breast Radvanyi | 9 | | | | | hsa-mir-661 | Causal | 0 |
| IDC Breast Radvanyi | 8 | hsa-mir-604 | | hsa-mir-1273 | | hsa-mir-631 | | 0 |
| | | | | hsa-mir-1273c | | | | |
| | | | | hsa-mir-1273d | | | | |
| | | | | hsa-mir-1273e | | | | |
| | | | | hsa-mir-1273f | | | | |
| | | | | hsa-mir-1273g | | | | |
| IDC Breast Radvanyi | 15 | | | hsa-mir-182 | Causal | hsa-mir-205 | Causal | 0 |
| IDC Breast Radvanyi | 14 | hsa-mir-590-3p | | hsa-mir-556-5p | | hsa-mir-323-3p | | 0 |
| IDC Breast Radvanyi | 39 | | | | | hsa-mir-125a-3p | | 0 |
| IDC Breast Radvanyi | 12 | | | | | hsa-mir-590-3p | | 0 |
| IDC Breast Radvanyi | 59 | hsa-mir-1469 | | | | | | 0 |
| IDC Breast Radvanyi | 16 | | | hsa-mir-9 | Causal | | | 0 |
| IDC Breast Radvanyi | 51 | | | hsa-mir-548l | | hsa-mir-548n | | 0 |
| IDC Breast Radvanyi | 35 | | | hsa-mir-488 | | | | 0 |
| IDC Breast Radvanyi | 34 | | | hsa-mir-532-5p | | | | 0 |
| OD Brain Bredel | 47 | | | | | hsa-mir-219-1-3p | | 0 |
| IDC Breast Radvanyi | 18 | | | | | hsa-mir-1207-3p | | 0 |
| IDC Breast Radvanyi | 50 | hsa-mir-1247 | | | | | | 0 |
| CA Colon Graudens | 28 | | | hsa-mir-566 | | hsa-mir-508-5p | | 0 |
| CA Colon Graudens | 29 | | | hsa-mir-337-3p | | | | 0 |
| CA Colon Graudens | 24 | | | hsa-mir-381 | | hsa-mir-522 | | 0 |
| | | | | hsa-mir-300 | | | | |
| CA Colon Graudens | 25 | | | hsa-mir-202 | | | | 0 |
| GCT Seminoma Korkola | 106 | | | | | hsa-mir-513b | | 0 |
| CA Colon Graudens | 40 | | | hsa-mir-507 | | hsa-mir-369-3p | | 0 |
| | | | | hsa-mir-557 | | | | |
| CA Colon Graudens | 41 | | | hsa-mir-1243 | | | | 0 |
| CA Colon Graudens | 0 | | | | | hsa-mir-138 | | 0 |
| CA Colon Graudens | 3 | | | hsa-mir-552 | | | | 0 |
| CA Colon Graudens | 2 | | | hsa-mir-551b | | hsa-mir-451b | Causal | 0 |
| | | | | hsa-mir-551a | | hsa-mir-451 | | |
| GCT Seminoma Korkola | 104 | | | | | hsa-mir-557 | | 0 |
| CA Colon Graudens | 4 | | | | | hsa-mir-144 | | 0 |
| CA Colon Graudens | 6 | | | | | hsa-mir-658 | | 0 |
| CA Colon Graudens | 9 | | | hsa-mir-338-3p | | | | 0 |
| CA Colon Graudens | 8 | | | hsa-mir-595 | | | | 0 |
| CA Colon Graudens | 39 | | | hsa-mir-873 | | | | 0 |
| CA Colon Graudens | 11 | hsa-mir-523 | | | | | | 0 |
| CA Colon Graudens | 10 | | | | | hsa-mir-1225-5p | | 0 |
| CA Colon Graudens | 38 | | | hsa-mir-320e | Dysregulated | hsa-mir-96 | Dysregulated | 0 |
| | | | | hsa-mir-320d | | | | |
| | | | | hsa-mir-320c | | | | |
| | | | | hsa-mir-320b | | | | |
| | | | | hsa-mir-320a | | | | |
| OD Brain Bredel | 41 | | | hsa-mir-1276 | | | | 0 |
| CA Colon Graudens | 14 | | | hsa-mir-1244 | | | | 0 |
| CA Colon Graudens | 17 | | | hsa-mir-642a | | hsa-mir-597 | | 0 |
| | | | | hsa-mir-642b | | | | |
| RCCC Renal Lenburg | 0 | | | | | hsa-mir-489 | Dysregulated | 0 |
| CA Colon Graudens | 32 | | | | | hsa-mir-296-5p | Dysregulated | 0 |
| CA Colon Graudens | 31 | | | | | hsa-mir-548o | | 0 |
| CA Colon Graudens | 30 | | | hsa-mir-125b | Dysregulated | | | 0 |
| | | | | hsa-mir-125a-5p | | | | |
| CA Colon Graudens | 37 | | | | | hsa-mir-32 | Dysregulated | 0 |
| CA Colon Graudens | 36 | | | hsa-mir-576-5p | | hsa-mir-512-3p | | 0 |
| CA Colon Graudens | 35 | | | hsa-mir-325 | | | | 0 |
| CA Colon Graudens | 19 | | | hsa-mir-148a | | | | 0 |
| H5CC Head-Neck Cromer | 25 | | | hsa-mir-1264 | | hsa-mir-495 | | 0 |
| H5CC Head-Neck Cromer | 26 | | | hsa-mir-199a-5p | Dysregulated | hsa-mir-885-5p | | 0 |
| | | | | hsa-mir-199b-5p | | | | |
| H5CC Head-Neck Cromer | 27 | | | | | hsa-mir-615-5p | | 0 |
| HSCC Head-Neck Cromer | 20 | | | hsa-mir-543 | | hsa-mir-567 | | 0 |
| HSCC Head-Neck Cromer | 22 | | | hsa-mir-218 | | hsa-mir-30b | Dysregulated | 0 |
| HSCC Head-Neck Cromer | 23 | | | hsa-mir-380 | | | | 0 |
| HSCC Head-Neck Cromer | 3 | | | hsa-mir-496 | | | | 0 |
| HSCC Head-Neck Cromer | 2 | | | | | hsa-mir-374b | Dysregulated | 0 |
| HSCC Head-Neck Cromer | 5 | | | | | hsa-mir-486-3p | | 0 |
| HSCC Head-Neck Cromer | 4 | | | | | hsa-mir-662 | | 0 |
| GCT Seminoma Korkola | 30 | | | | | hsa-mir-588 | | 0 |
| HSCC Head-Neck Cromer | 6 | | | | | hsa-mir-1275 | | 0 |
| HSCC Head-Neck Cromer | 8 | | | | | hsa-mir-142-5p | | 0 |
| HSCC Head-Neck Cromer | 11 | | | hsa-mir-129-5p | | | | 0 |
| HSCC Head-Neck Cromer | 10 | | | | | hsa-mir-520a-5p | | 0 |
| HSCC Head-Neck Cromer | 13 | | | hsa-mir-875-3p | | hsa-mir-622 | | 0 |
| HSCC Head-Neck Cromer | 12 | | | hsa-mir-1297 | Dysregulated | hsa-mir-513a-3p | | 0 |
| | | | | hsa-mir-26a | | | | |
| | | | | hsa-mir-26b | | | | |
| MM Myeloma Zhan | 10 | | | | | hsa-mir-1305 | | 0 |
| OD Brain Bredel | 7 | | | | | hsa-mir-23b | | 0 |
| HSCC Head-Neck Cromer | 18 | | | hsa-mir-214 | Dysregulated | hsa-mir-615-3p | | 0 |
| HSCC Head-Neck Chung | 10 | | | hsa-mir-1226 | | hsa-mir-607 | | 0 |
| HSCC Head-Neck Chung | 1 | hsa-mir-29b | Dysregulated | hsa-mir-767-5p | | hsa-mir-450b-5p | | 0 |
| | | hsa-mir-29c | | | | | | |
| | | hsa-mir-29a | | | | | | |
| IDC Breast Radvanyi | 6 | | | hsa-mir-652 | | | | 0 |
| HSCC Head-Neck Chung | 3 | | | | | hsa-mir-1286 | | 0 |
| HSCC Head-Neck Chung | 2 | | | hsa-mir-148a | Dysregulated | hsa-mir-944 | | 0 |
| | | | | hsa-mir-148b | | | | |
| | | | | hsa-mir-152 | | | | |
| HSCC Head-Neck Chung | 5 | | | | | hsa-mir-1202 | | 0 |
| HSCC Head-Neck Chung | 4 | | | | | hsa-mir-548l | | 0 |
| HSCC Head-Neck Chung | 7 | | | hsa-mir-487a | | hsa-mir-573 | | 0 |
| HSCC Head-Neck Chung | 6 | | | | | hsa-mir-590-3p | | 0 |
| HSCC Head-Neck Chung | 9 | | | | | hsa-mir-708 | | 0 |
| HSCC Head-Neck Chung | 8 | | | | | hsa-mir-410 | | 0 |
| B-CLL Leukemia Haslinger | 30 | | | hsa-mir-654-3p | | | | 0 |
| B-CLL Leukemia Haslinger | 28 | | | | | hsa-mir-376a | | 0 |
| | | | | | | hsa-mir-376b | | |
| | | | | | | hsa-mir-376c | | |
| GCT Seminoma Korkola | 60 | | | | | hsa-mir-551a | | 0 |
| B-CLL Leukemia Haslinger | 36 | | | | | hsa-mir-587 | | 0 |
| B-CLL Leukemia Haslinger | 61 | | | | | hsa-mir-588 | | 0 |
| B-CLL Leukemia Haslinger | 62 | | | hsa-mir-515-5p | | hsa-mir-548c-3p | | 0 |
| B-CLL Leukemia Haslinger | 64 | | | hsa-mir-1278 | | | | 0 |
| B-CLL Leukemia Haslinger | 65 | | | | | hsa-mir-1247 | | 0 |
| B-CLL Leukemia Haslinger | 66 | | | | | hsa-mir-939 | | 0 |
| B-CLL Leukemia Haslinger | 68 | | | hsa-mir-377 | | hsa-mir-520g | | 0 |
| B-CLL Leukemia Haslinger | 69 | hsa-mir-450a | | hsa-mir-532-5p | | hsa-mir-146a | Dysregulated | 0 |
| B-CLL Leukemia Haslinger | 52 | | | hsa-mir-323-3p | | hsa-mir-302a | | 0 |
| B-CLL Leukemia Haslinger | 25 | | | hsa-mir-1237 | | hsa-mir-548c-3p | | 0 |
| B-CLL Leukemia Haslinger | 27 | | | hsa-mir-760 | | | | 0 |
| B-CLL Leukemia Haslinger | 21 | | | hsa-mir-429 | Causal | hsa-mir-548c-3p | | 0 |
| | | | | hsa-mir-200b | | | | |
| | | | | hsa-mir-200c | | | | |
| | | | | hsa-mir-200a | | | | |
| B-CLL Leukemia Haslinger | 48 | | | hsa-mir-520d-5p | | | | 0 |
| | | | | hsa-mir-524-5p | | | | |
| B-CLL Leukemia Haslinger | 23 | | | | | hsa-mir-548o | | 0 |
| B-CLL Leukemia Haslinger | 46 | | | hsa-mir-1249 | | hsa-mir-1247 | | 0 |
| B-CLL Leukemia Haslinger | 44 | | | | | hsa-mir-200b | Causal | 0 |
| B-CLL Leukemia Haslinger | 45 | | | | | hsa-mir-889 | | 0 |
| B-CLL Leukemia Haslinger | 29 | | | hsa-mir-520f | | hsa-mir-1270 | | 0 |
| B-CLL Leukemia Haslinger | 40 | | | hsa-mir-582-3p | | | | 0 |
| B-CLL Leukemia Haslinger | 41 | | | | | hsa-mir-483-5p | | 0 |
| B-CLL Leukemia Haslinger | 1 | | | | | hsa-mir-487a | | 0 |
| B-CLL Leukemia Haslinger | 0 | | | | | hsa-mir-409-5p | | 0 |
| B-CLL Leukemia Haslinger | 5 | | | | | hsa-mir-622 | | 0 |
| B-CLL Leukemia Haslinger | 9 | | | | | hsa-mir-1245b-5p | | 0 |
| | | | | | | hsa-mir-1245 | | |
| B-CLL Leukemia Haslinger | 8 | | | | | hsa-mir-939 | | 0 |
| B-CLL Leukemia Haslinger | 56 | | | | | hsa-mir-548b-3p | | 0 |
| B-CLL Leukemia Haslinger | 43 | | | | | hsa-mir-483-3p | | 0 |
| B-CLL Leukemia Haslinger | 35 | | | hsa-mir-1294 | | hsa-mir-486-3p | | 0 |
| END Ovarian Hendrix | 77 | | | | | hsa-mir-590-3p | | 0 |
| B-CLL Leukemia Haslinger | 15 | | | hsa-mir-338-5p | | hsa-mir-654-3p | | 0 |
| B-CLL Leukemia Haslinger | 58 | hsa-mir-337-5p | | | | | | 0 |
| B-CLL Leukemia Haslinger | 11 | | | | | hsa-mir-34c-3p | | 0 |
| B-CLL Leukemia Haslinger | 10 | | | | | hsa-mir-454 | | 0 |
| B-CLL Leukemia Haslinger | 13 | | | | | hsa-mir-892b | | 0 |
| B-CLL Leukemia Haslinger | 38 | | | | | hsa-mir-548g | | 0 |
| B-CLL Leukemia Haslinger | 59 | | | hsa-mir-636 | | hsa-mir-1228 | | 0 |
| B-CLL Leukemia Haslinger | 17 | | | | | hsa-mir-590-3p | | 0 |
| B-CLL Leukemia Haslinger | 16 | | | | | hsa-mir-1285 | | 0 |
| B-CLL Leukemia Haslinger | 54 | hsa-mir-937 | | | | hsa-mir-1321 | | 0 |
| B-CLL Leukemia Haslinger | 49 | | | hsa-mir-660 | | hsa-mir-1227 | | 0 |
| B-CLL Leukemia Haslinger | 51 | | | | | hsa-mir-448 | | 0 |
| B-CLL Leukemia Haslinger | 50 | | | | | hsa-mir-296-5p | | 0 |
| B-CLL Leukemia Haslinger | 34 | | | hsa-mir-492 | | | | 0 |
| B-CLL Leukemia Haslinger | 55 | | | hsa-mir-1323 | | hsa-mir-200b | Causal | 0 |
| | | | | hsa-mir-548o | | | | |
| B-CLL Leukemia Haslinger | 37 | | | hsa-mir-545 | | hsa-mir-323-3p | | 0 |
| B-CLL Leukemia Haslinger | 31 | | | | | hsa-mir-1321 | | 0 |
| AD Lung Beer | 51 | | | hsa-mir-1202 | | | | 0 |
| AD Lung Beer | 22 | | | | | hsa-mir-525-5p | | 0 |
| AD Lung Beer | 34 | | | hsa-mir-193b | | hsa-mir-646 | | 0 |
| AD Lung Beer | 53 | | | hsa-mir-581 | | hsa-mir-548d-3p | | 0 |
| AD Lung Beer | 24 | | | hsa-mir-770-5p | | | | 0 |
| OD Brain Bredel | 15 | | | | | hsa-mir-765 | | 0 |
| AD Lung Beer | 27 | | | hsa-mir-320e | | | | 0 |
| | | | | hsa-mir-320d | | | | |
| | | | | hsa-mir-320c | | | | |
| | | | | hsa-mir-320b | | | | |
| | | | | hsa-mir-320a | | | | |
| GLB Brain Sun | 76 | | | hsa-mir-561 | | hsa-mir-139-5p | Dysregulated | 0 |
| AD Lung Beer | 46 | | | hsa-mir-874 | | hsa-mir-606 | | 0 |
| AD Lung Beer | 44 | | | hsa-mir-484 | | hsa-mir-452 | | 0 |
| AD Lung Beer | 42 | | | | | hsa-mir-651 | | 0 |
| AD Lung Beer | 43 | | | | | hsa-mir-1275 | | 0 |
| AD Lung Beer | 40 | | | hsa-mir-320e | | hsa-mir-561 | | 0 |
| | | | | hsa-mir-320d | | | | |
| | | | | hsa-mir-320c | | | | |
| | | | | hsa-mir-320b | | | | |
| | | | | hsa-mir-320a | | | | |
| AD Lung Beer | 1 | | | hsa-mir-766 | | | | 0 |
| AD Lung Beer | 0 | | | | | hsa-let-7d | Causal | 0 |
| AD Lung Beer | 3 | | | hsa-mir-338-5p | Dysregulated | | | 0 |
| GCT Seminoma Korkola | 93 | | | hsa-mir-193a-5p | | hsa-mir-640 | | 0 |
| AD Lung Beer | 5 | | | | | hsa-mir-1292 | | 0 |
| AD Lung Beer | 4 | | | | | hsa-mir-1183 | | 0 |
| AD Lung Beer | 7 | | | hsa-mir-548b-5p | | | | 0 |
| | | | | hsa-mir-548am | | | | |
| | | | | hsa-mir-548ab | | | | |
| | | | | hsa-mir-548aa | | | | |
| | | | | hsa-mir-548ag | | | | |
| | | | | hsa-mir-548ak | | | | |
| | | | | hsa-mir-548aj | | | | |
| | | | | hsa-mir-548ai | | | | |
| | | | | hsa-mir-548ah | | | | |
| | | | | hsa-mir-548an | | | | |
| | | | | hsa-mir-548al | | | | |
| | | | | hsa-mir-548ae | | | | |
| | | | | hsa-mir-548ad | | | | |
| | | | | hsa-mir-548a-5p | | | | |
| | | | | hsa-mir-548ac | | | | |
| | | | | hsa-mir-548d-5p | | | | |
| | | | | hsa-mir-548i | | | | |
| | | | | hsa-mir-548j | | | | |
| | | | | hsa-mir-548c-5p | | | | |
| | | | | hsa-mir-548h | | | | |
| AD Lung Beer | 6 | | | | | hsa-mir-548c-3p | | 0 |
| AD Lung Beer | 8 | | | hsa-mir-939 | | hsa-mir-637 | | 0 |
| AD Lung Beer | 28 | | | hsa-mir-147 | | hsa-mir-1245b-5p | | 0 |
| | | | | hsa-mir-147b | | hsa-mir-1245 | | |
| AD Lung Beer | 38 | | | | | hsa-mir-933 | | 0 |
| AD Lung Beer | 29 | | | hsa-mir-661 | | | | 0 |
| AD Lung Beer | 11 | | | hsa-mir-9 | Dysregulated | | | 0 |
| OD Brain Bredel | 18 | | | hsa-mir-1261 | | | | 0 |
| AD Lung Beer | 12 | | | hsa-mir-1208 | | hsa-mir-378b | | 0 |
| | | | | | | hsa-mir-378c | | |
| | | | | | | hsa-mir-378f | | |
| | | | | | | hsa-mir-378g | | |
| | | | | | | hsa-mir-378d | | |
| | | | | | | hsa-mir-378e | | |
| | | | | | | hsa-mir-378h | | |
| | | | | | | hsa-mir-378i | | |
| | | | | | | hsa-mir-378 | | |
| AD Lung Beer | 15 | | | | | hsa-mir-586 | | 0 |
| AD Lung Beer | 14 | | | hsa-mir-571 | | hsa-mir-542-5p | | 0 |
| AD Lung Beer | 32 | | | hsa-mir-656 | | | | 0 |
| MUC Ovarian Hendrix | 35 | | | | | hsa-mir-340 | | 0 |
| AD Lung Beer | 35 | | | hsa-mir-222 | Causal | | | 0 |
| AD Lung Beer | 52 | | | hsa-mir-372 | Causal | | | 0 |
| AD Lung Beer | 19 | hsa-mir-487b | | | | | | 0 |
| AD Lung Beer | 18 | | | hsa-mir-876-3p | | hsa-mir-19a | Causal | 0 |
| AD Lung Beer | 33 | | | hsa-mir-380 | | hsa-mir-379 | | 0 |
| GCT Seminoma Korkola | 96 | | | | | hsa-mir-29a | | 0 |
| CA Bladder Dyrskjot | 18 | | | | | hsa-mir-1291 | | 0 |
| AD Lung Bhattacharjee | 60 | | | hsa-mir-190b | | hsa-mir-495 | | 0 |
| | | | | hsa-mir-190 | | | | |
| AD Lung Bhattacharjee | 82 | | | | | hsa-mir-647 | | 0 |
| AD Lung Bhattacharjee | 64 | hsa-mir-150 | Dysregulated | | | hsa-mir-765 | | 0 |
| AD Lung Bhattacharjee | 65 | hsa-mir-3177-5p | | | | hsa-mir-486-3p | | 0 |
| AD Lung Bhattacharjee | 67 | | | hsa-mir-361-5p | | | | 0 |
| AD Lung Bhattacharjee | 69 | | | hsa-mir-23b | | hsa-mir-522 | | 0 |
| | | | | hsa-mir-23a | | | | |
| AD Lung Bhattacharjee | 81 | | | | | hsa-mir-516b | | 0 |
| AD Lung Bhattacharjee | 24 | | | | | hsa-mir-608 | | 0 |
| HSCC Head-Neck Cromer | 19 | | | | | hsa-mir-548l | | 0 |
| AD Lung Bhattacharjee | 27 | hsa-mir-487b | | | | hsa-mir-451b | Dysregulated | 0 |
| | | | | | | hsa-mir-451 | | |
| AD Lung Bhattacharjee | 20 | | | hsa-mir-599 | | hsa-mir-1245b-5p | | 0 |
| | | | | | | hsa-mir-1245 | | |
| AD Lung Bhattacharjee | 22 | | | hsa-mir-875-3p | | hsa-mir-619 | | 0 |
| AD Lung Bhattacharjee | 46 | | | hsa-mir-548a-3p | | hsa-mir-548c-3p | | 0 |
| | | | | hsa-mir-548e | | | | |
| | | | | hsa-mir-548f | | | | |
| AD Lung Bhattacharjee | 47 | | | hsa-mir-409-3p | | hsa-mir-1245b-5p | | 0 |
| | | | | | | hsa-mir-1245 | | |
| AD Lung Bhattacharjee | 44 | hsa-mir-655 | | hsa-mir-633 | | hsa-mir-1284 | | 0 |
| AD Lung Bhattacharjee | 45 | | | | | hsa-mir-340 | | 0 |
| AD Lung Bhattacharjee | 42 | | | hsa-mir-760 | | | | 0 |
| AD Lung Bhattacharjee | 41 | | | | | hsa-mir-296-5p | | 0 |
| AD Lung Bhattacharjee | 0 | | | | | hsa-mir-338-5p | Dysregulated | 0 |
| OD Brain Bredel | 61 | | | | | hsa-mir-937 | | 0 |
| AD Lung Bhattacharjee | 2 | | | hsa-mir-645 | | | | 0 |
| AD Lung Bhattacharjee | 4 | | | | | hsa-mir-1321 | | 0 |
| AD Lung Bhattacharjee | 7 | | | hsa-mir-570 | | hsa-mir-1305 | | 0 |
| AD Lung Bhattacharjee | 6 | hsa-mir-941 | | | | hsa-mir-602 | | 0 |
| AD Lung Bhattacharjee | 9 | | | | | hsa-mir-154 | | 0 |
| AD Lung Bhattacharjee | 8 | | | | | hsa-mir-1324 | | 0 |
| AD Lung Bhattacharjee | 52 | | | | | hsa-let-7a | Causal | 0 |
| AD Lung Bhattacharjee | 28 | | | hsa-mir-122 | | hsa-mir-548a-3p | | 0 |
| AD Lung Bhattacharjee | 49 | | | hsa-mir-567 | | | | 0 |
| GCT Seminoma Korkola | 16 | | | | | hsa-mir-1183 | | 0 |
| AD Lung Bhattacharjee | 39 | | | hsa-mir-525-3p | | | | 0 |
| | | | | hsa-mir-524-3p | | | | |
| AD Lung Bhattacharjee | 35 | | | | | hsa-mir-133a | | 0 |
| AD Lung Bhattacharjee | 76 | | | hsa-mir-371-3p | | | | 0 |
| AD Lung Bhattacharjee | 75 | | | hsa-mir-1270 | | hsa-mir-637 | | 0 |
| | | | | hsa-mir-620 | | | | |
| AD Lung Bhattacharjee | 74 | | | | | hsa-mir-543 | | 0 |
| AD Lung Bhattacharjee | 72 | | | hsa-mir-507 | | | | 0 |
| | | | | hsa-mir-557 | | | | |
| MPC Prostate Dhanasekaran | 36 | | | | | hsa-mir-942 | | 0 |
| AD Lung Bhattacharjee | 10 | | | | | hsa-mir-1321 | | 0 |
| GL Brain Bredel | 22 | | | | | hsa-mir-657 | | 0 |
| AD Lung Bhattacharjee | 59 | | | hsa-mir-29b | Causal | | | 0 |
| | | | | hsa-mir-29c | | | | |
| | | | | hsa-mir-29a | | | | |
| AD Lung Bhattacharjee | 14 | | | hsa-mir-517c | | hsa-mir-938 | | 0 |
| | | | | hsa-mir-517b | | | | |
| | | | | hsa-mir-517a | | | | |
| AD Lung Bhattacharjee | 17 | | | | | hsa-mir-491-5p | | 0 |
| GCT Seminoma Korkola | 50 | | | | | hsa-mir-376a | | 0 |
| | | | | | | hsa-mir-376b | | |
| | | | | | | hsa-mir-376c | | |
| AD Lung Bhattacharjee | 33 | | | hsa-mir-892a | | hsa-mir-490-5p | | 0 |
| AD Lung Bhattacharjee | 30 | | | hsa-mir-607 | | hsa-mir-548c-3p | | 0 |
| AD Lung Bhattacharjee | 50 | | | | | hsa-mir-642a | | 0 |
| | | | | | | hsa-mir-642b | | |
| AD Lung Bhattacharjee | 53 | | | hsa-mir-146b-5p | Dysregulated | | | 0 |
| | | | | hsa-mir-146a | | | | |
| AD Lung Bhattacharjee | 34 | | | | | hsa-mir-296-5p | | 0 |
| AD Lung Bhattacharjee | 19 | | | hsa-mir-1322 | | hsa-mir-1247 | | 0 |
| AD Lung Bhattacharjee | 55 | | | | | hsa-mir-1286 | | 0 |
| AD Lung Bhattacharjee | 12 | | | | | hsa-mir-590-3p | | 0 |
| GL Brain Bredel | 60 | | | hsa-mir-513a-5p | | hsa-mir-25 | Dysregulated | 0 |
| COID Lung Bhattacharjee | 24 | | | | | hsa-mir-342-5p | | 0 |
| COID Lung Bhattacharjee | 25 | | | hsa-mir-1276 | | hsa-mir-361-5p | | 0 |
| COID Lung Bhattacharjee | 20 | | | | | hsa-mir-615-3p | | 0 |
| COID Lung Bhattacharjee | 21 | | | hsa-mir-590-3p | | hsa-mir-944 | | 0 |
| COID Lung Bhattacharjee | 23 | | | | | hsa-mir-1228 | | 0 |
| COID Lung Bhattacharjee | 28 | | | hsa-mir-758 | | hsa-mir-608 | | 0 |
| COID Lung Bhattacharjee | 0 | | | hsa-mir-663b | | hsa-mir-518d-5p | | 0 |
| | | | | hsa-mir-663 | | | | |
| COID Lung Bhattacharjee | 8 | | | hsa-mir-607 | | hsa-mir-340 | | 0 |
| COID Lung Bhattacharjee | 58 | | | | | hsa-mir-23a | | 0 |
| CA Colon Graudens | 5 | | | | | hsa-mir-548h | | 0 |
| COID Lung Bhattacharjee | 51 | | | | | hsa-mir-656 | | 0 |
| COID Lung Bhattacharjee | 52 | | | | | hsa-mir-154 | | 0 |
| COID Lung Bhattacharjee | 89 | | | hsa-mir-1254 | | | | 0 |
| COID Lung Bhattacharjee | 80 | | | | | hsa-mir-548l | | 0 |
| COID Lung Bhattacharjee | 81 | | | hsa-mir-369-3p | | | | 0 |
| COID Lung Bhattacharjee | 86 | | | hsa-mir-580 | | hsa-mir-410 | | 0 |
| COID Lung Bhattacharjee | 87 | hsa-mir-1181 | | | | | | 0 |
| COID Lung Bhattacharjee | 84 | hsa-mir-615-5p | | | | | | 0 |
| COID Lung Bhattacharjee | 85 | | | | | hsa-mir-1180 | | 0 |
| COID Lung Bhattacharjee | 3 | | | hsa-mir-940 | | | | 0 |
| COID Lung Bhattacharjee | 7 | | | hsa-mir-431 | | hsa-mir-608 | | 0 |
| COID Lung Bhattacharjee | 39 | | | | | hsa-mir-608 | | 0 |
| COID Lung Bhattacharjee | 31 | | | hsa-mir-331-3p | | | | 0 |
| COID Lung Bhattacharjee | 30 | | | | | hsa-mir-423-5p | Dysregulated | 0 |
| CA Bladder Dyrskjot | 29 | | | | | hsa-mir-548c-3p | | 0 |
| COID Lung Bhattacharjee | 36 | | | | | hsa-mir-890 | | 0 |
| COID Lung Bhattacharjee | 60 | | | hsa-mir-505 | | hsa-mir-516a-3p | | 0 |
| COID Lung Bhattacharjee | 61 | | | hsa-mir-494 | | hsa-mir-34c-3p | | 0 |
| GCT Seminoma Korkola | 5 | | | | | hsa-mir-1204 | | 0 |
| COID Lung Bhattacharjee | 64 | | | | | hsa-mir-661 | | 0 |
| COID Lung Bhattacharjee | 66 | | | | | hsa-mir-507 | | 0 |
| COID Lung Bhattacharjee | 67 | | | hsa-mir-512-3p | | hsa-mir-181d | | 0 |
| COID Lung Bhattacharjee | 68 | | | | | hsa-mir-1272 | | 0 |
| COID Lung Bhattacharjee | 69 | | | | | hsa-mir-765 | | 0 |
| COID Lung Bhattacharjee | 2 | | | hsa-mir-548d-3p | | | | 0 |
| COID Lung Bhattacharjee | 6 | | | | | hsa-mir-874 | | 0 |
| COID Lung Bhattacharjee | 91 | | | hsa-mir-1275 | | | | 0 |
| COID Lung Bhattacharjee | 92 | | | | | hsa-mir-920 | | 0 |
| COID Lung Bhattacharjee | 11 | | | hsa-mir-501-5p | | hsa-mir-513b | | 0 |
| COID Lung Bhattacharjee | 10 | hsa-mir-138-2* | | hsa-mir-513a-3p | | hsa-mir-101 | Dysregulated | 0 |
| COID Lung Bhattacharjee | 12 | | | hsa-mir-509-5p | | hsa-mir-494 | | 0 |
| | | | | hsa-mir-509-3p | | | | |
| | | | | hsa-mir-509-3-5p | | | | |
| COID Lung Bhattacharjee | 14 | | | | | hsa-mir-557 | | 0 |
| COID Lung Bhattacharjee | 17 | | | hsa-mir-885-3p | | | | 0 |
| COID Lung Bhattacharjee | 16 | | | | | hsa-mir-376a | | 0 |
| COID Lung Bhattacharjee | 19 | | | hsa-mir-553 | | hsa-mir-615-5p | | 0 |
| COID Lung Bhattacharjee | 18 | | | | | hsa-mir-663b | | 0 |
| | | | | | | hsa-mir-663 | | |
| GL Brain Bredel | 25 | | | hsa-mir-338-5p | Dysregulated | hsa-mir-513b | | 0 |
| COID Lung Bhattacharjee | 47 | | | hsa-mir-574-3p | | | | 0 |
| COID Lung Bhattacharjee | 44 | | | hsa-mir-504 | | | | 0 |
| COID Lung Bhattacharjee | 45 | | | | | hsa-mir-615-5p | | 0 |
| COID Lung Bhattacharjee | 42 | | | | | hsa-mir-671-5p | | 0 |
| COID Lung Bhattacharjee | 43 | | | | | hsa-mir-658 | | 0 |
| COID Lung Bhattacharjee | 40 | | | hsa-mir-1181 | | hsa-mir-93 | Causal | 0 |
| COID Lung Bhattacharjee | 41 | | | hsa-mir-1303 | | | | 0 |
| COID Lung Bhattacharjee | 5 | | | hsa-mir-361-5p | | hsa-mir-548o | | 0 |
| COID Lung Bhattacharjee | 9 | | | hsa-mir-622 | | | | 0 |
| GCT Seminoma Korkola | 73 | | | | | hsa-mir-675 | | 0 |
| COID Lung Bhattacharjee | 75 | | | | | hsa-mir-874 | | 0 |
| COID Lung Bhattacharjee | 73 | | | hsa-mir-1262 | | | | 0 |
| COID Lung Bhattacharjee | 72 | | | | | hsa-mir-548n | | 0 |
| GCT Seminoma Korkola | 72 | | | hsa-mir-490-3p | | hsa-mir-760 | | 0 |
| COID Lung Bhattacharjee | 79 | | | | | hsa-mir-508-3p | | 0 |
| COID Lung Bhattacharjee | 78 | | | hsa-mir-920 | | hsa-mir-27b | Dysregulated | 0 |
| SQ Lung Bhattacharjee | 41 | | | hsa-mir-885-3p | | | | 0 |
| SQ Lung Bhattacharjee | 22 | | | hsa-mir-548n | | hsa-mir-607 | | 0 |
| SQ Lung Bhattacharjee | 35 | hsa-mir-449c* | | | | | | 0 |
| SQ Lung Bhattacharjee | 34 | hsa-mir-718 | | | | | | 0 |
| SQ Lung Bhattacharjee | 23 | | | | | hsa-mir-586 | | 0 |
| SQ Lung Bhattacharjee | 24 | | | | | hsa-mir-889 | | 0 |
| GCT Seminoma Korkola | 70 | | | | | hsa-mir-939 | | 0 |
| SQ Lung Bhattacharjee | 27 | | | | | hsa-mir-940 | | 0 |
| SQ Lung Bhattacharjee | 21 | | | | | hsa-mir-455-5p | | 0 |
| SQ Lung Bhattacharjee | 49 | | | | | hsa-mir-34a | Causal | 0 |
| SQ Lung Bhattacharjee | 46 | | | hsa-mir-650 | | | | 0 |
| SQ Lung Bhattacharjee | 44 | hsa-mir-4285 | | hsa-mir-767-5p | | | | 0 |
| SQ Lung Bhattacharjee | 45 | | | | | hsa-mir-654-5p | | 0 |
| SQ Lung Bhattacharjee | 28 | | | hsa-mir-616 | | | | 0 |
| SQ Lung Bhattacharjee | 29 | | | | | hsa-mir-486-3p | | 0 |
| SQ Lung Bhattacharjee | 40 | | | | | hsa-mir-339-3p | | 0 |
| SQ Lung Bhattacharjee | 42 | | | hsa-mir-637 | | | | 0 |
| SQ Lung Bhattacharjee | 0 | | | | | hsa-mir-662 | | 0 |
| GL Brain Bredel | 47 | | | | | hsa-mir-1236 | | 0 |
| SQ Lung Bhattacharjee | 5 | | | | | hsa-mir-595 | | 0 |
| SQ Lung Bhattacharjee | 4 | | | hsa-mir-423-5p | Dysregulated | hsa-mir-214 | Dysregulated | 0 |
| SQ Lung Bhattacharjee | 7 | | | | | hsa-mir-765 | | 0 |
| SQ Lung Bhattacharjee | 9 | | | | | hsa-mir-891a | | 0 |
| SQ Lung Bhattacharjee | 8 | hsa-mir-1203 | | | | | | 0 |
| SQ Lung Bhattacharjee | 43 | | | hsa-mir-597 | | hsa-mir-635 | | 0 |
| SQ Lung Bhattacharjee | 13 | | | | | hsa-mir-296-5p | | 0 |
| SQ Lung Bhattacharjee | 15 | | | hsa-mir-96 | Dysregulated | hsa-mir-569 | | 0 |
| SQ Lung Bhattacharjee | 14 | | | | | hsa-mir-486-3p | | 0 |
| SQ Lung Bhattacharjee | 16 | | | hsa-mir-802 | | | | 0 |
| SQ Lung Bhattacharjee | 19 | | | hsa-mir-423-5p | Dysregulated | hsa-mir-647 | | 0 |
| SQ Lung Bhattacharjee | 18 | | | hsa-mir-767-5p | | hsa-mir-410 | | 0 |
| SQ Lung Bhattacharjee | 30 | | | | | hsa-mir-1233 | | 0 |
| SQ Lung Bhattacharjee | 37 | | | hsa-mir-770-5p | | | | 0 |
| SQ Lung Bhattacharjee | 50 | | | | | hsa-mir-607 | | 0 |
| SQ Lung Bhattacharjee | 52 | | | hsa-mir-411 | | | | 0 |
| SQ Lung Bhattacharjee | 33 | | | hsa-mir-339-5p | Dysregulated | | | 0 |
| SQ Lung Bhattacharjee | 32 | | | hsa-mir-532-3p | | hsa-mir-210 | Dysregulated | 0 |
| SMCL Lung Bhattacharjee | 30 | | | hsa-mir-548n | | hsa-mir-655 | | 0 |
| SMCL Lung Bhattacharjee | 36 | | | | | hsa-mir-296-5p | | 0 |
| SMCL Lung Bhattacharjee | 60 | hsa-mir-671-3p | | | | | | 0 |
| SMCL Lung Bhattacharjee | 61 | | | | | hsa-mir-331-3p | | 0 |
| SMCL Lung Bhattacharjee | 35 | | | | | hsa-mir-212 | Dysregulated | 0 |
| SMCL Lung Bhattacharjee | 32 | | | | | hsa-mir-582-5p | | 0 |
| MUC Ovarian Hendrix | 13 | | | | | hsa-mir-1280 | | 0 |
| SMCL Lung Bhattacharjee | 25 | | | hsa-mir-101 | Dysregulated | | | 0 |
| SMCL Lung Bhattacharjee | 20 | | | | | hsa-mir-423-5p | Dysregulated | 0 |
| SMCL Lung Bhattacharjee | 21 | | | hsa-mir-570 | | | | 0 |
| SMCL Lung Bhattacharjee | 46 | | | hsa-mir-499a-5p | | hsa-mir-891b | | 0 |
| | | | | hsa-mir-499-5p | | | | |
| SMCL Lung Bhattacharjee | 23 | hsa-mir-3183 | | | | | | 0 |
| SMCL Lung Bhattacharjee | 45 | | | hsa-mir-432 | | hsa-mir-125a-5p | Causal | 0 |
| SMCL Lung Bhattacharjee | 28 | | | | | hsa-mir-744 | | 0 |
| SMCL Lung Bhattacharjee | 43 | | | | | hsa-mir-671-3p | | 0 |
| SMCL Lung Bhattacharjee | 41 | | | hsa-mir-512-3p | | | | 0 |
| SMCL Lung Bhattacharjee | 1 | | | hsa-mir-1237 | | | | 0 |
| SMCL Lung Bhattacharjee | 2 | | | hsa-mir-1266 | | hsa-mir-940 | | 0 |
| SMCL Lung Bhattacharjee | 5 | | | | | hsa-mir-1321 | | 0 |
| SMCL Lung Bhattacharjee | 7 | | | | | hsa-mir-615-5p | | 0 |
| SMCL Lung Bhattacharjee | 9 | | | hsa-mir-362-3p | | | | 0 |
| | | | | hsa-mir-329 | | | | |
| SMCL Lung Bhattacharjee | 34 | | | hsa-mir-1207-5p | | | | 0 |
| SMCL Lung Bhattacharjee | 47 | | | | | hsa-mir-302e | | 0 |
| SMCL Lung Bhattacharjee | 15 | | | hsa-mir-216b | Dysregulated | | | 0 |
| SMCL Lung Bhattacharjee | 29 | | | hsa-mir-513a-3p | | hsa-mir-548c-3p | | 0 |
| SMCL Lung Bhattacharjee | 11 | | | | | hsa-mir-1321 | | 0 |
| SMCL Lung Bhattacharjee | 10 | | | hsa-mir-720 | | | | 0 |
| SMCL Lung Bhattacharjee | 59 | | | | | hsa-mir-340 | | 0 |
| SMCL Lung Bhattacharjee | 58 | | | | | hsa-mir-423-5p | Dysregulated | 0 |
| SMCL Lung Bhattacharjee | 17 | | | hsa-mir-888 | | | | 0 |
| SMCL Lung Bhattacharjee | 55 | | | hsa-mir-590-3p | | | | 0 |
| SMCL Lung Bhattacharjee | 57 | | | | | hsa-mir-448 | | 0 |
| SMCL Lung Bhattacharjee | 56 | | | hsa-mir-888 | | | | 0 |
| SMCL Lung Bhattacharjee | 51 | hsa-mir-1273 | | | | | | 0 |
| | | hsa-mir-1273c | | | | | | |
| | | hsa-mir-1273d | | | | | | |
| | | hsa-mir-1273e | | | | | | |
| | | hsa-mir-1273f | | | | | | |
| | | hsa-mir-1273g | | | | | | |
| SMCL Lung Bhattacharjee | 50 | | | | | hsa-mir-664 | | 0 |
| SMCL Lung Bhattacharjee | 53 | | | | | hsa-mir-20a | Causal | 0 |
| SMCL Lung Bhattacharjee | 52 | | | hsa-mir-483-3p | | hsa-mir-939 | | 0 |
| SMCL Lung Bhattacharjee | 16 | hsa-mir-339-3p | | | | | | 0 |
| SMCL Lung Bhattacharjee | 18 | | | hsa-mir-624 | | hsa-mir-513c | | 0 |
| | | | | | | hsa-mir-513b | | |
| | | | | | | hsa-mir-513a-5p | | |
| SMCL Lung Bhattacharjee | 31 | | | | | hsa-mir-513a-3p | | 0 |
| AD Lung Stearman | 25 | | | hsa-mir-200a | Causal | hsa-mir-519a | | 0 |
| | | | | hsa-mir-141 | | | | |
| AD Lung Stearman | 27 | | | | | hsa-mir-300 | | 0 |
| AD Lung Stearman | 29 | hsa-mir-598 | | | | hsa-mir-425 | Dysregulated | 0 |
| CA Breast Richardson | 37 | | | | | hsa-mir-1289 | | 0 |
| AD Lung Stearman | 0 | | | hsa-mir-518a-5p | | hsa-mir-369-3p | | 0 |
| | | | | hsa-mir-527 | | | | |
| AD Lung Stearman | 5 | | | | | hsa-mir-342-3p | | 0 |
| AD Lung Stearman | 7 | | | hsa-mir-1269b | | hsa-mir-16 | Causal | 0 |
| | | | | hsa-mir-1269 | | | | |
| AD Lung Stearman | 6 | | | | | hsa-mir-380 | | 0 |
| AD Lung Stearman | 9 | | | hsa-mir-548g | | | | 0 |
| AD Lung Stearman | 13 | | | hsa-mir-455-5p | | hsa-mir-23a | | 0 |
| AD Lung Stearman | 19 | | | | | hsa-mir-370 | | 0 |
| AD Lung Stearman | 36 | | | | | hsa-mir-1269b | | 0 |
| | | | | | | hsa-mir-1269 | | |
| AD Lung Stearman | 35 | | | | | hsa-mir-668 | | 0 |
| AD Lung Stearman | 33 | | | hsa-mir-196b | | hsa-mir-1293 | | 0 |
| | | | | hsa-mir-196a | | | | |
| AD Lung Stearman | 32 | | | | | hsa-mir-588 | | 0 |
| FL Lymphoma Alizadeh | 11 | | | hsa-mir-148a | | hsa-mir-614 | | 0 |
| | | | | hsa-mir-148b | | | | |
| | | | | hsa-mir-152 | | | | |
| FL Lymphoma Alizadeh | 0 | | | | | hsa-mir-92a | Dysregulated | 0 |
| FL Lymphoma Alizadeh | 2 | hsa-mir-425* | | | | hsa-mir-149 | Dysregulated | 0 |
| FL Lymphoma Alizadeh | 5 | | | | | hsa-mir-581 | | 0 |
| FL Lymphoma Alizadeh | 4 | hsa-mir-4261 | | hsa-mir-1295 | | | | 0 |
| FL Lymphoma Alizadeh | 7 | | | | | hsa-mir-1282 | | 0 |
| FL Lymphoma Alizadeh | 6 | | | hsa-mir-338-5p | Dysregulated | | | 0 |
| FL Lymphoma Alizadeh | 9 | | | hsa-mir-767-3p | | hsa-mir-942 | | 0 |
| DLBCL Lymphoma Alizadeh | 11 | | | hsa-mir-1203 | | | | 0 |
| DLBCL Lymphoma Alizadeh | 10 | | | hsa-mir-325 | | | | 0 |
| DLBCL Lymphoma Alizadeh | 1 | | | hsa-mir-138 | | hsa-mir-16 | | 0 |
| DLBCL Lymphoma Alizadeh | 3 | | | | | hsa-mir-744 | | 0 |
| DLBCL Lymphoma Alizadeh | 2 | | | | | hsa-mir-660 | | 0 |
| DLBCL Lymphoma Alizadeh | 5 | hsa-mir-132* | | hsa-mir-198 | | | | 0 |
| DLBCL Lymphoma Alizadeh | 4 | | | | | hsa-mir-297 | | 0 |
| DLBCL Lymphoma Alizadeh | 7 | | | hsa-mir-532-3p | | hsa-mir-1301 | | 0 |
| DLBCL Lymphoma Alizadeh | 6 | | | hsa-mir-542-5p | | hsa-mir-483-3p | | 0 |
| DLBCL Lymphoma Alizadeh | 9 | | | hsa-mir-892b | | hsa-mir-1277 | | 0 |
| CLL Lymphoma Alizadeh | 13 | | | | | hsa-mir-423-5p | | 0 |
| CLL Lymphoma Alizadeh | 1 | | | | | hsa-mir-1204 | | 0 |
| CLL Lymphoma Alizadeh | 0 | | | hsa-mir-1283 | | hsa-mir-548j | | 0 |
| CLL Lymphoma Alizadeh | 3 | | | hsa-mir-513b | | | | 0 |
| CLL Lymphoma Alizadeh | 4 | | | | | hsa-mir-29b | Causal | 0 |
| CLL Lymphoma Alizadeh | 7 | | | hsa-mir-1263 | | | | 0 |
| CLL Lymphoma Alizadeh | 8 | | | hsa-mir-222 | | | | 0 |
| ME Melanoma Hoek | 25 | | | | | hsa-mir-26a | | 0 |
| ME Melanoma Hoek | 26 | | | | | hsa-mir-577 | | 0 |
| ME Melanoma Hoek | 20 | | | | | hsa-mir-331-5p | Dysregulated | 0 |
| OD Brain Bredel | 50 | | | | | hsa-mir-1254 | | 0 |
| ME Melanoma Hoek | 47 | | | hsa-mir-549 | | | | 0 |
| ME Melanoma Hoek | 44 | | | hsa-mir-1299 | | hsa-mir-1183 | | 0 |
| ME Melanoma Hoek | 41 | | | hsa-mir-938 | | hsa-mir-451b | | 0 |
| | | | | | | hsa-mir-451 | | |
| ME Melanoma Hoek | 0 | | | hsa-mir-34b | Dysregulated | hsa-mir-582-5p | | 0 |
| ME Melanoma Hoek | 3 | hsa-mir-4285 | | | | | | 0 |
| CA Bladder Dyrskjot | 9 | | | hsa-mir-452 | Dysregulated | hsa-mir-548c-3p | | 0 |
| ME Melanoma Hoek | 6 | | | hsa-mir-410 | | hsa-mir-186 | | 0 |
| ME Melanoma Hoek | 9 | | | hsa-mir-106b | Dysregulated | hsa-mir-494 | | 0 |
| | | | | hsa-mir-17 | | | | |
| | | | | hsa-mir-106a | | | | |
| | | | | hsa-mir-93 | | | | |
| | | | | hsa-mir-20b | | | | |
| | | | | hsa-mir-20a | | | | |
| ME Melanoma Hoek | 39 | | | hsa-mir-212 | | hsa-mir-944 | | 0 |
| | | | | hsa-mir-132 | | | | |
| ME Melanoma Hoek | 12 | | | hsa-mir-892a | | hsa-mir-935 | | 0 |
| ME Melanoma Hoek | 15 | | | hsa-mir-217 | | hsa-mir-1292 | | 0 |
| ME Melanoma Hoek | 16 | | | | | hsa-mir-631 | | 0 |
| ME Melanoma Hoek | 33 | | | | | hsa-mir-1265 | | 0 |
| ME Melanoma Hoek | 23 | | | hsa-mir-32 | | | | 0 |
| ME Melanoma Hoek | 37 | | | | | hsa-mir-380 | | 0 |
| ME Melanoma Hoek | 50 | | | hsa-mir-548c-3p | | | | 0 |
| GCT Seminoma Korkola | 92 | | | | | hsa-mir-564 | | 0 |
| ML Melanoma Talantov | 25 | | | hsa-mir-606 | | | | 0 |
| ML Melanoma Talantov | 26 | | | hsa-mir-607 | | hsa-mir-664 | | 0 |
| ML Melanoma Talantov | 27 | | | | | hsa-mir-96 | Dysregulated | 0 |
| ML Melanoma Talantov | 21 | | | | | hsa-mir-450b-5p | | 0 |
| ML Melanoma Talantov | 22 | | | hsa-mir-520d-5p | | hsa-mir-570 | | 0 |
| | | | | hsa-mir-524-5p | | | | |
| ML Melanoma Talantov | 23 | | | hsa-mir-548p | | | | 0 |
| ML Melanoma Talantov | 28 | hsa-mir-3175 | | | | hsa-mir-1207-5p | | 0 |
| ML Melanoma Talantov | 29 | | | | | hsa-mir-1257 | | 0 |
| ML Melanoma Talantov | 40 | | | | | hsa-mir-296-5p | | 0 |
| ML Melanoma Talantov | 0 | | | hsa-mir-603 | | | | 0 |
| ML Melanoma Talantov | 3 | | | hsa-mir-181a | Dysregulated | hsa-mir-548n | | 0 |
| | | | | hsa-mir-181d | | | | |
| | | | | hsa-mir-181b | | | | |
| | | | | hsa-mir-181c | | | | |
| ML Melanoma Talantov | 4 | | | | | hsa-mir-146b-3p | | 0 |
| ML Melanoma Talantov | 7 | | | hsa-mir-125b | Dysregulated | hsa-mir-296-5p | | 0 |
| | | | | hsa-mir-125a-5p | | | | |
| ML Melanoma Talantov | 9 | | | hsa-mir-151-3p | | hsa-mir-493 | | 0 |
| ML Melanoma Talantov | 8 | | | | | hsa-mir-199b-5p | Dysregulated | 0 |
| ML Melanoma Talantov | 13 | hsa-mir-337-5p | | | | | | 0 |
| ML Melanoma Talantov | 10 | | | hsa-mir-623 | | hsa-mir-1180 | | 0 |
| ML Melanoma Talantov | 12 | | | hsa-mir-125b | Dysregulated | hsa-mir-324-3p | | 0 |
| | | | | hsa-mir-125a-5p | | | | |
| ML Melanoma Talantov | 14 | | | hsa-mir-519a | | hsa-mir-548c-3p | | 0 |
| | | | | hsa-mir-519c-3p | | | | |
| | | | | hsa-mir-519b-3p | | | | |
| ML Melanoma Talantov | 19 | hsa-mir-302c* | | hsa-mir-421 | | hsa-mir-548c-3p | | 0 |
| ML Melanoma Talantov | 18 | | | | | hsa-mir-513a-3p | | 0 |
| ML Melanoma Talantov | 36 | | | | | hsa-mir-663b | | 0 |
| | | | | | | hsa-mir-663 | | |
| ML Melanoma Talantov | 34 | hsa-mir-548c-3p | | hsa-mir-767-3p | | hsa-mir-548n | | 0 |
| ML Melanoma Talantov | 33 | | | | | hsa-mir-605 | | 0 |
| MPM Mesothelioma Gordon | 56 | | | hsa-mir-595 | Dysregulated | hsa-mir-590-3p | | 0 |
| MPM Mesothelioma Gordon | 41 | | | hsa-mir-519e | | hsa-mir-381 | | 0 |
| MPM Mesothelioma Gordon | 42 | | | hsa-mir-183 | | hsa-mir-539 | | 0 |
| MPM Mesothelioma Gordon | 29 | | | | | hsa-mir-1265 | | 0 |
| MPM Mesothelioma Gordon | 61 | | | hsa-mir-545 | | hsa-mir-1287 | | 0 |
| MPM Mesothelioma Gordon | 62 | | | hsa-mir-1279 | | hsa-mir-708 | | 0 |
| MPM Mesothelioma Gordon | 36 | | | | | hsa-mir-487a | | 0 |
| MPM Mesothelioma Gordon | 64 | | | | | hsa-mir-655 | | 0 |
| MPM Mesothelioma Gordon | 65 | | | hsa-mir-590-3p | | hsa-mir-548n | | 0 |
| MPM Mesothelioma Gordon | 52 | | | | | hsa-mir-606 | | 0 |
| MPM Mesothelioma Gordon | 24 | | | | | hsa-mir-361-3p | | 0 |
| MPM Mesothelioma Gordon | 27 | | | hsa-mir-1301 | | hsa-mir-128 | | 0 |
| MPM Mesothelioma Gordon | 21 | | | | | hsa-mir-548c-3p | | 0 |
| MPM Mesothelioma Gordon | 22 | hsa-mir-1284 | | | | | | 0 |
| MPM Mesothelioma Gordon | 44 | | | | | hsa-mir-338-5p | | 0 |
| MPM Mesothelioma Gordon | 48 | | | hsa-mir-561 | | hsa-mir-606 | | 0 |
| MPM Mesothelioma Gordon | 28 | | | | | hsa-mir-590-3p | | 0 |
| MPM Mesothelioma Gordon | 40 | hsa-mir-3178 | | | | | | 0 |
| MPM Mesothelioma Gordon | 1 | | | | | hsa-mir-1224-5p | | 0 |
| MPM Mesothelioma Gordon | 2 | | | | | hsa-mir-1207-5p | | 0 |
| MPM Mesothelioma Gordon | 5 | | | | | hsa-mir-603 | | 0 |
| MPM Mesothelioma Gordon | 7 | | | hsa-mir-526b | | | | 0 |
| MPM Mesothelioma Gordon | 6 | | | | | hsa-mir-192 | | 0 |
| MPM Mesothelioma Gordon | 9 | hsa-mir-219-5p | | | | | | 0 |
| MPM Mesothelioma Gordon | 8 | | | | | hsa-mir-1180 | | 0 |
| MPM Mesothelioma Gordon | 35 | | | hsa-mir-371b-5p | | | | 0 |
| | | | | hsa-mir-371-5p | | | | |
| MPM Mesothelioma Gordon | 13 | hsa-mir-1250 | | | | hsa-mir-585 | | 0 |
| MPM Mesothelioma Gordon | 38 | | | hsa-mir-1276 | | hsa-mir-145 | | 0 |
| MPM Mesothelioma Gordon | 59 | | | | | hsa-mir-423-5p | Dysregulated | 0 |
| MPM Mesothelioma Gordon | 14 | | | hsa-mir-361-3p | | | | 0 |
| MPM Mesothelioma Gordon | 11 | | | hsa-mir-491-3p | | hsa-mir-889 | | 0 |
| MPM Mesothelioma Gordon | 15 | | | | | hsa-mir-1229 | | 0 |
| MPM Mesothelioma Gordon | 17 | | | | | hsa-mir-1296 | | 0 |
| MPM Mesothelioma Gordon | 16 | | | hsa-mir-548m | | hsa-mir-590-3p | | 0 |
| MPM Mesothelioma Gordon | 54 | | | | | hsa-mir-548h | | 0 |
| MPM Mesothelioma Gordon | 51 | | | hsa-mir-605 | | hsa-mir-376a | | 0 |
| MPM Mesothelioma Gordon | 53 | | | hsa-mir-1182 | | | | 0 |
| MPM Mesothelioma Gordon | 19 | hsa-mir-615-5p | | | | hsa-mir-423-5p | Dysregulated | 0 |
| MPM Mesothelioma Gordon | 63 | | | hsa-mir-548b-5p | | hsa-mir-548c-3p | | 0 |
| | | | | hsa-mir-548am | | | | |
| | | | | hsa-mir-548ab | | | | |
| | | | | hsa-mir-548aa | | | | |
| | | | | hsa-mir-548ag | | | | |
| | | | | hsa-mir-548ak | | | | |
| | | | | hsa-mir-548aj | | | | |
| | | | | hsa-mir-548ai | | | | |
| | | | | hsa-mir-548ah | | | | |
| | | | | hsa-mir-548an | | | | |
| | | | | hsa-mir-548al | | | | |
| | | | | hsa-mir-548ae | | | | |
| | | | | hsa-mir-548ad | | | | |
| | | | | hsa-mir-548a-5p | | | | |
| | | | | hsa-mir-548ac | | | | |
| | | | | hsa-mir-548d-5p | | | | |
| | | | | hsa-mir-548i | | | | |
| | | | | hsa-mir-548j | | | | |
| | | | | hsa-mir-548c-5p | | | | |
| | | | | hsa-mir-548h | | | | |
| MPM Mesothelioma Gordon | 32 | | | | | hsa-mir-541 | | 0 |
| MPM Mesothelioma Gordon | 31 | | | | | hsa-mir-127-5p | Causal | 0 |
| MM Myeloma Zhan | 45 | | | hsa-mir-595 | | | | 0 |
| MM Myeloma Zhan | 22 | | | hsa-mir-1236 | | | | 0 |
| MM Myeloma Zhan | 36 | | | | | hsa-mir-1273 | | 0 |
| | | | | | | hsa-mir-1273c | | |
| | | | | | | hsa-mir-1273d | | |
| | | | | | | hsa-mir-1273e | | |
| | | | | | | hsa-mir-1273f | | |
| | | | | | | hsa-mir-1273g | | |
| MM Myeloma Zhan | 24 | | | hsa-mir-140-3p | Dysregulated | | | 0 |
| MM Myeloma Zhan | 25 | | | hsa-mir-188-5p | | | | 0 |
| MM Myeloma Zhan | 26 | | | | | hsa-mir-1321 | | 0 |
| MM Myeloma Zhan | 20 | | | hsa-mir-1226 | | | | 0 |
| MM Myeloma Zhan | 21 | | | hsa-mir-183 | | | | 0 |
| MM Myeloma Zhan | 49 | | | | | hsa-mir-138 | | 0 |
| MM Myeloma Zhan | 46 | | | hsa-mir-425 | | | | 0 |
| MM Myeloma Zhan | 47 | | | hsa-mir-875-3p | | hsa-mir-662 | | 0 |
| MM Myeloma Zhan | 43 | | | hsa-mir-23b | | hsa-mir-124 | Causal | 0 |
| | | | | hsa-mir-23a | | | | |
| MM Myeloma Zhan | 40 | | | hsa-mir-382 | | hsa-mir-612 | | 0 |
| MM Myeloma Zhan | 41 | | | | | hsa-mir-339-5p | | 0 |
| MM Myeloma Zhan | 1 | | | hsa-mir-519e | | | | 0 |
| MM Myeloma Zhan | 0 | | | | | hsa-mir-1207-5p | | 0 |
| MM Myeloma Zhan | 3 | | | hsa-mir-590-3p | | hsa-mir-548c-3p | | 0 |
| MM Myeloma Zhan | 2 | | | hsa-mir-587 | | | | 0 |
| MM Myeloma Zhan | 4 | | | hsa-mir-361-3p | | hsa-mir-185 | | 0 |
| MM Myeloma Zhan | 7 | | | hsa-mir-510 | | hsa-mir-645 | | 0 |
| MM Myeloma Zhan | 6 | | | hsa-mir-516b | | hsa-mir-600 | | 0 |
| MM Myeloma Zhan | 9 | | | hsa-mir-9 | | hsa-mir-185 | | 0 |
| MM Myeloma Zhan | 39 | | | | | hsa-mir-647 | | 0 |
| MM Myeloma Zhan | 38 | | | | | hsa-mir-652 | | 0 |
| MM Myeloma Zhan | 29 | | | hsa-mir-760 | | | | 0 |
| MM Myeloma Zhan | 11 | | | | | hsa-mir-658 | | 0 |
| AD Lung Beer | 10 | | | | | hsa-mir-600 | | 0 |
| MM Myeloma Zhan | 13 | | | hsa-mir-655 | | hsa-mir-770-5p | | 0 |
| MM Myeloma Zhan | 15 | | | | | hsa-mir-922 | | 0 |
| CA Bladder Dyrskjot | 62 | | | | | hsa-mir-637 | | 0 |
| MM Myeloma Zhan | 17 | | | hsa-mir-320e | | hsa-mir-495 | | 0 |
| | | | | hsa-mir-320d | | | | |
| | | | | hsa-mir-320c | | | | |
| | | | | hsa-mir-320b | | | | |
| | | | | hsa-mir-320a | | | | |
| MM Myeloma Zhan | 16 | | | | | hsa-mir-134 | | 0 |
| MM Myeloma Zhan | 32 | | | hsa-mir-212 | | hsa-mir-379 | | 0 |
| | | | | hsa-mir-132 | | | | |
| MM Myeloma Zhan | 31 | | | hsa-mir-597 | | hsa-mir-512-3p | | 0 |
| MM Myeloma Zhan | 30 | | | hsa-mir-588 | | | | 0 |
| MM Myeloma Zhan | 50 | | | | | hsa-mir-28-5p | | 0 |
| MM Myeloma Zhan | 35 | | | hsa-mir-650 | | hsa-mir-129-5p | | 0 |
| MM Myeloma Zhan | 34 | | | | | hsa-mir-147 | | 0 |
| | | | | | | hsa-mir-147b | | |
| MM Myeloma Zhan | 33 | | | | | hsa-mir-507 | | 0 |
| AD Ovarian Welsh | 24 | hsa-mir-1469 | | | | | | 0 |
| AD Ovarian Welsh | 26 | | | hsa-mir-210 | | | | 0 |
| AD Ovarian Welsh | 20 | | | hsa-mir-767-5p | | | | 0 |
| AD Ovarian Welsh | 21 | | | hsa-mir-96 | | hsa-mir-606 | | 0 |
| AD Ovarian Welsh | 22 | | | | | hsa-mir-1207-5p | | 0 |
| AD Ovarian Welsh | 29 | hsa-mir-3194-5p | | hsa-mir-1280 | | hsa-mir-1225-3p | | 0 |
| AD Ovarian Welsh | 1 | | | | | hsa-mir-548d-3p | | 0 |
| AD Ovarian Welsh | 0 | | | hsa-mir-510 | | hsa-mir-200b | Causal | 0 |
| AD Ovarian Welsh | 3 | | | hsa-mir-1283 | | | | 0 |
| AD Ovarian Welsh | 2 | | | hsa-mir-122 | | | | 0 |
| AD Ovarian Welsh | 5 | | | hsa-mir-188-3p | | hsa-mir-455-5p | | 0 |
| AD Ovarian Welsh | 4 | | | | | hsa-mir-424 | Dysregulated | 0 |
| AD Ovarian Welsh | 7 | | | hsa-mir-650 | | | | 0 |
| AD Ovarian Welsh | 6 | hsa-let-7e* | | | | | | 0 |
| AD Ovarian Welsh | 9 | | | | | hsa-mir-524-5p | | 0 |
| AD Ovarian Welsh | 8 | | | | | hsa-mir-1260 | | 0 |
| | | | | | | hsa-mir-1260b | | |
| AD Ovarian Welsh | 11 | | | hsa-mir-1323 | | hsa-mir-1225-5p | | 0 |
| | | | | hsa-mir-548o | | | | |
| AD Ovarian Welsh | 10 | | | | | hsa-mir-921 | | 0 |
| AD Ovarian Welsh | 13 | hsa-mir-3178 | | | | | | 0 |
| CA Bladder Dyrskjot | 77 | | | | | hsa-mir-620 | | 0 |
| AD Ovarian Welsh | 15 | | | hsa-mir-1282 | | | | 0 |
| AD Ovarian Welsh | 14 | | | | | hsa-mir-338-5p | | 0 |
| AD Ovarian Welsh | 17 | | | | | hsa-mir-1321 | | 0 |
| AD Ovarian Welsh | 16 | | | | | hsa-mir-548l | | 0 |
| AD Ovarian Welsh | 31 | | | hsa-mir-380 | | hsa-mir-548c-3p | | 0 |
| AD Ovarian Welsh | 30 | | | hsa-mir-1207-5p | | hsa-mir-1204 | | 0 |
| CCC Ovarian Hendrix | 56 | | | hsa-mir-409-3p | | hsa-mir-587 | | 0 |
| CCC Ovarian Hendrix | 51 | | | hsa-mir-1279 | | hsa-mir-499-3p | | 0 |
| CCC Ovarian Hendrix | 60 | | | hsa-mir-105 | Dysregulated | hsa-mir-1305 | | 0 |
| CCC Ovarian Hendrix | 61 | | | | | hsa-mir-145 | Dysregulated | 0 |
| CCC Ovarian Hendrix | 24 | | | hsa-mir-19b | | | | 0 |
| | | | | hsa-mir-19a | | | | |
| CCC Ovarian Hendrix | 25 | | | hsa-mir-183 | Dysregulated | hsa-mir-744 | | 0 |
| CCC Ovarian Hendrix | 27 | | | | | hsa-mir-556-5p | | 0 |
| CCC Ovarian Hendrix | 20 | | | | | hsa-mir-495 | Causal | 0 |
| CCC Ovarian Hendrix | 23 | hsa-mir-523 | | hsa-mir-105 | Dysregulated | | | 0 |
| CCC Ovarian Hendrix | 46 | | | | | hsa-mir-340 | | 0 |
| CCC Ovarian Hendrix | 47 | | | hsa-mir-548p | | hsa-mir-122 | | 0 |
| CCC Ovarian Hendrix | 44 | | | | | hsa-mir-548m | | 0 |
| CCC Ovarian Hendrix | 45 | | | hsa-mir-382 | | hsa-mir-1250 | | 0 |
| CCC Ovarian Hendrix | 29 | | | | | hsa-mir-636 | | 0 |
| CCC Ovarian Hendrix | 1 | | | hsa-mir-1200 | | | | 0 |
| CCC Ovarian Hendrix | 0 | | | | | hsa-mir-194 | | 0 |
| CCC Ovarian Hendrix | 2 | | | | | hsa-mir-423-5p | | 0 |
| CCC Ovarian Hendrix | 7 | | | hsa-mir-532-5p | | hsa-mir-382 | | 0 |
| CCC Ovarian Hendrix | 9 | | | | | hsa-mir-1291 | | 0 |
| CCC Ovarian Hendrix | 43 | | | | | hsa-mir-1247 | | 0 |
| CCC Ovarian Hendrix | 14 | | | | | hsa-mir-1276 | | 0 |
| CCC Ovarian Hendrix | 11 | hsa-mir-638 | | hsa-mir-29b | Dysregulated | hsa-mir-602 | | 0 |
| | | | | hsa-mir-29c | | | | |
| | | | | hsa-mir-29a | | | | |
| CCC Ovarian Hendrix | 10 | | | | | hsa-mir-615-5p | | 0 |
| CCC Ovarian Hendrix | 13 | | | hsa-mir-143 | Dysregulated | hsa-mir-500b | | 0 |
| | | | | | | hsa-mir-500a | | |
| CCC Ovarian Hendrix | 12 | | | hsa-mir-876-5p | | hsa-mir-508-3p | | 0 |
| CCC Ovarian Hendrix | 59 | | | hsa-mir-552 | | hsa-mir-1258 | | 0 |
| CCC Ovarian Hendrix | 58 | | | | | hsa-mir-586 | | 0 |
| CCC Ovarian Hendrix | 55 | hsa-mir-1471 | | | | | | 0 |
| CCC Ovarian Hendrix | 30 | | | hsa-mir-590-5p | | | | 0 |
| CCC Ovarian Hendrix | 37 | | | hsa-mir-1302 | | hsa-mir-1237 | | 0 |
| CCC Ovarian Hendrix | 35 | | | hsa-mir-1283 | | hsa-mir-607 | | 0 |
| CCC Ovarian Hendrix | 33 | | | hsa-mir-127-5p | Causal | hsa-mir-770-5p | | 0 |
| CA Breast Sorlie | 23 | | | | | hsa-mir-423-5p | | 0 |
| CCC Ovarian Hendrix | 18 | | | | | hsa-mir-326 | | 0 |
| CCC Ovarian Hendrix | 57 | | | | | hsa-mir-590-5p | | 0 |
| MUC Ovarian Hendrix | 30 | | | | | hsa-mir-635 | Dysregulated | 0 |
| MUC Ovarian Hendrix | 54 | | | | | hsa-mir-921 | | 0 |
| MUC Ovarian Hendrix | 28 | | | | | hsa-mir-296-5p | Dysregulated | 0 |
| MUC Ovarian Hendrix | 45 | | | hsa-mir-765 | | | | 0 |
| MUC Ovarian Hendrix | 60 | | | hsa-mir-369-3p | | hsa-mir-574-3p | | 0 |
| MUC Ovarian Hendrix | 61 | | | hsa-mir-940 | | hsa-mir-410 | | 0 |
| B-CLL Leukemia Haslinger | 12 | | | | | hsa-mir-1302 | | 0 |
| MUC Ovarian Hendrix | 63 | | | hsa-mir-184 | Dysregulated | hsa-mir-1247 | | 0 |
| GCT Seminoma Korkola | 71 | | | | | hsa-mir-361-3p | | 0 |
| MUC Ovarian Hendrix | 65 | | | | | hsa-mir-380 | | 0 |
| MUC Ovarian Hendrix | 66 | | | | | hsa-mir-889 | | 0 |
| MUC Ovarian Hendrix | 67 | | | hsa-mir-1224-3p | | | | 0 |
| MUC Ovarian Hendrix | 68 | | | hsa-mir-606 | | hsa-mir-1305 | | 0 |
| MUC Ovarian Hendrix | 69 | | | | | hsa-mir-579 | | 0 |
| MUC Ovarian Hendrix | 52 | | | | | hsa-mir-541 | | 0 |
| MUC Ovarian Hendrix | 24 | | | | | hsa-mir-361-3p | | 0 |
| MUC Ovarian Hendrix | 25 | | | hsa-mir-548c-3p | | hsa-mir-582-5p | | 0 |
| MUC Ovarian Hendrix | 26 | | | | | hsa-mir-296-5p | Dysregulated | 0 |
| MUC Ovarian Hendrix | 27 | | | | | hsa-mir-423-5p | | 0 |
| MUC Ovarian Hendrix | 20 | | | | | hsa-mir-640 | | 0 |
| MUC Ovarian Hendrix | 21 | | | hsa-mir-216b | | hsa-mir-520d-5p | | 0 |
| MUC Ovarian Hendrix | 22 | | | | | hsa-mir-1293 | | 0 |
| MUC Ovarian Hendrix | 23 | | | | | hsa-mir-486-3p | | 0 |
| MUC Ovarian Hendrix | 47 | | | | | hsa-mir-654-5p | | 0 |
| MUC Ovarian Hendrix | 44 | | | hsa-mir-217 | | | | 0 |
| MUC Ovarian Hendrix | 48 | | | | | hsa-mir-608 | Dysregulated | 0 |
| MUC Ovarian Hendrix | 42 | hsa-mir-1181 | | | | | | 0 |
| MUC Ovarian Hendrix | 40 | | | | | hsa-mir-618 | | 0 |
| MUC Ovarian Hendrix | 41 | | | hsa-mir-34a | Causal | | | 0 |
| | | | | hsa-mir-34c-5p | | | | |
| | | | | hsa-mir-449a | | | | |
| | | | | hsa-mir-449b | | | | |
| MUC Ovarian Hendrix | 0 | | | hsa-mir-556-3p | | hsa-mir-410 | | 0 |
| MUC Ovarian Hendrix | 3 | | | | | hsa-mir-1205 | | 0 |
| MUC Ovarian Hendrix | 2 | | | hsa-mir-22 | | | | 0 |
| MUC Ovarian Hendrix | 7 | | | hsa-mir-607 | | hsa-mir-590-3p | | 0 |
| TU Prostate Lapointe | 32 | | | | | hsa-mir-608 | | 0 |
| MUC Ovarian Hendrix | 8 | | | hsa-mir-1226 | | hsa-mir-486-3p | | 0 |
| MUC Ovarian Hendrix | 56 | hsa-mir-508-3p | | | | hsa-mir-548c-3p | | 0 |
| MUC Ovarian Hendrix | 19 | | | | | hsa-mir-548g | | 0 |
| MUC Ovarian Hendrix | 76 | | | hsa-mir-144 | | hsa-mir-573 | | 0 |
| MUC Ovarian Hendrix | 75 | | | | | hsa-mir-512-3p | | 0 |
| MUC Ovarian Hendrix | 74 | | | hsa-mir-27a | | | | 0 |
| | | | | hsa-mir-27b | | | | |
| MUC Ovarian Hendrix | 73 | | | | | hsa-mir-361-3p | | 0 |
| MUC Ovarian Hendrix | 72 | | | | | hsa-mir-409-5p | | 0 |
| MUC Ovarian Hendrix | 71 | | | hsa-mir-361-3p | | | | 0 |
| MUC Ovarian Hendrix | 14 | | | hsa-mir-148a | Dysregulated | | | 0 |
| | | | | hsa-mir-148b | | | | |
| | | | | hsa-mir-152 | | | | |
| AD Pancreas Logsdon | 1 | | | | | hsa-mir-1247 | | 0 |
| MUC Ovarian Hendrix | 15 | | | hsa-mir-340 | | hsa-mir-944 | | 0 |
| MUC Ovarian Hendrix | 55 | | | | | hsa-mir-486-3p | | 0 |
| MUC Ovarian Hendrix | 32 | | | | | hsa-mir-490-3p | | 0 |
| MUC Ovarian Hendrix | 57 | | | hsa-mir-766 | | | | 0 |
| MUC Ovarian Hendrix | 49 | | | | | hsa-mir-200b | Causal | 0 |
| AD Lung Bhattacharjee | 16 | | | | | hsa-mir-361-3p | | 0 |
| MUC Ovarian Hendrix | 34 | | | | | hsa-mir-1291 | | 0 |
| MUC Ovarian Hendrix | 18 | | | hsa-mir-548n | | hsa-mir-606 | | 0 |
| MUC Ovarian Hendrix | 12 | | | | | hsa-mir-658 | | 0 |
| MUC Ovarian Hendrix | 31 | | | | | hsa-mir-342-5p | | 0 |
| MUC Ovarian Hendrix | 50 | | | | | hsa-mir-889 | | 0 |
| SRS Ovarian Hendrix | 36 | | | | | hsa-mir-890 | | 0 |
| SRS Ovarian Hendrix | 42 | | | | | hsa-mir-1298 | | 0 |
| SRS Ovarian Hendrix | 22 | | | hsa-mir-624 | | | | 0 |
| SRS Ovarian Hendrix | 60 | | | hsa-mir-188-3p | | hsa-mir-184 | Dysregulated | 0 |
| SRS Ovarian Hendrix | 64 | | | | | hsa-mir-377 | Causal | 0 |
| SRS Ovarian Hendrix | 66 | | | hsa-mir-892b | | hsa-mir-548c-3p | | 0 |
| SRS Ovarian Hendrix | 67 | | | | | hsa-mir-608 | Dysregulated | 0 |
| SRS Ovarian Hendrix | 68 | | | | | hsa-mir-200b | Causal | 0 |
| SRS Ovarian Hendrix | 83 | | | | | hsa-mir-637 | Dysregulated | 0 |
| SRS Ovarian Hendrix | 80 | | | | | hsa-mir-324-5p | | 0 |
| SRS Ovarian Hendrix | 81 | hsa-mir-3135b | | | | | | 0 |
| | | hsa-mir-3135 | | | | | | |
| SRS Ovarian Hendrix | 24 | | | | | hsa-mir-635 | Dysregulated | 0 |
| SRS Ovarian Hendrix | 26 | | | | | hsa-mir-325 | | 0 |
| SRS Ovarian Hendrix | 27 | | | hsa-mir-362-5p | | | | 0 |
| SRS Ovarian Hendrix | 20 | | | | | hsa-mir-296-5p | Dysregulated | 0 |
| SRS Ovarian Hendrix | 21 | | | | | hsa-mir-944 | | 0 |
| SRS Ovarian Hendrix | 48 | | | | | hsa-mir-1278 | | 0 |
| SRS Ovarian Hendrix | 44 | | | | | hsa-mir-584 | | 0 |
| SRS Ovarian Hendrix | 45 | | | hsa-mir-146b-3p | | | | 0 |
| SRS Ovarian Hendrix | 28 | | | | | hsa-mir-186 | | 0 |
| SRS Ovarian Hendrix | 43 | | | | | hsa-mir-299-3p | | 0 |
| SRS Ovarian Hendrix | 41 | | | hsa-mir-650 | | | | 0 |
| SRS Ovarian Hendrix | 1 | | | hsa-mir-641 | | hsa-mir-409-3p | | 0 |
| SRS Ovarian Hendrix | 3 | | | | | hsa-mir-146b-3p | | 0 |
| SRS Ovarian Hendrix | 2 | | | hsa-mir-505 | | | | 0 |
| SRS Ovarian Hendrix | 4 | | | | | hsa-mir-1250 | | 0 |
| SRS Ovarian Hendrix | 6 | | | | | hsa-mir-185 | | 0 |
| SRS Ovarian Hendrix | 9 | | | | | hsa-mir-1297 | | 0 |
| SRS Ovarian Hendrix | 19 | | | | | hsa-mir-135a | | 0 |
| SRS Ovarian Hendrix | 75 | hsa-mir-937 | | | | hsa-mir-372 | | 0 |
| OD Brain Bredel | 40 | | | | | hsa-mir-216b | | 0 |
| SRS Ovarian Hendrix | 17 | | | | | hsa-mir-193b | | 0 |
| SRS Ovarian Hendrix | 70 | | | hsa-mir-641 | | hsa-mir-433 | | 0 |
| SRS Ovarian Hendrix | 15 | | | hsa-mir-1293 | | hsa-let-7d | Causal | 0 |
| SRS Ovarian Hendrix | 69 | | | hsa-mir-330-3p | | hsa-mir-29b | | 0 |
| SRS Ovarian Hendrix | 79 | | | | | hsa-mir-944 | | 0 |
| SRS Ovarian Hendrix | 78 | | | | | hsa-mir-206 | Dysregulated | 0 |
| SRS Ovarian Hendrix | 11 | | | hsa-mir-361-5p | Dysregulated | hsa-mir-590-3p | | 0 |
| SRS Ovarian Hendrix | 10 | | | | | hsa-mir-34c-3p | | 0 |
| SRS Ovarian Hendrix | 38 | | | | | hsa-mir-17 | | 0 |
| SRS Ovarian Hendrix | 59 | | | | | hsa-mir-656 | | 0 |
| SRS Ovarian Hendrix | 14 | | | | | hsa-mir-1290 | | 0 |
| SRS Ovarian Hendrix | 61 | | | | | hsa-mir-1224-3p | | 0 |
| SRS Ovarian Hendrix | 54 | | | hsa-mir-448 | | | | 0 |
| SRS Ovarian Hendrix | 30 | | | hsa-mir-424 | Dysregulated | hsa-mir-582-5p | | 0 |
| SRS Ovarian Hendrix | 53 | | | hsa-mir-376b | Causal | hsa-mir-576-5p | | 0 |
| | | | | hsa-mir-376a | | | | |
| SRS Ovarian Hendrix | 52 | | | hsa-mir-626 | | | | 0 |
| SRS Ovarian Hendrix | 55 | | | hsa-mir-548l | | hsa-mir-338-5p | | 0 |
| SRS Ovarian Hendrix | 16 | | | | | hsa-mir-548k | | 0 |
| SRS Ovarian Hendrix | 32 | | | | | hsa-mir-331-3p | | 0 |
| SRS Ovarian Hendrix | 12 | | | | | hsa-mir-125a-3p | | 0 |
| SRS Ovarian Hendrix | 57 | | | hsa-mir-561 | | hsa-mir-548d-3p | | 0 |
| SRS Ovarian Hendrix | 72 | | | hsa-mir-1276 | | | | 0 |
| END Ovarian Hendrix | 37 | | | | | hsa-mir-125b | Dysregulated | 0 |
| END Ovarian Hendrix | 60 | | | hsa-mir-580 | | hsa-mir-570 | | 0 |
| END Ovarian Hendrix | 62 | | | | | hsa-mir-571 | | 0 |
| END Ovarian Hendrix | 66 | | | | | hsa-mir-101 | Dysregulated | 0 |
| END Ovarian Hendrix | 82 | hsa-let-7d* | | hsa-mir-505 | | hsa-mir-320b | | 0 |
| END Ovarian Hendrix | 32 | | | hsa-mir-802 | | hsa-mir-548d-3p | | 0 |
| END Ovarian Hendrix | 25 | | | hsa-mir-653 | | | | 0 |
| END Ovarian Hendrix | 26 | | | hsa-mir-548c-3p | | hsa-mir-656 | | 0 |
| END Ovarian Hendrix | 20 | | | | | hsa-mir-663b | Dysregulated | 0 |
| | | | | | | hsa-mir-663 | | |
| END Ovarian Hendrix | 48 | | | hsa-mir-125a-3p | | hsa-mir-1304 | | 0 |
| END Ovarian Hendrix | 23 | | | | | hsa-mir-22 | | 0 |
| END Ovarian Hendrix | 47 | hsa-mir-1471 | | | | | | 0 |
| END Ovarian Hendrix | 45 | hsa-mir-1471 | | | | | | 0 |
| END Ovarian Hendrix | 43 | | | | | hsa-mir-590-3p | | 0 |
| END Ovarian Hendrix | 40 | | | | | hsa-mir-944 | | 0 |
| END Ovarian Hendrix | 41 | | | | | hsa-mir-338-5p | | 0 |
| IDC Breast Radvanyi | 46 | | | | | hsa-mir-675 | | 0 |
| END Ovarian Hendrix | 0 | hsa-mir-598 | | | | | | 0 |
| END Ovarian Hendrix | 3 | | | hsa-mir-510 | | hsa-mir-1279 | | 0 |
| END Ovarian Hendrix | 4 | | | hsa-mir-608 | Dysregulated | hsa-mir-612 | | 0 |
| END Ovarian Hendrix | 7 | | | hsa-mir-1302 | | | | 0 |
| END Ovarian Hendrix | 6 | | | hsa-mir-29b | Dysregulated | hsa-mir-890 | | 0 |
| | | | | hsa-mir-29c | | | | |
| | | | | hsa-mir-29a | | | | |
| END Ovarian Hendrix | 9 | | | | | hsa-mir-486-3p | | 0 |
| END Ovarian Hendrix | 8 | | | hsa-mir-200a | Causal | | | 0 |
| | | | | hsa-mir-141 | | | | |
| END Ovarian Hendrix | 52 | hsa-mir-1538 | | | | | | 0 |
| END Ovarian Hendrix | 28 | | | | | hsa-mir-495 | Causal | 0 |
| END Ovarian Hendrix | 78 | | | | | hsa-mir-125a-5p | Causal | 0 |
| END Ovarian Hendrix | 19 | hsa-mir-3195 | | | | | | 0 |
| END Ovarian Hendrix | 39 | | | hsa-mir-508-5p | Dysregulated | | | 0 |
| CA Bladder Dyrskjot | 39 | | | | | hsa-mir-486-3p | | 0 |
| END Ovarian Hendrix | 76 | | | | | hsa-mir-888 | | 0 |
| END Ovarian Hendrix | 75 | | | | | hsa-mir-489 | | 0 |
| END Ovarian Hendrix | 72 | | | | | hsa-mir-548d-3p | | 0 |
| END Ovarian Hendrix | 70 | | | | | hsa-mir-939 | | 0 |
| END Ovarian Hendrix | 15 | | | | | hsa-mir-542-3p | Dysregulated | 0 |
| END Ovarian Hendrix | 69 | | | hsa-mir-607 | | hsa-mir-186 | | 0 |
| END Ovarian Hendrix | 29 | | | | | hsa-mir-944 | | 0 |
| END Ovarian Hendrix | 58 | hsa-mir-621 | | | | | | 0 |
| END Ovarian Hendrix | 11 | | | | | hsa-mir-940 | | 0 |
| END Ovarian Hendrix | 13 | | | hsa-mir-1276 | | hsa-mir-23a | | 0 |
| END Ovarian Hendrix | 38 | | | hsa-mir-944 | | hsa-mir-944 | | 0 |
| END Ovarian Hendrix | 22 | | | hsa-mir-15b | Dysregulated | hsa-mir-944 | | 0 |
| | | | | hsa-mir-15a | | | | |
| | | | | hsa-mir-16 | | | | |
| | | | | hsa-mir-497 | | | | |
| | | | | hsa-mir-195 | | | | |
| END Ovarian Hendrix | 17 | | | | | hsa-mir-30e | Dysregulated | 0 |
| END Ovarian Hendrix | 61 | | | | | hsa-mir-342-5p | | 0 |
| END Ovarian Hendrix | 31 | | | | | hsa-mir-1253 | | 0 |
| END Ovarian Hendrix | 49 | | | | | hsa-mir-145 | Dysregulated | 0 |
| END Ovarian Hendrix | 51 | | | hsa-mir-130b | | | | 0 |
| | | | | hsa-mir-301a | | | | |
| | | | | hsa-mir-301b | | | | |
| | | | | hsa-mir-130a | | | | |
| | | | | hsa-mir-454 | | | | |
| END Ovarian Hendrix | 50 | | | hsa-mir-539 | | hsa-mir-548c-3p | | 0 |
| END Ovarian Hendrix | 16 | | | | | hsa-mir-588 | | 0 |
| END Ovarian Hendrix | 18 | hsa-mir-487b | Dysregulated | hsa-mir-890 | | | | 0 |
| END Ovarian Hendrix | 65 | | | | | hsa-mir-331-3p | | 0 |
| PDC Pancreas Ishikawa | 1 | | | | | hsa-mir-106b | | 0 |
| PDC Pancreas Ishikawa | 3 | hsa-mir-369-5p | | | | | | 0 |
| PDC Pancreas Ishikawa | 2 | | | | | hsa-mir-448 | | 0 |
| AD Pancreas Logsdon | 10 | hsa-mir-1273 | | | | | | 0 |
| | | hsa-mir-1273c | | | | | | |
| | | hsa-mir-1273d | | | | | | |
| | | hsa-mir-1273e | | | | | | |
| | | hsa-mir-1273f | | | | | | |
| | | hsa-mir-1273g | | | | | | |
| AD Pancreas Logsdon | 13 | | | hsa-mir-486-5p | | | | 0 |
| AD Pancreas Logsdon | 12 | hsa-let-7d | Dysregulated | | | hsa-mir-933 | | 0 |
| | | hsa-let-7f | | | | | | |
| | | hsa-mir-98 | | | | | | |
| | | hsa-let-7b | | | | | | |
| | | hsa-let-7c | | | | | | |
| | | hsa-let-7a | | | | | | |
| | | hsa-let-7g | | | | | | |
| | | hsa-let-7e | | | | | | |
| | | hsa-let-7i | | | | | | |
| AD Pancreas Logsdon | 15 | | | hsa-mir-556-3p | | hsa-mir-130b | | 0 |
| AD Pancreas Logsdon | 14 | | | hsa-mir-517c | | | | 0 |
| | | | | hsa-mir-517b | | | | |
| | | | | hsa-mir-517a | | | | |
| AD Pancreas Logsdon | 16 | | | hsa-mir-561 | | | | 0 |
| CA Bladder Dyrskjot | 59 | | | | | hsa-mir-374a | | 0 |
| AD Pancreas Logsdon | 18 | | | | | hsa-mir-939 | | 0 |
| RCCC Renal Boer | 5 | | | | | hsa-mir-374a | | 0 |
| AD Pancreas Logsdon | 3 | hsa-mir-3178 | | | | | | 0 |
| AD Pancreas Logsdon | 4 | | | | | hsa-mir-22 | | 0 |
| MPC Prostate Dhanasekaran | 30 | | | hsa-mir-659 | | | | 0 |
| MPC Prostate Dhanasekaran | 29 | | | | | hsa-mir-663b | | 0 |
| | | | | | | hsa-mir-663 | | |
| MPC Prostate Dhanasekaran | 26 | | | hsa-mir-935 | | hsa-mir-548j | | 0 |
| MPC Prostate Dhanasekaran | 27 | | | | | hsa-mir-1271 | | 0 |
| MPC Prostate Dhanasekaran | 21 | | | hsa-mir-498 | Dysregulated | | | 0 |
| MPC Prostate Dhanasekaran | 48 | | | | | hsa-mir-625 | | 0 |
| MPC Prostate Dhanasekaran | 44 | | | hsa-mir-1323 | | hsa-mir-576-3p | | 0 |
| | | | | hsa-mir-548o | | | | |
| MPC Prostate Dhanasekaran | 45 | | | hsa-mir-490-3p | | | | 0 |
| MPC Prostate Dhanasekaran | 42 | hsa-mir-582-3p | | | | hsa-mir-331-5p | | 0 |
| MPC Prostate Dhanasekaran | 0 | hsa-mir-3074-5p | | hsa-mir-1323 | | hsa-mir-200b | Causal | 0 |
| | | | | hsa-mir-548o | | | | |
| MPC Prostate Dhanasekaran | 2 | | | | | hsa-mir-548c-3p | | 0 |
| MPC Prostate Dhanasekaran | 5 | | | | | hsa-mir-211 | | 0 |
| MPC Prostate Dhanasekaran | 7 | | | hsa-mir-330-5p | Causal | hsa-mir-296-5p | Dysregulated | 0 |
| | | | | hsa-mir-326 | | | | |
| MPC Prostate Dhanasekaran | 9 | | | hsa-mir-190b | | | | 0 |
| | | | | hsa-mir-190 | | | | |
| MPC Prostate Dhanasekaran | 8 | | | | | hsa-mir-219-1-3p | | 0 |
| MPC Prostate Dhanasekaran | 13 | | | hsa-mir-1257 | | | | 0 |
| MPC Prostate Dhanasekaran | 12 | | | hsa-mir-139-5p | | hsa-mir-340 | | 0 |
| MPC Prostate Dhanasekaran | 14 | | | hsa-mir-133b | | hsa-mir-502-5p | | 0 |
| | | | | hsa-mir-133a | | | | |
| MPC Prostate Dhanasekaran | 11 | | | | | hsa-mir-34a | Causal | 0 |
| MPC Prostate Dhanasekaran | 10 | | | | | hsa-mir-554 | | 0 |
| MPC Prostate Dhanasekaran | 22 | | | hsa-mir-105 | | hsa-mir-382 | | 0 |
| MPC Prostate Dhanasekaran | 17 | | | | | hsa-mir-1266 | | 0 |
| MPC Prostate Dhanasekaran | 16 | | | hsa-mir-944 | | hsa-mir-101 | Causal | 0 |
| MPC Prostate Dhanasekaran | 19 | | | hsa-mir-661 | | | | 0 |
| MPC Prostate Dhanasekaran | 23 | | | | | hsa-mir-302f | | 0 |
| GCT Seminoma Korkola | 32 | | | | | hsa-mir-608 | | 0 |
| MPC Prostate Dhanasekaran | 34 | | | hsa-mir-544 | | hsa-mir-1279 | | 0 |
| | | | | hsa-mir-544b | | | | |
| MPC Prostate Dhanasekaran | 18 | | | | | hsa-mir-1263 | | 0 |
| PPC Prostate Dhanasekaran | 29 | | | | | hsa-mir-509-3p | | 0 |
| | | | | | | hsa-mir-509-3-5p | | |
| PPC Prostate Dhanasekaran | 27 | | | hsa-mir-654-3p | | hsa-mir-1279 | | 0 |
| PPC Prostate Dhanasekaran | 20 | | | | | hsa-mir-1207-5p | | 0 |
| PPC Prostate Dhanasekaran | 22 | | | hsa-mir-802 | | hsa-mir-561 | | 0 |
| PPC Prostate Dhanasekaran | 23 | | | hsa-mir-376a | | hsa-mir-518a-5p | | 0 |
| | | | | hsa-mir-376b | | | | |
| | | | | hsa-mir-376c | | | | |
| PPC Prostate Dhanasekaran | 28 | | | hsa-mir-544 | | hsa-mir-944 | | 0 |
| | | | | hsa-mir-544b | | | | |
| PPC Prostate Dhanasekaran | 41 | | | hsa-mir-18a | | hsa-mir-369-3p | | 0 |
| | | | | hsa-mir-18b | | | | |
| PPC Prostate Dhanasekaran | 1 | | | hsa-mir-520d-5p | | hsa-mir-561 | | 0 |
| | | | | hsa-mir-524-5p | | | | |
| PPC Prostate Dhanasekaran | 0 | | | hsa-mir-558 | | | | 0 |
| PPC Prostate Dhanasekaran | 3 | | | hsa-mir-624 | | hsa-mir-578 | | 0 |
| PPC Prostate Dhanasekaran | 2 | | | hsa-mir-217 | | hsa-mir-641 | | 0 |
| PPC Prostate Dhanasekaran | 4 | | | hsa-mir-760 | | hsa-mir-939 | | 0 |
| PPC Prostate Dhanasekaran | 7 | | | | | hsa-mir-1202 | | 0 |
| PPC Prostate Dhanasekaran | 9 | | | hsa-mir-153 | | hsa-mir-661 | | 0 |
| PPC Prostate Dhanasekaran | 8 | | | | | hsa-mir-340 | | 0 |
| PPC Prostate Dhanasekaran | 12 | | | | | hsa-mir-342-3p | | 0 |
| PPC Prostate Dhanasekaran | 11 | | | hsa-mir-628-3p | | | | 0 |
| PPC Prostate Dhanasekaran | 10 | | | hsa-mir-421 | | hsa-mir-889 | | 0 |
| PPC Prostate Dhanasekaran | 39 | | | | | hsa-mir-139-3p | | 0 |
| PPC Prostate Dhanasekaran | 38 | | | | | hsa-mir-607 | | 0 |
| PPC Prostate Dhanasekaran | 14 | | | hsa-mir-34a | Causal | hsa-mir-1202 | | 0 |
| | | | | hsa-mir-34c-5p | | | | |
| | | | | hsa-mir-449a | | | | |
| | | | | hsa-mir-449b | | | | |
| PPC Prostate Dhanasekaran | 17 | | | | | hsa-mir-34a | Causal | 0 |
| PPC Prostate Dhanasekaran | 19 | hsa-mir-101* | | hsa-mir-548e | | hsa-mir-144 | | 0 |
| PPC Prostate Dhanasekaran | 31 | | | | | hsa-mir-515-3p | | 0 |
| PPC Prostate Dhanasekaran | 37 | | | | | hsa-mir-553 | | 0 |
| PPC Prostate Dhanasekaran | 35 | | | | | hsa-mir-663b | | 0 |
| | | | | | | hsa-mir-663 | | |
| PPC Prostate Dhanasekaran | 34 | | | | | hsa-mir-149 | Dysregulated | 0 |
| PPC Prostate Dhanasekaran | 33 | | | | | hsa-mir-548e | | 0 |
| PPC Prostate Dhanasekaran | 32 | | | | | hsa-mir-296-5p | Dysregulated | 0 |
| BPH Prostate Dhanasekaran | 20 | | | | | hsa-mir-1224-3p | | 0 |
| BPH Prostate Dhanasekaran | 1 | | | | | hsa-mir-579 | | 0 |
| BPH Prostate Dhanasekaran | 5 | | | hsa-mir-1283 | | hsa-mir-1290 | | 0 |
| BPH Prostate Dhanasekaran | 4 | | | hsa-mir-543 | | hsa-mir-1278 | | 0 |
| BPH Prostate Dhanasekaran | 7 | | | hsa-mir-508-5p | | hsa-mir-380 | | 0 |
| BPH Prostate Dhanasekaran | 6 | | | hsa-mir-648 | | | | 0 |
| BPH Prostate Dhanasekaran | 9 | | | | | hsa-mir-574-5p | | 0 |
| BPH Prostate Dhanasekaran | 8 | | | hsa-mir-891b | | hsa-mir-1246 | | 0 |
| BPH Prostate Dhanasekaran | 11 | | | hsa-mir-520d-5p | | hsa-mir-369-3p | | 0 |
| | | | | hsa-mir-524-5p | | | | |
| BPH Prostate Dhanasekaran | 10 | | | hsa-mir-1290 | | hsa-mir-624 | | 0 |
| BPH Prostate Dhanasekaran | 13 | hsa-mir-423-3p | | | | hsa-mir-922 | | 0 |
| BPH Prostate Dhanasekaran | 12 | | | hsa-mir-33b | | hsa-mir-939 | | 0 |
| | | | | hsa-mir-33a | | | | |
| BPH Prostate Dhanasekaran | 15 | | | hsa-mir-770-5p | | | | 0 |
| BPH Prostate Dhanasekaran | 16 | | | hsa-mir-548e | | hsa-mir-302f | | 0 |
| BPH Prostate Dhanasekaran | 18 | | | | | hsa-mir-513a-3p | | 0 |
| TU Prostate Lapointe | 24 | hsa-mir-487b | | hsa-mir-496 | | hsa-mir-548g | | 0 |
| TU Prostate Lapointe | 25 | | | | | hsa-mir-130a | | 0 |
| TU Prostate Lapointe | 26 | hsa-mir-566 | | | | hsa-mir-663b | | 0 |
| | | | | | | hsa-mir-663 | | |
| TU Prostate Lapointe | 21 | | | | | hsa-mir-608 | | 0 |
| TU Prostate Lapointe | 23 | | | | | hsa-mir-25 | Dysregulated | 0 |
| TU Prostate Lapointe | 29 | | | | | hsa-mir-522 | | 0 |
| TU Prostate Lapointe | 40 | hsa-mir-369-5p | | | | | | 0 |
| TU Prostate Lapointe | 1 | | | hsa-mir-1297 | Dysregulated | hsa-mir-590-3p | | 0 |
| | | | | hsa-mir-26a | | | | |
| | | | | hsa-mir-26b | | | | |
| TU Prostate Lapointe | 0 | | | hsa-mir-874 | | hsa-mir-219-2-3p | | 0 |
| OD Brain Bredel | 14 | | | | | hsa-mir-296-5p | Causal | 0 |
| TU Prostate Lapointe | 4 | | | hsa-mir-532-3p | | hsa-mir-185 | | 0 |
| TU Prostate Lapointe | 7 | | | | | hsa-mir-579 | | 0 |
| TU Prostate Lapointe | 6 | | | | | hsa-mir-876-3p | | 0 |
| TU Prostate Lapointe | 9 | | | hsa-mir-208a | | | | 0 |
| | | | | hsa-mir-208b | | | | |

SUPPLEMENTARY TABLE 9
GO Terms Mapping to the Hallmarks of Cancer
Self Sufficiency in Growth Signals

| | |
|---|---|
| GO:0009967 | Positive regulation of signal transduction |
| GO:0030307 | Positive regulation of cell growth |
| GO:0008284 | Positive regulation of cell proliferation |
| GO:0045787 | Positivie regulation of cell cycle |
| GO:0007165 | Signal transduction |

Insensitivity to Antigrowth Signals

| | |
|---|---|
| GO:0009968 | Negative regulation of signal transduction |
| GO:0030308 | Negative regulation of cell growth |
| GO:0008285 | Negative regulation of cell proliferation |
| GO:0045786 | Negative regulation of cell cycle |
| GO:0007165 | Signal transduction |

Evading Apoptosis

| | |
|---|---|
| GO:0043069 | Negative regulation of apoptosis |
| GO:0043066 | Positive regulation of anti-apoptosis |
| GO:0045768 | Negative regualtion of programmed cell death |

Limitless Replicative Potential

| | |
|---|---|
| GO:0001302 | Replicative cell aging |
| GO:0032206 | Positive regualtion of telomere maintenance |
| GO:0090398 | Cellular senescence |

Sustained Angiogenesis

| | |
|---|---|
| GO:0045765 | Positive regulation of angiogenesis |
| GO:0045766 | Regulation of angiogenesis |
| GO:0030949 | Positive regulation of vascular endothelial growth |
| | factor receptor signaling pathway |
| GO:0001570 | Vasculogenesis |

Tissue Invasion and Metastasis

| | |
|---|---|
| GO:0042060 | Wound healing |
| GO:0007162 | Negative regulation of cell adhesion |
| GO:0033631 | Cell-cell adhesion mediated by integrin |
| GO:0044331 | Cell-cell adhesion mediated by cadherin |
| GO:0001837 | Epithelial to mesenchymal transition |
| GO:0016477 | Cell migration |
| GO:0048870 | Cell motility |
| GO:0007155 | Cell adhesion |

Genome Instability and Mutation

| | |
|---|---|
| GO:0051276 | Chromosome organization |
| GO:0045005 | Maintenance of fidelity involved in DNA-dependent |
| | DNA replication |
| GO:0006281 | DNA repair |

Tumor Promoting Inflammation

| | |
|---|---|
| GO:0002419 | T-cell mediated cytotoxicity directed against |
| | tumor cell target |
| GO:0002420 | Natural killer cell mediated cytotoxicity directed |
| | against tumor cell target |
| GO:0002857 | Positive regualtion of natural killer cell mediated |
| | immune response to tumor cell |
| GO:0002842 | Positive regualtion of T-cell mediated immune |
| | response to tumor cell |
| GO:0002367 | Cytokine production involved in immune response |
| GO:0050776 | Regulation of immune response |

Reprogramming Energy Metabolism

| | |
|---|---|
| GO:0006096 | Glycolysis |
| GO:0071456 | Cellular response to hypoxia |

Evading Immune Detection

| | |
|---|---|
| GO:0002837 | Regulation of immune response to tumor cell |
| GO:0002418 | Immune response to tumor cells |
| GO:0002367 | Cytokine production involved in immune response |
| GO:0050776 | Regulation of immune response |

SUPPLEMENTARY TABLE 10

| | | miRvestiagtor | | PITA | TargetScan |
|---|---|---|---|---|---|
| Co-Expression Signature | miRvestigator miRNA | Validation | PITA miRNA | Validation | miRNA |
| GL Brain Rickman.31 | NA | NA | hsa-let-7e_hsa-let-7f_hsa-let-7g_hsa- | dysregulated | NA |
| | | | let-7a_hsa-let-7b_hsa-let-7d_hsa-let- | | |
| | | | 7i_hsa-mir-98_hsa-let-7c | | |
| B-CLL Leukemia Haslinger.9 | NA | NA | NA | NA | hsa-mir-1245b- |
| | | | | | 5p_hsa- |
| | | | | | mir-1245 |
| SRS Ovarian Hendrix.14 | NA | NA | NA | NA | hsa-mir-1290 |
| IDC Breast Radvanyi.24 | NA | NA | NA | NA | hsa-mir-1291 |
| TU Prostate Lapointe.37 | NA | NA | hsa-mir-199b-3p_hsa-mir-199a-3p | causal | hsa-mir-509- |
| | | | | | 3p_hsa- |
| | | | | | mir-509-3-5p |
| AD Ovarian Welsh.26 | NA | NA | hsa-mir-210 | NA | NA |
| AD Lung Beer.35 | NA | NA | hsa-mir-222 | causal | NA |
| AD Lung Bhattacharjee.41 | NA | NA | NA | NA | hsa-mir-296-5p |
| SMCL Lung Bhattacharjee.36 | NA | NA | NA | NA | hsa-mir-296-5p |
| SQ Lung 8hattacharjee.13 | NA | NA | NA | NA | hsa-mir-296-5p |
| AD Lung Bhattacharjee.59 | NA | NA | hsa-mir-29b_hsa-mir-29c_hsa-mir-29a | causal | NA |
| END Ovarian Hendrix.6 | NA | NA | hsa-mir-29b_hsa-mir-29c_hsa-mir-29a | dysregulated | hsa-mir-890 |
| CCC Ovarian Hendrix.11 | hsa-mir-638 | NA | hsa-mir-29b_hsa-mir-29c_hsa-mir-29a | dysregulated | hsa-mir-602 |
| AD Lung Beer.31 | hsa-mir-29b_hsa-mir- | causal | hsa-mir-29b_hsa-mir-29c_hsa-mir-29a | causal | hsa-mir-29b |
| | 29c_hsa-mir-29a | | | | |
| CA Colon Graudens.35 | NA | NA | hsa-mir-325 | NA | NA |
| SMCL Lung Bhattacharjee.61 | NA | NA | NA | NA | hsa-mir-331-3p |
| COID Lung Bhattacharjee.24 | NA | NA | NA | NA | hsa-mir-342-5p |
| AD Lung Stearman.19 | NA | NA | NA | NA | hsa-mir-370 |
| GCT Seminoma Korkola.4 | NA | NA | NA | NA | hsa-mir- |
| | | | | | 376a_hsa-mir- |
| | | | | | 376b_hsa-mir-376c |
| CA Breast Sorlie.23 | NA | NA | NA | NA | hsa-mir-423-5p |
| HSCC Head-Neck Chung.1 | hsa-mir-29b_hsa-mir- | dysregulated | hsa-mir-767-5p | NA | hsa-mir-450b-5p |
| | 29c_hsa-mir-29a | | | | |
| CA Breast Richardson.55 | NA | NA | hsa-mir-130b_hsa-mir-301a_hsa-mir- | NA | hsa-mir- |
| | | | 301b_hsa-mir-130a_hsa-mir-454 | | 544_hsa- |
| | | | | | mir-544b |
| MM Myeloma Zhan.33 | NA | NA | NA | NA | hsa-mir-507 |
| CA Bladder Dyrskjot.82 | NA | NA | hsa-mir-519d | NA | hsa-mir-656 |
| ME Melanoma Hoek.50 | NA | NA | hsa-mir-548c-3p | NA | NA |
| AD Ovarian Welsh.1 | NA | NA | NA | NA | hsa-mir-548d-3p |
| AD Ovarian Welsh.16 | NA | NA | NA | NA | hsa-mir-548I |
| CA Colon Graudens.40 | NA | NA | hsa-mir-507_hsa-mir-557 | NA | hsa-mir-369-3p |
| SQ Lung Bhattacharjee.23 | NA | NA | NA | NA | hsa-mir-586 |
| END Ovarian Hendrix.58 | hsa-mir-621 | NA | NA | NA | NA |
| GL Brain Rickman.8 | NA | NA | NA | NA | hsa-mir-634 |
| AC Brain Sun.5 | hsa-mir-523 | NA | NA | NA | hsa-mir-637 |
| AD Lung Beer.32 | NA | NA | hsa-mir-656 | NA | NA |
| GL Brain Rickman.2 | hsa-mir-718 | NA | NA | NA | NA |
| B-CLL Leukemia Haslinger.27 | NA | NA | hsa-mir-760 | NA | NA |
| AD Ovarian Welsh.20 | NA | NA | hsa-mir-767-5p | NA | NA |
| SQ Lung Bhattacharjee.44 | hsa-mir-4285 | NA | hsa-mir-767-5p | NA | NA |
| COID Lung Bhattacharjee.92 | NA | NA | NA | NA | hsa-mir-920 |
| END Ovarian Hendrix.22 | NA | NA | hsa-mir-15b_hsa-mir-15a_hsa-mir- | dysregulated | hsa-mir-944 |
| | | | 16_hsa-mir-497_hsa-mir-195 | | |

| Co-Expression Signature | TargetScan Validation | GO and miRNA Overlap |
|---|---|---|
| GL Brain Rickman.31 | NA | hsa-let-7c_GO: 0001501; hsa-let-7c_GO: 0001503; hsa-let-7c_GO: 0001934; hsa-let- |
| | | 7c_GO: 0001944; hsa-let-7c_GO: 0001957; hsa-let-7c_GO: 0005979; hsa-let- |
| | | 7c_GO: 0006928; hsa-let-7c_GO: 0007275; hsa-let-7c_GO: 0007530; hsa-let- |
| | | 7c_GO: 0008284; hsa-let-7c_GO: 0008544; hsa-let-7c_GO: 0008645; hsa-let- |
| | | 7c_GO: 0009653; hsa-let-7c_GO: 0009887; hsa-let-7c_GO: 0009888; hsa-let- |
| | | 7c_GO: 0015749; hsa-let-7c_GO: 0015758; hsa-let-7c_GO: 0016477; hsa-let- |
| | | 7c_GO: 0030154; hsa-let-7c_GO: 0030198; hsa-let-7c_GO: 0030199; hsa-let- |
| | | 7c_GO: 0030238; hsa-let-7c_GO: 0031017; hsa-let-7c_GO: 0032501; hsa-let- |
| | | 7c_GO: 0032502; hsa-let-7c_GO: 0032879; hsa-let-7c_GO: 0032885; hsa-let- |
| | | 7c_GO: 0032963; hsa-let-7c_GO: 0032964; hsa-let-7c_GO: 0033273; hsa-let- |
| | | 7c_GO: 0043062; hsa-let-7c_GO: 0043491; hsa-let-7c_GO: 0043588; hsa-let- |
| | | 7c_GO: 0044236; hsa-let-7c_GO: 0044259; hsa-let-7c_GO: 0048468; hsa-let- |
| | | 7c_GO: 0048513; hsa-let-7c_GO: 0048731; hsa-let-7c_GO: 0048856; hsa-let- |
| | | 7c_GO: 0048869; hsa-let-7c_GO: 0051291; hsa-let-7c_GO: 0060324; hsa-let- |
| | | 7c_GO: 0060343; hsa-let-7c_GO: 0060346; hsa-let-7c_GO: 0065008; hsa-let- |
| | | 7c_GO: 0070208 |
| B-CLL Leukemia Haslinger.9 | NA | hsa-mir-1245_GO: 0042035; hsa-mir-1245_GO: 0042089; hsa-mir-1245_GO: 0042107; hsa- |
| | | mir-1245_GO: 0042108; hsa-mir-1245_GO: 0045086 |
| SRS Ovarian Hendrix.14 | NA | hsa-mir-1290_GO: 0030193; hsa-mir-1290_GO: 0050818; hsa-mir-1290_GO: 0051917; hsa- |
| | | mir-1290_GO: 0051918 |
| IDC Breast Radvanyi.24 | NA | hsa-mir-1291_GO: 0001775; hsa-mir-1291_GO: 0002252; hsa-mir-1291_GO: 0002694; hsa- |
| | | mir-1291_GO: 0002696; hsa-mir-1291_GO: 0002831; hsa-mir-1291_GO: 0006950; hsa-mir- |
| | | 1291_GO: 0007596; hsa-mir-1291_GO: 0007599; hsa-mir-1291_GO: 0009615; hsa-mir- |
| | | 1291_GO: 0019932; hsa-mir-1291_GO: 0031294; hsa-mir-1291_GO: 0031295; hsa-mir- |
| | | 1291_GO: 0050688; hsa-mir-1291_GO: 0050690; hsa-mir-1291_GO: 0050817; hsa-mir- |
| | | 1291_GO: 0050848; hsa-mir-1291_GO: 0050849; hsa-mir-1291_GO: 0050863; hsa-mir- |
| | | 1291_GO: 0050865; hsa-mir-1291_GO: 0050867; hsa-mir-1291_GO: 0050870; hsa-mir- |
| | | 1291_GO: 0050878; hsa-mir-1291_GO: 0051249; hsa-mir-1291_GO: 0051251; hsa-mir- |
| | | 1291_GO: 0051346; hsa-mir-1291_GO: 0051707 |
| TU Prostate Lapointe.37 | NA | hsa-mir-199a-3p_GO: 0040012 |
| AD Ovarian Welsh.26 | NA | hsa-mir-210_GO: 0002521; hsa-mir-210_GO: 0006917; hsa-mir-210_GO: 0006928; hsa-mir- |
| | | 210_GO: 0012502; hsa-mir-210_GO: 0016477; hsa-mir-210_GO: 0032943; hsa-mir- |
| | | 210_GO: 0032944; hsa-mir-210_GO: 0040012; hsa-mir-210_GO: 0040017; hsa-mir- |
| | | 210_GO: 0043065; hsa-mir-210_GO: 0043068; hsa-mir-210_GO: 0045321; hsa-mir- |
| | | 210_GO: 0045672; hsa-mir-210_GO: 0048518; hsa-mir-210_GO: 0048870; hsa-mir- |
| | | 210_GO: 0050789; hsa-mir-210_GO: 0050794; hsa-mir-210_GO: 0051674; hsa-mir- |
| | | 210_GO: 0065007 |
| AD Lung Beer.35 | NA | hsa-mir-222_GO: 0007155; hsa-mir-222_GO: 0022610; hsa-mir-222_GO: 0030029 |
| AD Lung Bhattacharjee.41 | NA | hsa-mir-296-5p_GO: 0003007; hsa-mir-296-5p_GO: 0006928; hsa-mir-296- |
| | | 5p_GO: 0009653; hsa-mir-296-5p_GO: 0016043; hsa-mir-296-5p_GO: 0016477; hsa-mir- |
| | | 296-5p_GO: 0040011; hsa-mir-296-5p_GO: 0048518; hsa-mir-296-5p_GO: 0048870; hsa- |
| | | mir-296-5p_GO: 0051674 |
| SMCL Lung Bhattacharjee.36 | NA | hsa-mir-296-5p_GO: 0007517; hsa-mir-296-5p_GO: 0009653; hsa-mir-296- |
| | | 5p_GO: 0009887; hsa-mir-296-5p_GO: 0010269; hsa-mir-296-5p_GO: 0010562; hsa-mir- |
| | | 296-5p_GO: 0030509; hsa-mir-296-5p_GO: 0031960; hsa-mir-296-5p_GO: 0042127; hsa- |
| | | mir-296-5p_GO: 0045937; hsa-mir-296-5p_GO: 0051384 |
| SQ Lung 8hattacharjee.13 | NA | hsa-mir-296-5p_GO: 0010884; hsa-mir-296-5p_GO: 0016043; hsa-mir-296- |
| | | 5p_GO: 0032879 |
| AD Lung Bhattacharjee.59 | NA | hsa-mir-29a_GO: 0000904; hsa-mir-29a_GO: 0001501; hsa-mir-29a_GO: 0001568; hsa-mir- |
| | | 29a_GO: 0001944; hsa-mir-29a_GO: 0007229; hsa-mir-29a_GO: 0007275; hsa-mir- |
| | | 29a_GO: 0007409; hsa-mir-29a_GO: 0007411; hsa-mir-29a_GO: 0009611; hsa-mir- |
| | | 29a_GO: 0009653; hsa-mir-29a_GO: 0016477; hsa-mir-29a_GO: 0022008; hsa-mir- |
| | | 29a_GO: 0030199; hsa-mir-29a_GO: 0032501; hsa-mir-29a_GO: 0032502; hsa-mir- |
| | | 29a_GO: 0032990; hsa-mir-29a_GO: 0042060; hsa-mir-29a_GO: 0048667; hsa-mir- |
| | | 29a_GO: 0048812; hsa-mir-29a_GO: 0048858 |
| END Ovarian Hendrix.6 | NA | hsa-mir-29a_GO: 0001501; hsa-mir-29a_GO: 0001568; hsa-mir-29a_GO: 0001944; hsa-mir- |
| | | 29a_GO: 0007167; hsa-mir-29a_GO: 0007178; hsa-mir-29a_GO: 0007179; hsa-mir- |
| | | 29a_GO: 0007275; hsa-mir-29a_GO: 0009653; hsa-mir-29a_GO: 0030154; hsa-mir- |
| | | 29a_GO: 0030198; hsa-mir-29a_GO: 0030199; hsa-mir-29a_GO: 0032501; hsa-mir- |
| | | 29a_GO: 0032502; hsa-mir-29a_GO: 0043062; hsa-mir-29a_GO: 0048513; hsa-mir- |
| | | 29a_GO: 0048731; hsa-mir-29a_GO: 0048856; hsa-mir-29a_GO: 0051674 |
| CCC Ovarian Hendrix.11 | NA | hsa-mir-29a_GO: 0007409; hsa-mir-29a_GO: 0007411; hsa-mir-29a_GO: 0009887; hsa-mir- |
| | | 29a_GO: 0030198; hsa-mir-29a_GO: 0031175; hsa-mir-29a_GO: 0032990; hsa-mir- |
| | | 29a_GO: 0048513; hsa-mir-29a_GO: 0048667; hsa-mir-29a_GO: 0048812; hsa-mir- |
| | | 29a_GO: 0048858 |
| AD Lung Beer.31 | causal | hsa-mir-29b_GO: 0009404; hsa-mir-29b_GO: 0030199; hsa-mir-29b_GO: 0032963; hsa-mir- |
| | | 29b_GO: 0032964; hsa-mir-29b_GO: 0044236; hsa-mir-29b_GO: 0044259 |
| CA Colon Graudens.35 | NA | hsa-mir-325_GO: 0006520; hsa-mir-325_GO: 0051186 |
| SMCL Lung Bhattacharjee.61 | NA | hsa-mir-331-3p_GO: 0000902; hsa-mir-331-3p_GO: 0000904; hsa-mir-331- |
| | | 3p_GO: 0001775; hsa-mir-331-3p_GO: 0002376; hsa-mir-331-3p_GO: 0002520; hsa-mir- |
| | | 331-3p_GO: 0002682; hsa-mir-331-3p_GO: 0002684; hsa-mir-331-3p_GO: 0002694; hsa- |
| | | mir-331-3p_GO: 0002695; hsa-mir-331-3p_GO: 0002696; hsa-mir-331- |
| | | 3p_GO: 0006464; hsa-mir-331-3p_GO: 0006468; hsa-mir-331-3p_GO: 0006935; hsa-mir- |
| | | 331-3p_GO: 0007154; hsa-mir-331-3p_GO: 0007155; hsa-mir-331-3p_GO: 0007166; hsa- |
| | | mir-331-3p_GO: 0007167; hsa-mir-331-3p_GO: 0007169; hsa-mir-331- |
| | | 3p_GO: 0007264; hsa-mir-331-3p_GO: 0007265; hsa-mir-331-3p_GO: 0007267; hsa-mir- |
| | | 331-3p_GO: 0007268; hsa-mir-331-3p_GO: 0007399; hsa-mir-331-3p_GO: 0007507; hsa- |
| | | mir-331-3p_GO: 0007596; hsa-mir-331-3p_GO: 0007599; hsa-mir-331- |
| | | 3p_GO: 0009605; hsa-mir-331-3p_GO: 0009653; hsa-mir-331-3p_GO: 0009719; hsa-mir- |
| | | 331-3p_GO: 0009725; hsa-mir-331-3p_GO: 0009887; hsa-mir-331-3p_GO: 0010518; hsa- |
| | | mir-331-3p_GO: 0010863; hsa-mir-331-3p_GO: 0016310; hsa-mir-331- |
| | | 3p_GO: 0019226; hsa-mir-331-3p_GO: 0021700; hsa-mir-331-3p_GO: 0022008; hsa-mir- |
| | | 331-3p_GO: 0022610; hsa-mir-331-3p_GO: 0030029; hsa-mir-331-3p_GO: 0030097; hsa- |
| | | mir-331-3p_GO: 0030154; hsa-mir-331-3p_GO: 0030182; hsa-mir-331- |
| | | 3p_GO: 0030323; hsa-mir-331-3p_GO: 0030324; hsa-mir-331-3p_GO: 0031100; hsa-mir- |
| | | 331-3p_GO: 0031589; hsa-mir-331-3p_GO: 0032989; hsa-mir-331-3p_GO: 0040011; hsa- |
| | | mir-331-3p_GO: 0042060; hsa-mir-331-3p_GO: 0042110; hsa-mir-331- |
| | | 3p_GO: 0042330; hsa-mir-331-3p_GO: 0043412; hsa-mir-331-3p_GO: 0043434; hsa-mir- |
| | | 331-3p_GO: 0045321; hsa-mir-331-3p_GO: 0046578; hsa-mir-331-3p_GO: 0046649; hsa- |
| | | mir-331-3p_GO: 0048468; hsa-mir-331-3p_GO: 0048534; hsa-mir-331- |
| | | 3p_GO: 0048699; hsa-mir-331-3p_GO: 0048731; hsa-mir-331-3p_GO: 0048869; hsa-mir- |
| | | 331-3p_GO: 0050817; hsa-mir-331-3p_GO: 0050863; hsa-mir-331-3p_GO: 0050865; hsa- |
| | | mir-331-3p_GO: 0050866; hsa-mir-331-3p_GO: 0050867; hsa-mir-331- |
| | | 3p_GO: 0050870; hsa-mir-331-3p_GO: 0050878; hsa-mir-331-3p_GO: 0051249; hsa-mir- |
| | | 331-3p_GO: 0051251 |
| COID Lung Bhattacharjee.24 | NA | hsa-mir-342-5p_GO: 0007584; hsa-mir-342-5p_GO: 0009991; hsa-mir-342- |
| | | 5p_GO: 0010269; hsa-mir-342-5p_GO: 0030198; hsa-mir-342-5p_GO: 0031667; hsa-mir- |
| | | 342-5p_GO: 0031960; hsa-mir-342-5p_GO: 0043062; hsa-mir-342-5p_GO: 0050900; hsa- |
| | | mir-342-5p_GO: 0051384 |
| AD Lung Stearman.19 | NA | hsa-mir-370_GO: 0001570 |
| GCT Seminoma Korkola.4 | NA | hsa-mir-376c_GO: 0007157; hsa-mir-376c_GO: 0007166; hsa-mir-376c_GO: 0009605; hsa- |
| | | mir-376c_GO: 0016477; hsa-mir-376c_GO: 0030595; hsa-mir-376c_GO: 0032102; hsa-mir- |
| | | 376c_GO: 0032501; hsa-mir-376c_GO: 0032879; hsa-mir-376c_GO: 0040011; hsa-mir- |
| | | 376c_GO: 0040012; hsa-mir-376c_GO: 0045123; hsa-mir-376c_GO: 0048870; hsa-mir- |
| | | 376c_GO: 0050900; hsa-mir-376c_GO: 0050901; hsa-mir-376c_GO: 0051270; hsa-mir- |
| | | 376c_GO: 0051674 |
| CA Breast Sorlie.23 | NA | hsa-mir-423-5p_GO: 0000165; hsa-mir-423-5p_GO: 0006928; hsa-mir-423- |
| | | 5p_GO: 0007165; hsa-mir-423-5p_GO: 0007166; hsa-mir-423-5p_GO: 0007167; hsa-mir- |
| | | 423-5p_GO: 0007169; hsa-mir-423-5p_GO: 0007243; hsa-mir-423-5p_GO: 0007266; hsa- |
| | | mir-423-5p_GO: 0008283; hsa-mir-423-5p_GO: 0008284; hsa-mir-423- |
| | | 5p_GO: 0016043; hsa-mir-423-5p_GO: 0016192; hsa-mir-423-5p_GO: 0030334; hsa-mir- |
| | | 423-5p_GO: 0032570; hsa-mir-423-5p_GO: 0032879; hsa-mir-423-5p_GO: 0040011; hsa- |
| | | mir-423-5p_GO: 0040012; hsa-mir-423-5p_GO: 0042127; hsa-mir-423- |
| | | 5p_GO: 0043491; hsa-mir-423-5p_GO: 0045807; hsa-mir-423-5p_GO: 0048518; hsa-mir- |
| | | 423-5p_GO: 0048522; hsa-mir-423-5p_GO: 0048583; hsa-mir-423-5p_GO: 0051050; hsa- |
| | | mir-423-5p_GO: 0051270; hsa-mir-423-5p_GO: 0051272; hsa-mir-423-5p_GO: 0065007 |
| HSCC Head-Neck Chung.1 | NA | hsa-mir-450b-5p_GO: 0001503; hsa-mir-450b-5p_GO: 0001934; hsa-mir-450b- |
| | | 5p_GO: 0030154; hsa-mir-450b-5p_GO: 0030510; hsa-mir-450b-5p_GO: 0030514; hsa-mir- |
| | | 450b-5p_GO: 0033138; hsa-mir-450b-5p_GO: 0048869 |
| CA Breast Richardson.55 | NA | hsa-mir-454_GO: 0001701; hsa-mir-454_GO: 0009653; hsa-mir-454_GO: 0009790; hsa-mir- |
| | | 454_GO: 0048514 |
| MM Myeloma Zhan.33 | NA | hsa-mir-507_GO: 0006350; hsa-mir-507_GO: 0010556; hsa-mir-507_GO: 0031326; hsa-mir- |
| | | 507_GO: 0044237; hsa-mir-507_GO: 0044238; hsa-mir-507_GO: 0045449 |
| CA Bladder Dyrskjot.82 | NA | hsa-mir-519d_GO: 0030514; hsa-mir-519d_GO: 0046631; hsa-mir-519d_GO: 0048518; hsa- |
| | | mir-519d_GO: 0048583; hsa-mir-519d_GO: 0050789; hsa-mir-519d_GO: 0065007 |
| ME Melanoma Hoek.50 | NA | hsa-mir-548c-3p_GO: 0000902; hsa-mir-548c-3p_GO: 0000904; hsa-mir-548c- |
| | | 3p_GO: 0001525; hsa-mir-548c-3p_GO: 0001568; hsa-mir-548c-3p_GO: 0001944; hsa-mir- |
| | | 548c-3p_GO: 0006357; hsa-mir-548c-3p_GO: 0006366; hsa-mir-548c-3p_GO: 0006464; hsa- |
| | | mir-548c-3p_GO: 0006886; hsa-mir-548c-3p_GO: 0006913; hsa-mir-548c- |
| | | 3p_GO: 0006928; hsa-mir-548c-3p_GO: 0007154; hsa-mir-548c-3p_GO: 0007167; hsa-mir- |
| | | 548c-3p_GO: 0007169; hsa-mir-548c-3p_GO: 0007275; hsa-mir-548c-3p_GO: 0007389; hsa- |
| | | mir-548c-3p_GO: 0007399; hsa-mir-548c-3p_GO: 0007409; hsa-mir-548c- |
| | | 3p_GO: 0007411; hsa-mir-548c-3p_GO: 0007417; hsa-mir-548c-3p_GO: 0007611; hsa-mir- |
| | | 548c-3p_GO: 0008104; hsa-mir-548c-3p_GO: 0008283; hsa-mir-548c-3p_GO: 0008285; hsa- |
| | | mir-548c-3p_GO: 0009653; hsa-mir-548c-3p_GO: 0009790; hsa-mir-548c- |
| | | 3p_GO: 0009887; hsa-mir-548c-3p_GO: 0009888; hsa-mir-548c-3p_GO: 0009891; hsa-mir- |
| | | 548c-3p_GO: 0009893; hsa-mir-548c-3p_GO: 0009987; hsa-mir-548c-3p_GO: 0010001; hsa- |
| | | mir-548c-3p_GO: 0010557; hsa-mir-548c-3p_GO: 0010604; hsa-mir-548c- |
| | | 3p_GO: 0010628; hsa-mir-548c-3p_GO: 0014812; hsa-mir-548c-3p_GO: 0015031; hsa-mir- |
| | | 548c-3p_GO: 0016477; hsa-mir-548c-3p_GO: 0019538; hsa-mir-548c-3p_GO: 0022008; hsa- |
| | | mir-548c-3p_GO: 0030154; hsa-mir-548c-3p_GO: 0030182; hsa-mir-548c- |
| | | 3p_GO: 0030326; hsa-mir-548c-3p_GO: 0030334; hsa-mir-548c-3p_GO: 0031325; hsa-mir- |
| | | 548c-3p_GO: 0031328; hsa-mir-548c-3p_GO: 0032501; hsa-mir-548c-3p_GO: 0032502; hsa- |
| | | mir-548c-3p_GO: 0032990; hsa-mir-548c-3p_GO: 0033036; hsa-mir-548c- |
| | | 3p_GO: 0034613; hsa-mir-548c-3p_GO: 0035107; hsa-mir-548c-3p_GO: 0035108; hsa-mir- |
| | | 548c-3p_GO: 0035113; hsa-mir-548c-3p_GO: 0035295; hsa-mir-548c-3p_GO: 0040011; hsa- |
| | | mir-548c-3p_GO: 0040012; hsa-mir-548c-3p_GO: 0042063; hsa-mir-548c- |
| | | 3p_GO: 0042127; hsa-mir-548c-3p_GO: 0042733; hsa-mir-548c-3p_GO: 0043412; hsa-mir- |
| | | 548c-3p_GO: 0044238; hsa-mir-548c-3p_GO: 0044267; hsa-mir-548c-3p_GO: 0045184; hsa- |
| | | mir-548c-3p_GO: 0045595; hsa-mir-548c-3p_GO: 0045596; hsa-mir-548c- |
| | | 3p_GO: 0045597; hsa-mir-548c-3p_GO: 0045778; hsa-mir-548c-3p_GO: 0045893; hsa-mir- |
| | | 548c-3p_GO: 0045935; hsa-mir-548c-3p_GO: 0045941; hsa-mir-548c-3p_GO: 0045944; hsa- |
| | | mir-548c-3p_GO: 0046907; hsa-mir-548c-3p_GO: 0048468; hsa-mir-548c- |
| | | 3p_GO: 0048513; hsa-mir-548c-3p_GO: 0048514; hsa-mir-548c-3p_GO: 0048518; hsa-mir- |
| | | 548c-3p_GO: 0048519; hsa-mir-548c-3p_GO: 0048522; hsa-mir-548c-3p_GO: 0048523; hsa- |
| | | mir-548c-3p_GO: 0048598; hsa-mir-548c-3p_GO: 0048646; hsa-mir-548c- |
| | | 3p_GO: 0048667; hsa-mir-548c-3p_GO: 0048699; hsa-mir-548c-3p_GO: 0048705; hsa-mir- |
| | | 548c-3p_GO: 0048731; hsa-mir-548c-3p_GO: 0048736; hsa-mir-548c-3p_GO: 0048812; hsa- |
| | | mir-548c-3p_GO: 0048846; hsa-mir-548c-3p_GO: 0048856; hsa-mir-548c- |
| | | 3p_GO: 0048858; hsa-mir-548c-3p_GO: 0048869; hsa-mir-548c-3p_GO: 0048870; hsa-mir- |
| | | 548c-3p_GO: 0050789; hsa-mir-548c-3p_GO: 0050793; hsa-mir-548c-3p_GO: 0050794; hsa- |
| | | mir-548c-3p_GO: 0051093; hsa-mir-548c-3p_GO: 0051094; hsa-mir-548c- |
| | | 3p_GO: 0051101; hsa-mir-548c-3p_GO: 0051169; hsa-mir-548c-3p_GO: 0051239; hsa-mir- |
| | | 548c-3p_GO: 0051254; hsa-mir-548c-3p_GO: 0051270; hsa-mir-548c-3p_GO: 0051641; hsa- |
| | | mir-548c-3p_GO: 0051649; hsa-mir-548c-3p_GO: 0051674; hsa-mir-548c- |
| | | 3p_GO: 0060070; hsa-mir-548c-3p_GO: 0060173; hsa-mir-548c-3p_GO: 0060284; hsa-mir- |
| | | 548c-3p_GO: 0065007 |
| AD Ovarian Welsh.1 | NA | hsa-mir-548d-3p_GO: 0006950; hsa-mir-548d-3p_GO: 0007611; hsa-mir-548d- |
| | | 3p_GO: 0009605; hsa-mir-548d-3p_GO: 0009719; hsa-mir-548d-3p_GO: 0009725; hsa-mir- |
| | | 548d-3p_GO: 0009889; hsa-mir-548d-3p_GO: 0010468; hsa-mir-548d- |
| | | 3p_GO: 0010604; hsa-mir-548d-3p_GO: 0019219; hsa-mir-548d-3p_GO: 0019222; hsa-mir- |
| | | 548d-3p_GO: 0031323; hsa-mir-548d-3p_GO: 0031326; hsa-mir-548d- |
| | | 3p_GO: 0031440; hsa-mir-548d-3p_GO: 0031442; hsa-mir-548d-3p_GO: 0043066; hsa-mir- |
| | | 548d-3p_GO: 0043069; hsa-mir-548d-3p_GO: 0048168; hsa-mir-548d- |
| | | 3p_GO: 0050685; hsa-mir-548d-3p_GO: 0051252; hsa-mir-548d-3p_GO: 0060211; hsa-mir- |
| | | 548d-3p_GO: 0060213; hsa-mir-548d-3p_GO: 0060255 |
| AD Ovarian Welsh.16 | NA | hsa-mir-548I_GO: 0006066; hsa-mir-548I_GO: 0008202; hsa-mir-548I_GO: 0055114 |
| CA Colon Graudens.40 | NA | hsa-mir-557_GO: 0048519; hsa-mir-557_GO: 0048523 |
| SQ Lung Bhattacharjee.23 | NA | hsa-mir-586_GO: 0001910; hsa-mir-586_GO: 0001912; hsa-mir-586_GO: 0031343; hsa-mir- |
| | | 586_GO: 0042492; hsa-mir-586_GO: 0045577; hsa-mir-586_GO: 0045586; hsa-mir- |
| | | 586_GO: 0045588; hsa-mir-586_GO: 0046629; hsa-mir-586_GO: 0046643; hsa-mir- |
| | | 586_GO: 0046645 |
| END Ovarian Hendrix.58 | NA | hsa-mir-621_GO: 0006952; hsa-mir-621_GO: 0006954; hsa-mir-621_GO: 0009611 |
| GL Brain Rickman.8 | NA | hsa-mir-634_GO: 0001661; hsa-mir-634_GO: 0007611; hsa-mir-634_GO: 0007613; hsa-mir- |
| | | 634_GO: 0014070; hsa-mir-634_GO: 0031646; hsa-mir-634_GO: 0032225; hsa-mir- |
| | | 634_GO: 0050806; hsa-mir-634_GO: 0051971 |
| AC Brain Sun.5 | NA | hsa-mir-637_GO: 0002292; hsa-mir-637_GO: 0002293; hsa-mir-637_GO: 0002294; hsa-mir- |
| | | 637_GO: 0042093 |
| AD Lung Beer.32 | NA | hsa-mir-656_GO: 0001525; hsa-mir-656_GO: 0001568; hsa-mir-656_GO: 0001570; hsa-mir- |
| | | 656_GO: 0001944; hsa-mir-656_GO: 0003013; hsa-mir-656_GO: 0006928; hsa-mir- |
| | | 656_GO: 0007166; hsa-mir-656_GO: 0007275; hsa-mir-656_GO: 0007599; hsa-mir- |
| | | 656_GO: 0008015; hsa-mir-656_GO: 0008283; hsa-mir-656_GO: 0009653; hsa-mir- |
| | | 656_GO: 0016477; hsa-mir-656_GO: 0030154; hsa-mir-656_GO: 0030334; hsa-mir- |
| | | 656_GO: 0030335; hsa-mir-656_GO: 0032501; hsa-mir-656_GO: 0032502; hsa-mir- |
| | | 656_GO: 0032879; hsa-mir-656_GO: 0040011; hsa-mir-656_GO: 0040012; hsa-mir- |
| | | 656_GO: 0042060; hsa-mir-656_GO: 0042127; hsa-mir-656_GO: 0048514; hsa-mir- |
| | | 656_GO: 0048522; hsa-mir-656_GO: 0048583; hsa-mir-656_GO: 0048646; hsa-mir- |
| | | 656_GO: 0048731; hsa-mir-656_GO: 0048856; hsa-mir-656_GO: 0048870; hsa-mir- |
| | | 656_GO: 0050878; hsa-mir-656_GO: 0050900; hsa-mir-656_GO: 0051150; hsa-mir- |
| | | 656_GO: 0051270; hsa-mir-656_GO: 0051272; hsa-mir-656_GO: 0051385; hsa-mir- |
| | | 656_GO: 0051412; hsa-mir-656_GO: 0051674; hsa-mir-656_GO: 0070374 |
| GL Brain Rickman.2 | NA | hsa-mir-718_GO: 0002699; hsa-mir-718_GO: 0002703; hsa-mir-718_GO: 0006957; hsa-mir- |
| | | 718_GO: 0010886; hsa-mir-718_GO: 0019060; hsa-mir-718_GO: 0030581; hsa-mir- |
| | | 718_GO: 0046719; hsa-mir-718_GO: 0046967; hsa-mir-718_GO: 0051708 |
| B-CLL Leukemia | NA | hsa-mir-760_GO: 0006323; hsa-mir-760_GO: 0006333; hsa-mir-760_GO: 0006334; hsa-mir- |
| Haslinger.27 | | 760_GO: 0031497; hsa-mir-760_GO: 0034728; hsa-mir-760_GO: 0065004 |
| AD Ovarian Welsh.20 | NA | hsa-mir-767-5p_GO: 0000902; hsa-mir-767-5p_GO: 0000904; hsa-mir-767- |
| | | 5p_GO: 0001501; hsa-mir-767-5p_GO: 0001944; hsa-mir-767-5p_GO: 0007229; hsa-mir- |
| | | 767-5p_GO: 0007409; hsa-mir-767-5p_GO: 0007411; hsa-mir-767-5p_GO: 0009605; hsa- |
| | | mir-767-5p_GO: 0009888; hsa-mir-767-5p_GO: 0016043; hsa-mir-767- |
| | | 5p_GO: 0022008; hsa-mir-767-5p_GO: 0030030; hsa-mir-767-5p_GO: 0030198; hsa-mir- |
| | | 767-5p_GO: 0030199; hsa-mir-767-5p_GO: 0031175; hsa-mir-767-5p_GO: 0032501; hsa- |
| | | mir-767-5p_GO: 0032989; hsa-mir-767-5p_GO: 0032990; hsa-mir-767- |
| | | 5p_GO: 0040011; hsa-mir-767-5p_GO: 0042246; hsa-mir-767-5p_GO: 0043062; hsa-mir- |
| | | 767-5p_GO: 0043588; hsa-mir-767-5p_GO: 0048468; hsa-mir-767-5p_GO: 0048666; hsa- |
| | | mir-767-5p_GO: 0048667; hsa-mir-767-5p_GO: 0048731; hsa-mir-767- |
| | | 5p_GO: 0048812; hsa-mir-767-5p_GO: 0048856; hsa-mir-767-5p_GO: 0048858 |
| SQ Lung Bhattacharjee.44 | NA | hsa-mir-767-5p_GO: 0008544; hsa-mir-767-5p_GO: 0009888; hsa-mir-767- |
| | | 5p_GO: 0030198; hsa-mir-767-5p_GO: 0030199; hsa-mir-767-5p_GO: 0043062; hsa-mir- |
| | | 767-5p_GO: 0043588 |
| COID Lung Bhattacharjee.92 | NA | hsa-mir-920_GO: 0007154; hsa-mir-920_GO: 0030336; hsa-mir-920_GO: 0050865; hsa-mir- |
| | | 920_GO: 0051271 |
| END Ovarian Hendrix.22 | NA | hsa-mir-944_GO: 0010817 |

| | | | | | SUPPLEMENTARY TABLE 11 | | |
|---|---|---|---|---|---|---|---|
| | | | | | Co-Expresston Cluster | | |
| | Gene Ontology (GO) | | | | Num- | | PubMed |
| miRNA | ID | Term | Tissue | Dataset | ber | Full Cancer Name | ID |
| hsa-miR-9 | GO:0034641 | Cellular nitrogen compound metabolic process | Breast | IDC Breast Radvanyl | 16 | Invasive Ductal Carcinoma (Radvanyl et al., 2005) | 16043716 |
| | | | Lung | AD Lung Beer | 11 | Adenocarcinoma (Beer et al., 2002) | 12118244 |
| hsa-miR-23a/b | GO:0016071 | mRNA metabolic process | Lung | AD Lung Bhattacharjee | 69 | Adenocarcinoma (Bhattacharjee et al., 2001) | 11707567 |
| | | | Myeloma | MM Myeloma Zhan | 43 | Multiple Myeloma (Zhan et al., 2002) | 11861292 |
| hsa-miR-29a/b/c | GO:0006928 | Cellular component movement | Head & Neck | HSCC Head-Neck Chung | 1 | Head-Neck Squamous Cell Carcinoma (Chung et al., | 15144956 |
| | | | | | | 2004) | |
| | GO:0030198 | Extracellular matrix organization | Lung | AD Lung Beer | 31 | Adenocarcinoma (Beer et al., 2002) | 12118244 |
| | | | Lung | AD Lung Bhattacharjee | 59 | Adenocarcinoma (Bhattacharjee et al., 2001) | 11707567 |
| | | | Ovarian | CCC Ovarian HENDrix | 11 | Clear Cell Carcinoma (HENDrix et al., 2006) | 16452189 |
| | | | Ovarian | END Ovarian HENDrix | 6 | ENDometrioid Adenocarcinoma (HENDrix et al., 2006) | 16452189 |
| hsa-miR-130a | GO:0009611 | Response to wounding | Breast | CA Breast Richardson | 55 | Carcinoma (Richardson et al., 2006) | 16473279 |
| | | | Prostate | TU Prostate Lapointe | 25 | Primary Tumor (Lapointe et al., 2004) | 14711987 |
| hsa-miR-183 | GO:0001501 | Skeletal system development | Myeloma | MM Myeloma Zhan | 21 | Multiple Myeloma (Zhan et al., 7002) | 11861292 |
| | GO:0001503 | Ossification | Germ Cell | GCT Seminoma Korkola | 45 | Germ Cell Tumor (Korkola et al., 2006) | 16424014 |
| | GO:0060348 | Bone development | | | | | |
| hsa-miR-296-5p | GO:0006928 | Cellular component movement | Germ Cell | GCT Seminoma Korkola | 69 | Germ Cell Tumor (Korkola et al., 2006) | 16424014 |
| | GO:0007155 | Cell adhesion | Lung | AD Lung Bhattacharjee | 41 | Adenocarcinoma (Bhattacharjee et al., 2001) | 11707567 |
| | GO:0009653 | Anatomical structure morphogenesis | Lung | SMCL Lung Bhattacharjee | 36 | Small Cell Lung Cancer (Bhattacharjee et al., 2001) | 11707567 |
| | GO:0030036 | Actin cytoskeleton organization | Lung | SQ Lung Bhattacharjee | 13 | Squamous Cell Lung Carcinoma (Bhattacharjee et al., | 11707567 |
| | | | | | | 2001) | |
| | GO:0042127 | Regulation of cell proliferation | | | | | |
| | GO:0051384 | Response to glucocorticoid stimulus | | | | | |
| hsa-miR-338-5p | GO:0000075 | Cell cycle checkpoint | Germ Cell | GCT Seminoma Korkola | 88 | Germ Cell Tumor (Korkola et al., 2006) | 16424014 |
| | GO:0000278 | Mitotic cell cycle | Lung | AD Lung Bhattacharjee | 0 | Adenocarcinoma (Bhattacharjee et al., 2001) | 11707567 |
| | GO:0006974 | Response to DNA damage stimulus | Mesothelioma | MPM Mesothelioma Gordon | 44 | Malignant Mesothelioma (Gordon et al., 2005) | 15920167 |
| | GO:0008152 | Metabolic process | Ovarian | AD Ovarian Welsh | 14 | Adenocarcinoma (Welsh et al., 2001) | 11158614 |
| hsa-miR-369-5p | GO:0008152 | Metabolic process | Brain | GBM Brain Liang | 18 | Glioblastoma Multiforme (Liang et al., 2005) | 15827123 |
| | GO:0019538 | Protein metabolic process | Prostate | TU Prostate Lapointe | 40 | Primary Tumor (Lapointe et al., 2004) | 14711987 |
| | GO:0009987 | Cellular process | | | | | |
| | GO:0044237 | Cellular metabolic process | | | | | |
| hsa-miR-487b | GO:0031018 | ENDocrine pancreas development | Ovarian | END Ovarian HENDrix | 18 | ENDometrioid Adenocarcinoma (HENDrix et al., 2006) | 16452189 |
| | GO:0019083 | Viral transcription | Renal | RCCC Renal Lenburg | 2 | Clear Cell Renal Cell Carcinoma (Lenburg et al., 2003) | 14641932 |
| | GO:0019058 | Viral infectious cycle | | | | | |
| | GO:0006415 | Translational termination | | | | | |
| | GO:0006414 | Translational elongation | | | | | |
| hsa-miR-495 | GO:0006397 | mRNA procossing | Head & Neck | HSCC Head-Neck Cromer | 25 | Head-Neck Squamous Cell Carcinoma(Cromer et al., | 14676830 |
| | | | | | | 2004) | |
| | GO:0016071 | mRNA metabolic process | Lung | AD Lung Bhattacharjee | 60 | Adenocarcinoma (Bhattacharjee et al., 2001) | 11707567 |
| | | | Myeloma | MM Myeloma Zhan | 17 | Multiple Myeloma (Zhan et al., 2002) | 11861292 |
| hsa-miR-548c-3p | GO:0008152 | Metabolic process | Bladder | CA Bladder Dyrskjot | 9 | Bladder Carcinoma (Dyrskjot et al., 2004) | 15173019 |
| | GO:0010467 | Gene expression | Bladder | CA Bladder Dyrskjot | 13 | Bladder Carcinoma (Dyrskjot et al., 2004) | 15173019 |
| | GO:0016070 | RNA metabolic process | Bladder | CA Bladder Dyrskjot | 29 | Bladder Carcinoma (Dyrskjot et al., 2004) | 15173019 |
| | | | Brain | GLB Brain Sun | 50 | Glioblastoma (Sun et al., 2006) | 16616334 |
| | | | Brain | OD Brain Bredel | 21 | OligodENDroglioma (Bredel et al., 2005) | 16204036 |
| | | | Brain | ODGL Brain Sun | 50 | OligodENDroglioma (Sun et al., 2006) | 16616334 |
| | | | Breast | CA Breast Sorlie | 12 | Carcinoma (Sorlie et al., 2001) | 11553815 |
| | | | Germ Cell | GCT Seminoma Korkola | 95 | Germ Cell Tumor (Korkola el al., 2005) | 16424014 |
| | | | Leukemia | B-CLL Leukemia Haslinger | 62 | Chronic Lymphocytic Leukemia (Haslinger et al., 2004) | 15459216 |
| | | | Lung | AD Lung Beer | 6 | Adenocarcinoma (Beer et al., 2002) | 12118244 |
| | | | Lung | AD Lung Bhattacharjee | 30 | Adenocarcinoma (Bhattacharjee et al., 2001) | 11707567 |
| | | | Melanoma | ME Melanoma Hoek | 50 | Cutaneous melanoma (Hoek et al., 2006) | 16827748 |
| | | | Mesothelioma | MPM Mesothelioma Gordon | 63 | Malignant Mesothelioma (Gordon et al., 2005) | 15920167 |
| | | | Myeloma | MM Myeloma Zhan | 3 | Multiple Myeloma (Zhan et al., 2002) | 11861292 |
| hsa-miR-548n | GO:0010467 | Gene expression | Bladder | CA Bladder Dyrskjot | 37 | Bladder Carcinoma (Dyrskjot et al., 2004) | 15173019 |
| | | | Germ Cell | GCT Seminoma Korkola | 40 | Germ Cell Tumor (Korkola et al., 2006) | 16424014 |
| | | | Germ Cell | GCT Seminoma Korkola | 112 | Germ Cell Tumor (Korkola et al., 2006) | 16424014 |
| | | | Lung | SMCL Lung Bhattacharjee | 22 | Small Cell Lung Cancer (Bhattacharjee et at., 2001) | 11707567 |
| | | | Lung | SQ Lung Bhattacharjee | 22 | Squamous Cell Lung Carcinoma (Bhattacharjee et al., | 11707567 |
| | | | | | | 2001) | |
| | | | Melanoma | ML Melanoma Talantov | 3 | Melanoma (Talantov et al., 2005) | 16825504 |
| hsa-miR-590-3p | GO:0044237 | Cellular metabolic process | Leukemia | B-CLL Leukemia Haslinger | 17 | Chronic Lymphocytic Leukemia (Haslinger et al., 2004) | 15459216 |
| | | | Bladder | CA Bladder Dyrskjot | 64 | Bladder Carcinoma (Dyrskjot el al., 2004) | 15173019 |
| | | | Ovarian | CCC Ovarian HENDrix | 42 | Clear Cell Carcinoma (HENDrix el al., 2006) | 16452189 |
| | | | Germ Cell | GCT Seminoma Korkola | 112 | Germ Cell Tumor (Korkola et al., 2006) | 16424014 |
| | | | Brain | GLB Brain Sun | 27 | Glioblastoma (Sun et al., 2006) | 16616334 |
| | | | Head & Neck | HSCC Head-Neck Chung | 6 | Head-Neck Squamous Cell Carcinoma (Chung et al., | 15144956 |
| | | | | | | 2004) | |
| | | | Myeloma | MM Myeloma Zhan | 3 | Multiple Myeloma (Zhan et al., 2002) | 11861292 |
| | | | Mesothelioma | MPM Mesothelioma Gordon | 16 | Malignant Mesothelioma (Gordon et al., 2005) | 15920167 |
| | | | Mesothelioma | MPM Mesothelioma Gordon | 28 | Malignant Mesothelioma (Gordon et al., 2005) | 15920167 |
| | | | Prostate | TU Prostate Lapointe | 1 | Primary Tumor (Lapointe et al., 2004) | 14711987 |
| hsa-miR-607 | GO:0000398 | Nuclear mRNA splicing, via spliceosome | Bladder | CA Bladder Dyrskjot | 0 | Bladder Carcinoma (Dyrskjot et al., 2004) | 15173019 |
| | | | Lung | SQ Lung Bhattacharjee | 22 | Squamous Cell Lung Carcinoma (Bhattacharjee et al., | 11707567 |
| | | | | | | 2001) | |
| hsa-miR-656 | GO:0001525 | Angiogenesis | Bladder | CA Bladder Dyrskjot | 82 | Bladder Carcinoma (Dyrskjot et al., 2004) | 15173019 |
| | GO:0001568 | Blood vessel development | Lung | AD Lung Beer | 32 | Adenocarcinoma (Beer et al., 2002) | 12118244 |
| | GO:0001816 | Cytokine production | | | | | |
| | GO:0006928 | Cellular component movement | | | | | |
| | GO:0006935 | Chemotaxis | | | | | |
| | GO:0006952 | Defense response | | | | | |
| | GO:0006954 | Inflammatory response | | | | | |
| | GO:0006955 | Immune response | | | | | |
| | GO:0007166 | Cell surface receptor linked signaling pathway | | | | | |
| | GO:0008283 | Cell proliferation | | | | | |
| | GO:0009611 | Response to wounding | | | | | |
| | GO:0009653 | Anatomical structure morphogenesis | | | | | |
| | GO:0009887 | Organ morphogenesis | | | | | |
| | GO:0016477 | Cell migration | | | | | |
| | GO:0030334 | Regulation of cell migration | | | | | |
| | GO:0030335 | Positive regulation of cell migration | | | | | |
| | GO:0042060 | Wound healing | | | | | |
| | GO:0042127 | Regulation of cell proliferation | | | | | |
| | GO:0048514 | Blood vessel morphogenesis | | | | | |
| | GO:0048870 | Cell motility | | | | | |
| | GO:0050900 | Leukocyte migration | | | | | |
| | GO:0051272 | Positive regulation of cellular component movement | | | | | |
| hsa-miR-760 | GO:0006334 | Nucleosome assembly | Brain | GLB Brain Sun | 41 | Glioblastoma (Sun et al., 2006) | 16616334 |
| | | | Leukemia | B-CLL Leukemia Haslinger | 27 | Chronic Lymphocytic Leukemia (Haslinger et al., 2004) | 15459216 |
| | | | Lung | AD Lung Bhattacharjee | 42 | Adenocarcinoma (Bhattacharjee et al., 2001) | 11707567 |
| | | | Myeloma | MM Myeloma Zhan | 29 | Multiple Myeloma (Zhan et al., 2002) | 11861292 |
| hsa-miR-767-5p | GO:0006928 | Cellular component movement | Head & Neck | HSCC Head-Neck Chung | 1 | Head-Neck Squamous Cell Carcinoma (Chung et al., | 15144956 |
| | | | | | | 2004) | |
| | GO:0030198 | Extracellular matrix organization | Lung | SQ Lung Bhattacharjee | 18 | Squamous Cell Lung Carcinoma (Bhattacharjee et al., | 11707567 |
| | | | | | | 2001) | |
| | GO:0030199 | Collagen fibril organization | Lung | SQ lung Bhattacharjee | 44 | Squamous Cell Lung Carcinoma (Bhattacharjee et al., | 11707567 |
| | | | | | | 2001) | |
| | | | Ovarian | AD Ovarian Welsh | 20 | Adenocarcinoma (Walsh et al., 2001) | 11158614 |
| hsa-miR-890 | GO:0001568 | Blood vessel development | Lung | COID Lung Bhattacharjee | 36 | Carcinoid (Bhattacharjee et al., 2001) | 11707567 |
| | GO:0001822 | Kidney development | Ovarian | END Ovarian HENDrix | 6 | ENDometrioid Adenocarcinoma (HENDrix et al., 2006) | 16452189 |
| | GO:0007155 | Cell adhesion | | | | | |
| | GO:0007275 | Multicellular organismal development | | | | | |
| | GO:0017015 | Regulation of transforming growth factor beta | | | | | |
| | | receptor signaling pathway | | | | | |
| hsa-miR-939 | GO:0007267 | Cell-cell signaling | Brain | AC Brain Sun | 44 | Astrocytoma (Sun et al., 2006) | 16616334 |
| | GO:0007268 | Synaptic transmission | Brain | ODGL Brain Sun | 2 | OligodENDroglioma (Sun et al., 2006) | 16616334 |
| | GO:0050877 | Neurological system process | | | | | |
| hsa-miR-944 | GO:0003676 | Nucleic acid binding | Ovarian | END Ovarian HENDrix | 38 | ENDometrioid Adenocarcinoma (HENDrix et al., 2006) | 16452189 |
| | GO:0006139 | Nucleobase, nucleoside, nucleotide and nucleic acid | Ovarian | MUC Ovarian HENDrix | 15 | Mucinous Adenocarcinoma (HENDrix el al., 2006) | 16452189 |
| | | metabolic process | | | | | |
| | GO:0010467 | Gene expression | | | | | |
| hsa-miR-1207-5p | GO:0006370 | mRNA capping | Brain | GL Brain Rickman | 9 | Glioma (Rickman el al., 2001) | 11559565 |
| | | | Lung | SMCL Lung Bhattacharjee | 34 | Small Cell Lung Cancer (Bhattacharjee et al., 2001) | 11707567 |
| hsa-miR-1275 | GO:0007186 | G-protein coupled receptor protein signaling | Brain | GLB Brain Sun | 29 | Glioblastoma (Sun et al., 2006) | :6616334 |
| | | pathway | | | | | |
| | GO:0007267 | Cell-cell signaling | Lung | AD Lung Beer | 43 | Adenocarcinoma (Beer et al., 2002) | 12118244 |
| hsa-miR-1276 | GO:0006334 | Nucleosome assembly | Bladder | CA Bladder Dyrskjot | 51 | Bladder Carcinoma (Dyrskjot et al., 2004) | 15173019 |
| | | | Ovarian | END Ovarian HENDrix | 35 | ENDometrioid Adenocarcinoma (HENDrix et al., 2006) | 16452189 |
| | | | Ovarian | SRS Ovarian HENDrix | 72 | Serous Adenocarcinoma (HENDrix et al., 2006) | 16452189 |
| hsa-miR-1291 | GO:0007154 | Cell communication | Bladder | CA Bladder Dyrskjot | 18 | Bladder Carcinoma (Dyrskjot et al., 2004) | 15173019 |
| | | | Breast | IDC Breast Radvanyl | 24 | Invasive Ductal Carcinoma (Radvanyl et al., 2005) | 16043716 |

SUPPLEMENTARY TABLE 12

| | | | | | | | miRNA |
|---|---|---|---|---|---|---|---|
| | | | PCR | | | | Binding |
| | | | Fragment | | | | Site in |
| Gene | Forward Primer | Reverse Primer | Size | Chr | Start | Stop | Amplicon |
| COL3A1 | TCCATATGTGTTCCTCTTGTTCT | TTATGGGTGTCTGTAAGGAAAAA | 912 | 2 | 189584785 | 189585696 | Y |
| | SEQ ID NO: 318 | SEQ ID NO: 319 | | | | | |
| COL4A1 | CCTGACTCAGCTAATGTCACAA | GCAGCTTGTGCAGTAAGTTTCTT | 1366 | 13 | 109599362 | 109600565 | Y |
| | SEQ ID NO: 320 | SEQ ID NO: 321 | | | | | |
| COL4A2 | GGCCATTTTGGTGCTTATTC | CAGAACCAAGTTTTATTTTGTAGTCG | 805 | 13 | 109962559 | 109963363 | Y |
| | SEQ ID NO: 322 | SEQ ID NO: 323 | | | | | |
| COL5A2 | CAATGAGCACCACCATCAAT | TTGGAAGTCAAACAAAACTCACA | 2000 | 2 | 189604886 | 189607040 | Y |
| | SEQ ID NO: 324 | SEQ ID NO: 325 | | | | | |
| COL10A1 | TCTAAATCTTGTGCTAGAAAAAGCA | CTTTGAACAATGAAAAGCCTTG | 1107 | 6 | 116546778 | 116547928 | Y |
| | SEQ ID NO: 326 | SEQ ID NO: 327 | | | | | |
| FBN1 | TCACCATCCAGAGACCAAATA | CAAAGTGATTTTGGCTGAGTAAA | 2593 | 15 | 46487885 | 46490477 | Y |
| | SEQ ID NO: 328 | SEQ ID NO: 329 | | | | | |
| LOX | ATAAATCAGTGCCTGGTGTTCTG | ATGAGAATGCAAAGAGGAACA | 3450 | 5 | 121426789 | 121430336 | Y |
| | SEQ ID NO: 330 | SEQ ID NO: 331 | | | | | |
| MMP2 | CCTCTCCACTGCCTTCGATA | CCTCGAACAGATGCCACAAT | 1017 | 16 | 54096882 | 54097898 | Y |
| | SEQ ID NO: 332 | SEQ ID NO: 333 | | | | | |
| PDGFRB | TTTCTGCTCCTGACGTGTTG | TGAGTGAGAAGCACCAGGTTT | 1797 | 5 | 149473597 | 149475393 | Y |
| | SEQ ID NO: 334 | SEQ ID NO: 335 | | | | | |
| SERPINH1 | CTCAGGGTGCACACAGGAT | CACGCTCCAACAAAATGTCA | 712 | 11 | 74960780 | 74961491 | Y |
| | SEQ ID NO: 336 | SEQ ID NO: 337 | | | | | |
| SPARC | CTCTTTAACCCTCCCCTTCG | GAGGGGAAATGACATCTGGA | 2039 | 15 | 151021258 | 151023296 | Y |
| | SEQ ID NO: 338 | SEQ ID NO: 339 | | | | | |

Recombinant PCR Primers
MMP2_recomb_F GCCACACTTCAGGCTCTTCTC (SEQ ID NO: 340)
MMP2_bubble_del_R GAGAAGAGCCTGAAGTGTGGCcgacaacGGGCAGCCCAAAGCAGGGCTG (SEQ ID NO: 341)
SPARC_recomb_del_F CATAGATTTAAGTGAATACATTAACatgcggtAAAATGAAAATTCTAACCC (SEQ ID NO: 342)
SPARC_recomb_del_R TGTATTCACTTAAATCTATGTaccgcatTTGTCTCCAGGCAGAACAAC (SEQ ID NO: 343)

1. A method of calculating a risk score for developing cancer comprising (a) obtaining inputs about an individual comprising the level of biomarkers in at least one biological sample from said individual and (b) calculating a cancer risk score from said inputs, wherein said biomarkers comprise one or more miRNA biomarkers selected from FIGS. 12, 13 and 14.

2. A method of evaluating risk for developing cancer comprising (a) obtaining biomarker measurement data, wherein the biomarker measurement data is representative of measurements of biomarkers in a least one biological sample from an individual and (b) evaluating risk for developing cancer based on an output from a model, wherein the model is executed based on an input of the biomarker measurement data, wherein the biomarkers comprise one or more biomarkers selected form FIGS. 12, 13 and 14.

3. A method of evaluating risk for developing cancer comprising (a) obtaining biomarker measurements from at least one biological sample from an individual who is a subject that has not been previously diagnosed as having cancer, (b) comparing the biomarker measurement to normal control levels and (c) evaluating the risk for the individual developing a cancer from the comparison; wherein the biomarkers comprise one or more biomarkers selected from FIGS. 12, 13 and 14.

4. A method of evaluating risk for developing cancer comprising (a) obtaining biomarker measurement data, wherein the biomarker measurement data is representative of measurements of biomarkers in at least one biological sample from an individual and (b) evaluating risk for developing cancer based on an output from a model, wherein the model is executed based on an input of the biomarker measurement data; wherein said biomarkers the biomarkers comprise one or more biomarkers selected from FIGS. 12, 13 and 14.

5. A method of calculating a risk score for cancer progression comprising (a) obtaining inputs about an individual suffering from cancer comprising the level of biomarkers in a least one biological sample from said individual; and (b) calculating a cancer risk score form said inputs, wherein said biomarkers comprise one or more biomarkers selected from FIGS. 12,13 and 14.

6. The method of claim 1, 2, 3, 4, or 5 further comprising advising the individual of the individual's risk of developing cancer or risk of cancer progression.

7. A method of ranking or grouping a population of individuals according to cancer risk comprising (a) obtaining a cancer risk score for individuals comprised within said population, wherein said cancer risk score is calculated according to claim 1 and (b) ranking individuals within the population relative to the remaining individuals in the population or dividing the population into at least two groups, based on factors comprising said obtained cancer risk scores.

8. A diagnostic test system comprising (a) means for obtaining test results comprising levels of biomarkers in at least one biological sample; (b) means for collecting and tracking test results for one or more individual biological samples; (c) means for calculating an cancer risk score from inputs, wherein said inputs comprise measured levels of biomarkers, and further wherein said measured levels of biomarkers comprise the levels of one or more biomarkers selected from FIGS. 12, 13 and 14; and (d) means for reporting said cancer risk score.

9. A diagnostic test system comprising (a) means for obtaining test results data representing levels of multiple biomarkers in at least one biological sample, (b) means for collecting and tracking test results data for one or more individual biological samples (c) means for computing a cancer risk score from biomarker measurement data, wherein said biomarker measurement data is representative of measured levels of biomarkers, and further wherein said measured levels of biomarkers comprise the levels of a panel of one or more biomarkers selected from FIGS. 12, 13 and 14, and (d) means for reporting said index value.

10. A medical diagnostic test system for evaluating risk for developing a cancer or risk for cancer progression comprising (a) a data collection tool adapted to collect biomarker measurement data representative of measurements of one or more biomarkers in at least one biological sample from an individual, (b) an analysis tool comprising a statistical analysis engine adapted to generate a representation of a correlation between a risk for developing a cancer and measurements of the biomarkers, wherein the representation of the correlation is adapted to be executed to generate a result and (c) an index computation tool adapted to analyze the result to determine the individual's risk for developing a cancer or for cancer progression, and represent the result as a cancer risk score; wherein said one or more biomarkers are selected from FIGS. 12, 13 and 14.

11. A computer readable medium having computer executable instructions for evaluating risk for developing a cancer, the computer readable medium comprising (a) a routine, stored on the computer readable medium and adapted to be executed by a processor, to store biomarker measurement data representing a panel of one or more biomarkers and (b) a routine stored on the computer readable medium and adapted to be executed by a processor to analyze the biomarker measurement data to evaluate a risk for developing a cancer or for risk of cancer progression; wherein said biomarkers are one or more biomarkers selected from FIGS. 12, 13 and 14.

12. A kit comprising reagents for measuring a panel of one or more miRNA biomarkers selected from FIGS. 12, 13 and 14, wherein the reagents are primers for reverse transcription of miRNA into DNA, primers for amplification of the DNA, or both primers for reverse transcription of miRNA in the panel and primers for amplification of the reverse transcribed DNA.

13. A kit comprising reagents for detecting a panel of one or more miRNA biomarkers selected from FIGS. 12, 13 and 14, wherein the reagents hybridize to miRNA in the panel.

14. A system for diagnosing susceptibility to cancer in a human subject, the system comprising:
(a)at least one processor;
(b)at least one computer-readable medium;
(c) a susceptibility database operatively coupled to a computer-readable medium of the system and containing information associating measurements of one or more biomarkers selected from FIGS. 12, 13 and 14 and cancer in a population of humans;
(d) a measurement tool that receives an input about the human subject and generates information from the input about one or more biomarkers selected from FIGS. 12, 13 and 14 from the human subject; and
(e) an analysis tool (routine) that:
(i) is operatively coupled to the susceptibility database and the measurement tool,
(ii) is stored on a computer-readable medium of the system,
(iii) is adapted to be executed on a processor of the system, to compare the information about the human subject with the information about the population in the susceptibility database and generate a conclusion with respect to susceptibility to cancer in the human subject.

15. A system for diagnosing cancer in a human subject, the system comprising:
(a)at least one processor;
(b)at least one computer-readable medium;
(c) a susceptibility database operatively coupled to a computer-readable medium of the system and containing information associating measurements of biomarkers selected from FIGS. 12, 13 and 14 and cancer in a population of humans;
(d)a measurement tool that receives an input about the human subject and generates information from the input about one or more biomarkers selected from FIGS. 12, 13 and 14 from the human subject; and
(e)an analysis tool (routine) that:
(i) is operatively coupled to the susceptibility database and the measurement tool,
(ii) is stored on a computer-readable medium of the system,
(iii) is adapted to be executed on a processor of the system, to compare the information about the human subject with the information about the population in the susceptibility database and generate a conclusion with respect to the presence of cancer in the human subject.

16. The system according to claim 14 or 15, wherein the input about the human subject is a biological sample from the human subject, and wherein the measurement tool comprises a tool to measure one or more biomarkers selected from FIGS. 12, 13 and 14 in the biological sample, thereby generating biomarker measurements from a human subject.

17. The system of claim 16 wherein the biomarkers are measured by polymerase chain reaction or hybridization to a microarray.

18. The system of claim 14 or 15 further comprising a communication tool operatively coupled to the analysis tool, stored on a computer-readable medium of the system and adapted to be executed on a processor of the system to generate a communication for the human subject, or a medical practitioner for the subject, containing the conclusion with respect to cancer for the subject.

19. The system according to claim 18, wherein the communication tool is operatively connected to the analysis tool or routine and comprises a routine stored on a computer-readable medium of the system and adapted to be executed on a processor of the system, to:
generate a communication containing the conclusion; and
transmit the communication to the subject or the medical practitioner, or
enable the subject or medical practitioner to access the communication.

20. The system of claim 14, 15, 18 or 19 further comprising:
a medical protocol database operatively connected to a computer-readable medium of the system and containing information correlating the conclusion and medical protocols for human subjects at risk for or suffering from cancer; and
a medical protocol tool (or routine), operatively connected to the medical protocol database and the analysis tool or routine, stored on a computer-readable medium of the system, and adapted to be executed on a processor of the system, to compare the conclusion from the analysis routine with respect to cancer for the subject and the medical protocol database, and generate a protocol report with respect to the probability that one or more medical protocols in the database will:
reduce susceptibility to cancer; or
delay onset of cancer;
increase the likelihood of detecting cancer at an early stage to facilitate early treatment; or
treat the cancer.

21. The system according to claim 20, wherein the communication tool is operatively connected to the medical protocol tool or routine, and generates a communication that further includes the protocol report.

22. A method for the prophylactic treatment of a individual at risk for a cancer comprising (a) obtaining a cancer risk score for an individual based on one or more biomarkers selected from FIGS. 12, 13 and 14 and (b) generating prescription treatment data representing a prescription for a treatment regimen to delay or prevent the onset of cancer in the individual identified by the cancer risk score as being at elevated risk for cancer.

23. A method for the therapeutic treatment of a individual suffering from a cancer comprising (a) obtaining a cancer diagnosis based on one or more miRNA biomarkers selected from FIGS. 12, 13 and 14 and (b) generating prescription treatment data representing a prescription for a treatment regimen to treat the cancer in the individual identified by the cancer risk score as being at elevated risk for cancer.

24. The method of claim 22 or 23 wherein the treatment regimen comprises the standard of care for the cancer.

25. The method of claim 22, 23 or 24 wherein the treatment regimen comprises administering a drug that increases the amount of the one or more miRNAs selected from FIGS. 12, 13 and 14.

26. The method of claim 22, 23 or 24 wherein the treatment regimen comprises administering a drug to inhibit the one or more miRNAs or decrease the amount of the one or more miRNAs selected from FIGS. 12, 13 and 14.

27. The method of claim 22, 23, 24, 25 or 26 further comprising (c) treating the individual according to the treatment regimen.
